(19) United States
(12) Patent Application Publication
(10) Pub. No.: US 2022/0213211 A1
(43) Pub. Date: Jul. 7, 2022

(54) ANTIGEN RECOGNIZING RECEPTORS TARGETING CD371 AND USES THEREOF

(71) Applicants: MEMORIAL SLOAN-KETTERING CANCER CENTER, New York, NY (US); TRI-INSTITUTIONAL THERAPEUTICS DISCOVERY INSTITUTE, INC., New York, NY (US); SLOAN-KETTERING INSTITUTE FOR CANCER RESEARCH, New York, NY (US); MEMORIAL HOSPITAL FOR CANCER AND ALLIED DISEASES, New York, NY (US)

(72) Inventors: Anthony Daniyan, Somers, NY (US); Renier J. Brentjens, Short Hills, NJ (US); Ivo C. Lorenz, Brooklyn, NY (US); Mary Ann Pohl, Astoria, NY (US)

(73) Assignees: MEMORIAL SLOAN-KETTERING CANCER CENTER, New York, NY (US); TRI-INSTITUTIONAL THERAPEUTICS DISCOVERY INSTITUTE, INC., New York, NY (US); SLOAN-KETTERING INSTITUTE FOR CANCER RESEARCH, New York, NY (US); MEMORIAL HOSPITAL FOR CANCER AND ALLIED DISEASES, New York, NY (US)

(21) Appl. No.: 17/693,060

(22) Filed: Mar. 11, 2022

Related U.S. Application Data
(63) Continuation of application No. PCT/US2020/050386, filed on Sep. 11, 2020.
(60) Provisional application No. 62/900,141, filed on Sep. 13, 2019.
(60) Provisional application No. 62/936,951, filed on Nov. 18, 2019.

(51) Int. Cl. C07K 16/28; C07K 14/705; A61K 35/17; C07K 14/725; A61P 35/00

(52) U.S. Cl. CPC C07K 16/2896; C07K 14/70596; A61K 35/17; C07K 14/70521; C07K 14/7051; A61K 2039/5156; C07K 2319/03; C07K 2319/02; C07K 2319/30; C07K 2317/565; C07K 2317/622; A61P 35/00

(57) ABSTRACT

The presently disclosed subject matter provides for antigen-recognizing receptors that specifically target CD371 and cells comprising such CD371-targeted antigen-recognizing receptors. The presently disclosed subject matter further provides uses of the CD371-targeted antigen-recognizing receptors for treatment.

### CROSS-REFERENCE TO RELATED APPLICATIONS

This application is a continuation of International Application No. PCT/US2020/050386, filed Sep. 11, 2020, which claims priority to U.S. Provisional Application No. 62/900,141 filed Sep. 13, 2019 and U.S. Provisional Application No. 62/936,951 filed Nov. 18, 2019, the contents of each of which are incorporated by reference in their entireties herein, and priority to each of which is claimed.

### SEQUENCE LISTING

The present application contains a Sequence Listing which has been submitted in ASCII format via EFS-Web and is hereby incorporated by reference in its entirety. Said ASCII copy, created on Mar. 10, 2022, is named 0893390243 ST25.TXT and is 212,069 bytes in size.

### INTRODUCTION

The presently disclosed subject matter provides methods and compositions for immunotherapies. It relates to antigen-recognizing receptors (e.g., chimeric antigen receptors (CARs) or T-cell receptors (TCRs)) that specifically target CD371, cells comprising such receptors, and methods of using such cells for treatments.

### BACKGROUND OF THE INVENTION

Cell-based immunotherapy is a therapy with curative potential for the treatment of cancer. T cells and other immune cells may be modified to target tumor antigens through the introduction of genetic material coding for artificial or synthetic receptors for antigen, termed Chimeric Antigen Receptors (CARs), specific to selected antigens. Targeted T cell therapy using CARs has shown recent clinical success in treating hematologic malignancies.

Acute myeloid leukemia (AML) is the most common type of adult acute leukemia. It is characterized by the accumulation of immature myeloid cells in the bone marrow that results in dysfunction of hematopoiesis. Chemotherapy and hematopoietic stem cell transplantation (HSCT) are standard treatment of AML. However, a majority of patients eventually relapse and succumb to the disease.

AML is the most common acute leukemia in adults. The standard induction chemotherapy regimens have not changed substantially over the past 40 years and the overall survival remains very poor. Frequent recurring abnormalities involving genes coding for epigenetic modifiers have been identified. The development of CAR therapy for AML is hampered by the lack of suitable targets. Accordingly, there are needs for novel therapeutic strategies to design CARs targeting antigens that are highly expressed in AML cells and limited expression in normal tissues for treating AML, and for strategies capable of inducing potent cancer eradication with minimal toxicity and immunogenicity.

### SUMMARY OF THE INVENTION

The presently disclosed subject matter provides antigen-recognizing receptors that specifically target CD371 and cells comprising such CD371-targeted antigen-recognizing receptors. The presently disclosed subject matter further provides uses of the CD371-targeted antigen-recognizing receptors for treatment.

The presently disclosed subject matter provides an antigen-recognizing receptor, comprising an extracellular antigen-binding domain, a transmembrane domain, and an intracellular signaling domain, wherein the extracellular antigen-binding domain specifically binds to CD371. In certain embodiments, the extracellular antigen-binding domain is a single-chain variable fragment (scFv). In certain embodiments, the extracellular antigen-binding domain is a human scFv. In certain embodiments, the extracellular antigen-binding domain is a Fab, which is optionally crosslinked. In certain embodiments, the extracellular antigen-binding domain is a F(ab)₂. In certain embodiments, one or more of the scFv, Fab and F(ab)₂ are comprised in a fusion protein with a heterologous sequence to form the extracellular antigen-binding domain.

In certain embodiments, the extracellular antigen-binding domain comprises:

(a) a heavy chain variable region CDR1 comprising the amino acid sequence set forth in SEQ ID NO: 28 or a conservative modification thereof; a heavy chain variable region CDR2 comprising the amino acid sequence set forth in SEQ ID NO: 29 or a conservative modification thereof; and a heavy chain variable region CDR3 comprising the amino acid sequence set forth in SEQ ID NO: 30 or a conservative modification thereof;

(b) a heavy chain variable region CDR1 comprising the amino acid sequence set forth in SEQ ID NO: 34 or a conservative modification thereof; a heavy chain variable region CDR2 comprising the amino acid sequence set forth in SEQ ID NO: 35 or a conservative modification thereof; and a heavy chain variable region CDR3 comprising the amino acid sequence set forth in SEQ ID NO: 36 or a conservative modification thereof;

(c) a heavy chain variable region CDR1 comprising the amino acid sequence set forth in SEQ ID NO: 40 or a conservative modification thereof; a heavy chain variable region CDR2 comprising the amino acid sequence set forth in SEQ ID NO: 42 or a conservative modification thereof, and a heavy chain variable region CDR3 comprising the amino acid sequence set forth in SEQ ID NO: 43 or a conservative modification thereof;

(d) a heavy chain variable region CDR1 comprising the amino acid sequence set forth in SEQ ID NO: 462 or a conservative modification thereof; a heavy chain variable region CDR2 comprising the amino acid sequence set forth in SEQ ID NO: 47 or a conservative modification thereof, and a heavy chain variable region CDR3 comprising the amino acid sequence set forth in SEQ ID NO: 48 or a conservative modification thereof;

(e) a heavy chain variable region CDR1 comprising the amino acid sequence set forth in SEQ ID NO: 52 or a conservative modification thereof; a heavy chain variable region CDR2 comprising the amino acid sequence set forth in SEQ ID NO: 53 or a conservative modification thereof, and a heavy chain variable region CDR3 comprising the amino acid sequence set forth in SEQ ID NO: 54 or a conservative modification thereof; or

(f) a heavy chain variable region CDR1 comprising the amino acid sequence set forth in SEQ ID NO: 58 or a conservative modification thereof; a heavy chain variable region CDR2 comprising the amino acid sequence set forth in SEQ ID NO: 59 or a conservative modification thereof, and a heavy chain variable region CDR3 comprising the amino acid sequence set forth in SEQ ID NO: 60 or a conservative modification thereof.

In certain embodiments, the extracellular antigen-binding domain comprises: a heavy chain variable region CDR1 comprising the amino acid sequence set forth in SEQ ID NO: 28; a heavy chain variable region CDR2 comprising the amino acid sequence set forth in SEQ ID NO: 29; and a heavy chain variable region CDR3 comprising the amino acid sequence set forth in SEQ ID NO: 30.

In certain embodiments, the extracellular antigen-binding domain comprises:

(a) a light chain variable region CDR1 comprising the amino acid sequence set forth in SEQ ID NO: 31 or a conservative modification thereof; a light chain variable region CDR2 comprising the amino acid sequence set forth in SEQ ID NO: 32 or a conservative modification thereof; and a light chain variable region CDR3 comprising the amino acid sequence set forth in SEQ ID NO: 33 or a conservative modification thereof;

(b) a light chain variable region CDR1 comprising the amino acid sequence set forth in SEQ ID NO: 37 or a conservative modification thereof; a light chain variable region CDR2 comprising the amino acid sequence set forth in SEQ ID NO: 38 or a conservative modification thereof; and a light chain variable region CDR3 comprising SEQ ID NO: 39 or a conservative modification thereof;

(c) a light chain variable region CDR1 comprising the amino acid sequence set forth in SEQ ID NO: 43 or a conservative modification thereof; a light chain variable region CDR2 comprising the amino acid sequence set forth in SEQ ID NO: 44 or a conservative modification thereof; and a light chain variable region CDR3 comprising the amino acid sequence set forth in SEQ ID NO: 45 or a conservative modification thereof;

(d) a light chain variable region CDR1 comprising the amino acid sequence set forth in SEQ ID NO: 49 or a conservative modification thereof; a light chain variable region CDR2 comprising SEQ ID NO: 50 or a conservative modification thereof; and a light chain variable region CDR3 comprising the amino acid sequence set forth in SEQ ID NO: 51 or a conservative modification thereof;

(e) a light chain variable region CDR1 comprising the amino acid sequence set forth in SEQ ID NO: 55 or a conservative modification thereof; a light chain variable region CDR2 comprising the amino acid sequence set forth in SEQ ID NO: 56 or a conservative modification thereof; and a light chain variable region CDR3 comprising the amino acid sequence set forth in SEQ ID NO: 57 or a conservative modification thereof; or

(f) a light chain variable region CDR1 comprising the amino acid sequence set forth in SEQ ID NO: 61 or a conservative modification thereof; a light chain variable region CDR2 comprising the amino acid sequence set forth in SEQ ID NO: 62 or a conservative modification thereof; and a light chain variable region CDR3 comprising the amino acid sequence set forth in SEQ ID NO: 63 or a conservative modification thereof.

In certain embodiments, the extracellular antigen-binding domain comprises: a heavy chain variable region CDR1 comprising the amino acid sequence set forth in SEQ ID NO: 28; a heavy chain variable region CDR2 comprising the amino acid sequence set forth in SEQ ID NO: 29; and a heavy chain variable region CDR3 comprising the amino acid sequence set forth in SEQ ID NO: 30.

In certain embodiments, the extracellular antigen-binding domain comprises:

(a) a heavy chain variable region CDR1 comprising the amino acid sequence set forth in SEQ ID NO: 28; a heavy chain variable region CDR2 comprising the amino acid sequence set forth in SEQ ID NO: 29; a heavy chain variable region CDR3 comprising the amino acid sequence set forth in SEQ ID NO: 30; a light chain variable region CDR1 comprising the amino acid sequence set forth in SEQ ID NO: 31; a light chain variable region CDR2 comprising the amino acid sequence set forth in SEQ ID NO: 32; and a light chain variable region CDR3 comprising the amino acid sequence set forth in SEQ ID NO: 33;

(b) a heavy chain variable region CDR1 comprising the amino acid sequence set forth in SEQ ID NO: 34; a heavy chain variable region CDR2 comprising the amino acid sequence set forth in SEQ ID NO:35; a heavy chain variable region CDR3 comprising the amino acid sequence set forth in SEQ ID NO: 36; a light chain variable region CDR1 comprising the amino acid sequence set forth in SEQ ID NO: 37; a light chain variable region CDR2 comprising the amino acid sequence set forth in SEQ ID NO: 38; and a light chain variable region CDR3 comprising the amino acid sequence set forth in SEQ ID NO: 39;

(c) a heavy chain variable region CDR1 comprising the amino acid sequence set forth in SEQ ID NO: 40; a heavy chain variable region CDR2 comprising the amino acid sequence set forth in SEQ ID NO: 41; a heavy chain variable region CDR3 comprising the amino acid sequence set forth in SEQ ID NO: 42; a light chain variable region CDR1 comprising the amino acid sequence set forth in SEQ ID NO: 43; a light chain variable region CDR2 comprising the amino acid sequence set forth in SEQ ID NO: 44; and a light chain variable region CDR3 comprising the amino acid sequence set forth in SEQ ID NO: 45;

(d) a heavy chain variable region CDR1 comprising the amino acid sequence set forth in SEQ ID NO: 46; a heavy chain variable region CDR2 comprising the amino acid sequence set forth in SEQ ID NO: 47; a heavy chain variable region CDR3 comprising the amino acid sequence set forth in SEQ ID NO: 48; a light chain variable region CDR1 comprising the amino acid sequence set forth in SEQ ID NO: 49; a light chain variable region CDR2 comprising the amino acid sequence set forth in SEQ ID NO: 50; and a light chain variable region CDR3 comprising the amino acid sequence set forth in SEQ ID NO: 51;

(e) a heavy chain variable region CDR1 comprising the amino acid sequence set forth in SEQ ID NO: 52; a heavy chain variable region CDR2 comprising the amino acid sequence set forth in SEQ ID NO: 53; a heavy chain variable region CDR3 comprising the amino acid sequence set forth in SEQ ID NO: 54; a light chain variable region CDR1 comprising the amino acid sequence set forth in SEQ ID NO: 55; a light chain variable region CDR2 comprising the amino acid sequence set forth in SEQ ID NO: 56; and a light chain variable region CDR3 comprising the amino acid sequence set forth in SEQ ID NO: 57; or

(f) a heavy chain variable region CDR1 comprising the amino acid sequence set forth in SEQ ID NO: 58; a heavy chain variable region CDR2 comprising the amino acid sequence set forth in SEQ ID NO: 59; a heavy chain variable region CDR3 comprising the amino acid sequence set forth in SEQ ID NO: 60; a light chain variable region CDR1 comprising the amino acid sequence set forth in SEQ ID NO: 61; a light chain variable region CDR2 comprising the amino acid sequence set forth in SEQ ID NO: 62; and a light chain variable region CDR3 comprising the amino acid sequence set forth in SEQ ID NO: 63.

In certain embodiments, the extracellular antigen-binding domain comprises: a heavy chain variable region CDR1 comprising the amino acid sequence set forth in SEQ ID NO: 28; a heavy chain variable region CDR2 comprising the amino acid sequence set forth in SEQ ID NO: 29; a heavy chain variable region CDR3 comprising the amino acid sequence set forth in SEQ ID NO: 30; a light chain variable region CDR1 comprising the amino acid sequence set forth in SEQ ID NO: 31; a light chain variable region CDR2 comprising the amino acid sequence set forth in SEQ ID NO: 32; and a light chain variable region CDR3 comprising the amino acid sequence set forth in SEQ ID NO: 33.

In certain embodiments, the extracellular antigen-binding domain comprises a heavy chain variable region comprising an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 1, SEQ ID NO: 3, SEQ ID NO: 5, SEQ ID NO: 7, SEQ ID NO: 9, or SEQ ID NO: 11. In certain embodiments, the extracellular antigen-binding domain comprises a heavy chain variable region comprising the amino acid sequence set forth in SEQ ID NO: 1, SEQ ID NO: 3, SEQ ID NO: 5, SEQ ID NO: 7, SEQ ID NO: 9, or SEQ ID NO: 11. In certain embodiments, the extracellular antigen-binding domain comprises a heavy chain variable region comprising the amino acid sequence set forth in SEQ ID NO: 1.

In certain embodiments, the extracellular antigen-binding domain comprises a light chain variable region comprising an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 2, SEQ ID NO: 4, SEQ ID NO: 6, SEQ ID NO: 8, SEQ ID NO: 10, or SEQ ID NO: 12. In certain embodiments, the extracellular antigen-binding domain comprises a light chain variable region comprising the amino acid sequence set forth in SEQ ID NO: 2, SEQ ID NO: 4, SEQ ID NO: 6, SEQ ID NO: 8, SEQ ID NO: 10, or SEQ ID NO: 12. In certain embodiments, the extracellular antigen-binding domain comprises a light chain variable region comprising the amino acid sequence set forth in SEQ ID NO: 2.

In certain embodiments, the extracellular antigen-binding domain comprises: (a) a heavy chain variable region comprising an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 1, SEQ ID NO: 3, SEQ ID NO: 5, SEQ ID NO: 7, SEQ ID NO: 9, or SEQ ID NO: 11; and (b) a light chain variable region comprising an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in in SEQ ID NO: 2, SEQ ID NO: 4, SEQ ID NO: 6, SEQ ID NO: 8, SEQ ID NO: 10, or SEQ ID NO: 12. In certain embodiments, the extracellular antigen-binding domain comprises: (a) a heavy chain variable region comprising the amino acid sequence set forth in SEQ ID NO: 1, SEQ ID NO: 3, SEQ ID NO: 5, SEQ ID NO: 7, SEQ ID NO: 9, or SEQ ID NO: 11; and (b) a light chain variable region comprising the amino acid sequence set forth in SEQ ID NO: 2, SEQ ID NO: 4, SEQ ID NO: 6, SEQ ID NO: 8, SEQ ID NO: 10, or SEQ ID NO: 12.

In certain embodiments, the extracellular antigen-binding domain comprises:

(a) a heavy chain variable region comprising the amino acid sequence set forth in SEQ ID NO: 1, and a light chain variable region comprising the amino acid sequence set forth in SEQ ID NO: 2;

(b) a heavy chain variable region comprising the amino acid sequence set forth in SEQ ID NO: 3, and a light chain variable region comprising the amino acid sequence set forth in SEQ ID NO: 4;

(c) a heavy chain variable region comprising the amino acid sequence set forth in SEQ ID NO: 5, and a light chain variable region comprising the amino acid sequence set forth in SEQ ID NO: 6;

(d) a heavy chain variable region comprising the amino acid sequence set forth in SEQ ID NO: 7, and a light chain variable region comprising the amino acid sequence set forth in SEQ ID NO: 8;

(e) a heavy chain variable region comprising the amino acid sequence set forth in SEQ ID NO: 9, and a light chain variable region comprising the amino acid sequence set forth in SEQ ID NO: 10; or

(f) a heavy chain variable region comprising the amino acid sequence set forth in SEQ ID NO: 11, and a light chain variable region comprising the amino acid sequence set forth in SEQ ID NO: 12.

In certain embodiments, the extracellular antigen-binding domain comprises a heavy chain variable region comprising the amino acid sequence set forth in SEQ ID NO: 1; and a light chain variable region comprising the amino acid sequence set forth in SEQ ID NO: 2.

In certain embodiments, the extracellular antigen-binding domain comprises a linker between a heavy chain variable region and a light chain variable region of the extracellular antigen-binding domain. In certain embodiments, the linker has the amino acid sequence set forth in SEQ ID NO: 13, SEQ ID NO: 14, SEQ ID NO: 91, SEQ ID NO: 92, SEQ ID NO: 93, or SEQ ID NO: 94. In certain embodiments, the linker has the amino acid sequence set forth in SEQ ID NO: 13 or SEQ ID NO: 14.

In certain embodiments, the extracellular antigen-binding domain comprises a signal peptide that is covalently joined to the 5′ terminus of the extracellular antigen-binding domain. In certain embodiments, the extracellular antigen-binding domain comprises a heavy chain variable region and a light chain variable region, which are positioned from the N- to the C-terminus: V_{H}-V_{L}.

In certain embodiments, the extracellular antigen-binding domain binds to CD371 with a low binding affinity. In certain embodiments, the extracellular antigen-binding domain binds to CD371 with a dissociation constant (K_{d}) of 1×10⁻⁸ M or more.

In certain embodiments, the transmembrane domain comprises a CD28 polypeptide. In certain embodiments, the intracellular signaling domain comprises a CD3ζ polypeptide. In certain embodiments, the intracellular signaling domain further comprises at least one co-stimulatory signaling region. In certain embodiments, the at least one co-stimulatory signaling region comprises a CD28 polypeptide, a 4-1BB polypeptide, an OX40 polypeptide, an ICOS polypeptide, a DAP-10 polypeptide, or a combination thereof. In certain embodiments, the at least one co-stimulatory signaling region comprises a CD28 polypeptide or a 4-1BB polypeptide.

In certain embodiments, the antigen-recognizing receptor is a chimeric antigen receptor (CAR), a T-cell Receptor (TCR), or a T-cell like fusion protein. In certain embodiments, the antigen-recognizing receptor is a CAR.

In certain embodiments, the antigen-recognizing receptor is recombinantly expressed. In certain embodiments, the antigen-recognizing receptor is expressed from a vector. In certain embodiments, the vector is a y-retroviral rector.

The presently disclosed subject matter provides cells comprises a presently disclosed antigen-recognizing receptor. In certain embodiments, the cell is transduced with the antigen-recognizing receptor. In certain embodiment, the antigen-recognizing receptor is constitutively expressed on the surface of the cell.

In certain embodiments, the cell is engineered to express a cytokine or a fragment thereof. In certain embodiments, the cell further comprises an exogenous polypeptide of the cytokine or fragment thereof. In certain embodiments, the cell further comprises a nucleic acid molecule encoding the cytokine or fragment thereof. In certain embodiments, the cytokine is selected from the group consisting of IL-18, IL-33, IL-36, and combinations thereof. In certain embodiments, the cytokine is IL-18.

In certain embodiments, the cell is an immunoresponsive cell. In certain embodiments, the cell is a cell of the lymphoid lineage or a cell of the myeloid lineage. In certain embodiments, the cell is selected from the group consisting of a T cell, a Natural Killer (NK) cell, and a stem cell from which lymphoid cells may be differentiated. In certain embodiments, the cell is a T cell. In certain embodiments, the T cell is a cytotoxic T lymphocyte (CTL) or a regulatory T cell. In certain embodiments, the stem cell is a pluripotent stem cell. In certain embodiments, the pluripotent stem cell is an embryoid stem cell or an induced pluripotent stem cell.

The presently disclosed subject matter further provides nucleic acid molecules that encode a presently disclosed antigen-recognizing receptor. In certain embodiments, the nucleic acid molecule comprises the nucleotide sequence set forth in SEQ ID NO: 22, SEQ ID NO: 23, SEQ ID NO: 24, SEQ ID NO: 25, SEQ ID NO: 26, or SEQ ID NO: 27. In certain embodiments, the nucleic acid molecule comprises the nucleotide sequence set forth in SEQ ID NO: 22. The presently disclosed subject matter further provides vectors comprising the presently disclosed nucleic acid molecules. In certain embodiments, the vector is a viral vector. In certain embodiments, the vector is a y-retroviral rector.

In addition, the presently disclosed subject matter provides host cells expressing the nucleic acid molecule disclosed herein. In certain embodiments, the host cell is a T cell.

The presently disclosed subject matter further provides compositions comprising the cells disclosed herein. In certain embodiments, the composition is a pharmaceutical composition further comprising a pharmaceutically acceptable carrier.

The presently disclosed subject matter further provides methods of reducing tumor burden in a subject. In certain embodiments, the method comprises administering an effective amount of presently disclosed cells or composition to the subject. In certain embodiments, the method reduces the number of tumor cells, reduces tumor size, and/or eradicates the tumor in the subject. The presently disclosed subject matter further provides methods of increasing or lengthening survival of a subject having a tumor or neoplasm. In certain embodiments, the method comprises administering an effective amount of presently disclosed cells or composition to the subject. The presently disclosed subject matter further provides methods of treating and/or preventing a tumor or neoplasm in a subject. In certain embodiments, the method comprises administering to the subject an effective amount of the presently disclosed cells or composition. In certain embodiments, the tumor or neoplasm is selected from the group consisting of acute myeloid leukemia (AML), multiple myeloma, Non-Hodgkin Lymphoma, Hodgkin Lymphoma, Chronic Lymphocytic Leukemia (CLL), glioblastoma, myelodysplastic syndrome (MDS), and chronic myelogenous leukemia (CML). In certain embodiments, the tumor or neoplasm is acute myeloid leukemia (AML).

The presently disclosed subject matter further provides methods for producing a presently disclosed cell comprising a CD371-targeted antigen-recognizing receptor. In certain embodiments, the method comprises introducing into the cell a nucleic acid molecule that encodes the antigen-recognizing receptor.

In addition, the presently disclosed subject matter provides kits for reducing tumor burden in a subject, treating and/or preventing a tumor or neoplasm in a subject, and/or increasing or lengthening survival of a subject having a tumor or neoplasm. In certain embodiments, the kit comprises the cell described herein. In certain embodiments, the kit further comprises written instructions for using the cell for reducing tumor burden in a subject, treating and/or preventing a tumor or neoplasm in a subject, and/or increasing or lengthening survival of a subject having a tumor or neoplasm.

### BRIEF DESCRIPTION OF THE FIGURES

The following Detailed Description, given by way of example, but not intended to limit the invention to specific embodiments described, may be understood in conjunction with the accompanying drawings.

FIG. 1 depicts structures of antigen-recognizing receptors in accordance with the presently disclosed subject matter.

FIG. 2 depicts detection of CD371-targeted CAR on the surface of transduced human T cells. Antibodies against EGFRt (CETUXIMAB-APC) and Myc-tag (9B11-PE) detected surface expression of EGFRt and human anti-human CAR in B10-based CAR constructs. Controls included non-transduced human T cells which did not express EGFRt or MYC-tag, and non-MYC tag containing CAR constructs Etah19h28z (an anti-human CD19 CAR T cells) and EtC1HVh28z (anti-CD371 CAR T cells derived from a mouse anti-human CD371 antibody 107537).

FIG. 3 depicts tumor cell lysis activities of CD371-targeted CAR T cells. 4 day rested (4dR) human CD371-targeted CAR T cells were cocultured with CD33⁺/CD371⁺ U937 cells expressing GFP and firefly luciferase (U937gL) at different effector:tumor (E:T) ratios. Bioluminescence was measured 24 hours later and plotted as a percentage of signal detected in a coculture of a non-functional CAR T cells (based on B10L4H, but without a CD28 or CD3ζ zeta signaling domain) and U937gL. Et.C1HVh28z represents a CD371-targeted CAR derived from a mouse anti-human CD371 antibody 1075.7, and EtM195MTh28Z represents a CD33-targeted CAR T cell derived from a murine anti-CD33 antibody, M195 (see Zhao et al., Haematologica (2010):95:71-78 (2010)).

FIG. 4 depicts tumor cell lysis activities of CD371-targeted CAR T cells in 24 hour killing assays. Healthy donor-derived (referred to as “donor C”) CD371-targeted CAR T cells were cocultured with CD33⁺/CD371⁺ U937 cells expressing GFP and firefly luciferase (U937gL) at different effector:tumor (E:T) ratios. Bioluminescence was measured 24 hours later and plotted as a percentage of signal detected in a coculture of a non-functional CAR T cells (based on B10L4H, but without a CD28 or CD3ζ zeta signaling domain) and U937gL. Et.C1HVh28z represents a CD371-targeted CAR derived from a mouse anti-human CD371 antibody 1075.7, and EtM195Mth28z represents a CD33-targeted CAR T cell derived from murine anti-human CD33 antibody, M195.

FIG. 5 depicts tumor cell lysis activities of CD371-targeted CAR T cells in 24 hour killing assays. Healthy donor-derived (referred to as “donor D”) CD371-targeted CAR T cells were cocultured with CD33⁺/CD371⁺ U937 cells expressing GFP and firefly luciferase (U937gL) at different effector:tumor (E:T ratios). Bioluminescence was measured 24 hours later and plotted as a percentage of signal detected in a coculture of a non-functional CAR T cells (based on B10L4H, but without a CD28 or CD3ζ zeta signaling domain) and U937gL. Et.C1HVh28z represents a CD371-targeted CAR derived from a mouse anti-human CD371 antibody 1075.7, and EtM195Mth28z represents a CD33-targeted CAR T cell derived from a murine anti-human CD33 antibody, M195.

FIG. 6 depicts activities of CD371-targeted CAR T cells in a recursive stimulation assay. Healthy donor-derived (referred to as “donor C”) CD371-targeted CAR T cells were cocultured with CD33⁺/CD371⁺ U937gL cell at an E:T ratio of 1:12.5 and at a concentration of 30,000 CAR positive/ml. CAR T cells were counted approximately every 4-5 days and characterized by flow cytometry, and the starting number of tumor cells were added back into the culture (indicated by arrows). Et.B10LHdel represents a non-functional CAR T cells (lacking signaling domains); Et.C1HVh28z represents a CD371-targeted CAR derived from a mouse anti-human CD371 antibody 1075.7, and EtM195Mth28z represents a CD33-targeted CAR T cell derived from a murine anti-human CD33 antibody, M195

FIG. 7 depicts activities of CD371-targeted CAR T cells in a recursive stimulation assay. Healthy donor-derived (referred to as “donor D”) CD371-targeted CAR T cells were cocultured with CD33⁺/CD371⁺ U937gL cell at an E:T ratio of 1:12.5 and at a concentration of 30,000 CAR positive/ml. CAR T cells were counted approximately every 4-5 days and characterized by flow cytometry, and the starting number of tumor cells were added back into the culture (indicated by arrows). Et.B10LHdel represents a non-functional CAR T cells (lacking signaling domains); Et.C1HVh28z represents a CD371-targeted CAR derived from a mouse anti-human CD371 antibody 1075.7, and EtM195Mth28z represents a CD33-targeted CAR T cell derived from a murine anti-human CD33 antibody, M195.

FIGS. 8A and 8B depict in vitro anti-tumor activities of CD371-targeted CAR T cells (second generation B10HL-based CAR T cells and B10LH-based CAR T cells) in a U937gL 24-hour killing assay from different healthy donors. Healthy human donor-derived (referred to as “Donor A” and “Donor” B) CD371-targeted CAR T cells were cocultured with CD371-positive U937 cells expressing GFP and firefly luciferase (U937gL) at different effector:tumor ratios. Bioluminescence was measured 24 hours later and plotted as a percentage of signal detected in a coculture of a non-functional CD371-targeted CAR T cells (based on B10L4H, but without a CD28 or CD3ζ zeta signaling domain) and U937gL. FIG. 8A shows the results for Donor A. FIG. 8B shows the results for Donor B.

FIGS. 9A and 9B depict in vitro anti-tumor activities of CD371-targeted CAR T cells (second generation B10HL-based CAR T cells and B10LH-based CAR T cells) in HL60gL 24-hour killing assay from different healthy donors. Healthy human donor-derived (referred to as “Donor A” and “Donor “B”) CD371-targeted CAR T cells were cocultured with CD371-positive HL60 cells expressing GFP and firefly luciferase (HL60gL) at different effector:tumor ratios. Bioluminescence was measured 24 hours later and plotted as a percentage of signal detected in a coculture of a non-functional CD371-targeted CAR T cells (based on B10L4H, but without a CD28 or CD3ζ zeta signaling domain) and HL60gL. FIG. 9A shows the results for Donor A. FIG. 9B shows the results for Donor B.

FIGS. 10A and 10B depicts generation of human CD371 (hCD371) CRISPR knockout cell lines. Human CD371 was knocked out of HL60gL (FIG. 10A) and U937gL (FIG. 10B) with CRISPR. The knockout was confirmed by flow cytometry using anti-human CD371 APC-conjugated antibody. FIG. 10A shows the knockout of HL60gL. FIG. 10B shows the knockout of U937gL.

FIGS. 11A and 11B depict cytotoxicity activities of CD371-targeted CAR T cells (second generation B10-based CAR T cells, i.e., B10-HL- and B10LH-based CAR T cells) against antigen-negative U937gL in a 24-hour killing assay from different healthy donors. Healthy human donor-derived (referred to as “Donor A” and “Donor “B”) CD371-targeted CAR T cells were cocultured with CD371-negative U937 cells expressing RFP and cypridina luciferase (U937RFPcyp.371KO) at different effector:tumor ratios. Bioluminescence was measured 24 hours later and plotted as a percentage of signal detected in a coculture of a non-functional CD371-targeted CAR T cells (based on B10L4H, but without a CD28 or CD3ζ zeta signaling domain) and U937RFPcyp.371KO. FIG. 11A shows the results for Donor A. FIG. 11B shows the results for Donor B.

FIGS. 12A and 12B depict cytotoxicity activities of CD371-targeted CAR T cells (second generation B10-based CAR T cells, i.e., B10-HL- and B10LH-based CAR T cells) against antigen-negative HL60gL in a 24-hour killing assay from different healthy donors. Healthy human donor-derived (referred to as “Donor A” and “Donor “B”) CD371-targeted CAR T cells were cocultured with CD371-negative HL60 cells expressing GFP and firefly luciferase (HL60L.371KO) at different effector:tumor ratios. Bioluminescence was measured 24 hours later and plotted as a percentage of signal detected in a coculture of a non-functional CD371-targeted CAR T cells (based on B10L4H, but without a CD28 or CD3ζ zeta signaling domain) and HL60gL.371KO. FIG. 12A shows the results for Donor A. FIG. 12B shows the results for Donor B.

FIGS. 13A and 13B depict interferon gamma (IFN-γ) secretion of CD371-targeted CAR T cells (second generation B10-based CAR T cells, i.e., B10-HL- and B10LH-based CAR T cells). Human CD371-targeted CAR T cells were cocultured alone, with CD371-negative U937 cells, or CD371+ U937 cells at an effector:tumor ratio (E:T) of 1:1 (4.0×10⁴:4.0×10⁴ cells in 200 μl). 24 hours later, supernatant was collected and IFN-γ was measured utilizing a bead-based multiplex assay. FIG. 13A shows the results for Donor A. FIG. 13B shows the results for Donor B.

FIGS. 14A and 14B depict interleukin-2 (IL-2) secretion of CD371-targeted CAR T cells (second generation B10-based CAR T cells, i.e., B10-HL- and B10LH-based CAR T cells). Human CD371-targeted CAR T cells were cocultured alone, with CD371-negative U937 cells, or CD371+ U937 cells at an effector:tumor ratio (E:T) of 1:1 (4.0×10⁴:4.0×10⁴ cells in 200 μl). 24 hours later, supernatant was collected and IL-2 was measured using a bead-based multiplex assay. FIG. 14A shows the results for Donor A. FIG. 14B shows the results for Donor B.

FIGS. 15A and 15B depict tumor necrosis factor alpha (TNF-α) secretion of CD371-targeted CAR T cells (second generation B10-based CAR T cells, i.e., B10-HL- and B10LH-based CAR T cells). Human CD371-targeted CAR T cells were cocultured alone, with CD371-negative U937 cells, or CD371+ U937 cells at an effector:tumor ratio (E:T) of 1:1 (4.0×10⁴:4.0×10⁴ cells in 200 μl). 24 hours later, supernatant was collected and TNF-α was measured using a bead-based multiplex assay. FIG. 15A shows the results for Donor A. FIG. 15B shows the results for Donor B.

FIGS. 16A and 16B depicts cell proliferation of B10-based CAR T cells in a recursive stimulation assay. CD371-targeted CAR T cells were generated from two healthy donors (Donor A and Donor B). CAR T cells were cocultured with CD371+ U937gL at an E:T ratio of 1:5 and at a concentration of 50,000 CAR positive/ml. Approximately every 5 days, CAR T cells were counted and characterized by flow cytometry. The starting number of tumor cells were added back into the culture (indicated at arrows). FIG. 16A shows the results for Donor A. FIG. 16B shows the results for Donor B.

FIG. 17 illustrates a murine xenograft model of AML. NCG mice were inoculated with 5×10⁴ U937gL AML FAB-M5 cell lines via tail vein. CD371-targeted CAR T cells were administered to the mice three days later. Tumor kinetics measured non-invasively with bioluminescent imaging approximately every 5 days, and survival was monitored.

FIG. 18 depicts the survival of AML cell-line Xenografted mice (Donor 1) treated with human CD371-targeted CAR T cells. NCG mice were inoculated with 5×10⁴ U937gL tumor cells and treated with approximately 1.25×10⁶ human CD371-targeted CAR T cells three days later. Survival of mice were monitored.

FIG. 19 depicts the survival of AML cell-line Xenografted mice (Donor 2) treated with human CD371-targeted CAR T cells. NCG mice were inoculated with U937gL tumor cells and treated with approximately 1.25×10⁶ human CD371 targeted CAR T cells three days later. Survival of mice were monitored.

FIG. 20 depicts in vivo activity of B10-based CAR T cells. NCG mice were inoculated with 5×10⁴ U937gL tumor cells and treated with various doses of CD371-targeted CAR T cells (1.25×10⁵, 2.5×10⁵, 5.0×10⁵, and 1.0×10⁶) three days later. Survival of the mice was monitored within 55 days (FIG. 20A) and 60 days (FIG. 20B). FIG. 21 depicts binding of scFvs to HEK293 cells expressing human CD371.

FIG. 22 depicts the in vitro cytotoxicity of second-generation B10HL-based CAR T cells against CD371-positive targets. The B10HL-based CAR T cells have G4S linkers of varying lengths. Healthy human donor-derived (including Donors A & B) CD371-targeted CAR T cells were cocultured with antigen-positive U937 cells expressing GFP and firefly luciferase (U937gL) at different effector:tumor ratios. Bioluminescence was measured 24 hours later, and was plotted as a percentage of signal detected in a coculture of a non-functional CD371-targeted CAR T cells (based on B10L4H, but without a CD28 or CD3ζ zeta signaling domain) and U937gL.

FIG. 23 depicts in vivo activities of B10-based CAR T cells. NCG mice were inoculated with 5×10⁴ U937gL tumor cells, and were treated with 5×10⁵ of CAR T cells. Survival of the mice was monitored. B10H3L, B10H5L, and IL33-secreting B10H4L human CD371-targeted CAR T cells outperformed B10H4L-based CAR T cells in vivo.

FIG. 24 depicts in vivo activities of B10-based CAR T cells. NCG mice were inoculated with 5×10⁴ U937gL tumor cells, and were treated with 2.5×10⁵ of CAR T cells. Survival of the mice was monitored. IL18- and IL33-secreting B10H4L Human CD371-targeted CAR T cells outperformed B10H4L-based CAR T cells in vivo.

FIG. 25 depicts in vivo activities of B10-based CAR T cells. NCG mice were inoculated with 5×10⁴ U937gL tumor cells, and were treated with 1.0×10⁵ of CAR T cells. Survival of the mice was monitored. At a low dosage of 1.0×10⁵, IL18-secreting B10H4L human CD371-targeted CAR T cells outperformed all other constructs in vivo.

### DETAILED DESCRIPTION OF THE INVENTION

The presently disclosed subject matter provides antigen-recognizing receptors (e.g., chimeric antigen receptors (CARs) or T-cell receptors (TCRs)) that specifically target CD371. The presently disclosed subject matter further provides cells comprising such receptors. The cells can be immunoresponsive cells, e.g., genetically modified immunoresponsive cells (e.g., T cells or NK cells). The presently disclosed subject matter also provides methods of using such cells for treatments, e.g., for treating and/or preventing a tumor or neoplasm (e.g., AML).

Non-limiting embodiments of the present disclosure are described by the present specification and Examples.

For purposes of clarity of disclosure and not by way of limitation, the detailed description is divided into the following subsections:

5.1. Definitions;

5.2. CD371;

5.3. Antigen-Recognizing Receptors;

5.4. Cells;

5.5. Compositions and Vectors;

5.6. Polypeptides;

5.7. Formulations and Administration;

5.8. Methods of Treatment; and

5.9. Kits

### 5.1. Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

By “immunoresponsive cell” is meant a cell that functions in an immune response or a progenitor, or progeny thereof. In certain embodiments, the immunoresponsive cell is a cell of lymphoid lineage. Non-limiting examples of cells of lymphoid lineage include T cells, Natural Killer (NK) cells, B cells, and stem cells from which lymphoid cells may be differentiated. In certain embodiments, the immunoresponsive cell is a cell of myeloid lineage.

By “activates an immunoresponsive cell” is meant induction of signal transduction or changes in protein expression in the cell resulting in initiation of an immune response. For example, when CD3ζ Chains cluster in response to ligand binding and immunoreceptor tyrosine-based inhibition motifs (ITAMs) a signal transduction cascade is produced. In certain embodiments, when an endogenous TCR or an exogenous CAR binds to an antigen, a formation of an immunological synapse occurs that includes clustering of many molecules near the bound receptor (e.g. CD4 or CD8, CD3γ/δ/ε/ζ, etc.). This clustering of membrane bound signaling molecules allows for ITAM motifs contained within the CD3ζ chains to become phosphorylated. This phosphorylation in turn initiates a T cell activation pathway ultimately activating transcription factors, such as NF-κB and AP-1. These transcription factors induce global gene expression of the T cell to increase IL-2 production for proliferation and expression of master regulator T cell proteins in order to initiate a T cell mediated immune response.

By “stimulates an immunoresponsive cell” is meant a signal that results in a robust and sustained immune response. In various embodiments, this occurs after immune cell (e.g., T-cell) activation or concomitantly mediated through receptors including, but not limited to, CD28, CD137 (4-1BB), OX40, CD40 and ICOS. Receiving multiple stimulatory signals can be important to mount a robust and long-term T cell mediated immune response. T cells can quickly become inhibited and unresponsive to antigen. While the effects of these co-stimulatory signals may vary, they generally result in increased gene expression in order to generate long lived, proliferative, and anti-apoptotic T cells that robustly respond to antigen for complete and sustained eradication.

The term “antigen-recognizing receptor” as used herein refers to a receptor that is capable of recognizing a target antigen (e.g., CD371). In certain embodiments, the antigen-recognizing receptor is capable of activating an immune or immunoresponsive cell (e.g., a T cell) upon its binding to the target antigen.

As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)₂, and Fab fragments that lack the Fe fragment of intact antibody, clear more rapidly from the circulation, and may have less non-specific tissue binding of an intact antibody (Wahl et al., Nucl Med (1983); 24:316-325). As used herein, include whole native antibodies, bispecific antibodies; chimeric antibodies; Fab, Fab′, single chain V region fragments (scFv), fusion polypeptides, and unconventional antibodies. In certain embodiments, an antibody is a glycoprotein comprising at least two heavy (H) chains and two light (L) chains inter-connected by disulfide bonds. Each heavy chain is comprised of a heavy chain variable region (abbreviated herein as V_{H}) and a heavy chain constant (C_{H}) region. The heavy chain constant region is comprised of three domains, CH1, CH2 and CH3. Each light chain is comprised of a light chain variable region (abbreviated herein as V_{L}) and a light chain constant C_{L} region. The light chain constant region is comprised of one domain, C_{L}. The V_{H} and V_{L} regions can be further sub-divided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

As used herein, “CDRs” are defined as the complementarity determining region amino acid sequences of an antibody which are the hypervariable regions of immunoglobulin heavy and light chains. See, e.g., Kabat et al., Sequences of Proteins of Immunological Interest, 4th U. S. Department of Health and Human Services, National Institutes of Health (1987), or IMGT numbering system (Lefranc, The Immunologist (1999); 7:132-136; Lefranc et al., Dev. Comp. Immunol. (2003); 27:55-77). Generally, antibodies comprise three heavy chain and three light chain CDRs or CDR regions in the variable region. CDRs provide the majority of contact residues for the binding of the antibody to the antigen or epitope. In certain embodiments, the CDRs regions are delineated using the IMGT numbering system. In certain embodiments, the CDR regions are delineated using the IMGT numbering system accessible at http://www.imgt.org/IMGT_vquest/input.

As used herein, the term “single-chain variable fragment” or “scFv” is a fusion protein of the variable regions of the heavy (V_{H}) and light chains (V_{L}) of an immunoglobulin (e.g., mouse or human) covalently linked to form a V_{H}::VL heterodimer. The heavy (V_{H}) and light chains (V_{L}) are either joined directly or joined by a peptide-encoding linker (e.g., 10, 15, 20, 25 amino acids), which connects the N-terminus of the V_{H} with the C-terminus of the V_{L}, or the C-terminus of the V_{H} with the N-terminus of the V_{L}. The linker is usually rich in glycine for flexibility, as well as serine or threonine for solubility. The linker can link the heavy chain variable region and the light chain variable region of the extracellular antigen-binding domain. Non-limiting examples of linkers are disclosed in Shen et al., Anal. Chem. 80(6):1910-1917 (2008) and WO 2014/087010, the contents of which are hereby incorporated by reference in their entireties. In certain embodiments, the linker is a G4S linker.

In certain embodiments, the linker comprises the amino acid sequence set forth in SEQ ID NO: 13, which is provided below:

GGGGSGGGGSGGGSGGGGS [SEQ ID NO:13]

In certain embodiments, the linker comprise the amino acid sequence set forth in SEQ ID NO: 14, which is provided below:

GGGGSGGGGSGGGGS [SEQ ID NO: 14]

In certain embodiments, the linker comprises the amino acid sequence set forth in SEQ ID NO: 91, which is provided below:

GGGGSGGGGSGGGGSGGGSGGGGS [SEQ ID NO: 91]

In certain embodiments, the linker comprises the amino acid sequence set forth in SEQ ID NO: 92, which is provided below:

GGGGSGGGGSGGGGSGGGGSGGGSGGGGS [SEQ ID NO: 92]

In certain embodiments, the linker comprises the amino acid sequence set forth in SEQ ID NO: 93, which is provided below:

GGGGS [SEQ ID NO: 93]

In certain embodiments, the linker comprises the amino acid sequence set forth in SEQ ID NO: 94, which is provided below:

GGGGSGGGGS [SEQ ID NO: 94]

Despite removal of the constant regions and the introduction of a linker, scFv proteins retain the specificity of the original immunoglobulin. Single chain Fv polypeptide antibodies can be expressed from a nucleic acid comprising V_{H}- and V_{L}-encoding sequences as described by Huston, et al. Proc. Nat. Acad. Sci. USA, (1988); 85:5879-5883; U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hyrbidoma (Larchmt) (2008); 27(6):455-51; Peter et al., J Cachexia Sarcopenia Muscle (2012); August 12; Shieh et al., J Imunol (2009); 183(4):2277-85; Giomarelli et al., Thromb Haemost (2007); 97(6):955-63; Fife et al., J Clin Invst (2006); 116(8):2252-61; Brocks et al., Immunotechnology 1997 3(3):173-84; Moosmayer et al., Ther Immunol 1995 2(10:31-40). Agonistic scFvs having stimulatory activity have been described (Peter et al., J Biol Chern (2003); 25278(38):36740-7; Xie et al., Nat Biotech 1997 15(8):768-71; Ledbetter et al., Crit Rev Immunol (1997); 17(5-6):427-55; Ho et al., BioChim Biophys Acta (2003); 1638(3):257-66).

The term “chimeric antigen receptor” or “CAR” as used herein refers to a molecule comprising an extracellular antigen-binding domain that is fused to an intracellular signaling domain that is capable of activating or stimulating an immunoresponsive cell, and a transmembrane domain. In certain embodiments, the extracellular antigen-binding domain of a CAR comprises a scFv. The scFv can be derived from fusing the variable heavy and light regions of an antibody. Alternatively or additionally, the scFv may be derived from Fab's (instead of from an antibody, e.g., obtained from Fab libraries). In certain embodiments, the scFv is fused to the transmembrane domain and then to the intracellular signaling domain. By “substantially identical” or “substantially homologous” is meant a polypeptide or nucleic acid molecule exhibiting at least about 50% homologous or identical to a reference amino acid sequence (for example, any of the amino acid sequences described herein) or a reference nucleic acid sequence (for example, any of the nucleic acid sequences described herein). In certain embodiments, such a sequence is at least about 60%, at least about 65%, at least about 70%, at least about 75%, at least about 80%, at least about 85%, at least about 90%, at least about 95%, at least about 99%, or at least about 100% homologous or identical to the sequence of the amino acid or nucleic acid used for comparison.

Sequence identity can be measured by using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

An “effective amount” is an amount sufficient to affect a beneficial or desired clinical result upon treatment. An effective amount can be administered to a subject in one or more doses. In certain embodiments, an effective amount can be an amount that is sufficient to palliate, ameliorate, stabilize, reverse or slow the progression of the disease, or otherwise reduce the pathological consequences of the disease. The effective amount can be determined by a physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the cells administered.

As used herein, the term “endogenous” refers to a nucleic acid molecule or polypeptide that is normally expressed in a cell or tissue.

As used herein, the term “exogenous” refers to a nucleic acid molecule or polypeptide that is not endogenously present in a cell. The term “exogenous” would therefore encompass any recombinant nucleic acid molecule or polypeptide expressed in a cell, such as foreign, heterologous, and over-expressed nucleic acid molecules and polypeptides. By “exogenous” nucleic acid is meant a nucleic acid not present in a native wild-type cell; for example, an exogenous nucleic acid may vary from an endogenous counterpart by sequence, by position/location, or both. For clarity, an exogenous nucleic acid may have the same or different sequence relative to its native endogenous counterpart; it may be introduced by genetic engineering into the cell itself or a progenitor thereof, and may optionally be linked to alternative control sequences, such as a non-native promoter or secretory sequence.

By a “heterologous nucleic acid molecule or polypeptide” is meant a nucleic acid molecule (e.g., a cDNA, DNA or RNA molecule) or polypeptide that is not normally present in a cell or sample obtained from a cell. This nucleic acid may be from another organism, or it may be, for example, an mRNA molecule that is not normally expressed in a cell or sample.

By “modulate” is meant positively or negatively alter. Exemplary modulations include a about 1%, about 2%, about 5%, about 10%, about 25%, about 50%, about 75%, or about 100% change.

By “increase” is meant to alter positively by at least about 5%. An alteration may be by about 5%, about 10%, about 25%, about 30%, about 50%, about 75%, about 100% or more.

By “reduce” is meant to alter negatively by at least about 5%. An alteration may be by about 5%, about 10%, about 25%, about 30%, about 50%, about 75%, or even by about 100%.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated cell” is meant a cell that is separated from the molecular and/or cellular components that naturally accompany the cell.

The term “antigen-binding domain” as used herein refers to a domain capable of specifically binding a particular antigenic determinant or set of antigenic determinants present on a cell.

By “neoplasm” is meant a disease characterized by the pathological proliferation of a cell or tissue and its subsequent migration to or invasion of other tissues or organs. Neoplastic growth is typically uncontrolled and progressive, and occurs under conditions that would not elicit, or would cause cessation of, multiplication of normal cells. Neoplasm can affect a variety of cell types, tissues, or organs, including but not limited to an organ selected from the group consisting of bladder, bone, brain, breast, cartilage, glia, esophagus, fallopian tube, gallbladder, heart, intestines, kidney, liver, lung, lymph node, nervous tissue, ovaries, pancreas, prostate, skeletal muscle, skin, spinal cord, spleen, stomach, testes, thymus, thyroid, trachea, urogenital tract, ureter, urethra, uterus, and vagina, or a tissue or cell type thereof. Neoplasia include cancers, such as sarcomas, carcinomas, or plasmacytomas (malignant tumor of the plasma cells). The neoplasia can a primary tumor or primary cancer. In addition, the neoplasm can be in metastatic status.

By “receptor” is meant a polypeptide, or portion thereof, present on a cell membrane that selectively binds one or more ligand.

By “recognize” is meant selectively binds to a target. A T cell that recognizes a tumor can expresses a receptor (e.g., a TCR or CAR) that binds to a tumor antigen.

By “reference” or “control” is meant a standard of comparison. For example, the level of scFv-antigen binding by a cell expressing a CAR and an scFv may be compared to the level of scFv-antigen binding in a corresponding cell expressing CAR alone.

By “secreted” is meant a polypeptide that is released from a cell via the secretory pathway through the endoplasmic reticulum, Golgi apparatus, and as a vesicle that transiently fuses at the cell plasma membrane, releasing the proteins outside of the cell.

By “signal sequence” or “leader sequence” is meant a peptide sequence (e.g., 5, 10, 15, 20, 25 or 30 amino acids) present at the N-terminus of newly synthesized proteins that directs their entry to the secretory pathway

By “specifically binds” or “specifically binds to” or “specifically target” is meant a polypeptide or a fragment thereof that recognizes and/or binds to a biological molecule of interest (e.g., a polypeptide, e.g., a CD371 polypeptide), but which does not substantially recognize and/or bind other molecules in a sample, for example, a biological sample, which naturally includes a presently disclosed polypeptide (e.g., a CD371 polypeptide).

The terms “comprises”, “comprising”, and are intended to have the broad meaning ascribed to them in U.S. Patent Law and can mean “includes”, “including” and the like.

As used herein, “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

An “individual” or “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys. The term “immunocompromised” as used herein refers to a subject who has an immunodeficiency. The subject is very vulnerable to opportunistic infections, infections caused by organisms that usually do not cause disease in a person with a healthy immune system but can affect people with a poorly functioning or suppressed immune system.

Other aspects of the presently disclosed subject matter are described in the following disclosure and are within the ambit of the presently disclosed subject matter.

### 5.2. CD371

CD371 (CEC12A), also known as DCAL-2, MICL or CLL-1, is a 30 kD C-type lectin transmembrane glycoprotein. It is expressed on monocytes, granulocytes, natural killer (NK) cells, and basophils. CD371 is an immunoinhibitory receptor that recruits Src homology phosphatases SHP-1 and SHP-2 to its phosphorylated cytoplasmic immunoreceptor tyrosine-based inhibitory motif (ITIM) (Sancho et al., Annu Rev. Immunol (2012); 30:491-529; Yan et al., Front Immunol (2015); 6:408; Lahoud et al., J Immunol (2011); 187:842). CD371 has been implicated as a negative regulatory uric acid crystals (monosodium urate, MSU) receptor that controls autoimmunity and inflammatory disease (Neumann et al., Immunity (2014); 40:389-99). CD371 is a negative regulator of granulocyte and monocyte function (Marshall et al., J Biol Chem (2004); 279(15):14792-802; Pyz et al., Eur J Immunol (2008); 38(4):1157-63).

In certain embodiments, CD371 is a human CD371 comprising or consisting of the amino acid sequence with a NCBI Reference No: NP_612210.4 (SEQ ID NO: 15), or a fragment thereof.

SEQ ID NO: 15 is provided below:

[SEQ ID NO: 15]
MSEEVTYADL QFQNSSEMEK IPEIGKFGEK APPAPSHVWR
PAALFLTLLC LLLLIGLGVL ASMFHVTLKI EMKKMNKLQN
ISEELQRNIS LQLMSNMNIS NKIRNLSTTL QTIATKLCRE
LYSKEQEHKC KPCPRRWIWH KDSCYFLSDD VQTWQESKMA
CAAQNASLLK INNKNALEFI KSQSRSYDYW LGLSPEEDST
RGMRVDNIIN SSAWVIRNAP DLNNMYCGYI NRLYVQYYHC
TYKKRMICEK MANPVQLGST YFREA

In certain embodiments, the CD371 comprises or consists of an amino acid sequence that is at least about 80%, at least about 85%, at least about 90%, at least about 95%, at least about 96%, at least about 97%, at least about 98%, or at least about 99%, at least about 100% identical to the amino acid sequence set forth in SEQ ID NO: 15 or a fragment thereof.

### 5.3. Antigen-Recognizing Receptors

The presently disclosed antigen-recognizing receptors specifically target or binds to CD371. In certain embodiments, the antigen-recognizing receptor is a chimeric antigen receptor (CAR). In certain embodiments, the antigen-recognizing receptor is a T-cell receptor (TCR). In certain embodiments, the antigen-recognizing receptor is a TCR like fusion molecule.

The presently disclosed subject matter also provides nucleic acid molecules that encode the presently disclosed antigen-recognizing receptors. In certain embodiments, the nucleic acid molecule comprises a nucleotide sequence that encodes a polypeptide of a CD371-targeted antigen recognizing receptor disclosed herein.

5.3.1. T-Cell Receptor (TCR)

In certain embodiments, the antigen-recognizing receptor is a TCR. A TCR is a disulfide-linked heterodimeric protein consisting of two variable chains expressed as part of a complex with the invariant CD3ζ chain molecules. A TCR found on the surface of T cells is responsible for recognizing antigens as peptides bound to major histocompatibility complex (MHC) molecules. In certain embodiments, a TCR comprises an alpha chain and a beta chain (encoded by TRA and TRB, respectively). In certain embodiments, a TCR comprises a gamma chain and a delta chain (encoded by TRG and TRD, respectively).

Each chain of a TCR is composed of two extracellular domains: Variable (V) region and a Constant (C) region. The Constant region is proximal to the cell membrane, followed by a transmembrane region and a short cytoplasmic tail. The Variable region binds to the peptide/MHC complex. The variable domain of both chains each has three complementarity determining regions (CDRs).

In certain embodiments, a TCR can form a receptor complex with three dimeric signaling modules CD3δ/ε, CD3γ/ε and CD247 ζ/ζ or ζ/η. When a TCR complex engages with its antigen and MHC (peptide/MHC), the T cell expressing the TCR complex is activated.

In certain embodiments, the TCR is an endogenous TCR. In certain embodiments, the antigen-recognizing receptor is naturally occurring TCR.

In certain embodiments, the antigen-recognizing receptor is an exogenous TCR. In certain embodiments, the antigen-recognizing receptor is a recombinant TCR. In certain embodiments, the antigen-recognizing receptor is a non-naturally occurring TCR. In certain embodiments, the non-naturally occurring TCR differs from any naturally occurring TCR by at least one amino acid residue. In certain embodiments, the non-naturally occurring TCR differs from any naturally occurring TCR by at least about 2, about 3, about 4, about 5, about 6, about 7, about 8, about 9, about 10, about 11, about 12, about 13, about 14, about 15, about 20, about 25, about 30, about 40, about 50, about 60, about 70, about 80, about 90, about 100 or more amino acid residues. In certain embodiments, the non-naturally occurring TCR is modified from a naturally occurring TCR by at least one amino acid residue. In certain embodiments, the non-naturally occurring TCR is modified from a naturally occurring TCR by at least about 2, about 3, about 4, about 5, about 6, about 7, about 8, about 9, about 10, about 11, about 12, about 13, about 14, about 15, about 20, about 25, about 30, about 40, about 50, about 60, about 70, about 80, about 90, about 100 or more amino acid residues.

5.3.2. Chimeric Antigen Receptor (CAR)

In certain embodiments, the antigen-recognizing receptor is a CAR. CARs are engineered receptors, which graft or confer a specificity of interest onto an immune effector cell. CARs can be used to graft the specificity of a monoclonal antibody onto a T cell; with transfer of their coding sequence facilitated by retroviral vectors.

There are three generations of CARs. “First generation” CARs are typically composed of an extracellular antigen-binding domain (e.g., an scFv), which is fused to a transmembrane domain, which is fused to cytoplasmic/intracellular signaling domain. “First generation” CARs can provide de novo antigen recognition and cause activation of both CD4⁺ and CD8⁺ T cells through their CD3ζ chain signaling domain in a single fusion molecule, independent of HLA-mediated antigen presentation. “Second generation” CARs add intracellular signaling domains from various co-stimulatory molecules (e.g., CD28, 4-1BB, ICOS, OX40) to the cytoplasmic tail of the CAR to provide additional signals to the T cell. “Second generation” CARs comprise those that provide both co-stimulation (e.g., CD28 or 4-1BB) and activation (CD3ζ). “Third generation” CARs comprise those that provide multiple co-stimulation (e.g., CD28 and 4-1BB) and activation (CD3ζ). In certain embodiments, the antigen-recognizing receptor is a first-generation CAR. In certain embodiments, the antigen-recognizing receptor is a CAR that does not comprise an intracellular signaling domain of a co-stimulatory molecule or a fragment thereof. In certain embodiments, the antigen-recognizing receptor is a second-generation CAR.

In certain embodiments, the CAR comprises an extracellular antigen-binding domain that specifically binds to CD371, a transmembrane domain, and an intracellular signaling domain.

5.3.2.1. Extracellular Antigen-Binding Domain of A CAR

In certain embodiments, the extracellular antigen-binding domain is an scFv. In certain embodiments, the scFv is a human scFv. In certain embodiments, the scFv is a humanized scFv. In certain embodiments, the scFv is a murine scFv. In certain embodiments, the scFv is identified by screening scFv phage library with an antigen-Fc fusion protein.

In certain embodiments, the extracellular antigen-binding domain is a Fab. In certain embodiments, the Fab is crosslinked. In certain embodiments, the extracellular antigen-binding domain is a F(ab)₂.

Any of the foregoing molecules may be comprised in a fusion protein with a heterologous sequence to form the extracellular antigen-binding domain. In certain non-limiting embodiments, the extracellular antigen-binding domain of the CAR (embodied, for example, an scFv or an analog thereof) binds to CD371 (e.g., human CD371) with a dissociation constant (K_{d}) of about 1×10⁻⁶ M or less, e.g., about 1×10⁻⁷ M or less, about 1×10⁻⁸ M or less, about 1×10⁻⁹ M or less, about 1×10⁻¹⁰ M or less, or about 1×10⁻¹¹ M or less. In certain embodiments, the extracellular antigen-binding domain of the CAR binds to CD371 (e.g., human CD371) with a K_{d} of about 1×10⁻⁷ M or less. In certain embodiments, the extracellular antigen-binding domain of the CAR binds to CD371 (e.g., human CD371) with a K_{d} of about 1×10⁻⁸ M or less. In certain embodiments, the extracellular antigen-binding domain of the CAR binds to CD371 (e.g., human CD371) with a K_{d} of about 1.5×10⁻⁸ M or about 1×10⁻⁸ M. In certain embodiments, the extracellular antigen-binding domain of the CAR binds to CD371 (e.g., human CD371) with a K_{d} of between about 1×10⁻⁸ M and about 1×10⁻⁷ M.

In certain embodiments, the extracellular antigen-binding domain of the CAR binds to CD371 (e.g., human CD371) with a low binding affinity. In certain embodiments, the extracellular antigen-binding domain of the CAR binds to CD371 (e.g., human CD371) with a K_{d} of about 1.5×10⁻⁸ M or more, about 1×10⁻⁸ M or more, about 1×10⁻⁷ M or more, or about 1×10⁻⁶ M or more.

Binding of the extracellular antigen-binding domain of the CAR can be confirmed by, for example, enzyme-linked immunosorbent assay (ELISA), radioimmunoassay (RIA), FACS analysis, bioassay (e.g., growth inhibition), or Western Blot assay. Each of these assays generally detect the presence of protein-antibody complexes of particular interest by employing a labeled reagent (e.g., an antibody, or a scFv) specific for the complex of interest. For example, the scFv can be radioactively labeled and used in a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a γ counter or a scintillation counter or by autoradiography. In certain embodiments, the CD371-targeted extracellular antigen-binding domain is labeled with a fluorescent marker. Non-limiting examples of fluorescent markers include green fluorescent protein (GFP), blue fluorescent protein (e.g., EBFP, EBFP2, Azurite, and mKalama1), cyan fluorescent protein (e.g., ECFP, Cerulean, and CyPet), and yellow fluorescent protein (e.g., YFP, Citrine, Venus, and YPet). In one embodiment, the CD371-targeted human scFv is labeled with GFP.

In certain embodiments, the CDRs are identified according to the IMGT numbering system.

In certain embodiments, the extracellular antigen-binding domain of the CAR (e.g., an scFv) comprises a V_{H} comprising an amino acid sequence that is at least about 80% (e.g., at least about 85%, at least about 90%, or at least about 95%) homologous or identical to the amino sequence set forth in SEQ ID NO: 1. For example, the extracellular antigen-binding domain of the CAR (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 1. In certain embodiments, the extracellular antigen-binding domain comprises a V_{H} comprising the amino sequence set forth in SEQ ID NO: 1. SEQ ID NO: 1 is provided in Table 1 below.

In certain embodiments, the extracellular antigen-binding domain of the CAR (e.g., an scFv) comprises a V_{L} comprising an amino acid sequence that is at least about 80% (e.g., at least about 85%, at least about 90%, or at least about 95%) homologous or identical to the amino sequence set forth in SEQ ID NO: 2. For example, the extracellular antigen-binding domain of the CAR (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 2. In certain embodiments, the extracellular antigen-binding domain comprises a V_{L} comprising the amino sequence set forth in SEQ ID NO: 2. SEQ ID NO: 2 is provided in Table 1 below.

In certain embodiments, the extracellular antigen-binding domain of the CAR (e.g., an scFv) comprises a V_{H} CDR1 comprising the amino acid sequence set forth in SEQ ID NO: 28 or a conservative modification thereof, a V_{H} CDR2 comprising the amino acid sequence set forth in SEQ ID NO: 29 or a conservative modification thereof, and a V_{H} CDR3 comprising the amino acid sequence set forth in SEQ ID NO: 30 or a conservative modification thereof. SEQ ID NOs: 28-30 are provided in Table 1.

In certain embodiments, the extracellular antigen-binding domain of the CAR (e.g., an scFv) comprises a V_{L} CDR1 comprising the amino acid sequence set forth in SEQ ID NO: 31 or a conservative modification thereof, a V_{L} CDR2 comprising the amino acid sequence set forth in SEQ ID NO: 32 or a conservative modification thereof, and a V_{L} CDR3 comprising the amino acid sequence set forth in SEQ ID NO: 33 or a conservative modification thereof. SEQ ID NOs: 31-33 are provided in Table 1.

In certain embodiments, the extracellular antigen-binding domain of the CAR (e.g., an scFv) comprises a V_{H} CDR1 comprising the amino acid sequence set forth in SEQ ID NO: 28 or a conservative modification thereof, a V_{H} CDR2 comprising the amino acid sequence set forth in SEQ ID NO: 29 or a conservative modification thereof, a V_{H} CDR3 comprising the amino acid sequence set forth in SEQ ID NO: 30 or a conservative modification thereof, a V_{L} CDR1 comprising the amino acid sequence set forth in SEQ ID NO: 31 or a conservative modification thereof, a V_{L} CDR2 comprising the amino acid sequence set forth in SEQ ID NO: 32 or a conservative modification, and a V_{L} CDR3 comprising the amino acid sequence set forth in SEQ ID NO: 33 or a conservative modification thereof.

In certain embodiments, the extracellular antigen-binding domain of the CAR (e.g., an scFv) comprises a V_{H} CDR1 comprising the amino acid sequence set forth in SEQ ID NO: 28, a V_{H} CDR2 comprising the amino acid sequence set forth in SEQ ID NO: 29, a V_{H} CDR3 comprising the amino acid sequence set forth in SEQ ID NO: 30, a V_{L} CDR1 comprising the amino acid sequence set forth in SEQ ID NO: 31, a V_{L} CDR2 comprising the amino acid sequence set forth in SEQ ID NO: 32, and a V_{L} CDR3 comprising the amino acid sequence set forth in SEQ ID NO: 33.

In certain embodiments, the extracellular antigen-binding domain of the CAR (e.g., an scFv) comprises a V_{H} comprising the amino acid sequence set forth in SEQ ID NO: 1, and a V_{L} comprising the amino acid sequence set forth in SEQ ID NO: 2. In certain embodiments, the V_{H} and V_{L} are linked via a linker. In certain embodiments, the linker comprises the amino acid sequence set forth in SEQ ID NO: 13.

In certain embodiments, the variable regions within the extracellular antigen-binding domain of the CAR have to be linked one after another such that at the N-terminus of the extracellular antigen-binding domain, a heavy chain variable region (V_{H}) is positioned. In certain embodiments, if the extracellular antigen-binding domain of the CAR is an scFv, the variable regions are positioned from the N- to the C-terminus: V_{H}-V_{L}. In certain embodiments, the scFv comprises the amino acid sequence set forth in SEQ ID NO: 107, which is provided in Table 1. In certain embodiments, the scFv is designated as “B10H4L”.

In certain embodiments, the variable regions within the extracellular antigen-binding domain of the CAR have to be linked one after another such that at the N-terminus of the extracellular antigen-binding domain, a light chain variable region (V_{L}) is positioned. In certain embodiments, if the extracellular antigen-binding domain of the CAR is an scFv, the variable regions are positioned from the N- to the C-terminus: V_{L}-V_{H}. In certain embodiments, scFv comprises the amino acid sequence set forth in SEQ ID NO: 16. In certain embodiments, the scFv is designated as “B10L4H”. An exemplary nucleotide sequence encoding the amino acid sequence of SEQ ID NO: 16 is set forth in SEQ ID NO: 22. SEQ ID NOS: 16 and 22 are provided in Table 1 below.

TABLE 1

| | A CD371 polypeptide consisting of the amino acid sequence | | |
|---|---|---|---|
| Antigen | of SEQ ID NO: 15 or a fragment thereof | | |
| CDRs | 1 | 2 | 3 |
| V_{H} | GFTFSDYQ [SEQ ID | IQGGGGST [SEQ ID | AREMWRGDYYSGMDV |
| | NO: 28] | NO: 29] | [SEQ ID NO: 30] |
| V_{L} | QSVLDSYNNENN [SEQ | WAS [SEQ ID NO: | QQYTSEPIT [SEQ ID |
| | ID NO: 31] | 32] | NO: 33] |

| | |
|---|---|
| Full V_{H} | EVQLLESGGGLVQPGGSLRLSCAASGFTFSDYQMSWVRQAPGKGLEWVSGIQGGGGSTY |
| | YADSVKGRFTISRDNSKNTLYLQMNSLRAEDTAVYYCAREMWRGDYYSGMDVWGQGTTV |
| | TVSS [SEQ ID NO: 1] |
| Full V_{L} | DIVMTQSPDSLAVSLGERATINCKSSQSVLDSYNNENNLAWYQQKPGQPPKLLIYWAST |
| | RESGVPDRFSGSGSGTDFTLTISSLQAEDVAVYYCQQYTSEPITFGQGTKVEIK [SEQ |
| | ID NO: 2] |
| V_{L}-V_{H} | DIVMTQSPDSLAVSLGERATINCKSSQSVLDSYNNENNLAWYQQKPGQPPKLLIYWAST |
| scFv | RESGVPDRFSGSGSGTDFTLTISSLQAEDVAVYYCQQYTSEPITFGQGTKVEIKGGGGS |
| | GGGGSGGGSGGGGSEVQLLESGGGLVQPGGSLRLSCAASGFTFSDYQMSWVRQAPGKGL |
| | EWVSGIQGGGGSTYYADSVKGRFTISRDNSKNTLYLQMNSLRAEDTAVYYCAREMWRGD |
| | YYSGMDVWGQGTTVTVSS [SEQ ID NO: 16] |
| DNA for | GACATCGTGATGACCCAGTCTCCAGACTCCCTGGCTGTGTCTCTGGGCGAGCGTGCCAC |
| V_{L}-V_{H} | CATCAACTGCAAGTCCAGCCAGAGTGTTTTAGACAGCTATAACAATGAGAACAATTTAG |
| scFv | CTTGGTATCAGCAGAAACCAGGACAGCCTCCTAAGCTGCTCATTTACTGGGCATCTACC |
| | CGGGAATCCGGGGTCCCTGACCGATTCAGTGGCAGCGGGTCTGGGACAGATTTCACTCT |
| | CACCATCAGCAGCCTGCAGGCTGAAGATGTGGCAGTTTATTACTGTCAGCAATATACCA |
| | GCGAACCTATCACGTTCGGCCAAGGTACCAAGGTGGAAATCAAAGGTGGTGGTGGTTCA |
| | GGTGGTGGTGGTTCTGGCGGCGGCTCCGGTGGTGGTGGATCCGAGGTGCAGCTGTTGGA |
| | GTCTGGGGGAGGCTTGGTACAGCCTGGGGGGTCCCTGCGACTCTCCTGTGCAGCCTCTG |
| | GATTCACCTTTAGCGACTATCAGATGAGCTGGGTCCGCCAGGCTCCAGGGAAGGGGCTG |
| | GAGTGGGTGTCAGGCATTCAGGGTGGCGGTGGTAGCACATATTACGCAGACTCCGTGAA |
| | GGGCCGGTTCACCATCTCCCGTGACAATTCCAAGAACACGCTGTATCTGCAAATGAACA |
| | GCCTGCGTGCCGAGGACACGGCTGTGTATTACTGTGCGAGAGAGATGTGGCGTGGGGAC |
| | TACTACTCCGGTATGGACGTCTGGGGCCAGGGGACCACGGTCACCGTCTCCTCA [SEQ |
| | ID NO: 22] |
| V_{H}-V_{L} | EVQLLESGGGLVQPGGSLRLSCAASGFTFSDYQMSWVRQAPGKGLEWVSGIQGGGGSTY |
| scFv | YADSVKGRFTISRDNSKNTLYLQMNSLRAEDTAVYYCAREMWRGDYYSGMDVWGQGTTV |
| | TVSSGGGGSGGGGSGGGSGGGGSDIVMTQSPDSLAVSLGERATINCKSSQSVLDSYNNE |
| | NNLAWYQQKPGQPPKLLIYWASTRESGVPDRFSGSGSGTDFTLTISSLQAEDVAVYYCQ |
| | QYTSEPITFGQGTKVEIK [SEQ ID NO: 107] |

In certain embodiments, the extracellular antigen-binding domain of the CAR (e.g., an scFv) comprises a V_{H} comprising an amino acid sequence that is at least about 80% (e.g., at least about 85%, at least about 90%, or at least about 95%) homologous or identical to the amino sequence set forth in SEQ ID NO: 3. For example, the extracellular antigen-binding domain of the CAR (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 3. In certain embodiments, the extracellular antigen-binding domain comprises a V_{H} comprising the amino sequence set forth in SEQ ID NO: 3. SEQ ID NO: 3 is provided in Table 2 below.

In certain embodiments, the extracellular antigen-binding domain of the CAR (e.g., an scFv) comprises a V_{L} comprising an amino acid sequence that is at least about 80% (e.g., at least about 85%, at least about 90%, or at least about 95%) homologous or identical to the amino sequence set forth in SEQ ID NO: 4. For example, the extracellular antigen-binding domain of the CAR (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 4. In certain embodiments, the extracellular antigen-binding domain comprises a V_{L} comprising the amino sequence set forth in SEQ ID NO: 4. SEQ ID NO: 4 is provided in Table 2 below.

In certain embodiments, the extracellular antigen-binding domain of the CAR (e.g., an scFv) comprises a V_{H} comprising the amino acid sequence set forth in SEQ ID NO: 3, as shown in Table 2. In certain embodiments, the anti-CD371 scFv comprises a V_{L} comprising the amino acid sequence set forth in SEQ ID NO: 4. In certain embodiments, the anti-CD371 scFv comprises a V_{H} comprising the amino acid sequence set forth in SEQ ID NO: 3 and a V_{L} comprising the amino acid sequence set forth in SEQ ID NO: 4.

In certain embodiments, the extracellular antigen-binding domain of the CAR (e.g., an scFv) comprises a V_{H} CDR1 comprising the amino acid sequence set forth in SEQ ID NO: 34 or a conservative modification thereof, a V_{H} CDR2 comprising the amino acid sequence set forth in SEQ ID NO: 35 or a conservative modification thereof, and a V_{H} CDR3 comprising the amino acid sequence set forth in SEQ ID NO: 36 or a conservative modification thereof. SEQ ID NOs: 34-36 are provided in Table 2.

In certain embodiments, the extracellular antigen-binding domain of the CAR (e.g., an scFv) comprises a V_{L} CDR1 comprising the amino acid sequence set forth in SEQ ID NO: 37 or a conservative modification thereof, a V_{L} CDR2 comprising the amino acid sequence set forth in SEQ ID NO: 38 or a conservative modification thereof, and a V_{L} CDR3 comprising the amino acid sequence set forth in SEQ ID NO: 39 or a conservative modification thereof. SEQ ID NOs: 37-39 are provided in Table 2.

In certain embodiments, the extracellular antigen-binding domain of the CAR (e.g., an scFv) comprises a V_{H} CDR1 comprising the amino acid sequence set forth in SEQ ID NO: 34 or a conservative modification thereof, a V_{H} CDR2 comprising the amino acid sequence set forth in SEQ ID NO: 35 or a conservative modification thereof, a V_{H} CDR3 comprising the amino acid sequence set forth in SEQ ID NO: 36 or a conservative modification thereof, a V_{L} CDR1 comprising the amino acid sequence set forth in SEQ ID NO: 37 or a conservative modification thereof, a V_{L} CDR2 comprising the amino acid sequence set forth in SEQ ID NO: 38 or a conservative modification thereof, and a V_{L} CDR3 comprising the amino acid sequence set forth in SEQ ID NO: 39 or a conservative modification thereof.

In certain embodiments, the extracellular antigen-binding domain of the CAR (e.g., a scFv) comprises a V_{H} CDR1 comprising the amino acid sequence set forth in SEQ ID NO: 34, a V_{H} CDR2 comprising the amino acid sequence set forth in SEQ ID NO: 35, a V_{H} CDR3 comprising the amino acid sequence set forth in SEQ ID NO: 36, a V_{L} CDR1 comprising the amino acid sequence set forth in SEQ ID NO: 37, a V_{L} CDR2 comprising the amino acid sequence set forth in SEQ ID NO: 38, and a V_{L} CDR3 comprising the amino acid sequence set forth in SEQ ID NO: 39.

In certain embodiments, the extracellular antigen-binding domain of the CAR (e.g., an scFv) comprises a V_{H} comprising the amino acid sequence set forth in SEQ ID NO: 3, and a V_{L} comprising the amino acid sequence set forth in SEQ ID NO: 4. In certain embodiments, the V_{H} and V_{L} are linked via a linker. In certain embodiments, the linker comprises the amino acid sequence set forth in SEQ ID NO: 13.

In certain embodiments, the variable regions within the extracellular antigen-binding domain of the CAR have to be linked one after another such that at the N-terminus of the extracellular antigen-binding domain, a heavy chain variable region (V_{H}) is positioned. In certain embodiments, if the extracellular antigen-binding domain of the CAR is an scFv, the variable regions are positioned from the N- to the C-terminus: V_{H}-V_{L}. In certain embodiments, the scFv comprises the amino acid sequence set forth in SEQ ID NO: 108, which is provided in Table 2. In certain embodiments, the scFv is designated as “C3H4L”.

In certain embodiments, the variable regions within the extracellular antigen-binding domain of the CAR have to be linked one after another such that at the N-terminus of the extracellular antigen-binding domain, a light chain variable region (V_{L}) is positioned. In certain embodiments, if the extracellular antigen-binding domain of the CAR is an scFv, the variable regions are positioned from the N- to the C-terminus: V_{L}-V_{H}. In certain embodiments, scFv comprises the amino acid sequence set forth in SEQ ID NO: 17. In certain embodiments, the scFv is designated as “C3L4H”. An exemplary nucleotide sequence encoding the amino acid sequence of SEQ ID NO: 17 is set forth in SEQ ID NO: 23. SEQ ID NOS: 17 and 23 are provided in Table 2 below.

TABLE 2

| | A CD371 polypeptide consisting of the amino acid sequence | | |
|---|---|---|---|
| Antigen | of SEQ ID NO: 15 or a fragment thereof | | |
| CDRs | 1 | 2 | 3 |
| V_{H} | GFTFTSYA [SEQ ID | IDGSGGGT [SEQ ID | ARAYYDIL [SEQ ID |
| | NO: 34] | NO: 35] | NO: 36] |
| V_{L} | QSVLSSYNNENN | AAS [SEQ ID NO: | QQYYSEPYT [SEQ ID |
| | [SEQ ID NO: 37] | 38] | NO: 39] |

| | |
|---|---|
| Full V_{H} | EVQLLESGGGLVQPGGSLRLSCAASGFTFTSYAMSWVRQAPGKGLEWVSGIDGSGGGTNYA |
| | DSVKGRFTISRDNSKNTLYLQMNSLRAEDTAVYYCARAYYDILTGYPVDGMDVWGQGTTVT |
| | VSS [SEQ ID NO: 3] |
| Full V_{L} | DIVMTQSPDSLAVSLGERATINCKSSQSVLSSYNNENNLAWYQQKPGQPPKLLIYAASTRE |
| | SGVPDRFSGSGSGTDFTLTISSLQAEDVAVYYCQQYYSEPYTFGQGTKVEIK [SEQ ID |
| | NO: 4] |
| V_{L}-V_{H} | DIVMTQSPDSLAVSLGERATINCKSSQSVLSSYNNENNLAWYQQKPGQPPKLLIYAASTRE |
| scFv | SGVPDRFSGSGSGTDFTLTISSLQAEDVAVYYCQQYYSEPYTFGQGTKVEIKGGGGSGGGG |
| | SGGGSGGGGSEVQLLESGGGLVQPGGSLRLSCAASGFTFTSYAMSWVRQAPGKGLEWVSGI |
| | DGSGGGTNYADSVKGRFTISRDNSKNTLYLQMNSLRAEDTAVYYCARAYYDILTGYPVDGM |
| | DVWGQGTTVTVSS [SEQ ID NO: 17] |
| DNA for | GACATCGTGATGACCCAGTCTCCAGACTCCCTGGCTGTGTCTCTGGGCGAGCGTGCCACCA |
| V_{L}-V_{H} | TCAACTGCAAGTCCAGCCAGAGTGTTTTAAGCAGCTATAACAATGAGAACAATTTAGCTTG |
| scFv | GTATCAGCAGAAACCAGGACAGCCTCCTAAGCTGCTCATTTACGCCGCATCTACCCGGGAA |
| | TCCGGGGTCCCTGACCGATTCAGTGGCAGCGGGTCTGGGACAGATTTCACTCTCACCATCA |
| | GCAGCCTGCAGGCTGAAGATGTGGCAGTTTATTACTGTCAGCAATATTATAGCGAACCTTA |
| | TACGTTCGGCCAAGGTACCAAGGTGGAAATCAAAGGTGGTGGTGGTTCAGGTGGTGGTGGT |
| | TCTGGCGGCGGCTCCGGTGGTGGTGGATCCGAGGTGCAGCTGTTGGAGTCTGGGGGAGGCT |
| | TGGTACAGCCTGGGGGGTCCCTGCGACTCTCCTGTGCAGCCTCTGGATTCACCTTTACCAG |
| | CTATGCCATGAGCTGGGTCCGCCAGGCTCCAGGGAAGGGGCTGGAGTGGGTGTCAGGCATT |
| | GACGGTAGCGGTGGTGGCACAAATTACGCAGACTCCGTGAAGGGCCGGTTCACCATCTCCC |
| | GTGACAATTCCAAGAACACGCTGTATCTGCAAATGAACAGCCTGCGTGCCGAGGACACGGC |
| | TGTGTATTACTGTGCGAGAGCGTATTACGATATTTTGACTGGTTACCCCGTGGACGGTATG |
| | GACGTCTGGGGCCAAGGGACCACGGTCACCGTCTCCTCA [SEQ ID NO: 23] |
| V_{H}-V_{L} | EVQLLESGGGLVQPGGSLRLSCAASGFTFTSYAMSWVRQAPGKGLEWVSGIDGSGGGTNYA |
| scFv | DSVKGRFTISRDNSKNTLYLQMNSLRAEDTAVYYCARAYYDILTGYPVDGMDVWGQGTTVT |
| | VSSGGGGSGGGGSGGGSGGGGSDIVMTQSPDSLAVSLGERATINCKSSQSVLSSYNNENNL |
| | AWYQQKPGQPPKLLIYAASTRESGVPDRFSGSGSGTDFTLTISSLQAEDVAVYYCQQYYSE |
| | PYTFGQGTKVEIK [SEQ ID NO: 108] |

In certain embodiments, the extracellular antigen-binding domain of the CAR (e.g., an scFv) comprises a V_{H} comprising an amino acid sequence that is at least about 80% (e.g., at least about 85%, at least about 90%, or at least about 95%) homologous or identical to the amino sequence set forth in SEQ ID NO: 5. For example, the extracellular antigen-binding domain of the CAR (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 5. In certain embodiments, the extracellular antigen-binding domain comprises a V_{H} comprising the amino sequence set forth in SEQ ID NO: 5. SEQ ID NO: 5 is provided in Table 3 below.

In certain embodiments, the extracellular antigen-binding domain of the CAR (e.g., an scFv) comprises a V_{L} comprising an amino acid sequence that is at least about 80% (e.g., at least about 85%, at least about 90%, or at least about 95%) homologous or identical to the amino sequence set forth in SEQ ID NO: 6. For example, the extracellular antigen-binding domain of the CAR (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 6. In certain embodiments, the extracellular antigen-binding domain comprises a V_{L} comprising the amino sequence set forth in SEQ ID NO: 6. SEQ ID NO: 6 is provided in Table 3 below.

In certain embodiments, the extracellular antigen-binding domain of the CAR (e.g., an scFv) comprises a V_{H} CDR1 comprising the amino acid sequence set forth in SEQ ID NO: 40 or a conservative modification thereof, a V_{H} CDR2 comprising the amino acid sequence set forth in SEQ ID NO: 41 or a conservative modification thereof, and a V_{H} CDR3 comprising the amino acid sequence set forth in SEQ ID NO: 42 or a conservative modification thereof. SEQ ID NOS: 40-42 are provided in Table 3.

In certain embodiments, the extracellular antigen-binding domain of the CAR (e.g., an scFv) comprises a V_{L} CDR1 comprising the amino acid sequence set forth in SEQ ID NO: 43 or a conservative modification thereof, a V_{L} CDR2 comprising the amino acid sequence set forth in SEQ ID NO: 44 or a conservative modification thereof, and a V_{L} CDR3 comprising the amino acid sequence set forth in SEQ ID NO: 45 or a conservative modification thereof. SEQ ID NOs: 43-45 are provided in Table 3.

In certain embodiments, the extracellular antigen-binding domain of the CAR (e.g., an scFv) comprises a V_{H} CDR1 comprising the amino acid sequence set forth in SEQ ID NO: 40 or a conservative modification thereof, a V_{H} CDR2 comprising the amino acid sequence set forth in SEQ ID NO: 41 or a conservative modification thereof, a V_{H} CDR3 comprising the amino acid sequence set forth in SEQ ID NO: 42 or a conservative modification thereof, a V_{L} CDR1 comprising the amino acid sequence set forth in SEQ ID NO: 43 or a conservative modification thereof, a V_{L} CDR2 comprising the amino acid sequence set forth in SEQ ID NO: 44 or a conservative modification thereof, and a V_{L} CDR3 comprising the amino acid sequence set forth in SEQ ID NO: 45 or a conservative modification thereof.

In certain embodiments, the extracellular antigen-binding domain of the CAR (e.g., an scFv) comprises a V_{H} CDR1 comprising the amino acid sequence set forth in SEQ ID NO: 40, a V_{H} CDR2 comprising the amino acid sequence set forth in SEQ ID NO: 41, a V_{H} CDR3 comprising the amino acid sequence set forth in SEQ ID NO: 42, a V_{L} CDR1 comprising the amino acid sequence set forth in SEQ ID NO: 43, a V_{L} CDR2 comprising the amino acid sequence set forth in SEQ ID NO: 44, and a V_{L} CDR3 comprising the amino acid sequence set forth in SEQ ID NO: 45.

In certain embodiments, the extracellular antigen-binding domain of the CAR (e.g., an scFv) comprises a V_{H} comprising the amino acid sequence set forth in SEQ ID NO: 5, and a V_{L} comprising the amino acid sequence set forth in SEQ ID NO: 6. In certain embodiments, the V_{H} and V_{L} are linked via a linker. In certain embodiments, the linker comprises the amino acid sequence set forth in SEQ ID NO: 13.

In certain embodiments, the variable regions within the extracellular antigen-binding domain of the CAR have to be linked one after another such that at the N-terminus of the extracellular antigen-binding domain, a heavy chain variable region (V_{H}) is positioned. In certain embodiments, if the extracellular antigen-binding domain of the CAR is an scFv, the variable regions are positioned from the N- to the C-terminus: V_{H}-V_{L}. In certain embodiments, the scFv comprises the amino acid sequence set forth in SEQ ID NO: 109, which is provided in Table 3. In certain embodiments, the scFv is designated as “D6H4L”.

In certain embodiments, the variable regions within the extracellular antigen-binding domain of the CAR have to be linked one after another such that at the N-terminus of the extracellular antigen-binding domain, a light chain variable region (V_{L}) is positioned. In certain embodiments, if the extracellular antigen-binding domain of the CAR is an scFv, the variable regions are positioned from the N- to the C-terminus: V_{L}-V_{H}. In certain embodiments, scFv comprises the amino acid sequence set forth in SEQ ID NO: 18. In certain embodiments, the scFv is designated as “D6L4H”. An exemplary nucleotide sequence encoding the amino acid sequence of SEQ ID NO: 18 is set forth in SEQ ID NO: 24. SEQ ID NOS: 18 and 24 are provided in Table 3 below.

TABLE 3

| | A CD371 polypeptide consisting of the amino acid sequence | | |
|---|---|---|---|
| Antigen | of SEQ ID NO: 15 or a fragment thereof | | |
| CDRs | 1 | 2 | 3 |
| V_{H} | GFTFTDYA [SEQ | IDGSGGST [SEQ ID | ALELGATTVY [SEQ ID |
| | ID NO: 40] | NO: 41] | NO: 42] |
| V_{L} | QSVLRSSNNKNN | AAS [SEQ ID | QQYYREPLT [SEQ ID |
| | [SEQ ID NO: 43] | NO: 44] | NO: 45] |

| | |
|---|---|
| Full V_{H} | EVQLLESGGGLVQPGGSLRLSCAASGFTFTDYAMSWVRQAPGKGLEWVSDIDGSGGSTDYA |
| | DSVKGRFTISRDNSKNTLYLQMNSLRAEDTAVYYCALELGATTVYWGQGTLVTVSS [SEQ |
| | ID NO: 5] |
| Full V_{L} | DIVMTQSPDSLAVSLGERATINCKSSQSVLRSSNNKNNLAWYQQKPGQPPKLLIYAASTRE |
| | SGVPDRFSGSGSGTDFTLTISSLQAEDVAVYYCQQYYREPLTFGQGTKVEIK [SEQ ID |
| | NO: 6] |
| V_{L}-V_{H} | DIVMTQSPDSLAVSLGERATINCKSSQSVLRSSNNKNNLAWYQQKPGQPPKLLIYAASTRE |
| scFv | SGVPDRFSGSGSGTDFTLTISSLQAEDVAVYYCQQYYREPLTFGQGTKVEIKGGGGSGGGG |
| | SGGGSGGGGSEVQLLESGGGLVQPGGSLRLSCAASGFTFTDYAMSWVRQAPGKGLEWVSDI |
| | DGSGGSTDYADSVKGRFTISRDNSKNTLYLQMNSLRAEDTAVYYCALELGATTVYWGQGTL |
| | VTVSS [SEQ ID NO: 18] |
| DNA for | GACATCGTGATGACCCAGTCTCCAGACTCCCTGGCTGTGTCTCTGGGCGAGCGTGCCACCA |
| V_{L}-V_{H} | TCAACTGCAAGTCCAGCCAGAGTGTTTTACGCAGCAGCAACAATAAAAACAATTTAGCTTG |
| scFv | GTATCAGCAGAAACCAGGACAGCCTCCTAAGCTGCTCATTTACGCCGCATCTACCCGGGAA |
| | TCCGGGGTCCCTGACCGATTCAGTGGCAGCGGGTCTGGGACAGATTTCACTCTCACCATCA |
| | GCAGCCTGCAGGCTGAAGATGTGGCAGTTTATTACTGTCAGCAATATTATCGCGAACCTCT |
| | GACGTTCGGCCAAGGTACCAAGGTGGAAATCAAAGGTGGTGGTGGTTCAGGTGGTGGTGGT |
| | TCTGGCGGCGGCTCCGGTGGTGGTGGATCCGAGGTGCAGCTGTTGGAGTCTGGGGGAGGCT |
| | TGGTACAGCCTGGGGGGTCCCTGCGACTCTCCTGTGCAGCCTCTGGATTCACCTTTACCGA |
| | CTATGCCATGAGCTGGGTCCGCCAGGCTCCAGGGAAGGGGCTGGAGTGGGTGTCAGACATT |
| | GACGGTAGCGGTGGTAGCACAGACTACGCAGACTCCGTGAAGGGCCGGTTCACCATCTCCC |
| | GTGACAATTCCAAGAACACGCTGTATCTGCAAATGAACAGCCTGCGTGCCGAGGACACGGC |
| | TGTGTATTACTGTGCGCTAGAGCTGGGAGCTACTACCGTCTACTGGGGCCAGGGAACCCTG |
| | GTCACCGTCTCCTCA [SEQ ID NO: 24] |
| V_{H}-V_{L} | EVQLLESGGGLVQPGGSLRLSCAASGFTFTDYAMSWVRQAPGKGLEWVSDIDGSGGSTDYA |
| scFv | DSVKGRFTISRDNSKNTLYLQMNSLRAEDTAVYYCALELGATTVYWGQGTLVTVSSGGGGS |
| | GGGGSGGGSGGGGSDIVMTQSPDSLAVSLGERATINCKSSQSVLRSSNNKNNLAWYQQKPG |
| | QPPKLLIYAASTRESGVPDRFSGSGSGTDFTLTISSLQAEDVAVYYCQQYYREPLTFGQGT |
| | KVEIK [SEQ ID NO: 109] |

In certain embodiments, the extracellular antigen-binding domain of the CAR (e.g., an scFv) comprises a V_{H} comprising an amino acid sequence that is at least about 80% (e.g., at least about 85%, at least about 90%, or at least about 95%) homologous or identical to the amino sequence set forth in SEQ ID NO: 7. For example, the extracellular antigen-binding domain of the CAR (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 7. In certain embodiments, the extracellular antigen-binding domain comprises a V_{H} comprising the amino sequence set forth in SEQ ID NO: 7. SEQ ID NO: 7 is provided in Table 4 below.

In certain embodiments, the extracellular antigen-binding domain of the CAR (e.g., an scFv) comprises a V_{L} comprising an amino acid sequence that is at least about 80% (e.g., at least about 85%, at least about 90%, or at least about 95%) homologous or identical to the amino sequence set forth in SEQ ID NO: 8. For example, the extracellular antigen-binding domain of the CAR (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 8. In certain embodiments, the extracellular antigen-binding domain comprises a V_{L} comprising the amino sequence set forth in SEQ ID NO: 8. SEQ ID NO: 8 is provided in Table 4 below.

In certain embodiments, the extracellular antigen-binding domain of the CAR (e.g., an scFv) comprises a V_{H} CDR1 comprising the amino acid sequence set forth in SEQ ID NO: 46 or a conservative modification thereof, a V_{H} CDR2 comprising the amino acid sequence set forth in SEQ ID NO: 47 or a conservative modification thereof, and a V_{H} CDR3 comprising the amino acid sequence set forth in SEQ ID NO: 48 or a conservative modification thereof. SEQ ID NOs: 46-48 are provided in Table 4.

In certain embodiments, the extracellular antigen-binding domain of the CAR (e.g., an scFv) comprises a V_{L} CDR1 comprising the amino acid sequence set forth in SEQ ID NO: 49 or a conservative modification thereof, a V_{L} CDR2 comprising the amino acid sequence set forth in SEQ ID NO: 50 or a conservative modification thereof, and a V_{L} CDR3 comprising the amino acid sequence set forth in SEQ ID NO: 51 or a conservative modification thereof. SEQ ID NOs: 49-51 are provided in Table 4.

In certain embodiments, the extracellular antigen-binding domain of the CAR (e.g., an scFv) comprises a V_{H} CDR1 comprising the amino acid sequence set forth in SEQ ID NO: 46 or a conservative modification thereof, a V_{H} CDR2 comprising the amino acid sequence set forth in SEQ ID NO: 47 or a conservative modification thereof, a V_{H} CDR3 comprising the amino acid sequence set forth in SEQ ID NO: 48 or a conservative modification thereof, a V_{L} CDR1 comprising the amino acid sequence set forth in SEQ ID NO: 49 or a conservative modification thereof, a V_{L} CDR2 comprising the amino acid sequence set forth in SEQ ID NO: 50 or a conservative modification thereof, and a V_{L} CDR3 comprising the amino acid sequence set forth in SEQ ID NO: 51 or a conservative modification thereof.

In certain embodiments, the extracellular antigen-binding domain of the CAR (e.g., an scFv) comprises a V_{H} CDR1 comprising the amino acid sequence set forth in SEQ ID NO: 46, a V_{H} CDR2 comprising the amino acid sequence set forth in SEQ ID NO: 47, a V_{H} CDR3 comprising the amino acid sequence set forth in SEQ ID NO: 48, a V_{L} CDR1 comprising the amino acid sequence set forth in SEQ ID NO: 49, a V_{L} CDR2 comprising the amino acid sequence set forth in SEQ ID NO: 50, and a V_{L} CDR3 comprising the amino acid sequence set forth in SEQ ID NO: 51.

In certain embodiments, the extracellular antigen-binding domain of the CAR (e.g., an scFv) comprises a V_{H} comprising the amino acid sequence set forth in SEQ ID NO: 7, and a V_{L} comprising the amino acid sequence set forth in SEQ ID NO: 8. In certain embodiments, the V_{H} and V_{L} are linked via a linker. In certain embodiments, the linker comprises the amino acid sequence set forth in SEQ ID NO: 13.

In certain embodiments, the variable regions within the extracellular antigen-binding domain of the CAR have to be linked one after another such that at the N-terminus of the extracellular antigen-binding domain, a heavy chain variable region (V_{H}) is positioned. In certain embodiments, if the extracellular antigen-binding domain of the CAR is an scFv, the variable regions are positioned from the N- to the C-terminus: V_{H}-V_{L}. In certain embodiments, the scFv comprises the amino acid sequence set forth in SEQ ID NO: 110, which is provided in Table 4. In certain embodiments, the scFv is designated as “A11H4L”.

In certain embodiments, the variable regions within the extracellular antigen-binding domain of the CAR have to be linked one after another such that at the N-terminus of the extracellular antigen-binding domain, a light chain variable region (V_{L}) is positioned. In certain embodiments, if the extracellular antigen-binding domain of the CAR is an scFv, the variable regions are positioned from the N- to the C-terminus: V_{L}-V_{H}. In certain embodiments, scFv comprises the amino acid sequence set forth in SEQ ID NO: 19. In certain embodiments, the scFv is designated as “A11L4H”. An exemplary nucleotide sequence encoding the amino acid sequence of SEQ ID NO: 19 is set forth in SEQ ID NO: 25. SEQ ID NOS: 19 and 25 are provided in Table 4 below.

TABLE 4

| | A CD371 polypeptide consisting of the amino acid sequence | | |
|---|---|---|---|
| Antigen | of SEQ ID NO: 15 or a fragment thereof | | |
| CDRs | 1 | 2 | 3 |
| V_{H} | GFTFTSTQ [SEQ ID | ISGYGGST [SEQ ID | AKDTEVSGDAFDI [SEQ ID |
| | NO: 46] | NO: 47] | NO: 48] |
| V_{L} | QSVDSSN [SEQ ID | GAS [SEQ ID NO: 50] | QQYRSWPIT [SEQ ID NO: |
| | NO: 49] | | 51] |

| | |
|---|---|
| Full V_{H} | EVQLLESGGGLVQPGGSLRLSCAASGFTFTSTQMSWVRQAPGKGLEWVSEISGYGGSTYYA |
| | DSVKGRFTISRDNSKNTLYLQMNSLRAEDTAVYYCAKDTEVSGDAFDIWGQGTMVTVSS |
| | [SEQ ID NO: 7] |
| Full V_{L} | EIVLTQSPGTLSLSPGERATLSCRASQSVDSSNLAWYQQKPGQAPRLLIYGASSRATGIPD |
| | RFSGSGSGTDFTLTISRLEPEDFAVYYCQQYRSWPITFGQGTKVEIK [SEQ ID NO: |
| | 8] |
| V_{L}-V_{H} | EIVLTQSPGTLSLSPGERATLSCRASQSVDSSNLAWYQQKPGQAPRLLIYGASSRATGIPD |
| scFv | RFSGSGSGTDFTLTISRLEPEDFAVYYCQQYRSWPITFGQGTKVEIKGGGGSGGGGSGGGS |
| | GGGGSEVQLLESGGGLVQPGGSLRLSCAASGFTFTSTQMSWVRQAPGKGLEWVSEISGYGG |
| | STYYADSVKGRFTISRDNSKNTLYLQMNSLRAEDTAVYYCAKDTEVSGDAFDIWGQGTMVT |
| | VSS [SEQ ID NO: 19] |
| DNA for | GAAATTGTGTTGACGCAGTCTCCAGGCACCCTGTCTTTGTCTCCAGGGGAACGTGCCACCC |
| V_{L}-V_{H} | TCTCCTGCCGTGCCAGTCAGAGTGTTGACAGCAGCAATTTAGCCTGGTATCAGCAGAAACC |
| scFv | TGGCCAGGCTCCCCGACTCCTCATCTATGGCGCATCTAGCCGTGCCACTGGTATCCCAGAC |
| | CGTTTCAGTGGCAGTGGGTCTGGGACAGACTTCACTCTCACCATCAGCAGACTGGAGCCTG |
| | AAGATTTTGCAGTGTATTACTGTCAGCAGTATCGCAGCTGGCCTATCACGTTCGGCCAAGG |
| | TACCAAGGTGGAAATCAAAGGTGGTGGTGGTTCAGGTGGTGGTGGTTCTGGCGGCGGCTCC |
| | GGTGGTGGTGGATCCGAGGTGCAGCTGTTGGAGTCTGGGGGAGGCTTGGTACAGCCTGGGG |
| | GGTCCCTGCGACTCTCCTGTGCAGCCTCTGGATTCACCTTTACCAGCACCCAGATGAGCTG |
| | GGTCCGCCAGGCTCCAGGGAAGGGGCTGGAGTGGGTGTCAGAGATTAGCGGTTATGGTGGT |
| | AGCACATACTACGCAGACTCCGTGAAGGGCCGGTTCACCATCTCCCGTGACAATTCCAAGA |
| | ACACGCTGTATCTGCAAATGAACAGCCTGCGTGCCGAGGACACGGCTGTGTATTACTGTGC |
| | AAAAGACACGGAGGTTTCGGGAGATGCTTTTGATATCTGGGGCCAAGGGACAATGGTCACC |
| | GTCTCTTCA [SEQ ID NO: 25] |
| V_{H}-V_{L} | EVQLLESGGGLVQPGGSLRLSCAASGFTFTSTQMSWVRQAPGKGLEWVSEISGYGGSTYYA |
| scFv | DSVKGRFTISRDNSKNTLYLQMNSLRAEDTAVYYCAKDTEVSGDAFDIWGQGTMVTVSSGG |
| | GGSGGGGSGGGSGGGGSEIVLTQSPGTLSLSPGERATLSCRASQSVDSSNLAWYQQKPGQA |
| | PRLLIYGASSRATGIPDRFSGSGSGTDFTLTISRLEPEDFAVYYCQQYRSWPITFGQGTKV |
| | EIK [SEQ ID NO: 110] |

In certain embodiments, the extracellular antigen-binding domain of the CAR (e.g., an scFv) comprises a V_{H} comprising an amino acid sequence that is at least about 80% (e.g., at least about 85%, at least about 90%, or at least about 95%) homologous or identical to the amino sequence set forth in SEQ ID NO: 9. For example, the extracellular antigen-binding domain of the CAR (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 9. In certain embodiments, the extracellular antigen-binding domain comprises a V_{H} comprising the amino sequence set forth in SEQ ID NO: 9. SEQ ID NO: 9 is provided in Table 5 below.

In certain embodiments, the extracellular antigen-binding domain of the CAR (e.g., an scFv) comprises a V_{L} comprising an amino acid sequence that is at least about 80% (e.g., at least about 85%, at least about 90%, or at least about 95%) homologous or identical to the amino sequence set forth in SEQ ID NO: 10. For example, the extracellular antigen-binding domain of the CAR (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 10. In certain embodiments, the extracellular antigen-binding domain comprises a V_{L} comprising the amino sequence set forth in SEQ ID NO: 10. SEQ ID NO: 10 is provided in Table 5 below.

In certain embodiments, the extracellular antigen-binding domain of the CAR (e.g., an scFv) comprises a V_{H} CDR1 comprising the amino acid sequence set forth in SEQ ID NO: 52 or a conservative modification thereof, a V_{H} CDR2 comprising the amino acid sequence set forth in SEQ ID NO: 53 or a conservative modification thereof, and a V_{H} CDR3 comprising the amino acid sequence set forth in SEQ ID NO: 54 or a conservative modification thereof. SEQ ID NOs: 52-54 are provided in Table 5.

In certain embodiments, the extracellular antigen-binding domain of the CAR (e.g., an scFv) comprises a V_{L} CDR1 comprising the amino acid sequence set forth in SEQ ID NO: 55 or a conservative modification thereof, a V_{L} CDR2 comprising the amino acid sequence set forth in SEQ ID NO: 56 or a conservative modification thereof, and a V_{L} CDR3 comprising the amino acid sequence set forth in SEQ ID NO: 57 or a conservative modification thereof. SEQ ID NOs: 55-57 are provided in Table 5.

In certain embodiments, the extracellular antigen-binding domain of the CAR (e.g., an scFv) comprises a V_{H} CDR1 comprising the amino acid sequence set forth in SEQ ID NO: 52 or a conservative modification thereof, a V_{H} CDR2 comprising the amino acid sequence set forth in SEQ ID NO: 53 or a conservative modification thereof, a V_{H} CDR3 comprising the amino acid sequence set forth in SEQ ID NO: 54 or a conservative modification thereof, a V_{L} CDR1 comprising the amino acid sequence set forth in SEQ ID NO: 55 or a conservative modification thereof, a V_{L} CDR2 comprising the amino acid sequence set forth in SEQ ID NO: 56 or a conservative modification thereof, and a V_{L} CDR3 comprising the amino acid sequence set forth in SEQ ID NO: 57 or a conservative modification thereof.

In certain embodiments, the extracellular antigen-binding domain of the CAR (e.g., an scFv) comprises a V_{H} CDR1 comprising the amino acid sequence set forth in SEQ ID NO: 52, a V_{H} CDR2 comprising the amino acid sequence set forth in SEQ ID NO: 53, a V_{H} CDR3 comprising the amino acid sequence set forth in SEQ ID NO: 54, a V_{L} CDR1 comprising the amino acid sequence set forth in SEQ ID NO: 55, a V_{L} CDR2 comprising the amino acid sequence set forth in SEQ ID NO: 56, and a V_{L} CDR3 comprising the amino acid sequence set forth in SEQ ID NO: 57.

In certain embodiments, the extracellular antigen-binding domain of the CAR (e.g., an scFv) comprises a V_{H} comprising the amino acid sequence set forth in SEQ ID NO: 9, and a V_{L} comprising the amino acid sequence set forth in SEQ ID NO: 10. In certain embodiments, the V_{H} and V_{L} are linked via a linker. In certain embodiments, the linker comprises the amino acid sequence set forth in SEQ ID NO: 13.

In certain embodiments, the variable regions within the extracellular antigen-binding domain of the CAR have to be linked one after another such that at the N-terminus of the extracellular antigen-binding domain, a heavy chain variable region (V_{H}) is positioned. In certain embodiments, if the extracellular antigen-binding domain of the CAR is an scFv, the variable regions are positioned from the N- to the C-terminus: V_{H}-V_{L}. In certain embodiments, the scFv comprises the amino acid sequence set forth in SEQ ID NO: 111, which is provided in Table 5. In certain embodiments, the scFv is designated as “E4H4L”.

In certain embodiments, the variable regions within the extracellular antigen-binding domain of the CAR have to be linked one after another such that at the N-terminus of the extracellular antigen-binding domain, a light chain variable region (V_{L}) is positioned. In certain embodiments, if the extracellular antigen-binding domain of the CAR is an scFv, the variable regions are positioned from the N- to the C-terminus: V_{L}-V_{H}. In certain embodiments, scFv comprises the amino acid sequence set forth in SEQ ID NO: 20. In certain embodiments, the scFv is designated as “E4L4H”. An exemplary nucleotide sequence encoding the amino acid sequence of SEQ ID NO: 20 is set forth in SEQ ID NO: 26. SEQ ID NOS: 20 and 26 are provided in Table 5 below.

TABLE 5

| | A CD371 polypeptide consisting of the amino acid sequence | | |
|---|---|---|---|
| Antigen | of SEQ ID NO: 15 or a fragment thereof | | |
| CDRs | 1 | 2 | 3 |
| V_{H} | GFTFTSYY [SEQ ID | ISGSGDST [SEQ ID | AREAGGDYDSGAFDI [SEQ |
| | NO: 52] | NO: 53] | ID NO: 54] |
| V_{L} | QSVLYSGNNKNY [SEQ | GAS [SEQ ID NO: 56] | QQYDYAPFT [SEQ ID |
| | ID NO: 55] | | NO: 57] |

| | |
|---|---|
| Full V_{H} | EVQLLESGGGLVQPGGSLRLSCAASGFTFTSYYMSWVRQAPGKGLEWVSGISGSGDSTSYA |
| | DSVKGRFTISRDNSKNTLYLQMNSLRAEDTAVYYCAREAGGDYDSGAFDIWGQGTMVTVSS |
| | [SEQ ID NO: 9] |
| Full V_{L} | DIVMTQSPDSLAVSLGERATINCKSSQSVLYSGNNKNYLAWYQQKPGQPPKLLIYGASTRE |
| | SGVPDRFSGSGSGTDFTLTISSLQAEDVAVYYCQQYDYAPFTFGQGTKVEIK [SEQ ID |
| | NO: 10] |
| V_{L}-V_{H} | DIVMTQSPDSLAVSLGERATINCKSSQSVLYSGNNKNYLAWYQQKPGQPPKLLIYGASTRE |
| scFv | SGVPDRFSGSGSGTDFTLTISSLQAEDVAVYYCQQYDYAPFTFGQGTKVEIKGGGGSGGGG |
| | SGGGSGGGGSEVQLLESGGGLVQPGGSLRLSCAASGFTFTSYYMSWVRQAPGKGLEWVSGI |
| | SGSGDSTSYADSVKGRFTISRDNSKNTLYLQMNSLRAEDTAVYYCAREAGGDYDSGAFDIW |
| | GQGTMVTVSS [SEQ ID NO: 20] |
| DNA for | GACATCGTGATGACCCAGTCTCCAGACTCCCTGGCTGTGTCTCTGGGCGAGCGTGCCACCA |
| V_{L}-V_{H} | TCAACTGCAAGTCCAGCCAGAGTGTTTTATATAGCGGCAACAATAAAAACTATTTAGCTTG |
| scFv | GTATCAGCAGAAACCAGGACAGCCTCCTAAGCTGCTCATTTACGGCGCATCTACCCGGGAA |
| | TCCGGGGTCCCTGACCGATTCAGTGGCAGCGGGTCTGGGACAGATTTCACTCTCACCATCA |
| | GCAGCCTGCAGGCTGAAGATGTGGCAGTTTATTACTGTCAGCAATATGACTATGCCCCTTT |
| | TACGTTCGGCCAAGGTACCAAGGTGGAAATCAAAGGTGGTGGTGGTTCAGGTGGTGGTGGT |
| | TCTGGCGGCGGCTCCGGTGGTGGTGGATCCGAGGTGCAGCTGTTGGAGTCTGGGGGAGGCT |
| | TGGTACAGCCTGGGGGGTCCCTGCGACTCTCCTGTGCAGCCTCTGGATTCACCTTTACCAG |
| | CTATTATATGAGCTGGGTCCGCCAGGCTCCAGGGAAGGGGCTGGAGTGGGTGTCAGGCATT |
| | AGCGGTAGCGGTGACAGCACAAGCTACGCAGACTCCGTGAAGGGCCGGTTCACCATCTCCC |
| | GTGACAATTCCAAGAACACGCTGTATCTGCAAATGAACAGCCTGCGTGCCGAGGACACGGC |
| | TGTGTATTACTGTGCGAGAGAGGCAGGTGGTGACTACGATAGTGGTGCTTTTGATATCTGG |
| | GGCCAAGGGACAATGGTCACCGTCTCTTCA [SEQ ID NO: 26] |
| V_{H}-V_{L} | EVQLLESGGGLVQPGGSLRLSCAASGFTFTSYYMSWVRQAPGKGLEWVSGISGSGDSTSYA |
| scFv | DSVKGRFTISRDNSKNTLYLQMNSLRAEDTAVYYCAREAGGDYDSGAFDIWGQGTMVTVSS |
| | GGGGSGGGGSGGGSGGGGSDIVMTQSPDSLAVSLGERATINCKSSQSVLYSGNNKNYLAWY |
| | QQKPGQPPKLLIYGASTRESGVPDRFSGSGSGTDFTLTISSLQAEDVAVYYCQQYDYAPFT |
| | FGQGTKVEIK [SEQ ID NO: 111] |

In certain embodiments, the extracellular antigen-binding domain of the CAR (e.g., an scFv) comprises a V_{H} comprising an amino acid sequence that is at least about 80% (e.g., at least about 85%, at least about 90%, or at least about 95%) homologous or identical to the amino sequence set forth in SEQ ID NO: 11. For example, the extracellular antigen-binding domain of the CAR (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 11. In certain embodiments, the extracellular antigen-binding domain comprises a V_{H} comprising the amino sequence set forth in SEQ ID NO: 11. SEQ ID NO: 11 is provided in Table 6 below.

In certain embodiments, the extracellular antigen-binding domain of the CAR (e.g., an scFv) comprises a V_{L} comprising an amino acid sequence that is at least about 80% (e.g., at least about 85%, at least about 90%, or at least about 95%) homologous or identical to the amino sequence set forth in SEQ ID NO: 12. For example, the extracellular antigen-binding domain of the CAR (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 12. In certain embodiments, the extracellular antigen-binding domain comprises a V_{L} comprising the amino sequence set forth in SEQ ID NO: 12. SEQ ID NO: 12 is provided in Table 6 below.

In certain embodiments, the extracellular antigen-binding domain of the CAR (e.g., an scFv) comprises a V_{H} CDR1 comprising the amino acid sequence set forth in SEQ ID NO: 58 or a conservative modification thereof, a V_{H} CDR2 comprising the amino acid sequence set forth in SEQ ID NO: 59 or a conservative modification thereof, and a V_{H} CDR3 comprising the amino acid sequence set forth in SEQ ID NO: 60 or a conservative modification thereof. SEQ ID NOs: 58-60 are provided in Table 6.

In certain embodiments, the extracellular antigen-binding domain of the CAR (e.g., an scFv) comprises a V_{L} CDR1 comprising the amino acid sequence set forth in SEQ ID NO: 61 or a conservative modification thereof, a V_{L} CDR2 comprising the amino acid sequence set forth in SEQ ID NO: 62 or a conservative modification thereof, and a V_{L} CDR3 comprising the amino acid sequence set forth in SEQ ID NO: 63 or a conservative modification thereof. SEQ ID NOs: 61-63 are provided in Table 6.

In certain embodiments, the extracellular antigen-binding domain of the CAR (e.g., an scFv) comprises a V_{H} CDR1 comprising the amino acid sequence set forth in SEQ ID NO: 58 or a conservative modification thereof, a V_{H} CDR2 comprising the amino acid sequence set forth in SEQ ID NO: 59 or a conservative modification thereof, a V_{H} CDR3 comprising the amino acid sequence set forth in SEQ ID NO: 60 or a conservative modification thereof, a V_{L} CDR1 comprising the amino acid sequence set forth in SEQ ID NO: 61 or a conservative modification thereof, a V_{L} CDR2 comprising the amino acid sequence set forth in SEQ ID NO: 62 or a conservative modification thereof, and a V_{L} CDR3 comprising the amino acid sequence set forth in SEQ ID NO: 63 or a conservative modification thereof.

In certain embodiments, the extracellular antigen-binding domain of the CAR (e.g., an scFv) comprises a V_{H} CDR1 comprising the amino acid sequence set forth in SEQ ID NO: 58, a V_{H} CDR2 comprising the amino acid sequence set forth in SEQ ID NO: 59, a V_{H} CDR3 comprising the amino acid sequence set forth in SEQ ID NO: 60, a V_{L} CDR1 comprising the amino acid sequence set forth in SEQ ID NO: 61, a V_{L} CDR2 comprising the amino acid sequence set forth in SEQ ID NO: 62, and a V_{L} CDR3 comprising the amino acid sequence set forth in SEQ ID NO: 63.

In certain embodiments, the extracellular antigen-binding domain of the CAR (e.g., an scFv) comprises a V_{H} comprising the amino acid sequence set forth in SEQ ID NO: 11, and a V_{L} comprising the amino acid sequence set forth in SEQ ID NO: 12. In certain embodiments, the V_{H} and V_{L} are linked via a linker. In certain embodiments, the linker comprises the amino acid sequence set forth in SEQ ID NO: 13.

In certain embodiments, the variable regions within the extracellular antigen-binding domain of the CAR have to be linked one after another such that at the N-terminus of the extracellular antigen-binding domain, a heavy chain variable region (V_{H}) is positioned. In certain embodiments, if the extracellular antigen-binding domain of the CAR is an scFv, the variable regions are positioned from the N- to the C-terminus: V_{H}-V_{L}. In certain embodiments, the scFv comprises the amino acid sequence set forth in SEQ ID NO: 112, which is provided in Table 6. In certain embodiments, the scFv is designated as “E8H4L”.

In certain embodiments, the variable regions within the extracellular antigen-binding domain of the CAR have to be linked one after another such that at the N-terminus of the extracellular antigen-binding domain, a light chain variable region (V_{L}) is positioned. In certain embodiments, if the extracellular antigen-binding domain of the CAR is an scFv, the variable regions are positioned from the N- to the C-terminus: V_{L}-V_{H}. In certain embodiments, scFv comprises the amino acid sequence set forth in SEQ ID NO: 21. In certain embodiments, the scFv is designated as “E8L4H”. An exemplary nucleotide sequence encoding the amino acid sequence of SEQ ID NO: 21 is set forth in SEQ ID NO: 27. SEQ ID NOS: 21 and 27 are provided in Table 6 below.

TABLE 6

| | A CD371 polypeptide consisting of the amino acid sequence | | |
|---|---|---|---|
| Antigen | of SEQ ID NO: 15 or a fragment thereof | | |
| CDRs | 1 | 2 | 3 |
| V_{H} | GFTFSSYA [SEQ ID | IDGEGGYT [SEQ ID | AREGVDYDILTGYYPYGMDV |
| | NO: 58] | NO: 59] | [SEQ ID NO: 60] |
| V_{L} | QSVLDSSNNKNY [SEQ | DAS [SEQ ID NO: 62] | QQGTSSPLT [SEQ ID |
| | ID NO: 61] | | NO: 63] |

| | |
|---|---|
| Full V_{H} | EVQLLESGGGLVQPGGSLRLSCAASGFTFSSYAMSWVRQAPGKGLEWVSEIDGEGGYTNYAD |
| | SVKGRFTISRDNSKNTLYLQMNSLRAEDTAVYYCAREGVDYDILTGYYPYGMDVWGQGTTVT |
| | VSS [SEQ ID NO: 11] |
| Full V_{L} | DIVMTQSPDSLAVSLGERATINCKSSQSVLDSSNNKNYLAWYQQKPGQPPKLLIYDASTRES |
| | GVPDRFSGSGSGTDFTLTISSLQAEDVAVYYCQQGTSSPLTFGQGTKVEIK [SEQ ID |
| | NO: 12] |
| V_{L}-V_{H} | DIVMTQSPDSLAVSLGERATINCKSSQSVLDSSNNKNYLAWYQQKPGQPPKLLIYDASTRES |
| scFv | GVPDRFSGSGSGTDFTLTISSLQAEDVAVYYCQQGTSSPLTFGQGTKVEIKGGGGSGGGGSG |
| | GGSGGGGSEVQLLESGGGLVQPGGSLRLSCAASGFTFSSYAMSWVRQAPGKGLEWVSEIDGE |
| | GGYTNYADSVKGRFTISRDNSKNTLYLQMNSLRAEDTAVYYCAREGVDYDILTGYYPYGMDV |
| | WGQGTTVTVSS [SEQ ID NO: 21] |
| DNA for | GACATCGTGATGACCCAGTCTCCAGACTCCCTGGCTGTGTCTCTGGGCGAGCGTGCCACCAT |
| V_{L}-V_{H} | CAACTGCAAGTCCAGCCAGAGTGTTTTAGACAGCAGCAACAATAAAAACTATTTAGCTTGGT |
| scFv | ATCAGCAGAAACCAGGACAGCCTCCTAAGCTGCTCATTTACGACGCATCTACCCGGGAATCC |
| | GGGGTCCCTGACCGATTCAGTGGCAGCGGGTCTGGGACAGATTTCACTCTCACCATCAGCAG |
| | CCTGCAGGCTGAAGATGTGGCAGTTTATTACTGTCAGCAAGGCACCAGCAGCCCTCTGACGT |
| | TCGGCCAAGGTACCAAGGTGGAAATCAAAGGTGGTGGTGGTTCAGGTGGTGGTGGTTCTGGC |
| | GGCGGCTCCGGTGGTGGTGGATCCGAGGTGCAGCTGTTGGAGTCTGGGGGAGGCTTGGTACA |
| | GCCTGGGGGGTCCCTGCGACTCTCCTGTGCAGCCTCTGGATTCACCTTTAGCAGCTATGCCA |
| | TGAGCTGGGTCCGCCAGGCTCCAGGGAAGGGGCTGGAGTGGGTGTCAGAGATTGACGGTGAG |
| | GGTGGTTATACAAATTACGCAGACTCCGTGAAGGGCCGGTTCACCATCTCCCGTGACAATTC |
| | CAAGAACACGCTGTATCTGCAAATGAACAGCCTGCGTGCCGAGGACACGGCCGTGTATTACT |
| | GTGCGAGAGAAGGGGTAGATTACGATATTTTGACTGGTTATTATCCTTACGGTATGGACGTC |
| | TGGGGCCAAGGGACCACGGTCACCGTCTCCTCA [SEQ ID NO: 27] |
| V_{H}-V_{L} | EVQLLESGGGLVQPGGSLRLSCAASGFTFSSYAMSWVRQAPGKGLEWVSEIDGEGGYTNYAD |
| scFv | SVKGRFTISRDNSKNTLYLQMNSLRAEDTAVYYCAREGVDYDILTGYYPYGMDVWGQGTTVT |
| | VSSGGGGSGGGGSGGGSGGGGSDIVMTQSPDSLAVSLGERATINCKSSQSVLDSSNNKNYLA |
| | WYQQKPGQPPKLLIYDASTRESGVPDRFSGSGSGTDFTLTISSLQAEDVAVYYCQQGTSSPL |
| | TFGQGTKVEIK [SEQ ID NO: 112] |

As used herein, the term “a conservative sequence modification” refers to an amino acid modification that does not significantly affect or alter the binding characteristics of the presently disclosed mesothelin-targeted CAR (e.g., the extracellular antigen-binding domain of the CAR) comprising the amino acid sequence. Conservative modifications can include amino acid substitutions, additions and deletions. Modifications can be introduced into the extracellular antigen-binding domain of the presently disclosed CAR by standard techniques known in the art, such as site-directed mutagenesis and PCR-mediated mutagenesis. Amino acids can be classified into groups according to their physicochemical properties such as charge and polarity. Conservative amino acid substitutions are ones in which the amino acid residue is replaced with an amino acid within the same group. For example, amino acids can be classified by charge: positively-charged amino acids include lysine, arginine, histidine, negatively-charged amino acids include aspartic acid, glutamic acid, neutral charge amino acids include alanine, asparagine, cysteine, glutamine, glycine, isoleucine, leucine, methionine, phenylalanine, proline, serine, threonine, tryptophan, tyrosine, and valine. In addition, amino acids can be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. Thus, one or more amino acid residues within a CDR region can be replaced with other amino acid residues from the same group and the altered antibody can be tested for retained function (i.e., the functions set forth in (c) through (l) above) using the functional assays described herein. In certain embodiments, no more than one, no more than two, no more than three, no more than four, no more than five residues within a specified sequence or a CDR region are altered.

The V_{H} and/or V_{L} amino acid sequences having at least about 80%, at least about 80%, at least about 85%, at least about 90%, or at least about 95% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homology or identity to a specific sequence (e.g., SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12) may contain substitutions (e.g., conservative substitutions), insertions, or deletions relative to the specified sequence(s), but retain the ability to bind to a target antigen (e.g., mesothelin). In certain embodiments, a total of 1 to 10 amino acids are substituted, inserted and/or deleted in a specific sequence (e.g., SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12). In certain embodiments, substitutions, insertions, or deletions occur in regions outside the CDRs (e.g., in the FRs) of the extracellular antigen-binding domain. In certain embodiments, the extracellular antigen-binding domain comprises V_{H} and/or V_{L} sequence selected from SEQ ID NOs: 1-12, including post-translational modifications of that sequence (SEQ ID NO: 1, 2, 3, 4, 5, 6, 7, 8, 9, 10, 11, or 12).

As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm.

The percent homology between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the amino acids sequences of the presently disclosed subject matter can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the specified sequences (e.g., heavy and light chain variable region sequences of scFv m903, m904, m905, m906, and m900) disclosed herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

In certain embodiments, the extracellular antigen-binding domain of a presently disclosed CAR cross-competes for binding to CD371 (e.g., human CD371) with a reference antibody or an antigen-binding fragment thereof comprising the V_{H} CDR1, CDR2, and CDR3 sequences and the V_{L} CDR1, CDR2, and CDR3 sequences of, for example, any one of the presently disclosed scFvs (e.g., V10, C3, D6, A11, E4, and D8). In certain embodiments, the extracellular antigen-binding domain of a presently disclosed CAR cross-competes for binding to CD371 (e.g., human CD371) with a reference antibody or an antigen-binding portion thereof comprising the V_{H} and V_{L} sequences of, for example, any one of the presently disclosed scFvs (e.g., V10, C3, D6, A11, E4, and D8).

In certain embodiments, the extracellular antigen-binding domain of a presently disclosed CAR cross-competes for binding to CD371 (e.g., human CD371) with a reference antibody or an antigen-binding portion thereof comprising the V_{H} CDR1, CDR2, and CDR3 sequences and the V_{L} CDR1, CDR2, and CDR3 sequences of scFv B10. For example, the extracellular antigen-binding domain of a presently disclosed CAR cross-competes for binding to CD371 (e.g., human CD371) with a reference antibody or an antigen-binding portion thereof comprising a V_{H} CDR1 comprising the amino acid sequence set forth in SEQ ID NO: 28, a V_{H} CDR2 comprising the amino acid sequence set forth in SEQ ID NO: 29; a V_{H} CDR3 comprising the amino acid sequence set forth in SEQ ID NO: 30; a V_{L} CDR1 comprising the amino acid sequence set forth in SEQ ID NO: 31; a V_{L} CDR2 comprising amino acids having the sequence set forth in SEQ ID NO: 32; and a V_{L} CDR3 comprising amino acids having the sequence set forth in SEQ ID NO: 33. In certain embodiments, the extracellular antigen-binding domain of a presently disclosed CAR cross-competes for binding to CD371 (e.g., human CD371) with a reference antibody or an antigen-binding portion thereof comprising the V_{H} and V_{L} sequences of scFv B10. For example, the extracellular antigen-binding domain of a presently disclosed CAR cross-competes for binding to CD371 (e.g., human CD371) with a reference antibody or an antigen-binding portion thereof comprising a V_{H} comprising amino acids having the sequence set forth in SEQ ID NO: 1, and a V_{L} comprising amino acids having the sequence set forth in SEQ ID NO: 2.

In certain embodiments, the extracellular antigen-binding domain binds to the same epitope on CD371 (e.g., human CD371) as the reference antibody or antigen-binding portion thereof. For example, the extracellular antigen-binding domain of a presently disclosed CAR binds to the same epitope on CD371 (e.g., human CD371) as a reference antibody or an antigen-binding portion thereof comprising the V_{H} CDR1, CDR2, and CDR3 sequences and the V_{L} CDR1, CDR2, and CDR3 sequences of, for example, any one of the presently disclosed scFvs (e.g., B10, C3, D6, A11, E4, and E8).

In certain embodiments, the extracellular antigen-binding domain of a presently disclosed CAR binds to the same epitope on EMR2 (e.g., human EMR2) as a reference antibody or an antigen-binding portion thereof comprising the V_{H} and V_{L} sequences of, for example, any one of the presently disclosed scFvs (e.g., B10, C3, D6, A11, E4, and E8).

In certain embodiments, the extracellular antigen-binding domain of a presently disclosed CAR binds to the same epitope on CD371 (e.g., human CD371) as a reference antibody or an antigen-binding fragment thereof comprising the V_{H} CDR1, CDR2, and CDR3 sequences and the V_{L} CDR1, CDR2, and CDR3 sequences of scFv B10. For example, the extracellular antigen-binding domain of a presently disclosed CAR binds to the same epitope on CD371 (e.g., human CD371) as a reference antibody or an antigen-binding fragment thereof comprising a V_{H} CDR1 comprising the amino acid sequence set forth in SEQ ID NO: 28; a V_{H} CDR2 comprising the amino acid sequence set forth in SEQ ID NO: 29; a V_{H} CDR3 comprising the amino acid sequence set forth in SEQ ID NO: 30; a V_{L} CDR1 comprising the amino acid sequence set forth in SEQ ID NO: 31; a V_{L} CDR2 comprising the amino acid sequence set forth in SEQ ID NO: 32; and a V_{L} CDR3 comprising the amino acid sequence set forth in SEQ ID NO: 33. In certain embodiments, the extracellular antigen-binding domain of a presently disclosed CAR binds to the same or substantially the same epitope on CD371 (e.g., human CD371) as a reference antibody or an antigen-binding fragment thereof comprising the V_{H} and V_{L} sequences of scFv B10. For example, the extracellular antigen-binding domain of a presently disclosed CAR binds to the same epitope on CD371 (e.g., human CD371) as a reference antibody or an antigen-binding fragment thereof comprising a V_{H} comprising the amino acid sequence set forth in SEQ ID NO: 1, and a V_{L} comprising the amino acid sequence set forth in SEQ ID NO: 2.

Extracellular antigen-binding domains that cross-compete or compete with the reference antibody or antigen-binding portions thereof for binding to CD371 (e.g., human CD371) can be identified by using routine methods known in the art, including, but not limited to, ELISAs, radioimmunoassays (RIAs), Biacore, flow cytometry, Western blotting, and any other suitable quantitative or qualitative antibody-binding assays. Competition ELISA is described in Morris, “Epitope Mapping of Protein Antigens by Competition ELISA”, The Protein Protocols Handbook (1996), pp 595-600, edited by J. Walker, which is incorporated by reference in its entirety. In certain embodiments, the antibody-binding assay comprises measuring an initial binding of a reference antibody to a CD371 polypeptide, admixing the reference antibody with a test extracellular antigen-binding domain, measuring a second binding of the reference antibody to the CD371 polypeptide in the presence of the test extracellular antigen-binding domain, and comparing the initial binding with the second binding of the reference antibody, wherein a decreased second binding of the reference antibody to the CD371 polypeptide in comparison to the initial binding indicates that the test extracellular antigen-binding domain cross-competes with the reference antibody for binding to CD371, e.g., one that recognizes the same or substantially the same epitope, an overlapping epitope, or an adjacent epitope. In certain embodiments, the reference antibody is labeled, e.g., with a fluorochrome, biotin, or peroxidase. In certain embodiments, the CD371 polypeptide is expressed in cells, e.g., in a flow cytometry test. In certain embodiments, the CD371 polypeptide is immobilized onto a surface, including a Biacore ship (e.g., in a Biacore test), or other media suitable for surface plasmon resonance analysis. The binding of the reference antibody in the presence of a completely irrelevant antibody (that does not bind to CD371) can serve as the control high value. The control low value can be obtained by incubating a labeled reference antibody with an unlabeled reference antibody, where competition and reduced binding of the labeled reference antibody would occur. In certain embodiments, a test extracellular antigen-binding domain that reduces the binding of the reference antibody to a CD371 polypeptide by at least about 20%, at least about 30%, at least about 40%, at least about 50%, at least about 60%, at least about 70%, at least about 80%, at least about 90%, or at least about 95% is considered to be an extracellular antigen-binding domain that cross-competes with the reference antibody for binding to CD371. In certain embodiments, the assays are performed at room temperature.

In certain embodiments, the antibody-binding assay comprises measuring an initial binding of a test extracellular antigen-binding domain to a CD371 polypeptide, admixing the test extracellular antigen-binding domain with a reference antibody, measuring a second binding of the test extracellular antigen-binding domain to the CD371 polypeptide in the presence of the reference antibody, and comparing the initial binding with the second binding of the test extracellular antigen-binding domain, where a decreased second binding of the test extracellular antigen-binding domain to the CD371 polypeptide in comparison to the initial binding indicates that the test extracellular antigen-binding domain cross-competes with the reference antibody for binding to CD371, e.g., one that recognizes the same or substantially the same epitope, an overlapping epitope, or an adjacent epitope. In certain embodiments, the test extracellular antigen-binding domain is labeled, e.g., with a fluorochrome, biotin, or peroxidase. In certain embodiments, the CD371 polypeptide is expressed in cells, e.g., in a flow cytometry test. In certain embodiments, the CD371 polypeptide is immobilized onto a surface, including a Biacore ship (e.g., in a Biacore test), or other media suitable for surface plasmon resonance analysis. The binding of the test extracellular antigen-binding domain in the presence of a completely irrelevant antibody (that does not bind to CD371) can serve as the control high value. The control low value can be obtained by incubating a labeled test extracellular antigen-binding domain with an unlabeled test extracellular antigen-binding domain, where competition and reduced binding of the labeled test extracellular antigen-binding domain would occur. In certain embodiments, a test extracellular antigen-binding domain, whose binding to a CD371 polypeptide is decreased by at least about 20%, at least about 30%, at least about 40%, at least about 50%, at least about 60%, at least about 70%, at least about 80%, at least about 90%, or at least about 95% in the presence of a reference antibody, is considered to be an extracellular antigen-binding domain that cross-competes with the reference antibody for binding to CD371. In certain embodiments, the assays are performed at room temperature.

In certain non-limiting embodiments, the extracellular antigen-binding domain of the presently disclosed CAR comprises a linker connecting the heavy chain variable region and light chain variable region of the extracellular antigen-binding domain. In certain embodiments, the linker comprises the amino acid sequence set forth in SEQ ID NO: 13. In certain embodiments, the linker comprises the amino acid sequence set forth in SEQ ID NO: 14. In certain embodiments, the linker comprises the amino acid sequence set forth in SEQ ID NO: 91. In certain embodiments, the linker comprises the amino acid sequence set forth in SEQ ID NO: 92. In certain embodiments, the linker comprises the amino acid sequence set forth in SEQ ID NO: 93. In certain embodiments, the linker comprises the amino acid sequence set forth in SEQ ID NO: 94.

In certain embodiments, the variable regions within the extracellular antigen-binding domain of the CAR have to be linked one after another such that at the N-terminus of the extracellular antigen-binding domain, a heavy chain variable region (V_{H}) is positioned. In certain embodiments, if the extracellular antigen-binding domain of the CAR is an scFv, the variable regions are positioned from the N- to the C-terminus: V_{H}-V_{L}.

In certain embodiments, the variable regions within the extracellular antigen-binding domain of the CAR have to be linked one after another such that at the N-terminus of the extracellular antigen-binding domain, a light chain variable region (V_{L}) is positioned. In certain embodiments, if the extracellular antigen-binding domain of the CAR is an scFv, the variable regions are positioned from the N- to the C-terminus: V_{L}-V_{H}.

In addition, the extracellular antigen-binding domain can comprise a leader or a signal peptide that directs the nascent protein into the endoplasmic reticulum. Signal peptide or leader can be essential if the CAR is to be glycosylated and anchored in the cell membrane. The signal sequence or leader can be a peptide sequence (about 5, about 10, about 15, about 20, about 25, or about 30 amino acids long) present at the N-terminus of newly synthesized proteins that directs their entry to the secretory pathway. In certain embodiments, the signal peptide is covalently joined to the 5′ terminus of the extracellular antigen-binding domain. In certain embodiments, the signal peptide comprises a CD8 polypeptide, e.g., the CAR comprises a truncated CD8 signal peptide.

5.3.2.2. Transmembrane Domain of a CAR

In certain non-limiting embodiments, the transmembrane domain of the CAR comprises a hydrophobic alpha helix that spans at least a portion of the membrane. Different transmembrane domains result in different receptor stability. After antigen recognition, receptors cluster and a signal are transmitted to the cell. In accordance with the presently disclosed subject matter, the transmembrane domain of the CAR can comprise a native or modified transmembrane domain of CD8 or a fragment thereof, a native or modified transmembrane domain of CD28 or a fragment thereof, a native or modified transmembrane domain of CD3ζ or a fragment thereof, a native or modified transmembrane domain of CD4 or a fragment thereof, a native or modified transmembrane domain of 4-1BB or a fragment thereof, a native or modified transmembrane domain of OX40 or a fragment thereof, a native or modified transmembrane domain of ICOS or a fragment thereof, a native or modified transmembrane domain of CD84 or a fragment thereof, a native or modified transmembrane domain of CD166 or a fragment thereof, a native or modified transmembrane domain of CD8a or a fragment thereof, a native or modified transmembrane domain of CD8b or a fragment thereof, a native or modified transmembrane domain of ICAM-1 or a fragment thereof, a native or modified transmembrane domain of CTLA-4 or a fragment thereof, a native or modified transmembrane domain of CD27 or a fragment thereof, a native or modified transmembrane domain of CD40 or a fragment thereof, NKGD2 or a fragment thereof, or a combination thereof.

In certain embodiments, the transmembrane domain of the CAR comprises a CD8 polypeptide (e.g., a transmembrane domain of CD8 or a fragment thereof). In certain embodiments, the transmembrane domain of the CAR comprises a CD8 polypeptide (e.g., a transmembrane domain of CD8 or a fragment thereof). In certain embodiments, the transmembrane domain of the CAR comprises a CD8 polypeptide (e.g., a transmembrane domain of human CD8 or a fragment thereof). In certain embodiments, the CD8 polypeptide comprises or consists of an amino acid sequence that is at least about 85%, about 90%, about 95%, about 96%, about 97%, about 98%, about 99% or about 100% homologous or identical to the amino acid sequence having a NCBI Reference No: NP_001139345.1 (SEQ ID NO: 64) or a fragments thereof, and/or may optionally comprise up to one or up to two or up to three conservative amino acid substitutions. In certain embodiments, the CD8 polypeptide comprises or consists of an amino acid sequence that is a consecutive portion of SEQ ID NO: 64, which is at least 20, or at least 30, or at least 40, or at least 50, and up to 235 amino acids in length. Alternatively or additionally, in non-limiting various embodiments, the CD8 polypeptide comprises or consists of an amino acid sequence of amino acids 1 to 235, 1 to 50, 50 to 100, 100 to 150, 150 to 200, 137 to 209 or 200 to 235 of SEQ ID NO: 64. In certain embodiments, the transmembrane domain of the CAR comprises a CD8 polypeptide comprising or consisting of amino acids 137 to 209 of SEQ ID NO: 64. SEQ ID NO: 64 is provided below.

[SEQ ID NO: 64]
MALPVTALLLPLALLLHAARPSQFRVSPLDRTWNLGETVELKCQVLLSNPT
SGCSWLFQPRGAAASPTFLLYLSQNKPKAAEGLDTQRFSGKRLGDTFVLTL
SDFRRENEGYYFCSALSNSIMYFSHFVPVFLPAKPTTTPAPRPPTPAPTIA
SQPLSLRPEACRPAAGGAVHTRGLDFACDIYIWAPLAGTCGVLLLSLVITL
YCNHRNRRRVCKCPRPVVKSGDKPSLSARYV

In certain embodiments, the transmembrane domain of the CAR comprises a CD8 polypeptide (e.g., a transmembrane domain of mouse CD8 or a fragment thereof). In certain embodiments, the CD8 polypeptide comprises or consists of an amino acid sequence that is at least about 85%, about 90%, about 95%, about 96%, about 97%, about 98%, about 99% or about 100% homologous or identical to the amino acid sequence having a NCBI Reference No: AAA92533.1 (SEQ ID NO: 65) or a fragment thereof, and/or may optionally comprise up to one or up to two or up to three conservative amino acid substitutions. In certain embodiments, the CD8 polypeptide comprises or consists of an amino acid sequence that is a consecutive portion of SEQ ID NO: 65, which is at least about 20, or at least about 30, or at least about 40, or at least about 50, or at least about 60, or at least about 70, or at least about 100, or at least about 200, and up to 247 amino acids in length. Alternatively or additionally, in non-limiting various embodiments, the CD8 polypeptide comprises or consists of an amino acid sequence of amino acids 1 to 247, 1 to 50, 50 to 100, 100 to 150, 150 to 200, 151 to 219, or 200 to 247 of SEQ ID NO: 65. In certain embodiments, the transmembrane domain of the CAR comprises a CD8 polypeptide comprising or consisting of amino acids 151 to 219 of SEQ ID NO: 65. SEQ ID NO: 65 is provided below.

| [SEQ ID NO: 65] | |
|---|---|
| 1 | MASPLTRFLS LNLLLMGESI ILGSGEAKPQ APELRIFPKK MDAELGQKVD LVCEVLGSVS |
| 61 | QGCSWLFQNS SSKLPQPTFV VYMASSHNKI TWDEKLNSSK LFSAVRDTNN KYVLTLNKFS |
| 121 | KENEGYYFCS VISNSVMYFS SVVPVLQKVN STTTKPVLRT PSPVHPTGTS QPQRPEDCRP |
| 181 | RGSVKGTGLD FACDIYIWAP LAGICVAPLL SLIITLICYH RSRKRVCKCP RPLVRQEGKP |
| 241 | RPSEKIV |

In certain embodiments, the transmembrane domain of a presently disclosed CAR comprises a CD28 polypeptide (e.g., a transmembrane domain of CD28 or a fragment thereof).

In certain embodiments, the transmembrane domain of the CAR comprises a CD28 polypeptide (e.g., a transmembrane domain of human CD28 or a fragment thereof). In certain embodiments, the CD28 polypeptide comprises or consists of an amino acid sequence that is at least about 85%, about 90%, about 95%, about 96%, about 97%, about 98%, about 99% or 100% homologous or identical to the amino acid sequence having a NCBI Reference No: NP_006130 (SEQ ID No: 66) or a fragment thereof, and/or may optionally comprise up to one or up to two or up to three conservative amino acid substitutions. In non-limiting certain embodiments, the CD28 polypeptide comprises or consists of an amino acid sequence that is a consecutive portion of SEQ ID NO: 66 which is at least 20, or at least 30, or at least 40, or at least 50, and up to 220 amino acids in length. Alternatively or additionally, in non-limiting various embodiments, the CD28 polypeptide comprises or consists of an amino acid sequence of amino acids 1 to 220, 1 to 50, 50 to 100, 100 to 150, 150 to 200, 153 to 179, or 200 to 220 of SEQ ID NO: 66. In certain embodiments, the transmembrane domain of the CAR comprises a CD28 polypeptide comprising or consisting of amino acids 153 to 179 of SEQ ID NO: 66. SEQ ID NO: 66 is provided below:

| [SEQ ID NO: 66] | |
|---|---|
| 1 | MLRLLLALNL FPSIQVTGNK ILVKQSPMLV AYDNAVNLSC KYSYNLFSRE FRASLHKGLD |
| 61 | SAVEVCVVYG NYSQQLQVYS KTGFNCDGKL GNESVTFYLQ NLYVNQTDIY FCKIEVMYPP |
| 121 | PYLDNEKSNG TIIHVKGKHL CPSPLFPGPS KPFWVLVVVG GVLACYSLLV TVAFIIFWVR |
| 181 | SKRSRLLHSD YMNMTPRRPG PTRKHYQPYA PPRDFAAYRS |

An exemplary nucleotide sequence encoding amino acid 153 to 179 of SEQ ID NO: 66 is set forth in SEQ ID NO: 67, which is provided below.

[SEQ ID NO: 67]
TTTTGGGTGCTGGTGGTGGTTGGTGGAGTCCTGGCTTGCTATAGCTTGCT
AGTAACAGTGGCCTTTATTATTTTCTGGGTG

In certain embodiments, the transmembrane domain of the CAR comprises a CD28 polypeptide (e.g., a transmembrane domain of mouse CD28 or a fragment thereof). In certain embodiments, the CD28 polypeptide comprises or consists of an amino acid sequence that is at least about 85%, about 90%, about 95%, about 96%, about 97%, about 98%, about 99% or 100% homologous or identical to the amino acid sequence having a NCBI Reference No: NP_031668.3 (SEQ ID No: 68) or a fragment thereof, and/or may optionally comprise up to one or up to two or up to three conservative amino acid substitutions. In non-limiting certain embodiments, the CD28 polypeptide comprises or consists of an amino acid sequence that is a consecutive portion of SEQ ID NO: 66 which is at least 20, or at least 30, or at least 40, or at least 50, and up to 218 amino acids in length. Alternatively or additionally, in non-limiting various embodiments, the CD28 polypeptide comprises or consists of an amino acid sequence of amino acids 1 to 220, 1 to 50, 50 to 100, 100 to 150, 150 to 200, 151 to 177, or 200 to 218 of SEQ ID NO: 68. In certain embodiments, the transmembrane domain of the CAR comprises a CD28 polypeptide comprising or consisting of amino acids 151 to 177 of SEQ ID NO: 68. SEQ ID NO: 68 is provided below:

| [SEQ ID NO: 68] | |
|---|---|
| 1 | MTLRLLFLAL NFFSVQVTEN KILVKQSPLL VVDSNEVSLS CRYSYNLLAK EFRASLYKGV |
| 61 | NSDVEVCVGN GNFTYQPQFR SNAEFNCDGD FDNETVTFRL WNLHVNHTDI YFCKIEFMYP |
| 121 | PPYLDNERSN GTIIHIKEKH LCHTQSSPKL FWALVVVAGV LFCYGLLVTV ALCVIWTNSR |
| 181 | RNRLLQSDYM NMTPRRPGLT RKPYQPYAPA RDFAAYRP |

In certain non-limiting embodiments, the CAR further comprises a spacer region that links the extracellular antigen-binding domain to the transmembrane domain. The spacer region can be flexible enough to allow the antigen binding domain to orient in different directions to facilitate antigen recognition while preserving the activating activity of the CAR.

In certain embodiments, the hinge/spacer region of the CAR comprises a native or modified hinge region of CD8 or a fragment thereof, a native or modified hinge region of CD28 or a fragment thereof, a native or modified hinge region of CD3ζ or a fragment thereof, a native or modified hinge region of CD40 or a fragment thereof, a native or modified hinge region of 4-1BB or a fragment thereof, a native or modified hinge region of OX40 or a fragment thereof, a native or modified hinge region of CD84 or a fragment thereof, a native or modified hinge region of CD166 or a fragment thereof, a native or modified hinge region of CD8a or a fragment thereof, a native or modified hinge region of CD8b or a fragment thereof, a native or modified hinge region of ICOS or a fragment thereof, a native or modified hinge region of ICAM-1 or a fragment thereof, a native or modified hinge region of CTLA-4 or a fragment thereof, a native or modified hinge region of CD27 or a fragment thereof, a native or modified hinge region of CD40 or a fragment thereof, a native or modified hinge region of NKGD2 or a fragment thereof, a synthetic polypeptide (not based on a protein associated with the immune response), or a combination thereof. The hinge/spacer region can be the hinge region from IgG1, or the CH₂CH₃ region of immunoglobulin and portions of CD3, a portion of a CD28 polypeptide (e.g., a portion of SEQ ID NO: 66 or 68), a portion of a CD8 polypeptide (e.g., a portion of SEQ ID NO: 64 or 65), a variation of any of the foregoing which is at least about 80%, at least about 85%, at least about 90%, at least about 95%, or at least about 100% homologous or identical thereto, or a synthetic spacer sequence.

5.3.2.3. Intracellular Signaling Domain of a CAR

In certain embodiments, the CAR comprises an intracellular signaling domain. In certain non-limiting embodiments, the intracellular signaling domain of the CAR comprises a CD3ζ polypeptide. CD3ζ can activate or stimulate a cell (e.g., a cell of the lymphoid lineage, e.g., a T cell). Wild type (“native”) CD3ζ comprises three functional immunoreceptor tyrosine-based activation motifs (ITAMs), three functional basic-rich stretch (BRS) regions (BRS1, BRS2 and BRS3). CD3ζ transmits an activation signal to the cell (e.g., a cell of the lymphoid lineage, e.g., a T cell) after antigen is bound. The intracellular signaling domain of the CD3ζ-chain is the primary transmitter of signals from endogenous TCRs.

In certain embodiments, the intracellular signaling domain of the CAR comprises a native CD3ζ. In certain embodiments, the CD3ζ polypeptide comprises or consists of an amino acid sequence that is at least about 85%, about 90%, about 95%, about 96%, about 97%, about 98%, about 99% or about 100% homologous or identical to the amino acid sequence having a NCBI Reference No: NP_932170 (SEQ ID NO: 69) or a fragment thereof, and/or may optionally comprise up to one or up to two or up to three conservative amino acid substitutions. In certain non-limiting embodiments, the CD3ζ polypeptide comprises or consists of an amino acid sequence that is a consecutive portion of SEQ ID NO: 69, which is at least 20, or at least 30, or at least 40, or at least 50, and up to 164 amino acids in length. Alternatively or additionally, in non-limiting various embodiments, the CD3ζ polypeptide comprises or consists of an amino acid sequence of amino acids 1 to 164, 1 to 50, 50 to 100, 52 to 164, 100 to 150, or 150 to 164 of SEQ ID NO: 69. In certain embodiments, the intracellular signaling domain of the CAR comprises a CD3ζ polypeptide comprising or consisting of amino acids 52 to 164 of SEQ ID NO: 69. SEQ ID NO: 69 is provided below:

| [SEQ ID NO: 69] | |
|---|---|
| 1 | MKWKALFTAA ILQAQLPITE AQSFGLLDPK LCYLLDGILF IYGVILTALF LRVKFSRSAD |
| 61 | APAYQQGQNQ LYNELNLGRR EEYDVLDKRR GRDPEMGGKP QRRKNPQEGL YNELQKDKMA |
| 121 | EAYSEIGMKG ERRRGKGHDG LYQGLSTATK DTYDALHMQA LPPR |

In certain embodiments, the intracellular signaling domain of the CAR comprises a CD3 polypeptide comprising or consisting of the amino acid sequence set forth in SEQ ID NO: 89. SEQ ID NO: 89 is provided below.

[SEQ ID NO: 89]
RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR

An exemplary nucleotide sequence encoding the amino acid sequence of SEQ ID NO: 89 is set forth in SEQ ID NO: 90, which is as provided below.

[SEQ ID NO: 90]
AGAGTGAAGTTCAGCAGGAGCGCAGACGCCCCCGCGTACCAGCAGGGCCA
GAACCAGCTCTATAACGAGCTCAATCTAGGACGAAGAGAGGAGTACGATG
TTTTGGACAAGAGACGTGGCCGGGACCCTGAGATGGGGGGAAAGCCGAGA
AGGAAGAACCCTCAGGAAGGCCTGTACAATGAACTGCAGAAAGATAAGAT
GGCGGAGGCCTACAGTGAGATTGGGATGAAAGGCGAGCGCCGGAGGGGCA
AGGGGCACGATGGCCTTTACCAGGGTCTCAGTACAGCCACCAAGGACACC
TACGACGCCCTTCACATGCAGGCCCTGCCCCCTCGC

In certain non-limiting embodiments, the intracellular signaling domain of the CAR further comprises at least a co-stimulatory signaling region. In certain embodiments, the co-stimulatory signaling region comprises at least one co-stimulatory molecule or a fragment thereof. In certain embodiments, the co-stimulatory signaling region comprises an intracellular domain of at least one co-stimulatory molecule or a fragment thereof.

As used herein, a “co-stimulatory molecule” refers to a cell surface molecule other than antigen receptor or its ligand that can provide an efficient response of lymphocytes to an antigen. In certain embodiments, a co-stimulatory molecule can provide optimal lymphocyte activation. Non-limiting examples of co-stimulatory molecules include CD28, 4-1BB, OX40, ICOS, DAP-10, CD27, CD40, NKGD2, CD2, FN14, HVEM, LTBR, CD28H, TNFR1, TNFR2, BAFF-R, BCMA, TACI, TROY, RANK, CD40, CD27, CD30, EDAR, XEDAR, GITR, DR6, and NGFR, and combinations thereof. The co-stimulatory molecule can bind to a co-stimulatory ligand, which is a protein expressed on cell surface that upon binding to its receptor produces a co-stimulatory response, i.e., an intracellular response that effects the stimulation provided when an antigen-recognizing receptor (e.g., a chimeric antigen receptor (CAR)) binds to its target antigen. As one example, a 4-1BB ligand (i.e., 4-1BBL) may bind to 4-1BB for providing an intracellular signal that in combination with a CAR signal induces an effector cell function of the CAR⁺ T cell.

In certain embodiments, the intracellular signaling domain of the CAR comprises a co-stimulatory signaling region that comprises a CD28 polypeptide, e.g., an intracellular domain of CD28 or a fragment thereof. The CD28 polypeptide can comprise or have an amino acid sequence that is at least about 80%, at least about 85%, at least about 90%, at least about 95%, at least about 96%, at least about 97%, at least about 98%, or at least about 99%, at least about 100% homologous or identical to the amino acid sequence set forth in SEQ ID NO: 66 or a fragment thereof, and/or may optionally comprise up to one or up to two or up to three conservative amino acid substitutions. In non-limiting certain embodiments, the CD28 polypeptide comprises or consists of an amino acid sequence that is a consecutive portion of SEQ ID NO: 66, which is at least 20, or at least 30, or at least 40, or at least 50, and up to 220 amino acids in length. Alternatively or additionally, in non-limiting various embodiments, the CD28 polypeptide comprises or consists of an amino acid sequence of amino acids 1 to 220, 1 to 50, 50 to 100, 100 to 150, 114 to 220, 150 to 200, 180 to 220, or 200 to 220 of SEQ ID NO: 66. In certain embodiments, the intracellular signaling domain of the CAR comprises a co-stimulatory signaling region that comprises a CD28 polypeptide comprising or consisting of an amino acid sequence of amino acids 180 to 220 of SEQ ID NO: 66.

An exemplary nucleic acid sequence encoding amino acids 180 to 220 of SEQ ID NO: 66 is set forth in SEQ ID NO: 70, which is provided below.

[SEQ ID NO: 70]
AGGAGTAAGAGGAGCAGGCTCCTGCACAGTGACTACATGAACATGACTCC
CCGCCGCCCCGGGCCCACCCGCAAGCATTACCAGCCCTATGCCCCACCAC
GCGACTTCGCAGCCTATCGCTCC

In certain embodiments, the CD28 polypeptide comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence set forth in SEQ ID NO: 68 or a fragment thereof, and/or may optionally comprise up to one or up to two or up to three conservative amino acid substitutions. In non-limiting certain embodiments, the CD28 polypeptide comprises or consists of an amino acid sequence that is a consecutive portion of SEQ ID NO: 68 which is at least about 20, or at least about 30, or at least about 40, or at least about 50, and up to 218 amino acids in length. Alternatively or additionally, in non-limiting various embodiments, the CD28 polypeptide comprises or consists of an amino acid sequence of amino acids 1 to 218, 1 to 50, 50 to 100, 100 to 150, 150 to 218, 178 to 218, or 200 to 218 of SEQ ID NO: 68. In certain embodiments, the co-stimulatory signaling region of a presently disclosed CAR comprises a CD28 polypeptide that comprises or consists of the amino acids 178 to 218 of SEQ ID NO: 68.

In certain embodiments, the intracellular signaling domain of the CAR comprises a co-stimulatory signaling region that comprises a 4-1BB polypeptide, e.g., an intracellular domain of 4-1BB or a fragment thereof. The 4-1BB polypeptide can comprise or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence having a NCBI Ref. No. NP_001552 (SEQ ID NO: 71) or a fragment thereof, and/or may optionally comprise up to one or up to two or up to three conservative amino acid substitutions. In non-limiting certain embodiments, the 4-1BB polypeptide comprises or consists of an amino acid sequence that is a consecutive portion of SEQ ID NO: 71, which is at least 20, or at least 30, or at least 40, or at least 50, or at least 100, or at least 150, or at least 150, and up to 255 amino acids in length. Alternatively or additionally, in non-limiting various embodiments, the 4-1BB polypeptide comprises or consists of an amino acid sequence of amino acids 1 to 255, 1 to 50, 50 to 100, 100 to 150, 150 to 200, or 200 to 255 of SEQ ID NO: 71. In certain embodiments, the intracellular signaling domain of the CAR comprises a co-stimulatory signaling region that comprises a 4-1BB polypeptide comprising or consisting of an amino acid sequence of amino acids 214 to 255 of SEQ ID NO: 71. SEQ ID NO: 71 is provided below.

| [SEQ ID NO: 71] | |
|---|---|
| 1 | MGNSCYNIVA TLLLVLNFER TRSLQDPCSN CPAGTFCDNN RNQICSPCPP NSFSSAGGQR |
| 61 | TCDICRQCKG VFRTRKECSS TSNAECDCTP GFHCLGAGCS MCEQDCKQGQ ELTKKGCKDC |
| 121 | CFGTFNDQKR GICRPWTNCS LDGKSVLVNG TKERDVVCGP SPADLSPGAS SVTPPAPARE |
| 181 | PGHSPQIISF FLALTSTALL FLLFFLTLRF SVVKRGRKKL LYIFKQPFMR PVQTTQEEDG |
| 241 | CSCRFPEEEE GGCEL |

An exemplary nucleic acid sequence encoding amino acids 214 to 255 of SEQ ID NO: 71 is set forth in SEQ ID NO: 72, which is provided below.

[SEQ ID NO: 72]
AAACGGGGCAGAAAGAAACTCCTGTATATATTCAAACAACCATTTATGAG
ACCAGTACAAACTACTCAAGAGGAAGATGGCTGTAGCTGCCGATTTCCAG
AAGAAGAAGAAGGAGGATGTGAACTG

In certain embodiments, the intracellular signaling domain of the CAR comprises a co-stimulatory signaling region that comprises intracellular domains of two or more co-stimulatory molecules or portions thereof, e.g., an intracellular domain of CD28 or a fragment thereof and an intracellular domain of 4-1BB or a fragment thereof, or an intracellular domain of CD28 or a fragment thereof and an intracellular domain of OX40 or a fragment thereof.

5.3.2.4. Exemplified CARs

In certain embodiments, the CAR is a CD371-targeted CAR. In certain embodiments, the CAR comprises (a) an extracellular antigen-binding domain comprising (i) a V_{H} that comprises a CDR1 comprising the amino acid sequence set forth in SEQ ID NO: 28, a CDR2 comprising the amino acid sequence set forth in SEQ ID NO: 29, and a V_{H} CDR3 comprising the amino acid sequence set forth in SEQ ID NO: 30, and (ii) a V_{L} that comprises a CDR1 comprising the amino acid sequence set forth in SEQ ID NO: 31, a CDR2 comprising the amino acid sequence set forth in SEQ ID NO: 32, and a V_{L} CDR3 comprising the amino acid sequence set forth in SEQ ID NO: 33; (b) a transmembrane domain comprising a CD28 polypeptide (e.g., a transmembrane domain of human CD28 or a fragment thereof), and (c) an intracellular signaling domain comprising (i) a CD3ζ polypeptide, and (ii) a co-stimulatory signaling region comprising a CD28 polypeptide (e.g., an intracellular domain of human CD28 or a fragment thereof). In certain embodiments, the V_{H} and V_{L} are linked via a linker comprising or consisting of the amino acid sequence set forth in SEQ ID NO: 14. In certain embodiments, the V_{H} and V_{L} are positioned from the N- to the C-terminus: V_{L}-V_{H}. In certain embodiments, the CAR is designed as “Et.B10L3H_MT_h28Z”. In certain embodiments, the CAR comprises the amino acid sequence set forth in SEQ ID NO: 73, which is provided below.

[SEQ ID NO: 73]
MLLLVTSLLLCELPHPAFLLIPRKVCNGIGIGEFKDSLSINATNIKHFKN
CTSISGDLHILPVAFRGDSFTHTPPLDPQELDILKTVKEITGFLLIQAWP
ENRTDLHAFENLEIIRGRTKQHGQFSLAVVSLNITSLGLRSLKEISDGDV
IISGNKNLCYANTINWKKLFGTSGQKTKIISNRGENSCKATGQVCHALCS
PEGCWGPEPRDCVSCRNVSRGRECVDKCNLLEGEPREFVENSECIQCHPE
CLPQAMNITCTGRGPDNCIQCAHYIDGPHCVKTCPAGVMGENNTLVWKYA
DAGHVCHLCHPNCTYGCTGPGLEGCPTNGPKIPSIATGMVGALLLLLVVA
LGIGLFMGSGEGRGSLLTCGDVEENPGPMALPVTALLLPLALLLHADIVM
TQSPDSLAVSLGERATINCKSSQSVLDSYNNENNLAWYQQKPGQPPKLLI
YWASTRESGVPDRFSGSGSGTDFTLTISSLQAEDVAVYYCQQYTSEPITF
GQGTKVEIKGGGGSGGGGSGGGGSEVQLLESGGGLVQPGGSLRLSCAASG
FTFSDYQMSWVRQAPGKGLEWVSGIQGGGGSTYYADSVKGRFTISRDNSK
NTLYLQMNSLRAEDTAVYYCAREMWRGDYYSGMDVWGQGTTVTVSSEQKL
ISEEDLAAAIEVMYPPPYLDNEKSNGTIIHVKGKHLCPSPLFPGPSKPFW
VLVVVGGVLACYSLLVTVAFIIFWVRSKRSRLLHSDYMNMTPRRPGPTRK
HYQPYAPPRDFAAYRSRVKFSRSADAPAYQQGQNQLYNELNLGRREEYDV
LDKRRGRDPEMGGKPRRKNPQEGLYNELQKDKMAEAYSEIGMKGERRRGK
GHDGLYQGLSTATKDTYDALHMQALPPR

An exemplary nucleic acid sequence the amino acid sequence of SEQ ID NO: 73 is set forth in SEQ ID NO: 74, which is provided below.

[SEQ ID NO: 74]
ATGCTTCTCCTGGTGACAAGCCTTCTGCTCTGTGAGTTACCACACCCAGCA
TTCCTCCTGATCCCACGCAAAGTGTGTAACGGAATAGGTATTGGTGAATTT
AAAGACTCACTCTCCATAAATGCTACGAATATTAAACACTTCAAAAACTGC
ACCTCCATCAGTGGCGATCTCCACATCCTGCCGGTGGCATTTAGGGGTGAC
TCCTTCACACATACTCCTCCTCTGGACCCACAGGAACTGGATATTCTGAAA
ACCGTAAAGGAAATCACAGGGTTTTTGCTGATTCAGGCTTGGCCTGAAAAC
AGGACGGACCTCCATGCCTTTGAGAACCTAGAAATCATACGCGGCAGGACC
AAGCAACATGGTCAGTTTTCTCTTGCAGTCGTCAGCCTGAACATAACATCC
TTGGGATTACGCTCCCTCAAGGAGATAAGTGATGGAGATGTGATAATTTCA
GGAAACAAAAATTTGTGCTATGCAAATACAATAAACTGGAAAAAACTGTTT
GGGACCTCCGGTCAGAAAACCAAAATTATAAGCAACAGAGGTGAAAACAGC
TGCAAGGCCACAGGCCAGGTCTGCCATGCCTTGTGCTCCCCCGAGGGCTGC
TGGGGCCCGGAGCCCAGGGACTGCGTCTCTTGCCGGAATGTCAGCCGAGGC
AGGGAATGCGTGGACAAGTGCAACCTTCTGGAGGGTGAGCCAAGGGAGTTT
GTGGAGAACTCTGAGTGCATACAGTGCCACCCAGAGTGCCTGCCTCAGGCC
ATGAACATCACCTGCACAGGACGGGGACCAGACAACTGTATCCAGTGTGCC
CACTACATTGACGGCCCCCACTGCGTCAAGACCTGCCCGGCAGGAGTCATG
GGAGAAAACAACACCCTGGTCTGGAAGTACGCAGACGCCGGCCATGTGTGC
CACCTGTGCCATCCAAACTGCACCTACGGATGCACTGGGCCAGGTCTTGAA
GGCTGTCCAACGAATGGGCCTAAGATCCCGTCCATCGCCACTGGGATGGTG
GGGGCCCTCCTCTTGCTGCTGGTGGTGGCCCTGGGGATCGGCCTCTTCATG
GGTTCCGGTGAGGGACGGGGGTCACTGCTCACCTGCGGAGATGTAGAAGAG
AATCCCGGTCCCATGGCTCTCCCAGTGACTGCCCTACTGCTTCCCCTAGCG
CTTCTCCTGCATGCAGACATCGTGATGACCCAGTCTCCAGACTCCCTGGCT
GTGTCTCTGGGCGAGCGTGCCACCATCAACTGCAAGTCCAGCCAGAGTGTT
TTAGACAGCTATAACAATGAGAACAATTTAGCTTGGTATCAGCAGAAACCA
GGACAGCCTCCTAAGCTGCTCATTTACTGGGCATCTACCCGGGAATCCGGG
GTCCCTGACCGATTCAGTGGCAGCGGGTCTGGGACAGATTTCACTCTCACC
ATCAGCAGCCTGCAGGCTGAAGATGTGGCAGTTTATTACTGTCAGCAATAT
ACCAGCGAACCTATCACGTTCGGCCAAGGTACCAAGGTGGAAATCAAAGGC
GGGGGTGGTAGTGGCGGTGGAGGTAGCGGAGGTGGCGGGTCTGAGGTGCAG
CTGTTGGAGTCTGGGGGAGGCTTGGTACAGCCTGGGGGGTCCCTGCGACTC
TCCTGTGCAGCCTCTGGATTCACCTTTAGCGACTATCAGATGAGCTGGGTC
CGCCAGGCTCCAGGGAAGGGGCTGGAGTGGGTGTCAGGCATTCAGGGTGGC
GGTGGTAGCACATATTACGCAGACTCCGTGAAGGGCCGGTTCACCATCTCC
CGTGACAATTCCAAGAACACGCTGTATCTGCAAATGAACAGCCTGCGTGCC
GAGGACACGGCTGTGTATTACTGTGCGAGAGAGATGTGGCGTGGGGACTAC
TACTCCGGTATGGACGTCTGGGGCCAGGGGACCACGGTCACCGTCTCCTCA
GAACAGAAACTGATCTCTGAAGAAGACCTGGCGGCCGCAATTGAAGTTATG
TATCCTCCTCCTTACCTAGACAATGAGAAGAGCAATGGAACCATTATCCAT
GTGAAAGGGAAACACCTTTGTCCAAGTCCCCTATTTCCCGGACCTTCTAAG
CCCTTTTGGGTGCTGGTGGTGGTTGGTGGAGTCCTGGCTTGCTATAGCTTG
CTAGTAACAGTGGCCTTTATTATTTTCTGGGTGAGGAGTAAGAGGAGCAGG
CTCCTGCACAGTGACTACATGAACATGACTCCCCGCCGCCCCGGGCCCACC
CGCAAGCATTACCAGCCCTATGCCCCACCACGCGACTTCGCAGCCTATCGC
TCCAGAGTGAAGTTCAGCAGGAGCGCAGACGCCCCCGCGTACCAGCAGGGC
CAGAACCAGCTCTATAACGAGCTCAATCTAGGACGAAGAGAGGAGTACGAT
GTTTTGGACAAGAGACGTGGCCGGGACCCTGAGATGGGGGGAAAGCCGAGA
AGGAAGAACCCTCAGGAAGGCCTGTACAATGAACTGCAGAAAGATAAGATG
GCGGAGGCCTACAGTGAGATTGGGATGAAAGGCGAGCGCCGGAGGGGCAAG
GGGCACGATGGCCTTTACCAGGGTCTCAGTACAGCCACCAAGGACACCTAC
GACGCCCTTCACATGCAGGCCCTGCCCCCTCGCTAG

In certain embodiments, the CAR is a CD371-targeted CAR. In certain embodiments, the CAR comprises (a) an extracellular antigen-binding domain comprising (i) a V_{H} that comprises a CDR1 comprising the amino acid sequence set forth in SEQ ID NO: 28, a CDR2 comprising the amino acid sequence set forth in SEQ ID NO: 29, and a V_{H} CDR3 comprising the amino acid sequence set forth in SEQ ID NO: 30, and (ii) a V_{L} that comprises a CDR1 comprising the amino acid sequence set forth in SEQ ID NO: 31, a CDR2 comprising the amino acid sequence set forth in SEQ ID NO: 32, and a V_{L} CDR3 comprising the amino acid sequence set forth in SEQ ID NO: 33; (b) a transmembrane domain comprising a CD28 polypeptide (e.g., a transmembrane domain of human CD28 or a portion thereof), and (c) an intracellular signaling domain comprising (i) a CD3ζ polypeptide, and (ii) a co-stimulatory signaling region comprising a 4-1BB polypeptide (e.g., an intracellular domain of human 4-1BB or a portion thereof). In certain embodiments, the V_{H} and V_{L} are linked via a linker comprising or consisting of the amino acid sequence set forth in SEQ ID NO: 13. In certain embodiments, the V_{H} and V_{L} are positioned from the N- to the C-terminus: V_{L}-V_{H}. In certain embodiments, the CAR is designed as “Et.B10L4H_MT_hBBZ”. In certain embodiments, the CAR comprises the amino acid sequence set forth in SEQ ID NO: 75, which is provided below.

[SEQ ID NO: 75]
MLLLVTSLLLCELPHPAFLLIPRKVCNGIGIGEFKDSLSINATNIKHFKN
CTSISGDLHILPVAFRGDSFTHTPPLDPQELDILKTVKEITGELLIQAWP
ENRTDLHAFENLEIIRGRTKQHGQFSLAVVSLNITSLGLRSLKEISDGDV
IISGNKNLCYANTINWKKLFGTSGQKTKIISNRGENSCKATGQVCHALCS
PEGCWGPEPRDCVSCRNVSRGRECVDKCNLLEGEPREFVENSECIQCHPE
CLPQAMNITCTGRGPDNCIQCAHYIDGPHCVKTCPAGVMGENNTLVWKYA
DAGHVCHLCHPNCTYGCTGPGLEGCPTNGPKIPSIATGMVGALLLLLVVA
LGIGLFMGSGEGRGSLLTCGDVEENPGPMALPVTALLLPLALLLHADIVM
TQSPDSLAVSLGERATINCKSSQSVLDSYNNENNLAWYQQKPGQPPKLLI
YWASTRESGVPDRFSGSGSGTDFTLTISSLQAEDVAVYYCQQYTSEPTTF
GQGTKVEIKGGGGSGGGGSGGGSGGGGSEVQLLESGGGLVQPGGSLRLSC
AASGFTFSDYQMSWVRQAPGKGLEWVSGIQGGGGSTYYADSVKGRFTISR
DNSKNTLYLQMNSLRAEDTAVYYCAREMWRGDYYSGMDVWGQGTTVTVSS
EQKLISEEDLAAAIEVMYPPPYLDNEKSNGTIIHVKGKHLCPSPLFPGPS
KPFWVLVVVGGVLACYSLLVTVAFIIFWVKRGRKKLLYIFKQPFMRPVQT
TQEEDGCSCRFPEEEEGGCELRVKFSRSADAPAYQQGQNQLYNELNLGRR
EEYDVLDKRRGRDPEMGGKPRRKNPQEGLYNELQKDKMAEAYSEIGMKGE
RRRGKGHDGLYQGLSTATKDTYDALHMQALPPR

An exemplary nucleic acid sequence the amino acid sequence of SEQ ID NO: 75 is set forth in SEQ ID NO: 76, which is provided below.

[SEQ ID NO: 76]
ATGCTTCTCCTGGTGACAAGCCTTCTGCTCTGTGAGTTACCACACCCAGCA
TTCCTCCTGATCCCACGCAAAGTGTGTAACGGAATAGGTATTGGTGAATTT
AAAGACTCACTCTCCATAAATGCTACGAATATTAAACACTTCAAAAACTGC
ACCTCCATCAGTGGCGATCTCCACATCCTGCCGGTGGCATTTAGGGGTGAC
TCCTTCACACATACTCCTCCTCTGGACCCACAGGAACTGGATATTCTGAAA
ACCGTAAAGGAAATCACAGGGTTTTTGCTGATTCAGGCTTGGCCTGAAAAC
AGGACGGACCTCCATGCCTTTGAGAACCTAGAAATCATACGCGGCAGGACC
AAGCAACATGGTCAGTTTTCTCTTGCAGTCGTCAGCCTGAACATAACATCC
TTGGGATTACGCTCCCTCAAGGAGATAAGTGATGGAGATGTGATAATTTCA
GGAAACAAAAATTTGTGCTATGCAAATACAATAAACTGGAAAAAACTGTTT
GGGACCTCCGGTCAGAAAACCAAAATTATAAGCAACAGAGGTGAAAACAGC
TGCAAGGCCACAGGCCAGGTCTGCCATGCCTTGTGCTCCCCCGAGGGCTGC
TGGGGCCCGGAGCCCAGGGACTGCGTCTCTTGCCGGAATGTCAGCCGAGGC
AGGGAATGCGTGGACAAGTGCAACCTTCTGGAGGGTGAGCCAAGGGAGTTT
GTGGAGAACTCTGAGTGCATACAGTGCCACCCAGAGTGCCTGCCTCAGGCC
ATGAACATCACCTGCACAGGACGGGGACCAGACAACTGTATCCAGTGTGCC
CACTACATTGACGGCCCCCACTGCGTCAAGACCTGCCCGGCAGGAGTCATG
GGAGAAAACAACACCCTGGTCTGGAAGTACGCAGACGCCGGCCATGTGTGC
CACCTGTGCCATCCAAACTGCACCTACGGATGCACTGGGCCAGGTCTTGAA
GGCTGTCCAACGAATGGGCCTAAGATCCCGTCCATCGCCACTGGGATGGTG
GGGGCCCTCCTCTTGCTGCTGGTGGTGGCCCTGGGGATCGGCCTCTTCATG
GGTTCCGGTGAGGGACGGGGGTCACTGCTCACCTGCGGAGATGTAGAAGAG
AATCCCGGTCCCATGGCTCTCCCAGTGACTGCCCTACTGCTTCCCCTAGCG
CTTCTCCTGCATGCAGACATCGTGATGACCCAGTCTCCAGACTCCCTGGCT
GTGTCTCTGGGCGAGCGTGCCACCATCAACTGCAAGTCCAGCCAGAGTGTT
TTAGACAGCTATAACAATGAGAACAATTTAGCTTGGTATCAGCAGAAACCA
GGACAGCCTCCTAAGCTGCTCATTTACTGGGCATCTACCCGGGAATCCGGG
GTCCCTGACCGATTCAGTGGCAGCGGGTCTGGGACAGATTTCACTCTCACC
ATCAGCAGCCTGCAGGCTGAAGATGTGGCAGTTTATTACTGTCAGCAATAT
ACCAGCGAACCTATCACGTTCGGCCAAGGTACCAAGGTGGAAATCAAAGGT
GGTGGTGGTTCAGGTGGTGGTGGTTCTGGCGGCGGCTCCGGTGGTGGTGGA
TCCGAGGTGCAGCTGTTGGAGTCTGGGGGAGGCTTGGTACAGCCTGGGGGG
TCCCTGCGACTCTCCTGTGCAGCCTCTGGATTCACCTTTAGCGACTATCAG
ATGAGCTGGGTCCGCCAGGCTCCAGGGAAGGGGCTGGAGTGGGTGTCAGGC
ATTCAGGGTGGCGGTGGTAGCACATATTACGCAGACTCCGTGAAGGGCCGG
TTCACCATCTCCCGTGACAATTCCAAGAACACGCTGTATCTGCAAATGAAC
AGCCTGCGTGCCGAGGACACGGCTGTGTATTACTGTGCGAGAGAGATGTGG
CGTGGGGACTACTACTCCGGTATGGACGTCTGGGGCCAGGGGACCACGGTC
ACCGTCTCCTCAGAACAGAAACTGATCTCTGAAGAAGACCTGGCGGCCGCA
ATTGAAGTTATGTATCCTCCTCCTTACCTAGACAATGAGAAGAGCAATGGA
ACCATTATCCATGTGAAAGGGAAACACCTTTGTCCAAGTCCCCTATTTCCC
GGACCTTCTAAGCCCTTTTGGGTGCTGGTGGTGGTTGGTGGAGTCCTGGCT
TGCTATAGCTTGCTAGTAACAGTGGCCTTTATTATTTTCTGGGTGAAACGG
GGCAGAAAGAAACTCCTGTATATATTCAAACAACCATTTATGAGACCAGTA
CAAACTACTCAAGAGGAAGATGGCTGTAGCTGCCGATTTCCAGAAGAAGAA
GAAGGAGGATGTGAACTGAGAGTGAAGTTCAGCAGGAGCGCAGACGCCCCC
GCGTACCAGCAGGGCCAGAACCAGCTCTATAACGAGCTCAATCTAGGACGA
AGAGAGGAGTACGATGTTTTGGACAAGAGACGTGGCCGGGACCCTGAGATG
GGGGGAAAGCCGAGAAGGAAGAACCCTCAGGAAGGCCTGTACAATGAACTG
CAGAAAGATAAGATGGCGGAGGCCTACAGTGAGATTGGGATGAAAGGCGAG
CGCCGGAGGGGCAAGGGGCACGATGGCCTTTACCAGGGTCTCAGTACAGCC
ACCAAGGACACCTACGACGCCCTTCACATGCAGGCCCTGCCCCCTCGCTAG

In certain embodiments, the CAR is a CD371-targeted CAR. In certain embodiments, the CAR comprises (a) an extracellular antigen-binding domain comprising (i) a V_{H} that comprises a CDR1 comprising the amino acid sequence set forth in SEQ ID NO: 28, a CDR2 comprising the amino acid sequence set forth in SEQ ID NO: 29, and a V_{H} CDR3 comprising the amino acid sequence set forth in SEQ ID NO: 30, and (ii) a V_{L} that comprises a CDR1 comprising the amino acid sequence set forth in SEQ ID NO: 31, a CDR2 comprising the amino acid sequence set forth in SEQ ID NO: 32, and a V_{L} CDR3 comprising the amino acid sequence set forth in SEQ ID NO: 33; (b) a transmembrane domain comprising a CD28 polypeptide (e.g., a transmembrane domain of human CD28 or a portion thereof), and (c) an intracellular signaling domain comprising (i) a CD3ζ polypeptide, and (ii) a co-stimulatory signaling region comprising a CD28 polypeptide (e.g., an intracellular domain of human CD28 or a portion thereof). In certain embodiments, the V_{H} and V_{L} are linked via a linker comprising or consisting of the amino acid sequence set forth in SEQ ID NO: 13. In certain embodiments, the V_{H} and V_{L} are positioned from the N- to the C-terminus: V_{L}-V_{H}. In certain embodiments, the CAR is designed as “Et.B10L4H_MT_h28Z”. In certain embodiments, the CAR comprises the amino acid sequence set forth in SEQ ID NO: 77, which is provided below.

[SEQ ID NO: 77]
MLLLVTSLLLCELPHPAFLLIPRKVCNGIGIGEFKDSLSINATNIKHFKN
CTSISGDLHILPVAFRGDSFTHTPPLDPQELDILKTVKEITGFLLIQAWP
ENRTDLHAFENLEIIRGRTKQHGQFSLAVVSLNITSLGLRSLKEISDGDV
IISGNKNLCYANTINWKKLFGTSGQKTKIISNRGENSCKATGQVCHALCS
PEGCWGPEPRDCVSCRNVSRGRECVDKCNLLEGEPREFVENSECIQCHPE
CLPQAMNITCTGRGPDNCIQCAHYIDGPHCVKTCPAGVMGENNTLVWKYA
DAGHVCHLCHPNCTYGCTGPGLEGCPTNGPKIPSIATGMVGALLLLLVVA
LGIGLFMGSGEGRGSLLTCGDVEENPGPMALPVTALLLPLALLLHADIVM
TQSPDSLAVSLGERATINCKSSQSVLDSYNNENNLAWYQQKPGQPPKLLI
YWASTRESGVPDRFSGSGSGTDFTLTISSLQAEDVAVYYCQQYTSEPITF
GQGTKVEIKGGGGSGGGGSGGGSGGGGSEVQLLESGGGLVQPGGSLRLSC
AASGFTFSDYQMSWVRQAPGKGLEWVSGIQGGGGSTYYADSVKGRFTISR
DNSKNTLYLQMNSLRAEDTAVYYCAREMWRGDYYSGMDVWGQGTTVTVSS
EQKLISEEDLAAAIEVMYPPPYLDNEKSNGTIIHVKGKHLCPSPLFPGPS
KPFWVLVVVGGVLACYSLLVTVAFIIFWVRSKRSRLLHSDYMNMTPRRPG
PTRKHYQPYAPPRDFAAYRSRVKFSRSADAPAYQQGQNQLYNELNLGRRE
EYDVLDKRRGRDPEMGGKPRRKNPQEGLYNELQKDKMAEAYSEIGMKGER
RRGKGHDGLYQGLSTATKDTYDALHMQALPPR

An exemplary nucleic acid sequence the amino acid sequence of SEQ ID NO: 77 is set forth in SEQ ID NO: 78, which is provided below.

[SEQ ID NO: 78]
ATGCTTCTCCTGGTGACAAGCCTTCTGCTCTGTGAGTTACCACACCCAGCA
TTCCTCCTGATCCCACGCAAAGTGTGTAACGGAATAGGTATTGGTGAATTT
AAAGACTCACTCTCCATAAATGCTACGAATATTAAACACTTCAAAAACTGC
ACCTCCATCAGTGGCGATCTCCACATCCTGCCGGTGGCATTTAGGGGTGAC
TCCTTCACACATACTCCTCCTCTGGACCCACAGGAACTGGATATTCTGAAA
ACCGTAAAGGAAATCACAGGGTTTTTGCTGATTCAGGCTTGGCCTGAAAAC
AGGACGGACCTCCATGCCTTTGAGAACCTAGAAATCATACGCGGCAGGACC
AAGCAACATGGTCAGTTTTCTCTTGCAGTCGTCAGCCTGAACATAACATCC
TTGGGATTACGCTCCCTCAAGGAGATAAGTGATGGAGATGTGATAATTTCA
GGAAACAAAAATTTGTGCTATGCAAATACAATAAACTGGAAAAAACTGTTT
GGGACCTCCGGTCAGAAAACCAAAATTATAAGCAACAGAGGTGAAAACAGC
TGCAAGGCCACAGGCCAGGTCTGCCATGCCTTGTGCTCCCCCGAGGGCTGC
TGGGGCCCGGAGCCCAGGGACTGCGTCTCTTGCCGGAATGTCAGCCGAGGC
AGGGAATGCGTGGACAAGTGCAACCTTCTGGAGGGTGAGCCAAGGGAGTTT
GTGGAGAACTCTGAGTGCATACAGTGCCACCCAGAGTGCCTGCCTCAGGCC
ATGAACATCACCTGCACAGGACGGGGACCAGACAACTGTATCCAGTGTGCC
CACTACATTGACGGCCCCCACTGCGTCAAGACCTGCCCGGCAGGAGTCATG
GGAGAAAACAACACCCTGGTCTGGAAGTACGCAGACGCCGGCCATGTGTGC
CACCTGTGCCATCCAAACTGCACCTACGGATGCACTGGGCCAGGTCTTGAA
GGCTGTCCAACGAATGGGCCTAAGATCCCGTCCATCGCCACTGGGATGGTG
GGGGCCCTCCTCTTGCTGCTGGTGGTGGCCCTGGGGATCGGCCTCTTCATG
GGTTCCGGTGAGGGACGGGGGTCACTGCTCACCTGCGGAGATGTAGAAGAG
AATCCCGGTCCCATGGCTCTCCCAGTGACTGCCCTACTGCTTCCCCTAGCG
CTTCTCCTGCATGCAGACATCGTGATGACCCAGTCTCCAGACTCCCTGGCT
GTGTCTCTGGGCGAGCGTGCCACCATCAACTGCAAGTCCAGCCAGAGTGTT
TTAGACAGCTATAACAATGAGAACAATTTAGCTTGGTATCAGCAGAAACCA
GGACAGCCTCCTAAGCTGCTCATTTACTGGGCATCTACCCGGGAATCCGGG
GTCCCTGACCGATTCAGTGGCAGCGGGTCTGGGACAGATTTCACTCTCACC
ATCAGCAGCCTGCAGGCTGAAGATGTGGCAGTTTATTACTGTCAGCAATAT
ACCAGCGAACCTATCACGTTCGGCCAAGGTACCAAGGTGGAAATCAAAGGT
GGTGGTGGTTCAGGTGGTGGTGGTTCTGGCGGCGGCTCCGGTGGTGGTGGA
TCCGAGGTGCAGCTGTTGGAGTCTGGGGGAGGCTTGGTACAGCCTGGGGGG
TCCCTGCGACTCTCCTGTGCAGCCTCTGGATTCACCTTTAGCGACTATCAG
ATGAGCTGGGTCCGCCAGGCTCCAGGGAAGGGGCTGGAGTGGGTGTCAGGC
ATTCAGGGTGGCGGTGGTAGCACATATTACGCAGACTCCGTGAAGGGCCGG
TTCACCATCTCCCGTGACAATTCCAAGAACACGCTGTATCTGCAAATGAAC
AGCCTGCGTGCCGAGGACACGGCTGTGTATTACTGTGCGAGAGAGATGTGG
CGTGGGGACTACTACTCCGGTATGGACGTCTGGGGCCAGGGGACCACGGTC
ACCGTCTCCTCAGAACAGAAACTGATCTCTGAAGAAGACCTGGCGGCCGCA
ATTGAAGTTATGTATCCTCCTCCTTACCTAGACAATGAGAAGAGCAATGGA
ACCATTATCCATGTGAAAGGGAAACACCTTTGTCCAAGTCCCCTATTTCCC
GGACCTTCTAAGCCCTTTTGGGTGCTGGTGGTGGTTGGTGGAGTCCTGGCT
TGCTATAGCTTGCTAGTAACAGTGGCCTTTATTATTTTCTGGGTGAGGAGT
AAGAGGAGCAGGCTCCTGCACAGTGACTACATGAACATGACTCCCCGCCGC
CCCGGGCCCACCCGCAAGCATTACCAGCCCTATGCCCCACCACGCGACTTC
GCAGCCTATCGCTCCAGAGTGAAGTTCAGCAGGAGCGCAGACGCCCCCGCG
TACCAGCAGGGCCAGAACCAGCTCTATAACGAGCTCAATCTAGGACGAAGA
GAGGAGTACGATGTTTTGGACAAGAGACGTGGCCGGGACCCTGAGATGGGG
GGAAAGCCGAGAAGGAAGAACCCTCAGGAAGGCCTGTACAATGAACTGCAG
AAAGATAAGATGGCGGAGGCCTACAGTGAGATTGGGATGAAAGGCGAGCGC
CGGAGGGGCAAGGGGCACGATGGCCTTTACCAGGGTCTCAGTACAGCCACC
AAGGACACCTACGACGCCCTTCACATGCAGGCCCTGCCCCCTCGCTAG

In certain embodiments, the CAR is a CD371-targeted CAR. In certain embodiments, the CAR comprises (a) an extracellular antigen-binding domain comprising (i) a V_{H} that comprises a CDR1 comprising the amino acid sequence set forth in SEQ ID NO: 28, a CDR2 comprising the amino acid sequence set forth in SEQ ID NO: 29, and a V_{H} CDR3 comprising the amino acid sequence set forth in SEQ ID NO: 30, and (ii) a V_{L} that comprises a CDR1 comprising the amino acid sequence set forth in SEQ ID NO: 31, a CDR2 comprising the amino acid sequence set forth in SEQ ID NO: 32, and a V_{L} CDR3 comprising the amino acid sequence set forth in SEQ ID NO: 33; (b) a transmembrane domain comprising a CD28 polypeptide (e.g., a transmembrane domain of human CD28 or a portion thereof), and (c) an intracellular signaling domain comprising (i) a CD3ζ polypeptide, and (ii) a co-stimulatory signaling region comprising a CD28 polypeptide (e.g., an intracellular domain of human CD28 or a portion thereof). In certain embodiments, the V_{H} and V_{L} are linked via a linker comprising or consisting of the amino acid sequence set forth in SEQ ID NO: 14. In certain embodiments, the V_{H} and V_{L} are positioned from the N- to the C-terminus: V_{H}-V_{L}. In certain embodiments, the CAR is designed as “Et.B10H3L_MT_h28Z”. In certain embodiments, the CAR comprises the amino acid sequence set forth in SEQ ID NO: 79, which is provided below.

[SEQ ID NO: 79]
MLLLVTSLLLCELPHPAFLLIPRKVCNGIGIGEFKDSLSINATNIKHFKN
CTSISGDLHILPVAFRGDSFTHTPPLDPQELDILKTVKEITGFLLIQAWP
ENRTDLHAFENLEIIRGRTKQHGQFSLAVVSLNITSLGLRSLKEISDGDV
IISGNKNLCYANTINWKKLFGTSGQKTKIISNRGENSCKATGQVCHALCS
PEGCWGPEPRDCVSCRNVSRGRECVDKCNLLEGEPREFVENSECIQCHPE
CLPQAMNITCTGRGPDNCIQCAHYIDGPHCVKTCPAGVMGENNTLVWKYA
DAGHVCHLCHPNCTYGCTGPGLEGCPTNGPKIPSIATGMVGALLLLLVVA
LGIGLFMGSGEGRGSLLTCGDVEENPGPMALPVTALLLPLALLLHAEVQL
LESGGGLVQPGGSLRLSCAASGFTFSDYQMSWVRQAPGKGLEWVSGIQGG
GGSTYYADSVKGRFTISRDNSKNTLYLQMNSLRAEDTAVYYCAREMWRGD
YYSGMDVWGQGTTVTVSSGGGGSGGGGSGGGGSDIVMTQSPDSLAVSLGE
RATINCKSSQSVLDSYNNENNLAWYQQKPGQPPKLLIYWASTRESGVPDR
FSGSGSGTDFTLTISSLQAEDVAVYYCQQYTSEPITFGQGTKVEIKEQKL
ISEEDLAAAIEVMYPPPYLDNEKSNGTIIHVKGKHLCPSPLFPGPSKPFW
VLVVVGGVLACYSLLVTVAFIIFWVRSKRSRLLHSDYMNMTPRRPGPTRK
HYQPYAPPRDFAAYRSRVKFSRSADAPAYQQGQNQLYNELNLGRREEYDV
LDKRRGRDPEMGGKPRRKNPQEGLYNELQKDKMAEAYSEIGMKGERRRGK
GHDGLYQGLSTATKDTYDALHMQALPPR

An exemplary nucleic acid sequence the amino acid sequence of SEQ ID NO: 79 is set forth in SEQ ID NO: 80, which is provided below.

[SEQ ID NO: 80]
ATGCTTCTCCTGGTGACAAGCCTTCTGCTCTGTGAGTTACCACACCCAGC
ATTCCTCCTGATCCCACGCAAAGTGTGTAACGGAATAGGTATTGGTGAAT
TTAAAGACTCACTCTCCATAAATGCTACGAATATTAAACACTTCAAAAAC
TGCACCTCCATCAGTGGCGATCTCCACATCCTGCCGGTGGCATTTAGGGG
TGACTCCTTCACACATACTCCTCCTCTGGACCCACAGGAACTGGATATTC
TGAAAACCGTAAAGGAAATCACAGGGTTTTTGCTGATTCAGGCTTGGCCT
GAAAACAGGACGGACCTCCATGCCTTTGAGAACCTAGAAATCATACGCGG
CAGGACCAAGCAACATGGTCAGTTTTCTCTTGCAGTCGTCAGCCTGAACA
TAACATCCTTGGGATTACGCTCCCTCAAGGAGATAAGTGATGGAGATGTG
ATAATTTCAGGAAACAAAAATTTGTGCTATGCAAATACAATAAACTGGAA
AAAACTGTTTGGGACCTCCGGTCAGAAAACCAAAATTATAAGCAACAGAG
GTGAAAACAGCTGCAAGGCCACAGGCCAGGTCTGCCATGCCTTGTGCTCC
CCCGAGGGCTGCTGGGGCCCGGAGCCCAGGGACTGCGTCTCTTGCCGGAA
TGTCAGCCGAGGCAGGGAATGCGTGGACAAGTGCAACCTTCTGGAGGGTG
AGCCAAGGGAGTTTGTGGAGAACTCTGAGTGCATACAGTGCCACCCAGAG
TGCCTGCCTCAGGCCATGAACATCACCTGCACAGGACGGGGACCAGACAA
CTGTATCCAGTGTGCCCACTACATTGACGGCCCCCACTGCGTCAAGACCT
GCCCGGCAGGAGTCATGGGAGAAAACAACACCCTGGTCTGGAAGTACGCA
GACGCCGGCCATGTGTGCCACCTGTGCCATCCAAACTGCACCTACGGATG
CACTGGGCCAGGTCTTGAAGGCTGTCCAACGAATGGGCCTAAGATCCCGT
CCATCGCCACTGGGATGGTGGGGGCCCTCCTCTTGCTGCTGGTGGTGGCC
CTGGGGATCGGCCTCTTCATGGGTTCCGGTGAGGGACGGGGGTCACTGCT
CACCTGCGGAGATGTAGAAGAGAATCCCGGTCCCATGGCTCTCCCAGTGA
CTGCCCTACTGCTTCCCCTAGCGCTTCTCCTGCATGCAGAGGTGCAGCTG
TTGGAGTCTGGGGGAGGCTTGGTACAGCCTGGGGGGTCCCTGCGACTCTC
CTGTGCAGCCTCTGGATTCACCTTTAGCGACTATCAGATGAGCTGGGTCC
GCCAGGCTCCAGGGAAGGGGCTGGAGTGGGTGTCAGGCATTCAGGGTGGC
GGTGGTAGCACATATTACGCAGACTCCGTGAAGGGCCGGTTCACCATCTC
CCGTGACAATTCCAAGAACACGCTGTATCTGCAAATGAACAGCCTGCGTG
CCGAGGACACGGCTGTGTATTACTGTGCGAGAGAGATGTGGCGTGGGGAC
TACTACTCCGGTATGGACGTCTGGGGCCAGGGGACCACGGTCACCGTCTC
CTCAGGCGGGGGTGGTAGTGGCGGTGGAGGTAGCGGAGGTGGCGGGTCTG
ACATCGTGATGACCCAGTCTCCAGACTCCCTGGCTGTGTCTCTGGGCGAG
CGTGCCACCATCAACTGCAAGTCCAGCCAGAGTGTTTTAGACAGCTATAA
CAATGAGAACAATTTAGCTTGGTATCAGCAGAAACCAGGACAGCCTCCTA
AGCTGCTCATTTACTGGGCATCTACCCGGGAATCCGGGGTCCCTGACCGA
TTCAGTGGCAGCGGGTCTGGGACAGATTTCACTCTCACCATCAGCAGCCT
GCAGGCTGAAGATGTGGCAGTTTATTACTGTCAGCAATATACCAGCGAAC
CTATCACGTTCGGCCAAGGTACCAAGGTGGAAATCAAAGAACAGAAACTG
ATCTCTGAAGAAGACCTGGCGGCCGCAATTGAAGTTATGTATCCTCCTCC
TTACCTAGACAATGAGAAGAGCAATGGAACCATTATCCATGTGAAAGGGA
AACACCTTTGTCCAAGTCCCCTATTTCCCGGACCTTCTAAGCCCTTTTGG
GTGCTGGTGGTGGTTGGTGGAGTCCTGGCTTGCTATAGCTTGCTAGTAAC
AGTGGCCTTTATTATTTTCTGGGTGAGGAGTAAGAGGAGCAGGCTCCTGC
ACAGTGACTACATGAACATGACTCCCCGCCGCCCCGGGCCCACCCGCAAG
CATTACCAGCCCTATGCCCCACCACGCGACTTCGCAGCCTATCGCTCCAG
AGTGAAGTTCAGCAGGAGCGCAGACGCCCCCGCGTACCAGCAGGGCCAGA
ACCAGCTCTATAACGAGCTCAATCTAGGACGAAGAGAGGAGTACGATGTT
TTGGACAAGAGACGTGGCCGGGACCCTGAGATGGGGGGAAAGCCGAGAAG
GAAGAACCCTCAGGAAGGCCTGTACAATGAACTGCAGAAAGATAAGATGG
CGGAGGCCTACAGTGAGATTGGGATGAAAGGCGAGCGCCGGAGGGGCAAG
GGGCACGATGGCCTTTACCAGGGTCTCAGTACAGCCACCAAGGACACCTA
CGACGCCCTTCACATGCAGGCCCTGCCCCCTCGCTAG

In certain embodiments, the CAR is a CD371-targeted CAR. In certain embodiments, the CAR comprises (a) an extracellular antigen-binding domain comprising (i) a V_{H} that comprises a CDR1 comprising the amino acid sequence set forth in SEQ ID NO: 28, a CDR2 comprising the amino acid sequence set forth in SEQ ID NO: 29, and a V_{H} CDR3 comprising the amino acid sequence set forth in SEQ ID NO: 30, and (ii) a V_{L} that comprises a CDR1 comprising the amino acid sequence set forth in SEQ ID NO: 31, a CDR2 comprising the amino acid sequence set forth in SEQ ID NO: 32, and a V_{L} CDR3 comprising the amino acid sequence set forth in SEQ ID NO: 33; (b) a transmembrane domain comprising a CD28 polypeptide (e.g., a transmembrane domain of human CD28 or a portion thereof), and (c) an intracellular signaling domain comprising (i) a CD3ζ polypeptide, and (ii) a co-stimulatory signaling region comprising a CD28 polypeptide (e.g., an intracellular domain of human CD28 or a portion thereof). In certain embodiments, the V_{H} and V_{L} are linked via a linker comprising or consisting of the amino acid sequence set forth in SEQ ID NO: 13. In certain embodiments, the V_{H} and V_{L} are positioned from the N- to the C-terminus: V_{H}-V_{L}. In certain embodiments, the CAR is designated as “Et.B10H4L_MT_h28Z”. In certain embodiments, the CAR comprises the amino acid sequence set forth in SEQ ID NO: 81, which is provided below.

[SEQ ID NO: 81]
MLLLVTSLLLCELPHPAFLLIPRKVCNGIGIGEFKDSLSINATNIKHFKN
CTSISGDLHILPVAFRGDSFTHTPPLDPQELDILKTVKEITGFLLIQAWP
ENRTDLHAFENLEIIRGRTKQHGQFSLAVVSLNITSLGLRSLKEISDGDV
IISGNKNLCYANTINWKKLFGTSGQKTKIISNRGENSCKATGQVCHALCS
PEGCWGPEPRDCVSCRNVSRGRECVDKCNLLEGEPREFVENSECIQCHPE
CLPQAMNITCTGRGPDNCIQCAHYIDGPHCVKTCPAGVMGENNTLVWKYA
DAGHVCHLCHPNCTYGCTGPGLEGCPTNGPKIPSIATGMVGALLLLLVVA
LGIGLFMGSGEGRGSLLTCGDVEENPGPMALPVTALLLPLALLLHAEVQL
LESGGGLVQPGGSLRLSCAASGFTFSDYQMSWVRQAPGKGLEWVSGIQGG
GGSTYYADSVKGRFTISRDNSKNTLYLQMNSLRAEDTAVYYCAREMWRGD
YYSGMDVWGQGTTVTVSSGGGGSGGGGSGGGSGGGGSDIVMTQSPDSLAV
SLGERATINCKSSQSVLDSYNNENNLAWYQQKPGQPPKLLIYWASTRESG
VPDRFSGSGSGTDFTLTISSLQAEDVAVYYCQQYTSEPITFGQGTKVEIK
EQKLISEEDLAAAIEVMYPPPYLDNEKSNGTIIHVKGKHLCPSPLFPGPS
KPFWVLVVVGGVLACYSLLVTVAFIIFWVRSKRSRLLHSDYMNMTPRRPG
PTRKHYQPYAPPRDFAAYRSRVKFSRSADAPAYQQGQNQLYNELNLGRRE
EYDVLDKRRGRDPEMGGKPRRKNPQEGLYNELQKDKMAEAYSEIGMKGER
RRGKGHDGLYQGLSTATKDTYDALHMQALPPR

An exemplary nucleic acid sequence the amino acid sequence of SEQ ID NO: 81 is set forth in SEQ ID NO: 82, which is provided below.

[SEQ ID NO: 82]
ATGCTTCTCCTGGTGACAAGCCTTCTGCTCTGTGAGTTACCACACCCAGC
ATTCCTCCTGATCCCACGCAAAGTGTGTAACGGAATAGGTATTGGTGAAT
TTAAAGACTCACTCTCCATAAATGCTACGAATATTAAACACTTCAAAAAC
TGCACCTCCATCAGTGGCGATCTCCACATCCTGCCGGTGGCATTTAGGGG
TGACTCCTTCACACATACTCCTCCTCTGGACCCACAGGAACTGGATATTC
TGAAAACCGTAAAGGAAATCACAGGGTTTTTGCTGATTCAGGCTTGGCCT
GAAAACAGGACGGACCTCCATGCCTTTGAGAACCTAGAAATCATACGCGG
CAGGACCAAGCAACATGGTCAGTTTTCTCTTGCAGTCGTCAGCCTGAACA
TAACATCCTTGGGATTACGCTCCCTCAAGGAGATAAGTGATGGAGATGTG
ATAATTTCAGGAAACAAAAATTTGTGCTATGCAAATACAATAAACTGGAA
AAAACTGTTTGGGACCTCCGGTCAGAAAACCAAAATTATAAGCAACAGAG
GTGAAAACAGCTGCAAGGCCACAGGCCAGGTCTGCCATGCCTTGTGCTCC
CCCGAGGGCTGCTGGGGCCCGGAGCCCAGGGACTGCGTCTCTTGCCGGAA
TGTCAGCCGAGGCAGGGAATGCGTGGACAAGTGCAACCTTCTGGAGGGTG
AGCCAAGGGAGTTTGTGGAGAACTCTGAGTGCATACAGTGCCACCCAGAG
TGCCTGCCTCAGGCCATGAACATCACCTGCACAGGACGGGGACCAGACAA
CTGTATCCAGTGTGCCCACTACATTGACGGCCCCCACTGCGTCAAGACCT
GCCCGGCAGGAGTCATGGGAGAAAACAACACCCTGGTCTGGAAGTACGCA
GACGCCGGCCATGTGTGCCACCTGTGCCATCCAAACTGCACCTACGGATG
CACTGGGCCAGGTCTTGAAGGCTGTCCAACGAATGGGCCTAAGATCCCGT
CCATCGCCACTGGGATGGTGGGGGCCCTCCTCTTGCTGCTGGTGGTGGCC
CTGGGGATCGGCCTCTTCATGGGTTCCGGTGAGGGACGGGGGTCACTGCT
CACCTGCGGAGATGTAGAAGAGAATCCCGGTCCCATGGCTCTCCCAGTGA
CTGCCCTACTGCTTCCCCTAGCGCTTCTCCTGCATGCAGAGGTGCAGCTG
TTGGAGTCTGGGGGAGGCTTGGTACAGCCTGGGGGGTCCCTGCGACTCTC
CTGTGCAGCCTCTGGATTCACCTTTAGCGACTATCAGATGAGCTGGGTCC
GCCAGGCTCCAGGGAAGGGGCTGGAGTGGGTGTCAGGCATTCAGGGTGGC
GGTGGTAGCACATATTACGCAGACTCCGTGAAGGGCCGGTTCACCATCTC
CCGTGACAATTCCAAGAACACGCTGTATCTGCAAATGAACAGCCTGCGTG
CCGAGGACACGGCTGTGTATTACTGTGCGAGAGAGATGTGGCGTGGGGAC
TACTACTCCGGTATGGACGTCTGGGGCCAGGGGACCACGGTCACCGTCTC
CTCAGGTGGTGGTGGTTCAGGTGGTGGTGGTTCTGGCGGCGGCTCCGGTG
GTGGTGGATCCGACATCGTGATGACCCAGTCTCCAGACTCCCTGGCTGTG
TCTCTGGGCGAGCGTGCCACCATCAACTGCAAGTCCAGCCAGAGTGTTTT
AGACAGCTATAACAATGAGAACAATTTAGCTTGGTATCAGCAGAAACCAG
GACAGCCTCCTAAGCTGCTCATTTACTGGGCATCTACCCGGGAATCCGGG
GTCCCTGACCGATTCAGTGGCAGCGGGTCTGGGACAGATTTCACTCTCAC
CATCAGCAGCCTGCAGGCTGAAGATGTGGCAGTTTATTACTGTCAGCAAT
ATACCAGCGAACCTATCACGTTCGGCCAAGGTACCAAGGTGGAAATCAAA
GAACAGAAACTGATCTCTGAAGAAGACCTGGCGGCCGCAATTGAAGTTAT
GTATCCTCCTCCTTACCTAGACAATGAGAAGAGCAATGGAACCATTATCC
ATGTGAAAGGGAAACACCTTTGTCCAAGTCCCCTATTTCCCGGACCTTCT
AAGCCCTTTTGGGTGCTGGTGGTGGTTGGTGGAGTCCTGGCTTGCTATAG
CTTGCTAGTAACAGTGGCCTTTATTATTTTCTGGGTGAGGAGTAAGAGGA
GCAGGCTCCTGCACAGTGACTACATGAACATGACTCCCCGCCGCCCCGGG
CCCACCCGCAAGCATTACCAGCCCTATGCCCCACCACGCGACTTCGCAGC
CTATCGCTCCAGAGTGAAGTTCAGCAGGAGCGCAGACGCCCCCGCGTACC
AGCAGGGCCAGAACCAGCTCTATAACGAGCTCAATCTAGGACGAAGAGAG
GAGTACGATGTTTTGGACAAGAGACGTGGCCGGGACCCTGAGATGGGGGG
AAAGCCGAGAAGGAAGAACCCTCAGGAAGGCCTGTACAATGAACTGCAGA
AAGATAAGATGGCGGAGGCCTACAGTGAGATTGGGATGAAAGGCGAGCGC
CGGAGGGGCAAGGGGCACGATGGCCTTTACCAGGGTCTCAGTACAGCCAC
CAAGGACACCTACGACGCCCTTCACATGCAGGCCCTGCCCCCTCGCTAG

In certain embodiments, the CAR is a CD371-targeted CAR. In certain embodiments, the CAR comprises (a) an extracellular antigen-binding domain comprising (i) a V_{H} that comprises a CDR1 comprising the amino acid sequence set forth in SEQ ID NO: 34, a CDR2 comprising the amino acid sequence set forth in SEQ ID NO: 35, and a V_{H} CDR3 comprising the amino acid sequence set forth in SEQ ID NO: 36, and (ii) a V_{L} that comprises a CDR1 comprising the amino acid sequence set forth in SEQ ID NO: 37, a CDR2 comprising the amino acid sequence set forth in SEQ ID NO: 38, and a V_{L} CDR3 comprising the amino acid sequence set forth in SEQ ID NO: 39; (b) a transmembrane domain comprising a CD28 polypeptide (e.g., a transmembrane domain of human CD28 or a portion thereof), and (c) an intracellular signaling domain comprising (i) a CD3ζ polypeptide, and (ii) a co-stimulatory signaling region comprising a CD28 polypeptide (e.g., an intracellular domain of human CD28 or a portion thereof). In certain embodiments, the V_{H} and V_{L} are linked via a linker comprising or consisting of the amino acid sequence set forth in SEQ ID NO: 14. In certain embodiments, the V_{H} and V_{L} are positioned from the N- to the C-terminus: V_{H}-V_{L}. In certain embodiments, the CAR is designated as “Et.C3H3L_MT_h28Z”. In certain embodiments, the CAR comprises the amino acid sequence set forth in SEQ ID NO: 83, which is provided below.

[SEQ ID NO: 83]
MLLLVTSLLLCELPHPAFLLIPRKVCNGIGIGEFKDSLSINATNIKHFKN
CTSISGDLHILPVAFRGDSFTHTPPLDPQELDILKTVKEITGFLLIQAWP
ENRTDLHAFENLEIIRGRTKQHGQFSLAVVSLNITSLGLRSLKEISDGDV
IISGNKNLCYANTINWKKLFGTSGQKTKIISNRGENSCKATGQVCHALCS
PEGCWGPEPRDCVSCRNVSRGRECVDKCNLLEGEPREFVENSECIQCHPE
CLPQAMNITCTGRGPDNCIQCAHYIDGPHCVKTCPAGVMGENNTLVWKYA
DAGHVCHLCHPNCTYGCTGPGLEGCPTNGPKIPSIATGMVGALLLLLVVA
LGIGLFMGSGEGRGSLLTCGDVEENPGPMALPVTALLLPLALLLHAEVQL
LESGGGLVQPGGSLRLSCAASGFTFTSYAMSWVRQAPGKGLEWVSGIDGS
GGGTNYADSVKGRFTISRDNSKNTLYLQMNSLRAEDTAVYYCARAYYDIL
TGYPVDGMDVWGQGTTVTVSSGGGGSGGGGSGGGGSDIVMTQSPDSLAVS
LGERATINCKSSQSVLSSYNNENNLAWYQQKPGQPPKLLIYAASTRESGV
PDRFSGSGSGTDFTLTISSLQAEDVAVYYCQQYYSEPYTFGQGTKVEIKE
QKLISEEDLAAAIEVMYPPPYLDNEKSNGTIIHVKGKHLCPSPLFPGPSK
PFWVLVVVGGVLACYSLLVTVAFIIFWVRSKRSRLLHSDYMNMTPRRPGP
TRKHYQPYAPPRDFAAYRSRVKFSRSADAPAYQQGQNQLYNELNLGRREE
YDVLDKRRGRDPENGGKPRRKNPQEGLYNELQKDKMAEAYSEIGMKGERR
RGKGHDGLYQGLSTATKDTYDALHMQALPPR

An exemplary nucleic acid sequence the amino acid sequence of SEQ ID NO: 83 is set forth in SEQ ID NO: 84, which is provided below.

[SEQ ID NO: 84]
ATGCTTCTCCTGGTGACAAGCCTTCTGCTCTGTGAGTTACCACACCCAGC
ATTCCTCCTGATCCCACGCAAAGTGTGTAACGGAATAGGTATTGGTGAAT
TTAAAGACTCACTCTCCATAAATGCTACGAATATTAAACACTTCAAAAAC
TGCACCTCCATCAGTGGCGATCTCCACATCCTGCCGGTGGCATTTAGGGG
TGACTCCTTCACACATACTCCTCCTCTGGACCCACAGGAACTGGATATTC
TGAAAACCGTAAAGGAAATCACAGGGTTTTTGCTGATTCAGGCTTGGCCT
GAAAACAGGACGGACCTCCATGCCTTTGAGAACCTAGAAATCATACGCGG
CAGGACCAAGCAACATGGTCAGTTTTCTCTTGCAGTCGTCAGCCTGAACA
TAACATCCTTGGGATTACGCTCCCTCAAGGAGATAAGTGATGGAGATGTG
ATAATTTCAGGAAACAAAAATTTGTGCTATGCAAATACAATAAACTGGAA
AAAACTGTTTGGGACCTCCGGTCAGAAAACCAAAATTATAAGCAACAGAG
GTGAAAACAGCTGCAAGGCCACAGGCCAGGTCTGCCATGCCTTGTGCTCC
CCCGAGGGCTGCTGGGGCCCGGAGCCCAGGGACTGCGTCTCTTGCCGGAA
TGTCAGCCGAGGCAGGGAATGCGTGGACAAGTGCAACCTTCTGGAGGGTG
AGCCAAGGGAGTTTGTGGAGAACTCTGAGTGCATACAGTGCCACCCAGAG
TGCCTGCCTCAGGCCATGAACATCACCTGCACAGGACGGGGACCAGACAA
CTGTATCCAGTGTGCCCACTACATTGACGGCCCCCACTGCGTCAAGACCT
GCCCGGCAGGAGTCATGGGAGAAAACAACACCCTGGTCTGGAAGTACGCA
GACGCCGGCCATGTGTGCCACCTGTGCCATCCAAACTGCACCTACGGATG
CACTGGGCCAGGTCTTGAAGGCTGTCCAACGAATGGGCCTAAGATCCCGT
CCATCGCCACTGGGATGGTGGGGGCCCTCCTCTTGCTGCTGGTGGTGGCC
CTGGGGATCGGCCTCTTCATGGGTTCCGGTGAGGGACGGGGGTCACTGCT
CACCTGCGGAGATGTAGAAGAGAATCCCGGTCCCATGGCTCTCCCAGTGA
CTGCCCTACTGCTTCCCCTAGCGCTTCTCCTGCATGCAGAGGTGCAGCTG
TTGGAGTCTGGGGGAGGCTTGGTACAGCCTGGGGGGTCCCTGCGACTCTC
CTGTGCAGCCTCTGGATTCACCTTTACCAGCTATGCCATGAGCTGGGTCC
GCCAGGCTCCAGGGAAGGGGCTGGAGTGGGTGTCAGGCATTGACGGTAGC
GGTGGTGGCACAAATTACGCAGACTCCGTGAAGGGCCGGTTCACCATCTC
CCGTGACAATTCCAAGAACACGCTGTATCTGCAAATGAACAGCCTGCGTG
CCGAGGACACGGCTGTGTATTACTGTGCGAGAGCGTATTACGATATTTTG
ACTGGTTACCCCGTGGACGGTATGGACGTCTGGGGCCAAGGGACCACGGT
CACCGTCTCCTCAGGCGGGGGTGGTAGTGGCGGTGGAGGTAGCGGAGGTG
GCGGGTCTGACATCGTGATGACCCAGTCTCCAGACTCCCTGGCTGTGTCT
CTGGGCGAGCGTGCCACCATCAACTGCAAGTCCAGCCAGAGTGTTTTAAG
CAGCTATAACAATGAGAACAATTTAGCTTGGTATCAGCAGAAACCAGGAC
AGCCTCCTAAGCTGCTCATTTACGCCGCATCTACCCGGGAATCCGGGGTC
CCTGACCGATTCAGTGGCAGCGGGTCTGGGACAGATTTCACTCTCACCAT
CAGCAGCCTGCAGGCTGAAGATGTGGCAGTTTATTACTGTCAGCAATATT
ATAGCGAACCTTATACGTTCGGCCAAGGTACCAAGGTGGAAATCAAAGAA
CAGAAACTGATCTCTGAAGAAGACCTGGCGGCCGCAATTGAAGTTATGTA
TCCTCCTCCTTACCTAGACAATGAGAAGAGCAATGGAACCATTATCCATG
TGAAAGGGAAACACCTTTGTCCAAGTCCCCTATTTCCCGGACCTTCTAAG
CCCTTTTGGGTGCTGGTGGTGGTTGGTGGAGTCCTGGCTTGCTATAGCTT
GCTAGTAACAGTGGCCTTTATTATTTTCTGGGTGAGGAGTAAGAGGAGCA
GGCTCCTGCACAGTGACTACATGAACATGACTCCCCGCCGCCCCGGGCCC
ACCCGCAAGCATTACCAGCCCTATGCCCCACCACGCGACTTCGCAGCCTA
TCGCTCCAGAGTGAAGTTCAGCAGGAGCGCAGACGCCCCCGCGTACCAGC
AGGGCCAGAACCAGCTCTATAACGAGCTCAATCTAGGACGAAGAGAGGAG
TACGATGTTTTGGACAAGAGACGTGGCCGGGACCCTGAGATGGGGGGAAA
GCCGAGAAGGAAGAACCCTCAGGAAGGCCTGTACAATGAACTGCAGAAAG
ATAAGATGGCGGAGGCCTACAGTGAGATTGGGATGAAAGGCGAGCGCCGG
AGGGGCAAGGGGCACGATGGCCTTTACCAGGGTCTCAGTACAGCCACCAA
GGACACCTACGACGCCCTTCACATGCAGGCCCTGCCCCCTCGCTAG

In certain embodiments, the CAR is a CD371-targeted CAR. In certain embodiments, the CAR comprises (a) an extracellular antigen-binding domain comprising (i) a V_{H} that comprises a CDR1 comprising the amino acid sequence set forth in SEQ ID NO: 34, a CDR2 comprising the amino acid sequence set forth in SEQ ID NO: 35, and a V_{H} CDR3 comprising the amino acid sequence set forth in SEQ ID NO: 36, and (ii) a V_{L} that comprises a CDR1 comprising the amino acid sequence set forth in SEQ ID NO: 37, a CDR2 comprising the amino acid sequence set forth in SEQ ID NO: 38, and a V_{L} CDR3 comprising the amino acid sequence set forth in SEQ ID NO: 39; (b) a transmembrane domain comprising a CD28 polypeptide (e.g., a transmembrane domain of human CD28 or a portion thereof), and (c) an intracellular signaling domain comprising (i) a CD3ζ polypeptide, and (ii) a co-stimulatory signaling region comprising a CD28 polypeptide (e.g., an intracellular domain of human CD28 or a portion thereof). In certain embodiments, the V_{H} and V_{L} are linked via a linker comprising or consisting of the amino acid sequence set forth in SEQ ID NO: 14. In certain embodiments, the V_{H} and V_{L} are positioned from the N- to the C-terminus: V_{L}-V_{H}. In certain embodiments, the CAR is designated as “Et.C3L3H_MT_h28Z”. In certain embodiments, the CAR comprises the amino acid sequence set forth in SEQ ID NO: 85, which is provided below.

[SEQ ID NO: 85]
MLLLVTSLLLCELPHPAFLLIPRKVCNGIGIGEFKDSLSINATNIKHFKNC
TSISGDLHILPVAFRGDSFTHTPPLDPQELDILKTVKEITGFLLIQAWPEN
RTDLHAFENLEIIRGRTKQHGQFSLAVVSLNITSLGLRSLKEISDGDVIIS
GNKNLCYANTINWKKLFGTSGQKTKIISNRGENSCKATGQVCHALCSPEGC
WGPEPRDCVSCRNVSRGRECVDKCNLLEGEPREFVENSECIQCHPECLPQA
MNITCTGRGPDNCIQCAHYIDGPHCVKTCPAGVMGENNTLVWKYADAGHVC
HLCHPNCTYGCTGPGLEGCPTNGPKIPSIATGMVGALLLLLVVALGIGLFM
GSGEGRGSLLTCGDVEENPGPMALPVTALLLPLALLLHADIVMTQSPDSLA
VSLGERATINCKSSQSVLSSYNNENNLAWYQQKPGQPPKLLIYAASTRESG
VPDRFSGSGSGTDFTLTISSLQAEDVAVYYCQQYYSEPYTFGQGTKVEIKG
GGGSGGGGSGGGGSEVQLLESGGGLVQPGGSLRLSCAASGFTFTSYAMSWV
RQAPGKGLEWVSGIDGSGGGTNYADSVKGRFTISRDNSKNTLYLQMNSLRA
EDTAVYYCARAYYDILTGYPVDGMDVWGQGTTVTVSSEQKLISEEDLAAAI
EVMYPPPYLDNEKSNGTIIHVKGKHLCPSPLFPGPSKPFWVLVVVGGVLAC
YSLLVTVAFIIFWVRSKRSRLLHSDYMNMTPRRPGPTRKHYQPYAPPRDFA
AYRSRVKFSRSADAPAYQQGQNQLYNELNLGRREEYDVLDKRRGRDPENGG
KPRRKNPQEGLYNELQKDKMAEAYSEIGMKGERRRGKGHDGLYQGLSTATK
DTYDALHMQALPPR

An exemplary nucleic acid sequence the amino acid sequence of SEQ ID NO: 85 is set forth in SEQ ID NO: 86, which is provided below.

[SEQ ID NO: 86]
ATGCTTCTCCTGGTGACAAGCCTTCTGCTCTGTGAGTTACCACACCCAGC
ATTCCTCCTGATCCCACGCAAAGTGTGTAACGGAATAGGTATTGGTGAAT
TTAAAGACTCACTCTCCATAAATGCTACGAATATTAAACACTTCAAAAAC
TGCACCTCCATCAGTGGCGATCTCCACATCCTGCCGGTGGCATTTAGGGG
TGACTCCTTCACACATACTCCTCCTCTGGACCCACAGGAACTGGATATTC
TGAAAACCGTAAAGGAAATCACAGGGTTTTTGCTGATTCAGGCTTGGCCT
GAAAACAGGACGGACCTCCATGCCTTTGAGAACCTAGAAATCATACGCGG
CAGGACCAAGCAACATGGTCAGTTTTCTCTTGCAGTCGTCAGCCTGAACA
TAACATCCTTGGGATTACGCTCCCTCAAGGAGATAAGTGATGGAGATGTG
ATAATTTCAGGAAACAAAAATTTGTGCTATGCAAATACAATAAACTGGAA
AAAACTGTTTGGGACCTCCGGTCAGAAAACCAAAATTATAAGCAACAGAG
GTGAAAACAGCTGCAAGGCCACAGGCCAGGTCTGCCATGCCTTGTGCTCC
CCCGAGGGCTGCTGGGGCCCGGAGCCCAGGGACTGCGTCTCTTGCCGGAA
TGTCAGCCGAGGCAGGGAATGCGTGGACAAGTGCAACCTTCTGGAGGGTG
AGCCAAGGGAGTTTGTGGAGAACTCTGAGTGCATACAGTGCCACCCAGAG
TGCCTGCCTCAGGCCATGAACATCACCTGCACAGGACGGGGACCAGACAA
CTGTATCCAGTGTGCCCACTACATTGACGGCCCCCACTGCGTCAAGACCT
GCCCGGCAGGAGTCATGGGAGAAAACAACACCCTGGTCTGGAAGTACGCA
GACGCCGGCCATGTGTGCCACCTGTGCCATCCAAACTGCACCTACGGATG
CACTGGGCCAGGTCTTGAAGGCTGTCCAACGAATGGGCCTAAGATCCCGT
CCATCGCCACTGGGATGGTGGGGGCCCTCCTCTTGCTGCTGGTGGTGGCC
CTGGGGATCGGCCTCTTCATGGGTTCCGGTGAGGGACGGGGGTCACTGCT
CACCTGCGGAGATGTAGAAGAGAATCCCGGTCCCATGGCTCTCCCAGTGA
CTGCCCTACTGCTTCCCCTAGCGCTTCTCCTGCATGCAGACATCGTGATG
ACCCAGTCTCCAGACTCCCTGGCTGTGTCTCTGGGCGAGCGTGCCACCAT
CAACTGCAAGTCCAGCCAGAGTGTTTTAAGCAGCTATAACAATGAGAACA
ATTTAGCTTGGTATCAGCAGAAACCAGGACAGCCTCCTAAGCTGCTCATT
TACGCCGCATCTACCCGGGAATCCGGGGTCCCTGACCGATTCAGTGGCAG
CGGGTCTGGGACAGATTTCACTCTCACCATCAGCAGCCTGCAGGCTGAAG
ATGTGGCAGTTTATTACTGTCAGCAATATTATAGCGAACCTTATACGTTC
GGCCAAGGTACCAAGGTGGAAATCAAAGGCGGGGGTGGTAGTGGCGGTGG
AGGTAGCGGAGGTGGCGGGTCTGAGGTGCAGCTGTTGGAGTCTGGGGGAG
GCTTGGTACAGCCTGGGGGGTCCCTGCGACTCTCCTGTGCAGCCTCTGGA
TTCACCTTTACCAGCTATGCCATGAGCTGGGTCCGCCAGGCTCCAGGGAA
GGGGCTGGAGTGGGTGTCAGGCATTGACGGTAGCGGTGGTGGCACAAATT
ACGCAGACTCCGTGAAGGGCCGGTTCACCATCTCCCGTGACAATTCCAAG
AACACGCTGTATCTGCAAATGAACAGCCTGCGTGCCGAGGACACGGCTGT
GTATTACTGTGCGAGAGCGTATTACGATATTTTGACTGGTTACCCCGTGG
ACGGTATGGACGTCTGGGGCCAAGGGACCACGGTCACCGTCTCCTCAGAA
CAGAAACTGATCTCTGAAGAAGACCTGGCGGCCGCAATTGAAGTTATGTA
TCCTCCTCCTTACCTAGACAATGAGAAGAGCAATGGAACCATTATCCATG
TGAAAGGGAAACACCTTTGTCCAAGTCCCCTATTTCCCGGACCTTCTAAG
CCCTTTTGGGTGCTGGTGGTGGTTGGTGGAGTCCTGGCTTGCTATAGCTT
GCTAGTAACAGTGGCCTTTATTATTTTCTGGGTGAGGAGTAAGAGGAGCA
GGCTCCTGCACAGTGACTACATGAACATGACTCCCCGCCGCCCCGGGCCC
ACCCGCAAGCATTACCAGCCCTATGCCCCACCACGCGACTTCGCAGCCTA
TCGCTCCAGAGTGAAGTTCAGCAGGAGCGCAGACGCCCCCGCGTACCAGC
AGGGCCAGAACCAGCTCTATAACGAGCTCAATCTAGGACGAAGAGAGGAG
TACGATGTTTTGGACAAGAGACGTGGCCGGGACCCTGAGATGGGGGGAAA
GCCGAGAAGGAAGAACCCTCAGGAAGGCCTGTACAATGAACTGCAGAAAG
ATAAGATGGCGGAGGCCTACAGTGAGATTGGGATGAAAGGCGAGCGCCGG
AGGGGCAAGGGGCACGATGGCCTTTACCAGGGTCTCAGTACAGCCACCAA
GGACACCTACGACGCCCTTCACATGCAGGCCCTGCCCCCTCGCTAG

In certain embodiments, the CAR is a CD371-targeted CAR. In certain embodiments, the CAR comprises (a) an extracellular antigen-binding domain comprising (i) a V_{H} that comprises a CDR1 comprising the amino acid sequence set forth in SEQ ID NO: 40, a CDR2 comprising the amino acid sequence set forth in SEQ ID NO: 41, and a V_{H} CDR3 comprising the amino acid sequence set forth in SEQ ID NO: 42, and (ii) a V_{L} that comprises a CDR1 comprising the amino acid sequence set forth in SEQ ID NO: 43, a CDR2 comprising the amino acid sequence set forth in SEQ ID NO: 44, and a V_{L} CDR3 comprising the amino acid sequence set forth in SEQ ID NO: 45; (b) a transmembrane domain comprising a CD28 polypeptide (e.g., a transmembrane domain of human CD28 or a portion thereof), and (c) an intracellular signaling domain comprising (i) a CD3ζ polypeptide, and (ii) a co-stimulatory signaling region comprising a CD28 polypeptide (e.g., an intracellular domain of human CD28 or a portion thereof). In certain embodiments, the V_{H} and V_{L} are linked via a linker comprising or consisting of the amino acid sequence set forth in SEQ ID NO: 14. In certain embodiments, the V_{H} and V_{L} are positioned from the N- to the C-terminus: V_{L}-V_{H}. In certain embodiments, the CAQR is designated as “Et.D6L3H_MT_h28Z”. In certain embodiments, the CAR comprises the amino acid sequence set forth in SEQ ID NO: 87, which is provided below.

[SEQ ID NO: 87]
MLLLVTSLLLCELPHPAFLLIPRKVCNGIGIGEFKDSLSINATNIKHFKNC
TSISGDLHILPVAFRGDSFTHTPPLDPQELDILKTVKEITGFLLIQAWPEN
RTDLHAFENLEIIRGRTKQHGQFSLAVVSLNITSLGLRSLKEISDGDVIIS
GNKNLCYANTINWKKLFGTSGQKTKIISNRGENSCKATGQVCHALCSPEGC
WGPEPRDCVSCRNVSRGRECVDKCNLLEGEPREFVENSECIQCHPECLPQA
MNITCTGRGPDNCIQCAHYIDGPHCVKTCPAGVMGENNTLVWKYADAGHVC
HLCHPNCTYGCTGPGLEGCPTNGPKIPSIATGMVGALLLLLVVALGIGLFM
GSGEGRGSLLTCGDVEENPGPMALPVTALLLPLALLLHADIVMTQSPDSLA
VSLGERATINCKSSQSVLRSSNNKNNLAWYQQKPGQPPKLLIYAASTRESG
VPDRFSGSGSGTDFTLTISSLQAEDVAVYYCQQYYREPLTFGQGTKVEIKG
GGGSGGGGSGGGGSEVQLLESGGGLVQPGGSLRLSCAASGFTFTDYAMSWV
RQAPGKGLEWVSDIDGSGGSTDYADSVKGRFTISRDNSKNTLYLQMNSLRA
EDTAVYYCALELGATTVYWGQGTLVTVSSEQKLISEEDLAAAIEVMYPPPY
LDNEKSNGTIIHVKGKHLCPSPLFPGPSKPFWVLVVVGGVLACYSLLVTVA
FIIFWVRSKRSRLLHSDYMNMTPRRPGPTRKHYQPYAPPRDFAAYRSRVKF
SRSADAPAYQQGQNQLYNELNLGRREEYDVLDKRRGRDPEMGGKPRRKNPQ
EGLYNELQKDKMAEAYSEIGMKGERRRGKGHDGLYQGLSTATKDTYDALHM
QALPPR

An exemplary nucleic acid sequence the amino acid sequence of SEQ ID NO: 87 is set forth in SEQ ID NO: 88, which is provided below.

[SEQ ID NO: 88]
ATGCTTCTCCTGGTGACAAGCCTTCTGCTCTGTGAGTTACCACACCCAGC
ATTCCTCCTGATCCCACGCAAAGTGTGTAACGGAATAGGTATTGGTGAAT
TTAAAGACTCACTCTCCATAAATGCTACGAATATTAAACACTTCAAAAAC
TGCACCTCCATCAGTGGCGATCTCCACATCCTGCCGGTGGCATTTAGGGG
TGACTCCTTCACACATACTCCTCCTCTGGACCCACAGGAACTGGATATTC
TGAAAACCGTAAAGGAAATCACAGGGTTTTTGCTGATTCAGGCTTGGCCT
GAAAACAGGACGGACCTCCATGCCTTTGAGAACCTAGAAATCATACGCGG
CAGGACCAAGCAACATGGTCAGTTTTCTCTTGCAGTCGTCAGCCTGAACA
TAACATCCTTGGGATTACGCTCCCTCAAGGAGATAAGTGATGGAGATGTG
ATAATTTCAGGAAACAAAAATTTGTGCTATGCAAATACAATAAACTGGAA
AAAACTGTTTGGGACCTCCGGTCAGAAAACCAAAATTATAAGCAACAGAG
GTGAAAACAGCTGCAAGGCCACAGGCCAGGTCTGCCATGCCTTGTGCTCC
CCCGAGGGCTGCTGGGGCCCGGAGCCCAGGGACTGCGTCTCTTGCCGGAA
TGTCAGCCGAGGCAGGGAATGCGTGGACAAGTGCAACCTTCTGGAGGGTG
AGCCAAGGGAGTTTGTGGAGAACTCTGAGTGCATACAGTGCCACCCAGAG
TGCCTGCCTCAGGCCATGAACATCACCTGCACAGGACGGGGACCAGACAA
CTGTATCCAGTGTGCCCACTACATTGACGGCCCCCACTGCGTCAAGACCT
GCCCGGCAGGAGTCATGGGAGAAAACAACACCCTGGTCTGGAAGTACGCA
GACGCCGGCCATGTGTGCCACCTGTGCCATCCAAACTGCACCTACGGATG
CACTGGGCCAGGTCTTGAAGGCTGTCCAACGAATGGGCCTAAGATCCCGT
CCATCGCCACTGGGATGGTGGGGGCCCTCCTCTTGCTGCTGGTGGTGGCC
CTGGGGATCGGCCTCTTCATGGGTTCCGGTGAGGGACGGGGGTCACTGCT
CACCTGCGGAGATGTAGAAGAGAATCCCGGTCCCATGGCTCTCCCAGTGA
CTGCCCTACTGCTTCCCCTAGCGCTTCTCCTGCATGCAGACATCGTGATG
ACCCAGTCTCCAGACTCCCTGGCTGTGTCTCTGGGCGAGCGTGCCACCAT
CAACTGCAAGTCCAGCCAGAGTGTTTTACGCAGCAGCAACAATAAAAACA
ATTTAGCTTGGTATCAGCAGAAACCAGGACAGCCTCCTAAGCTGCTCATT
TACGCCGCATCTACCCGGGAATCCGGGGTCCCTGACCGATTCAGTGGCAG
CGGGTCTGGGACAGATTTCACTCTCACCATCAGCAGCCTGCAGGCTGAAG
ATGTGGCAGTTTATTACTGTCAGCAATATTATCGCGAACCTCTGACGTTC
GGCCAAGGTACCAAGGTGGAAATCAAAGGCGGGGGTGGTAGTGGCGGTGG
AGGTAGCGGAGGTGGCGGGTCTGAGGTGCAGCTGTTGGAGTCTGGGGGAG
GCTTGGTACAGCCTGGGGGGTCCCTGCGACTCTCCTGTGCAGCCTCTGGA
TTCACCTTTACCGACTATGCCATGAGCTGGGTCCGCCAGGCTCCAGGGAA
GGGGCTGGAGTGGGTGTCAGACATTGACGGTAGCGGTGGTAGCACAGACT
ACGCAGACTCCGTGAAGGGCCGGTTCACCATCTCCCGTGACAATTCCAAG
AACACGCTGTATCTGCAAATGAACAGCCTGCGTGCCGAGGACACGGCTGT
GTATTACTGTGCGCTAGAGCTGGGAGCTACTACCGTCTACTGGGGCCAGG
GAACCCTGGTCACCGTCTCCTCAGAACAGAAACTGATCTCTGAAGAAGAC
CTGGCGGCCGCAATTGAAGTTATGTATCCTCCTCCTTACCTAGACAATGA
GAAGAGCAATGGAACCATTATCCATGTGAAAGGGAAACACCTTTGTCCAA
GTCCCCTATTTCCCGGACCTTCTAAGCCCTTTTGGGTGCTGGTGGTGGTT
GGTGGAGTCCTGGCTTGCTATAGCTTGCTAGTAACAGTGGCCTTTATTAT
TTTCTGGGTGAGGAGTAAGAGGAGCAGGCTCCTGCACAGTGACTACATGA
ACATGACTCCCCGCCGCCCCGGGCCCACCCGCAAGCATTACCAGCCCTAT
GCCCCACCACGCGACTTCGCAGCCTATCGCTCCAGAGTGAAGTTCAGCAG
GAGCGCAGACGCCCCCGCGTACCAGCAGGGCCAGAACCAGCTCTATAACG
AGCTCAATCTAGGACGAAGAGAGGAGTACGATGTTTTGGACAAGAGACGT
GGCCGGGACCCTGAGATGGGGGGAAAGCCGAGAAGGAAGAACCCTCAGGA
AGGCCTGTACAATGAACTGCAGAAAGATAAGATGGCGGAGGCCTACAGTG
AGATTGGGATGAAAGGCGAGCGCCGGAGGGGCAAGGGGCACGATGGCCTT
TACCAGGGTCTCAGTACAGCCACCAAGGACACCTACGACGCCCTTCACAT
GCAGGCCCTGCCCCCTCGCTAG

In certain embodiments, the CAR is a CD371-targeted CAR. In certain embodiments, the CAR comprises (a) an extracellular antigen-binding domain comprising (i) a V_{H} that comprises a CDR1 comprising the amino acid sequence set forth in SEQ ID NO: 28, a CDR2 comprising the amino acid sequence set forth in SEQ ID NO: 29, and a V_{H} CDR3 comprising the amino acid sequence set forth in SEQ ID NO: 30, and (ii) a V_{L} that comprises a CDR1 comprising the amino acid sequence set forth in SEQ ID NO: 31, a CDR2 comprising the amino acid sequence set forth in SEQ ID NO: 32, and a V_{L} CDR3 comprising the amino acid sequence set forth in SEQ ID NO: 33; (b) a transmembrane domain comprising a CD28 polypeptide (e.g., a transmembrane domain of human CD28 or a fragment thereof), and (c) an intracellular signaling domain comprising (i) a CD3ζ polypeptide, and (ii) a co-stimulatory signaling region comprising a CD28 polypeptide (e.g., an intracellular domain of human CD28 or a fragment thereof). In certain embodiments, the V_{H} and V_{L} are linked via a linker comprising or consisting of the amino acid sequence set forth in SEQ ID NO: 93. In certain embodiments, the V_{H} and V_{L} are positioned from the N- to the C-terminus: V_{H}-V_{L}. In certain embodiments, the CAR is designed as “Et.B10H1L_MT_h28Z”. In certain embodiments, the CAR comprises the amino acid sequence set forth in SEQ ID NO: 95, which is provided below.

[SEQ ID NO: 95]
MLLLVTSLLLCELPHPAFLLIPRKVCNGIGIGEFKDSLSINATNIKHFKN
CTSISGDLHILPVAFRGDSFTHTPPLDPQELDILKTVKEITGFLLIQAWP
ENRTDLHAFENLEIIRGRTKQHGQFSLAVVSLNITSLGLRSLKEISDGDV
IISGNKNLCYANTINWKKLFGTSGQKTKIISNRGENSCKATGQVCHALCS
PEGCWGPEPRDCVSCRNVSRGRECVDKCNLLEGEPREFVENSECIQCHPE
CLPQAMNITCTGRGPDNCIQCAHYIDGPHCVKTCPAGVMGENNTLVWKYA
DAGHVCHLCHPNCTYGCTGPGLEGCPTNGPKIPSIATGMVGALLLLLVVA
LGIGLFMGSGEGRGSLLTCGDVEENPGPMALPVTALLLPLALLLHAEVQL
LESGGGLVQPGGSLRLSCAASGETFSDYQMSWVRQAPGKGLEWVSGIQGG
GGSTYYADSVKGRFTISRDNSKNTLYLQMNSLRAEDTAVYYCAREMWRGD
YYSGMDVWGQGTTVTVSSGGGGSDIVMTQSPDSLAVSLGERATINCKSSQ
SVLDSYNNENNLAWYQQKPGQPPKLLIYWASTRESGVPDRFSGSGSGTDF
TLTISSLQAEDVAVYYCQQYTSEPITFGQGTKVEIKEQKLISEEDLAAAI
EVMYPPPYLDNEKSNGTIIHVKGKHLCPSPLFPGPSKPFWVLVVVGGVLA
CYSLLVTVAFIIFWVRSKRSRLLHSDYMNMTPRRPGPTRKHYQPYAPPRD
FAAYRSRVKFSRSADAPAYQQGQNQLYNELNLGRREEYDVLDKRRGRDPE
MGGKPRRKNPQEGLYNELQKDKMAEAYSEIGMKGERRRGKGHDGLYQGLS
TATKDTYDALHMQALPPR

An exemplary nucleic acid sequence the amino acid sequence of SEQ ID NO: 95 is set forth in SEQ ID NO: 96, which is provided below.

[SEQ ID NO: 96]
ATGCTTCTCCTGGTGACAAGCCTTCTGCTCTGTGAGTTACCACACCCAGCA
TTCCTCCTGATCCCACGCAAAGTGTGTAACGGAATAGGTATTGGTGAATTT
AAAGACTCACTCTCCATAAATGCTACGAATATTAAACACTTCAAAAACTGC
ACCTCCATCAGTGGCGATCTCCACATCCTGCCGGTGGCATTTAGGGGTGAC
TCCTTCACACATACTCCTCCTCTGGACCCACAGGAACTGGATATTCTGAAA
ACCGTAAAGGAAATCACAGGGTTTTTGCTGATTCAGGCTTGGCCTGAAAAC
AGGACGGACCTCCATGCCTTTGAGAACCTAGAAATCATACGCGGCAGGACC
AAGCAACATGGTCAGTTTTCTCTTGCAGTCGTCAGCCTGAACATAACATCC
TTGGGATTACGCTCCCTCAAGGAGATAAGTGATGGAGATGTGATAATTTCA
GGAAACAAAAATTTGTGCTATGCAAATACAATAAACTGGAAAAAACTGTTT
GGGACCTCCGGTCAGAAAACCAAAATTATAAGCAACAGAGGTGAAAACAGC
TGCAAGGCCACAGGCCAGGTCTGCCATGCCTTGTGCTCCCCCGAGGGCTGC
TGGGGCCCGGAGCCCAGGGACTGCGTCTCTTGCCGGAATGTCAGCCGAGGC
AGGGAATGCGTGGACAAGTGCAACCTTCTGGAGGGTGAGCCAAGGGAGTTT
GTGGAGAACTCTGAGTGCATACAGTGCCACCCAGAGTGCCTGCCTCAGGCC
ATGAACATCACCTGCACAGGACGGGGACCAGACAACTGTATCCAGTGTGCC
CACTACATTGACGGCCCCCACTGCGTCAAGACCTGCCCGGCAGGAGTCATG
GGAGAAAACAACACCCTGGTCTGGAAGTACGCAGACGCCGGCCATGTGTGC
CACCTGTGCCATCCAAACTGCACCTACGGATGCACTGGGCCAGGTCTTGAA
GGCTGTCCAACGAATGGGCCTAAGATCCCGTCCATCGCCACTGGGATGGTG
GGGGCCCTCCTCTTGCTGCTGGTGGTGGCCCTGGGGATCGGCCTCTTCATG
GGTTCCGGTGAGGGACGGGGGTCACTGCTCACCTGCGGAGATGTAGAAGAG
AATCCCGGTCCCATGGCTCTCCCAGTGACTGCCCTACTGCTTCCCCTAGCG
CTTCTCCTGCATGCAGAGGTGCAGCTGTTGGAGTCTGGGGGAGGCTTGGTA
CAGCCTGGGGGGTCCCTGCGACTCTCCTGTGCAGCCTCTGGATTCACCTTT
AGCGACTATCAGATGAGCTGGGTCCGCCAGGCTCCAGGGAAGGGGCTGGAG
TGGGTGTCAGGCATTCAGGGTGGCGGTGGTAGCACATATTACGCAGACTCC
GTGAAGGGCCGGTTCACCATCTCCCGTGACAATTCCAAGAACACGCTGTAT
CTGCAAATGAACAGCCTGCGTGCCGAGGACACGGCTGTGTATTACTGTGCG
AGAGAGATGTGGCGTGGGGACTACTACTCCGGTATGGACGTCTGGGGCCAG
GGGACCACGGTCACCGTCTCCTCAGGCGGGGGTGGTAGTGACATCGTGATG
ACCCAGTCTCCAGACTCCCTGGCTGTGTCTCTGGGCGAGCGTGCCACCATC
AACTGCAAGTCCAGCCAGAGTGTTTTAGACAGCTATAACAATGAGAACAAT
TTAGCTTGGTATCAGCAGAAACCAGGACAGCCTCCTAAGCTGCTCATTTAC
TGGGCATCTACCCGGGAATCCGGGGTCCCTGACCGATTCAGTGGCAGCGGG
TCTGGGACAGATTTCACTCTCACCATCAGCAGCCTGCAGGCTGAAGATGTG
GCAGTTTATTACTGTCAGCAATATACCAGCGAACCTATCACGTTCGGCCAA
GGTACCAAGGTGGAAATCAAAGAACAGAAACTGATCTCTGAAGAAGACCTG
GCGGCCGCAATTGAAGTTATGTATCCTCCTCCTTACCTAGACAATGAGAAG
AGCAATGGAACCATTATCCATGTGAAAGGGAAACACCTTTGTCCAAGTCCC
CTATTTCCCGGACCTTCTAAGCCCTTTTGGGTGCTGGTGGTGGTTGGTGGA
GTCCTGGCTTGCTATAGCTTGCTAGTAACAGTGGCCTTTATTATTTTCTGG
GTGAGGAGTAAGAGGAGCAGGCTCCTGCACAGTGACTACATGAACATGACT
CCCCGCCGCCCCGGGCCCACCCGCAAGCATTACCAGCCCTATGCCCCACCA
CGCGACTTCGCAGCCTATCGCTCCAGAGTGAAGTTCAGCAGGAGCGCAGAC
GCCCCCGCGTACCAGCAGGGCCAGAACCAGCTCTATAACGAGCTCAATCTA
GGACGAAGAGAGGAGTACGATGTTTTGGACAAGAGACGTGGCCGGGACCCT
GAGATGGGGGGAAAGCCGAGAAGGAAGAACCCTCAGGAAGGCCTGTACAAT
GAACTGCAGAAAGATAAGATGGCGGAGGCCTACAGTGAGATTGGGATGAAA
GGCGAGCGCCGGAGGGGCAAGGGGCACGATGGCCTTTACCAGGGTCTCAGT
ACAGCCACCAAGGACACCTACGACGCCCTTCACATGCAGGCCCTGCCCCCT
CGCTAG

In certain embodiments, the CAR is a CD371-targeted CAR. In certain embodiments, the CAR comprises (a) an extracellular antigen-binding domain comprising (i) a V_{H} that comprises a CDR1 comprising the amino acid sequence set forth in SEQ ID NO: 28, a CDR2 comprising the amino acid sequence set forth in SEQ ID NO: 29, and a V_{H} CDR3 comprising the amino acid sequence set forth in SEQ ID NO: 30, and (ii) a V_{L} that comprises a CDR1 comprising the amino acid sequence set forth in SEQ ID NO: 31, a CDR2 comprising the amino acid sequence set forth in SEQ ID NO: 32, and a V_{L} CDR3 comprising the amino acid sequence set forth in SEQ ID NO: 33; (b) a transmembrane domain comprising a CD28 polypeptide (e.g., a transmembrane domain of human CD28 or a fragment thereof), and (c) an intracellular signaling domain comprising (i) a CD3ζ polypeptide, and (ii) a co-stimulatory signaling region comprising a CD28 polypeptide (e.g., an intracellular domain of human CD28 or a fragment thereof). In certain embodiments, the V_{H} and V_{L} are linked via a linker comprising or consisting of the amino acid sequence set forth in SEQ ID NO: 94. In certain embodiments, the V_{H} and V_{L} are positioned from the N- to the C-terminus: V_{H}-V_{L}. In certain embodiments, the CAR is designed as “Et.B10H2L_MT_h28Z”. In certain embodiments, the CAR comprises the amino acid sequence set forth in SEQ ID NO: 97, which is provided below.

[SEQ ID NO: 97]
MLLLVTSLLLCELPHPAFLLIPRKVCNGIGIGEFKDSLSINATNIKHFKN
CTSISGDLHILPVAFRGDSFTHTPPLDPQELDILKTVKEITGFLLIQAWP
ENRTDLHAFENLEIIRGRTKQHGQFSLAVVSLNITSLGLRSLKEISDGDV
IISGNKNLCYANTINWKKLFGTSGQKTKIISNRGENSCKATGQVCHALCS
PEGCWGPEPRDCVSCRNVSRGRECVDKCNLLEGEPREFVENSECIQCHPE
CLPQAMNITCTGRGPDNCIQCAHYIDGPHCVKTCPAGVMGENNTLVWKYA
DAGHVCHLCHPNCTYGCTGPGLEGCPTNGPKIPSIATGMVGALLLLLVVA
LGIGLFMGSGEGRGSLLTCGDVEENPGPMALPVTALLLPLALLLHAEVQL
LESGGGLVQPGGSLRLSCAASGFTFSDYQMSWVRQAPGKGLEWVSGIQGG
GGSTYYADSVKGRFTISRDNSKNTLYLQMNSLRAEDTAVYYCAREMWRGD
YYSGMDVWGQGTTVTVSSGGGGSGGGGSDIVMTQSPDSLAVSLGERATIN
CKSSQSVLDSYNNENNLAWYQQKPGQPPKLLIYWASTRESGVPDRFSGSG
SGTDFTLTISSLQAEDVAVYYCQQYTSEPITFGQGTKVEIKEQKLISEED
LAAAIEVMYPPPYLDNEKSNGTIIHVKGKHLCPSPLFPGPSKPFWVLVVV
GGVLACYSLLVTVAFIIFWVRSKRSRLLHSDYMNMTPRRPGPTRKHYQPY
APPRDFAAYRSRVKFSRSADAPAYQQGQNQLYNELNLGRREEYDVLDKRR
GRDPENGGKPRRKNPQEGLYNELQKDKMAEAYSEIGMKGERRRGKGHDGL
YQGLSTATKDTYDALHMQALPPR

An exemplary nucleic acid sequence the amino acid sequence of SEQ ID NO: 97 is set forth in SEQ ID NO: 98, which is provided below.

[SEQ ID NO: 98]
ATGCTTCTCCTGGTGACAAGCCTTCTGCTCTGTGAGTTACCACACCCAGCATTCCTCCTGATCCCACGCAA
AGTGTGTAACGGAATAGGTATTGGTGAATTTAAAGACTCACTCTCCATAAATGCTACGAATATTAAACACT
TCAAAAACTGCACCTCCATCAGTGGCGATCTCCACATCCTGCCGGTGGCATTTAGGGGTGACTCCTTCACA
CATACTCCTCCTCTGGACCCACAGGAACTGGATATTCTGAAAACCGTAAAGGAAATCACAGGGTTTTTGCT
GATTCAGGCTTGGCCTGAAAACAGGACGGACCTCCATGCCTTTGAGAACCTAGAAATCATACGCGGCAGGA
CCAAGCAACATGGTCAGTTTTCTCTTGCAGTCGTCAGCCTGAACATAACATCCTTGGGATTACGCTCCCTC
AAGGAGATAAGTGATGGAGATGTGATAATTTCAGGAAACAAAAATTTGTGCTATGCAAATACAATAAACTG
GAAAAAACTGTTTGGGACCTCCGGTCAGAAAACCAAAATTATAAGCAACAGAGGTGAAAACAGCTGCAAGG
CCACAGGCCAGGTCTGCCATGCCTTGTGCTCCCCCGAGGGCTGCTGGGGCCCGGAGCCCAGGGACTGCGTC
TCTTGCCGGAATGTCAGCCGAGGCAGGGAATGCGTGGACAAGTGCAACCTTCTGGAGGGTGAGCCAAGGGA
GTTTGTGGAGAACTCTGAGTGCATACAGTGCCACCCAGAGTGCCTGCCTCAGGCCATGAACATCACCTGCA
CAGGACGGGGACCAGACAACTGTATCCAGTGTGCCCACTACATTGACGGCCCCCACTGCGTCAAGACCTGC
CCGGCAGGAGTCATGGGAGAAAACAACACCCTGGTCTGGAAGTACGCAGACGCCGGCCATGTGTGCCACCT
GTGCCATCCAAACTGCACCTACGGATGCACTGGGCCAGGTCTTGAAGGCTGTCCAACGAATGGGCCTAAGA
TCCCGTCCATCGCCACTGGGATGGTGGGGGCCCTCCTCTTGCTGCTGGTGGTGGCCCTGGGGATCGGCCTC
TTCATGGGTTCCGGTGAGGGACGGGGGTCACTGCTCACCTGCGGAGATGTAGAAGAGAATCCCGGTCCCAT
GGCTCTCCCAGTGACTGCCCTACTGCTTCCCCTAGCGCTTCTCCTGCATGCAGAGGTGCAGCTGTTGGAGT
CTGGGGGAGGCTTGGTACAGCCTGGGGGGTCCCTGCGACTCTCCTGTGCAGCCTCTGGATTCACCTTTAGC
GACTATCAGATGAGCTGGGTCCGCCAGGCTCCAGGGAAGGGGCTGGAGTGGGTGTCAGGCATTCAGGGTGG
CGGTGGTAGCACATATTACGCAGACTCCGTGAAGGGCCGGTTCACCATCTCCCGTGACAATTCCAAGAACA
CGCTGTATCTGCAAATGAACAGCCTGCGTGCCGAGGACACGGCTGTGTATTACTGTGCGAGAGAGATGTGG
CGTGGGGACTACTACTCCGGTATGGACGTCTGGGGCCAGGGGACCACGGTCACCGTCTCCTCAGGCGGGGG
TGGTAGTGGAGGTGGCGGGTCTGACATCGTGATGACCCAGTCTCCAGACTCCCTGGCTGTGTCTCTGGGCG
AGCGTGCCACCATCAACTGCAAGTCCAGCCAGAGTGTTTTAGACAGCTATAACAATGAGAACAATTTAGCT
TGGTATCAGCAGAAACCAGGACAGCCTCCTAAGCTGCTCATTTACTGGGCATCTACCCGGGAATCCGGGGT
CCCTGACCGATTCAGTGGCAGCGGGTCTGGGACAGATTTCACTCTCACCATCAGCAGCCTGCAGGCTGAAG
ATGTGGCAGTTTATTACTGTCAGCAATATACCAGCGAACCTATCACGTTCGGCCAAGGTACCAAGGTGGAA
ATCAAAGAACAGAAACTGATCTCTGAAGAAGACCTGGCGGCCGCAATTGAAGTTATGTATCCTCCTCCTTA
CCTAGACAATGAGAAGAGCAATGGAACCATTATCCATGTGAAAGGGAAACACCTTTGTCCAAGTCCCCTAT
TTCCCGGACCTTCTAAGCCCTTTTGGGTGCTGGTGGTGGTTGGTGGAGTCCTGGCTTGCTATAGCTTGCTA
GTAACAGTGGCCTTTATTATTTTCTGGGTGAGGAGTAAGAGGAGCAGGCTCCTGCACAGTGACTACATGAA
CATGACTCCCCGCCGCCCCGGGCCCACCCGCAAGCATTACCAGCCCTATGCCCCACCACGCGACTTCGCAG
CCTATCGCTCCAGAGTGAAGTTCAGCAGGAGCGCAGACGCCCCCGCGTACCAGCAGGGCCAGAACCAGCTC
TATAACGAGCTCAATCTAGGACGAAGAGAGGAGTACGATGTTTTGGACAAGAGACGTGGCCGGGACCCTGA
GATGGGGGGAAAGCCGAGAAGGAAGAACCCTCAGGAAGGCCTGTACAATGAACTGCAGAAAGATAAGATGG
CGGAGGCCTACAGTGAGATTGGGATGAAAGGCGAGCGCCGGAGGGGCAAGGGGCACGATGGCCTTTACCAG
GGTCTCAGTACAGCCACCAAGGACACCTACGACGCCCTTCACATGCAGGCCCTGCCCCCTCGCTAG

In certain embodiments, the CAR is a CD371-targeted CAR. In certain embodiments, the CAR comprises (a) an extracellular antigen-binding domain comprising (i) a V_{H} that comprises a CDR1 comprising the amino acid sequence set forth in SEQ ID NO: 28, a CDR2 comprising the amino acid sequence set forth in SEQ ID NO: 29, and a V_{H} CDR3 comprising the amino acid sequence set forth in SEQ ID NO: 30, and (ii) a V_{L} that comprises a CDR1 comprising the amino acid sequence set forth in SEQ ID NO: 31, a CDR2 comprising the amino acid sequence set forth in SEQ ID NO: 32, and a V_{L} CDR3 comprising the amino acid sequence set forth in SEQ ID NO: 33; (b) a transmembrane domain comprising a CD28 polypeptide (e.g., a transmembrane domain of human CD28 or a fragment thereof), and (c) an intracellular signaling domain comprising (i) a CD3ζ polypeptide, and (ii) a co-stimulatory signaling region comprising a CD28 polypeptide (e.g., an intracellular domain of human CD28 or a fragment thereof). In certain embodiments, the V_{H} and V_{L} are linked via a linker comprising or consisting of the amino acid sequence set forth in SEQ ID NO: 91. In certain embodiments, the V_{H} and V_{L} are positioned from the N- to the C-terminus: V_{H}-V_{L}. In certain embodiments, the CAR is designed as “Et.B10H5L_MT_h28Z”. In certain embodiments, the CAR comprises the amino acid sequence set forth in SEQ ID NO: 99, which is provided below.

[SEQ ID NO: 99]
MLLLVTSLLLCELPHPAFLLIPRKVCNGIGIGEFKDSLSINATNIKHFKN
CTSISGDLHILPVAFRGDSFTHTPPLDPQELDILKTVKEITGFLLIQAWP
ENRTDLHAFENLEIIRGRTKQHGQFSLAVVSLNITSLGLRSLKEISDGDV
IISGNKNLCYANTINWKKLFGTSGQKTKIISNRGENSCKATGQVCHALCS
PEGCWGPEPRDCVSCRNVSRGRECVDKCNLLEGEPREFVENSECIQCHPE
CLPQAMNITCTGRGPDNCIQCAHYIDGPHCVKTCPAGVMGENNTLVWKYA
DAGHVCHLCHPNCTYGCTGPGLEGCPTNGPKIPSIATGMVGALLLLLVVA
LGIGLFMGSGEGRGSLLTCGDVEENPGPMALPVTALLLPLALLLHAEVQL
LESGGGLVQPGGSLRLSCAASGFTFSDYQMSWVRQAPGKGLEWVSGIQGG
GGSTYYADSVKGRFTISRDNSKNTLYLQMNSLRAEDTAVYYCAREMWRGD
YYSGMDVWGQGTTVTVSSGGGGSGGGGSGGGGSGGGSGGGGSDIVMTQSP
DSLAVSLGERATINCKSSQSVLDSYNNENNLAWYQQKPGQPPKLLIYWAS
TRESGVPDRFSGSGSGTDFTLTISSLQAEDVAVYYCQQYTSEPITFGQGT
KVEIKEQKLISEEDLAAAIEVMYPPPYLDNEKSNGTIIHVKGKHLCPSPL
FPGPSKPFWVLVVVGGVLACYSLLVTVAFIIFWVRSKRSRLLHSDYMNMT
PRRPGPTRKHYQPYAPPRDFAAYRSRVKFSRSADAPAYQQGQNQLYNELN
LGRREEYDVLDKRRGRDPENGGKPRRKNPQEGLYNELQKDKMAEAYSEIG
MKGERRRGKGHDGLYQGLSTATKDTYDALHMQALPPR

An exemplary nucleic acid sequence the amino acid sequence of SEQ ID NO: 99 is set forth in SEQ ID NO: 100, which is provided below.

[SEQ ID NO: 100]
ATGCTTCTCCTGGTGACAAGCCTTCTGCTCTGTGAGTTACCACACCCAGC
ATTCCTCCTGATCCCACGCAAAGTGTGTAACGGAATAGGTATTGGTGAAT
TTAAAGACTCACTCTCCATAAATGCTACGAATATTAAACACTTCAAAAAC
TGCACCTCCATCAGTGGCGATCTCCACATCCTGCCGGTGGCATTTAGGGG
TGACTCCTTCACACATACTCCTCCTCTGGACCCACAGGAACTGGATATTC
TGAAAACCGTAAAGGAAATCACAGGGTTTTTGCTGATTCAGGCTTGGCCT
GAAAACAGGACGGACCTCCATGCCTTTGAGAACCTAGAAATCATACGCGG
CAGGACCAAGCAACATGGTCAGTTTTCTCTTGCAGTCGTCAGCCTGAACA
TAACATCCTTGGGATTACGCTCCCTCAAGGAGATAAGTGATGGAGATGTG
ATAATTTCAGGAAACAAAAATTTGTGCTATGCAAATACAATAAACTGGAA
AAAACTGTTTGGGACCTCCGGTCAGAAAACCAAAATTATAAGCAACAGAG
GTGAAAACAGCTGCAAGGCCACAGGCCAGGTCTGCCATGCCTTGTGCTCC
CCCGAGGGCTGCTGGGGCCCGGAGCCCAGGGACTGCGTCTCTTGCCGGAA
TGTCAGCCGAGGCAGGGAATGCGTGGACAAGTGCAACCTTCTGGAGGGTG
AGCCAAGGGAGTTTGTGGAGAACTCTGAGTGCATACAGTGCCACCCAGAG
TGCCTGCCTCAGGCCATGAACATCACCTGCACAGGACGGGGACCAGACAA
CTGTATCCAGTGTGCCCACTACATTGACGGCCCCCACTGCGTCAAGACCT
GCCCGGCAGGAGTCATGGGAGAAAACAACACCCTGGTCTGGAAGTACGCA
GACGCCGGCCATGTGTGCCACCTGTGCCATCCAAACTGCACCTACGGATG
CACTGGGCCAGGTCTTGAAGGCTGTCCAACGAATGGGCCTAAGATCCCGT
CCATCGCCACTGGGATGGTGGGGGCCCTCCTCTTGCTGCTGGTGGTGGCC
CTGGGGATCGGCCTCTTCATGGGTTCCGGTGAGGGACGGGGGTCACTGCT
CACCTGCGGAGATGTAGAAGAGAATCCCGGTCCCATGGCTCTCCCAGTGA
CTGCCCTACTGCTTCCCCTAGCGCTTCTCCTGCATGCAGAGGTGCAGCTG
TTGGAGTCTGGGGGAGGCTTGGTACAGCCTGGGGGGTCCCTGCGACTCTC
CTGTGCAGCCTCTGGATTCACCTTTAGCGACTATCAGATGAGCTGGGTCC
GCCAGGCTCCAGGGAAGGGGCTGGAGTGGGTGTCAGGCATTCAGGGTGGC
GGTGGTAGCACATATTACGCAGACTCCGTGAAGGGCCGGTTCACCATCTC
CCGTGACAATTCCAAGAACACGCTGTATCTGCAAATGAACAGCCTGCGTG
CCGAGGACACGGCTGTGTATTACTGTGCGAGAGAGATGTGGCGTGGGGAC
TACTACTCCGGTATGGACGTCTGGGGCCAGGGGACCACGGTCACCGTCTC
CTCAGGTGGTGGTGGTTCAGGTGGTGGTGGTTCTGGAGGGGGCGGTTCTG
GCGGCGGCTCCGGTGGTGGTGGATCCGACATCGTGATGACCCAGTCTCCA
GACTCCCTGGCTGTGTCTCTGGGCGAGCGTGCCACCATCAACTGCAAGTC
CAGCCAGAGTGTTTTAGACAGCTATAACAATGAGAACAATTTAGCTTGGT
ATCAGCAGAAACCAGGACAGCCTCCTAAGCTGCTCATTTACTGGGCATCT
ACCCGGGAATCCGGGGTCCCTGACCGATTCAGTGGCAGCGGGTCTGGGAC
AGATTTCACTCTCACCATCAGCAGCCTGCAGGCTGAAGATGTGGCAGTTT
ATTACTGTCAGCAATATACCAGCGAACCTATCACGTTCGGCCAAGGTACC
AAGGTGGAAATCAAAGAACAGAAACTGATCTCTGAAGAAGACCTGGCGGC
CGCAATTGAAGTTATGTATCCTCCTCCTTACCTAGACAATGAGAAGAGCA
ATGGAACCATTATCCATGTGAAAGGGAAACACCTTTGTCCAAGTCCCCTA
TTTCCCGGACCTTCTAAGCCCTTTTGGGTGCTGGTGGTGGTTGGTGGAGT
CCTGGCTTGCTATAGCTTGCTAGTAACAGTGGCCTTTATTATTTTCTGGG
TGAGGAGTAAGAGGAGCAGGCTCCTGCACAGTGACTACATGAACATGACT
CCCCGCCGCCCCGGGCCCACCCGCAAGCATTACCAGCCCTATGCCCCACC
ACGCGACTTCGCAGCCTATCGCTCCAGAGTGAAGTTCAGCAGGAGCGCAG
ACGCCCCCGCGTACCAGCAGGGCCAGAACCAGCTCTATAACGAGCTCAAT
CTAGGACGAAGAGAGGAGTACGATGTTTTGGACAAGAGACGTGGCCGGGA
CCCTGAGATGGGGGGAAAGCCGAGAAGGAAGAACCCTCAGGAAGGCCTGT
ACAATGAACTGCAGAAAGATAAGATGGCGGAGGCCTACAGTGAGATTGGG
ATGAAAGGCGAGCGCCGGAGGGGCAAGGGGCACGATGGCCTTTACCAGGG
TCTCAGTACAGCCACCAAGGACACCTACGACGCCCTTCACATGCAGGCCC
TGCCCCCTCGCTAG

In certain embodiments, the CAR is a CD371-targeted CAR. In certain embodiments, the CAR comprises (a) an extracellular antigen-binding domain comprising (i) a V_{H} that comprises a CDR1 comprising the amino acid sequence set forth in SEQ ID NO: 28, a CDR2 comprising the amino acid sequence set forth in SEQ ID NO: 29, and a V_{H} CDR3 comprising the amino acid sequence set forth in SEQ ID NO: 30, and (ii) a V_{L} that comprises a CDR1 comprising the amino acid sequence set forth in SEQ ID NO: 31, a CDR2 comprising the amino acid sequence set forth in SEQ ID NO: 32, and a V_{L} CDR3 comprising the amino acid sequence set forth in SEQ ID NO: 33; (b) a transmembrane domain comprising a CD28 polypeptide (e.g., a transmembrane domain of human CD28 or a fragment thereof), and (c) an intracellular signaling domain comprising (i) a CD3ζ polypeptide, and (ii) a co-stimulatory signaling region comprising a CD28 polypeptide (e.g., an intracellular domain of human CD28 or a fragment thereof). In certain embodiments, the V_{H} and V_{L} are linked via a linker comprising or consisting of the amino acid sequence set forth in SEQ ID NO: 92. In certain embodiments, the V_{H} and V_{L} are positioned from the N- to the C-terminus: V_{H}-V_{L}. In certain embodiments, the CAR is designed as “Et.B10H6L_MT_h28Z”. In certain embodiments, the CAR comprises the amino acid sequence set forth in SEQ ID NO: 101, which is provided below.

[SEQ ID NO: 101]
MLLLVTSLLLCELPHPAFLLIPRKVCNGIGIGEFKDSLSINATNIKHFKN
CTSISGDLHILPVAFRGDSFTHTPPLDPQELDILKTVKEITGFLLIQAWP
ENRTDLHAFENLEIIRGRTKQHGQFSLAVVSLNITSLGLRSLKEISDGDV
IISGNKNLCYANTINWKKLFGTSGQKTKIISNRGENSCKATGQVCHALCS
PEGCWGPEPRDCVSCRNVSRGRECVDKCNLLEGEPREFVENSECIQCHPE
CLPQAMNITCTGRGPDNCIQCAHYIDGPHCVKTCPAGVMGENNTLVWKYA
DAGHVCHLCHPNCTYGCTGPGLEGCPTNGPKIPSIATGMVGALLLLLVVA
LGIGLFMGSGEGRGSLLTCGDVEENPGPMALPVTALLLPLALLLHAEVQL
LESGGGLVQPGGSLRLSCAASGFTFSDYQMSWVRQAPGKGLEWVSGIQGG
GGSTYYADSVKGRFTISRDNSKNTLYLQMNSLRAEDTAVYYCAREMWRGD
YYSGMDVWGQGTTVTVSSGGGGSGGGGSGGGGSGGGGSGGGSGGGGSDIV
MTQSPDSLAVSLGERATINCKSSQSVLDSYNNENNLAWYQQKPGQPPKLL
IYWASTRESGVPDRFSGSGSGTDFTLTISSLQAEDVAVYYCQQYTSEPIT
FGQGTKVEIKEQKLISEEDLAAAIEVMYPPPYLDNEKSNGTIIHVKGKHL
CPSPLFPGPSKPFWVLVVVGGVLACYSLLVTVAFIIFWVRSKRSRLLHSD
YMNMTPRRPGPTRKHYQPYAPPRDFAAYRSRVKFSRSADAPAYQQGQNQL
YNELNLGRREEYDVLDKRRGRDPEMGGKPRRKNPQEGLYNELQKDKMAEA
YSEIGMKGERRRGKGHDGLYQGLSTATKDTYDALHMQALPPR

An exemplary nucleic acid sequence the amino acid sequence of SEQ ID NO: 101 is set forth in SEQ ID NO: 102, which is provided below.

[SEQ ID NO: 102]
ATGCTTCTCCTGGTGACAAGCCTTCTGCTCTGTGAGTTACCACACCCAGC
ATTCCTCCTGATCCCACGCAAAGTGTGTAACGGAATAGGTATTGGTGAAT
TTAAAGACTCACTCTCCATAAATGCTACGAATATTAAACACTTCAAAAAC
TGCACCTCCATCAGTGGCGATCTCCACATCCTGCCGGTGGCATTTAGGGG
TGACTCCTTCACACATACTCCTCCTCTGGACCCACAGGAACTGGATATTC
TGAAAACCGTAAAGGAAATCACAGGGTTTTTGCTGATTCAGGCTTGGCCT
GAAAACAGGACGGACCTCCATGCCTTTGAGAACCTAGAAATCATACGCGG
CAGGACCAAGCAACATGGTCAGTTTTCTCTTGCAGTCGTCAGCCTGAACA
TAACATCCTTGGGATTACGCTCCCTCAAGGAGATAAGTGATGGAGATGTG
ATAATTTCAGGAAACAAAAATTTGTGCTATGCAAATACAATAAACTGGAA
AAAACTGTTTGGGACCTCCGGTCAGAAAACCAAAATTATAAGCAACAGAG
GTGAAAACAGCTGCAAGGCCACAGGCCAGGTCTGCCATGCCTTGTGCTCC
CCCGAGGGCTGCTGGGGCCCGGAGCCCAGGGACTGCGTCTCTTGCCGGAA
TGTCAGCCGAGGCAGGGAATGCGTGGACAAGTGCAACCTTCTGGAGGGTG
AGCCAAGGGAGTTTGTGGAGAACTCTGAGTGCATACAGTGCCACCCAGAG
TGCCTGCCTCAGGCCATGAACATCACCTGCACAGGACGGGGACCAGACAA
CTGTATCCAGTGTGCCCACTACATTGACGGCCCCCACTGCGTCAAGACCT
GCCCGGCAGGAGTCATGGGAGAAAACAACACCCTGGTCTGGAAGTACGCA
GACGCCGGCCATGTGTGCCACCTGTGCCATCCAAACTGCACCTACGGATG
CACTGGGCCAGGTCTTGAAGGCTGTCCAACGAATGGGCCTAAGATCCCGT
CCATCGCCACTGGGATGGTGGGGGCCCTCCTCTTGCTGCTGGTGGTGGCC
CTGGGGATCGGCCTCTTCATGGGTTCCGGTGAGGGACGGGGGTCACTGCT
CACCTGCGGAGATGTAGAAGAGAATCCCGGTCCCATGGCTCTCCCAGTGA
CTGCCCTACTGCTTCCCCTAGCGCTTCTCCTGCATGCAGAGGTGCAGCTG
TTGGAGTCTGGGGGAGGCTTGGTACAGCCTGGGGGGTCCCTGCGACTCTC
CTGTGCAGCCTCTGGATTCACCTTTAGCGACTATCAGATGAGCTGGGTCC
GCCAGGCTCCAGGGAAGGGGCTGGAGTGGGTGTCAGGCATTCAGGGTGGC
GGTGGTAGCACATATTACGCAGACTCCGTGAAGGGCCGGTTCACCATCTC
CCGTGACAATTCCAAGAACACGCTGTATCTGCAAATGAACAGCCTGCGTG
CCGAGGACACGGCTGTGTATTACTGTGCGAGAGAGATGTGGCGTGGGGAC
TACTACTCCGGTATGGACGTCTGGGGCCAGGGGACCACGGTCACCGTCTC
CTCAGGTGGTGGTGGTTCAGGTGGTGGTGGTTCTGGTGGGGGCGGTAGTG
GAGGGGGCGGTTCTGGCGGCGGCTCCGGTGGTGGTGGATCCGACATCGTG
ATGACCCAGTCTCCAGACTCCCTGGCTGTGTCTCTGGGCGAGCGTGCCAC
CATCAACTGCAAGTCCAGCCAGAGTGTTTTAGACAGCTATAACAATGAGA
ACAATTTAGCTTGGTATCAGCAGAAACCAGGACAGCCTCCTAAGCTGCTC
ATTTACTGGGCATCTACCCGGGAATCCGGGGTCCCTGACCGATTCAGTGG
CAGCGGGTCTGGGACAGATTTCACTCTCACCATCAGCAGCCTGCAGGCTG
AAGATGTGGCAGTTTATTACTGTCAGCAATATACCAGCGAACCTATCACG
TTCGGCCAAGGTACCAAGGTGGAAATCAAAGAACAGAAACTGATCTCTGA
AGAAGACCTGGCGGCCGCAATTGAAGTTATGTATCCTCCTCCTTACCTAG
ACAATGAGAAGAGCAATGGAACCATTATCCATGTGAAAGGGAAACACCTT
TGTCCAAGTCCCCTATTTCCCGGACCTTCTAAGCCCTTTTGGGTGCTGGT
GGTGGTTGGTGGAGTCCTGGCTTGCTATAGCTTGCTAGTAACAGTGGCCT
TTATTATTTTCTGGGTGAGGAGTAAGAGGAGCAGGCTCCTGCACAGTGAC
TACATGAACATGACTCCCCGCCGCCCCGGGCCCACCCGCAAGCATTACCA
GCCCTATGCCCCACCACGCGACTTCGCAGCCTATCGCTCCAGAGTGAAGT
TCAGCAGGAGCGCAGACGCCCCCGCGTACCAGCAGGGCCAGAACCAGCTC
TATAACGAGCTCAATCTAGGACGAAGAGAGGAGTACGATGTTTTGGACAA
GAGACGTGGCCGGGACCCTGAGATGGGGGGAAAGCCGAGAAGGAAGAACC
CTCAGGAAGGCCTGTACAATGAACTGCAGAAAGATAAGATGGCGGAGGCC
TACAGTGAGATTGGGATGAAAGGCGAGCGCCGGAGGGGCAAGGGGCACGA
TGGCCTTTACCAGGGTCTCAGTACAGCCACCAAGGACACCTACGACGCCC
TTCACATGCAGGCCCTGCCCCCTCGCTAG

In certain embodiments, a presently disclosed CAR further comprises an inducible promoter, for expressing nucleic acid sequences in human cells. Promoters for use in expressing CAR genes can be a constitutive promoter, such as ubiquitin C (UbiC) promoter.

5.3.3. TCR Like Fusion Molecules

In certain embodiments, the antigen-recognizing receptor is a TCR like fusion molecule. Non-limiting examples of TCR fusion molecules include HLA-Independent TCR-based Chimeric Antigen Receptor (also known as “HIT-CAR”, e.g., those disclosed in International Patent Application No. PCT/US19/017525, which is incorporated by reference in its entirety), and T cell receptor fusion constructs (TRuCs) (e.g., those disclosed in Baeuerle et al., “Synthetic TRuC receptors engaging the complete T cell receptor for potent anti-tumor response,” Nature Communications volume 10, Article number: 2087 (2019), which is incorporated by reference in its entirety).

In certain embodiments, the TCR like fusion molecule comprises an antigen binding chain that comprises an extracellular antigen-binding domain and a constant domain, wherein the TCR like fusion molecule binds to an antigen in an HLA-independent manner. In certain embodiments, the constant domain comprises a T cell receptor constant region selected from the group consisting of a native or modified TRAC peptide, a native or modified TRBC peptide, a native or modified TRDC peptide, a native or modified TRGC peptide and any variants or functional fragments thereof. In certain embodiments, the constant domain comprises a native or modified TRAC peptide. In certain embodiments, the constant domain comprises a native or modified TRBC peptide. In certain embodiments, the constant domain is capable of forming a homodimer or a heterodimer with another constant domain. In certain embodiments, the antigen binding chain is capable of associating with a CD3ζ polypeptide. In certain embodiments, the antigen binding chain, upon binding to an antigen, is capable of activating the CD3ζ polypeptide associated to the antigen binding chain. In certain embodiments, the activation of the CD3ζ polypeptide is capable of activating an immunoresponsive cell. In certain embodiments, the TCR like fusion molecule is capable of integrating with a CD3 complex and providing HLA-independent antigen recognition. In certain embodiments, the TCR like fusion molecule replaces an endogenous TCR in a CD3/TCR complex. In certain embodiments, the extracellular antigen-binding domain of the TCR like fusion molecule is capable of dimerizing with another extracellular antigen-binding domain. In certain embodiments, the extracellular antigen-binding domain of the TCR like fusion molecule comprises a ligand for a cell-surface receptor, a receptor for a cell surface ligand, an antigen binding portion of an antibody or a fragment thereof or an antigen binding portion of a TCR. In certain embodiments, the extracellular antigen-binding domain of the TCR like fusion molecule comprises one or two immunoglobulin variable region(s). In certain embodiments, the extracellular antigen-binding domain of the TCR like fusion molecule comprises a heavy chain variable region (V_{H}) of an antibody. In certain embodiments, the extracellular antigen-binding domain of the TCR like fusion molecule comprises a light chain variable region (V_{L}) of an antibody. In certain embodiments, the extracellular antigen-binding domain of the TCR like fusion molecule is capable of dimerizing with another extracellular antigen-binding domain. In certain embodiments, the extracellular antigen-binding domain of the TCR like fusion molecule comprises a V_{H} of an antibody, wherein the V_{H} is capable of dimerizing with another extracellular antigen-binding domain comprising a V_{L} of the antibody and form a fragment variable (Fv). In certain embodiments, the extracellular antigen-binding domain of the TCR like fusion molecule comprises a V_{L} of an antibody, wherein the V_{L} is capable of dimerizing with another extracellular antigen-binding domain comprising a V_{H} of the antibody and form a fragment variable (Fv).

The TCR like fusion molecule can bind to a tumor antigen or a pathogen antigen. In certain embodiments, the TCR like fusion molecule binds to a tumor antigen.

### 5.4. Cells

The presently disclosed subject matter provides cells comprising a presently disclosed CD371-targeted antigen-recognizing receptor (e.g., one disclosed in Section 5.3). In certain embodiments, the cell is selected from the group consisting of cells of lymphoid lineage and cells of myeloid lineage. In certain embodiments, the cell is an immunoresponsive cell. In certain embodiments, the immunoresponsive cell is a cell of lymphoid lineage.

In certain embodiments, the cell is a cell of the lymphoid lineage. Cells of the lymphoid lineage can provide production of antibodies, regulation of cellular immune system, detection of foreign agents in the blood, detection of cells foreign to the host, and the like. Non-limiting examples of cells of the lymphoid lineage include T cells, Natural Killer (NK) cells, B cells, dendritic cells, stem cells from which lymphoid cells may be differentiated. In certain embodiments, the stem cell is a pluripotent stem cell (e.g., embryonic stem cell).

In certain embodiments, the cell is a T cell. T cells can be lymphocytes that mature in the thymus and are chiefly responsible for cell-mediated immunity. T cells are involved in the adaptive immune system. The T cells of the presently disclosed subject matter can be any type of T cells, including, but not limited to, helper T cells, cytotoxic T cells, memory T cells (including central memory T cells, stem-cell-like memory T cells (or stem-like memory T cells), and two types of effector memory T cells: e.g., TEM cells and TEMRA cells, Regulatory T cells (also known as suppressor T cells), tumor-infiltrating lymphocyte (TIL), Natural killer T cells, Mucosal associated invariant T cells, and γδ T cells. Cytotoxic T cells (CTL or killer T cells) are a subset of T lymphocytes capable of inducing the death of infected somatic or tumor cells. A patient's own T cells may be genetically modified to target specific antigens through the introduction of an antigen-recognizing receptor, e.g., a CAR or a TCR. In certain embodiments, the immunoresponsive cell is a T cell. The T cell can be a CD4⁺ T cell or a CD8⁺ T cell. In certain embodiments, the T cell is a CD4⁺ T cell. In certain embodiments, the T cell is a CD8⁺ T cell.

In certain embodiments, the cell is a NK cell. Natural killer (NK) cells can be lymphocytes that are part of cell-mediated immunity and act during the innate immune response. NK cells do not require prior activation in order to perform their cytotoxic effect on target cells.

Types of human lymphocytes of the presently disclosed subject matter include, without limitation, peripheral donor lymphocytes. e.g., those disclosed in Sadelain et al., Nat Rev Cancer (2003); 3:35-45 (disclosing peripheral donor lymphocytes genetically modified to express CARs), in Morgan, R. A., et al. 2006 Science 314:126-129 (disclosing peripheral donor lymphocytes genetically modified to express a full-length tumor antigen-recognizing T cell receptor complex comprising the α and β heterodimer), in Panelli et al., J Immunol (2000); 164:495-504; Panelli et al., J Immunol (2000); 164:4382-4392 (disclosing lymphocyte cultures derived from tumor infiltrating lymphocytes (TILs) in tumor biopsies), and in Dupont et al., Cancer Res (2005); 65:5417-5427; Papanicolaou et al., Blood (2003); 102:2498-2505 (disclosing selectively in vitro-expanded antigen-specific peripheral blood leukocytes employing artificial antigen-presenting cells (AAPCs) or pulsed dendritic cells).

The cells (e.g., T cells) can be autologous, non-autologous (e.g., allogeneic), or derived in vitro from engineered progenitor or stem cells.

The cells of the presently disclosed subject matter can be cells of the myeloid lineage. Non-limiting examples of cells of the myeloid lineage include monocytes, macrophages, neutrophils, dendritic cells, basophils, neutrophils, eosinophils, megakaryocytes, mast cell, erythrocyte, thrombocytes, and stem cells from which myeloid cells may be differentiated. In certain embodiments, the stem cell is a pluripotent stem cell (e.g., an embryonic stem cell or an induced pluripotent stem cell).

In certain embodiments, the presently disclosed cells are capable of modulating the tumor microenvironment. Tumors have a microenvironment that is hostile to the host immune response involving a series of mechanisms by malignant cells to protect themselves from immune recognition and elimination. This “hostile tumor microenvironment” comprises a variety of immune suppressive factors including infiltrating regulatory CD4⁺ T cells (Tregs), myeloid derived suppressor cells (MDSCs), tumor associated macrophages (TAMs), immune suppressive cytokines including TGF-β, and expression of ligands targeted to immune suppressive receptors expressed by activated T cells (CTLA-4 and PD-1). These mechanisms of immune suppression play a role in the maintenance of tolerance and suppressing inappropriate immune responses, however within the tumor microenvironment these mechanisms prevent an effective anti-tumor immune response. Collectively these immune suppressive factors can induce either marked anergy or apoptosis of adoptively transferred CAR modified T cells upon encounter with targeted tumor cells.

In certain embodiments, the cells can be transduced with the presently disclosed CD371-targeted antigen-recognizing receptor such that the cells express the antigen-recognizing receptor.

In certain embodiments, the cell further comprises a soluble single-chain variable fragment (scFv) that binds a polypeptide that has immunosuppressive activity or immunostimulatory activity. In certain embodiments, immunosuppressive activity refers to induction of signal transduction or changes in protein expression in a cell (e.g., an activated immunoresponsive cell) resulting in a decrease in an immune response. Polypeptides known to suppress or decrease an immune response via their binding include CD47, PD-1, CTLA-4, and their corresponding ligands, including SIRPa, PD-L1, PD-L2, B7-1, and B7-2. Such polypeptides are present in the tumor microenvironment and inhibit immune responses to neoplastic cells. In various embodiments, inhibiting, blocking, or antagonizing the interaction of immunosuppressive polypeptides and/or their ligands enhances the immune response of the immunoresponsive cell.

In certain embodiments, immunostimulatory activity refers to induction of signal transduction or changes in protein expression in a cell (e.g., an activated immunoresponsive cell) resulting in an increase in an immune response. Immunostimulatory activity may include pro-inflammatory activity. Polypeptides known to stimulate or increase an immune response via their binding include CD28, OX-40, 4-IBB, and their corresponding ligands, including B7-1, B7-2, OX-40L, and 4-1BBL. Such polypeptides are present in the tumor microenvironment and activate immune responses to neoplastic cells. In various embodiments, promoting, stimulating, or agonizing pro-inflammatory polypeptides and/or their ligands enhances the immune response of the immunoresponsive cell.

Cells comprising an antigen-recognizing receptor (e.g., a CAR) and a soluble scFv that binds a polypeptide that has immunosuppressive activity or immunostimulatory activity are disclosed in International Patent Publication No. WO 2014/134165, which is incorporated by reference in its entirety.

In certain embodiments, the cell further comprises an exogenous CD40L. Cells comprising an antigen-recognizing receptor (e.g., a CAR) and an exogenous CD40L are disclosed in International Patent Publication No. WO 2014/134165.

Furthermore, in certain embodiments, the cell is engineered to express IL-18. In certain embodiments, the cell further comprises an exogenous IL-18 polypeptide. In certain embodiments, the exogenous IL-18 polypeptide comprises or consists of the amino acid sequence set forth in SEQ ID NO: 103, which is provided below.

[SEQ ID NO: 103]
MGYRMQLLSCIALSLALVTNSGYFGKLESKLSVIRNLNDQVLFIDQGNRP
LFEDMTDSDCRDNAPRTIFIISMYKDSQPRGMAVTISVKCEKISTLSCEN
KIISFKEMNPPDNIKDTKSDIIFFQRSVPGHDNKMQFESSSYEGYFLACE
KERDLFKLILKKEDELGDRSIMFTVQNED

In certain embodiments, the cells further comprise a nucleic acid molecule encoding an IL-18 polypeptide. In certain embodiments, the nucleic acid molecule comprises the nucleotide sequence set forth in SEQ ID NO: 104, which is provided below.

[SEQ ID NO: 104]
ATGGGTTACAGGATGCAACTCCTGTCTTGCATTGCACTAAGTCTTGCACT
TGTCACAAACAGTGGCTACTTTGGCAAGCTTGAATCTAAATTATCAGTCA
TAAGAAATTTGAATGACCAAGTTCTCTTCATTGACCAAGGAAATCGGCCT
CTATTTGAAGATATGACTGATTCTGACTGTAGAGATAATGCACCCCGGAC
CATATTTATTATAAGTATGTATAAAGATAGCCAGCCTAGAGGTATGGCTG
TAACTATCTCTGTGAAGTGTGAGAAAATTTCAACTCTCTCCTGTGAGAAC
AAAATTATTTCCTTTAAGGAAATGAATCCTCCTGATAACATCAAGGATAC
AAAAAGTGACATCATATTCTTTCAGAGAAGTGTCCCAGGACATGATAATA
AGATGCAATTTGAATCTTCATCATACGAAGGATACTTTCTAGCTTGTGAA
AAAGAGAGAGACCTTTTTAAACTCATTTTGAAAAAAGAGGATGAATTGGG
GGATAGATCTATAATGTTCACTGTTCAAAACGAAGACTAG

Alternatively, in certain embodiments, the cell further comprises a modified promoter/enhancer at an IL-18 gene locus, which can increase IL-18 gene expression, e.g., a constitutive or inducible promoter is placed to drive IL-18 gene expression.

Cells comprising an antigen-recognizing receptor (e.g., a CAR) and engineered to express IL-18, e.g., comprising an exogenous IL-18 polypeptide or a modified promoter/enhancer at an IL-18 gene locus are disclosed in International Patent Publication No. WO2018/027155, which is incorporated by reference in its entirety.

Additionally or alternatively, the cell is engineered to express IL-33. In certain embodiments, the cell further comprises an exogenous IL-33 polypeptide. In certain embodiments, the exogenous IL-33 polypeptide comprises or consists of the amino acid sequence set forth in SEQ ID NO: 105, which is provided below.

[SEQ ID NO: 105]
MYRMQLLSCIALSLALVTNSSITGISPITEYLASLSTYNDQSITFALEDE
SYEIYVEDLKKDEKKDKVLLSYYESQHPSNESGDGVDGKMLMVTLSPTKD
FWLHANNKEHSVELHKCEKPLPDQAFFVLHNMHSNCVSFECKTDPGVFIG
VKDNHLALIKVDSSENLCTENILFKLSET

In certain embodiments, the cells further comprise a nucleic acid molecule encoding an IL-33 polypeptide. In certain embodiments, the nucleic acid molecule comprises the nucleotide sequence set forth in SEQ ID NO: 106, which is provided below.

[SEQ ID NO: 106]
ATGTACAGGATGCAACTCCTGTCTTGCATTGCACTAAGTCTTGCACTTGT
CACAAACAGTAGTATCACAGGAATTTCACCTATTACAGAGTATCTTGCTT
CTCTAAGCACATACAATGATCAATCCATTACTTTTGCTTTGGAGGATGAA
AGTTATGAGATATATGTTGAAGACTTGAAAAAAGATGAAAAGAAAGATAA
GGTGTTACTGAGTTACTATGAGTCTCAACACCCCTCAAATGAATCAGGTG
ACGGTGTTGATGGTAAGATGTTAATGGTAACCCTGAGTCCTACAAAAGAC
TTCTGGTTGCATGCCAACAACAAGGAACACTCTGTGGAGCTCCATAAGTG
TGAAAAACCACTGCCAGACCAGGCCTTCTTTGTCCTTCATAATATGCACT
CCAACTGTGTTTCATTTGAATGCAAGACTGATCCTGGAGTGTTTATAGGT
GTAAAGGATAATCATCTTGCTCTGATTAAAGTAGACTCTTCTGAGAATTT
GTGTACTGAAAATATCTTGTTTAAGCTCTCTGAAACTTAG

Alternatively, in certain embodiments, the cell further comprises a modified promoter/enhancer at an IL-33 gene locus, which can increase IL-33 gene expression, e.g., a constitutive or inducible promoter placed to drive IL-33 gene expression. Cells comprising an antigen-recognizing receptor (e.g., a CAR) and engineered to express IL-33, e.g., comprising an exogenous IL-33 polypeptide or a modified promoter/enhancer at an IL-33 gene locus are disclosed in International Patent Publication No. WO2019/099479, which is incorporated by reference in its entirety.

Additionally or alternatively, the cell is engineered to express IL-36. In certain embodiments, the cell further comprises an exogenous IL-36 polypeptide. In certain embodiments, the cell further comprises a modified promoter/enhancer at an IL-36 gene locus, which can increase IL-36 gene expression, e.g., a constitutive or inducible promoter placed to drive IL-36 gene expression. Cells comprising an antigen-recognizing receptor (e.g., a CAR) and engineered to express IL-36, e.g., comprising an exogenous IL-36 polypeptide or a modified promoter/enhancer at an IL-36 gene locus are disclosed in International Patent Publication No. WO2019/099483, which is incorporated by reference in its entirety.

### 5.5. Compositions and Vectors

The presently disclosed subject matter provides compositions comprising a presently disclosed CD371-targeted antigen-recognizing receptor (e.g., one disclosed in Section 5.3). Also provided are cells comprising such compositions.

In certain embodiments, the presently disclosed CD371-targeted antigen-recognizing receptor is operably linked to a promoter.

Furthermore, the present discloses subject matter provides nuclei acid compositions comprising a polynucleotide encoding a presently disclosed CD371-targeted antigen-recognizing receptor (e.g., one disclosed in Section 5.3). Also provided are cells comprising such nucleic acid compositions.

In certain embodiments, the nucleic acid composition further comprises a promoter that is operably linked to the presently disclosed CD371-targeted antigen-recognizing receptor.

In certain embodiments, the promoter is endogenous or exogenous. In certain embodiments, the exogenous promoter is selected from an elongation factor (EF)-1 promoter, a cytomegalovirus immediate-early promoter (CMV) promoter, a simian virus 40 early promoter (SV40) promoter, a phosphoglycerate kinase (PGK) promoter, and a metallothionein promoter. In certain embodiments, the promoter is an inducible promoter. In certain embodiment, the inducible promoter is selected from a NFAT transcriptional response element (TRE) promoter, a CD69 promoter, a CD25 promoter, and an IL-2 promoter.

The compositions and nucleic acid compositions can be administered to subjects or and/delivered into cells by art-known methods or as described herein. Genetic modification of a cell (e.g., a T cell or a NK cell) can be accomplished by transducing a substantially homogeneous cell composition with a recombinant DNA construct. In certain embodiments, a retroviral vector (e.g., gamma-retroviral vector or lentiviral vector) is employed for the introduction of the DNA construct into the cell. For example, a polynucleotide encoding an antigen-recognizing receptor can be cloned into a retroviral vector and expression can be driven from its endogenous promoter, from the retroviral long terminal repeat, or from a promoter specific for a target cell type of interest. Non-viral vectors may be used as well.

For initial genetic modification of a cell to include a presently disclosed CD371-targeted antigen-recognizing receptor (e.g., a CAR or a TCR), a retroviral vector can be employed for transduction, however any other suitable viral vector or non-viral delivery system can be used. The antigen-recognizing receptor can be constructed in a single, multicistronic expression cassette, in multiple expression cassettes of a single vector, or in multiple vectors. Examples of elements that create polycistronic expression cassette include, but is not limited to, various viral and non-viral Internal Ribosome Entry Sites (IRES, e.g., FGF-1 IRES, FGF-2 IRES, VEGF IRES, IGF-II IRES, NF-κB IRES, RUNX1 IRES, p53 IRES, hepatitis A IRES, hepatitis C IRES, pestivirus IRES, aphthovirus IRES, picornavirus IRES, poliovirus IRES and encephalomyocarditis virus IRES) and cleavable linkers (e.g., 2A peptides , e.g., P2A, T2A, E2A and F2A peptides). Combinations of retroviral vector and an appropriate packaging line are also suitable, where the capsid proteins will be functional for infecting human cells. Various amphotropic virus-producing cell lines are known, including, but not limited to, PA12 (Miller et al., (1985) Mol Cell Biol (1985); 5:431-437); PA317 (Miller., et al., Mol Cell Biol (1986); 6:2895-2902); and CRIP (Danos et al., Proc Natl Acad Sci USA (1988); 85:6460-6464). Non-amphotropic particles are suitable too, e.g., particles pseudotyped with VSVG, RD114 or GALV envelope and any other known in the art.

Possible methods of transduction also include direct co-culture of the cells with producer cells (Bregni et al., Blood (1992); 80:1418-1422), or culturing with viral supernatant alone or concentrated vector stocks with or without appropriate growth factors and polycations (Xu et al., Exp Hemat (1994); 22:223-230; and Hughes et al. J Clin Invest (1992); 89:1817).

Other transducing viral vectors can be used to modify a cell. In certain embodiments, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Other viral vectors that can be used include, for example, adenoviral, lentiviral, and adena-associated viral vectors, vaccinia virus, a bovine papilloma virus, or a herpes virus, such as Epstein-Barr Virus (also see, for example, the vectors of Miller, Human Gene Thera (1990); 15-14; Friedman, Science 244:1275-1281, 1989; Eglitis et al., BioTechniques (1988); 6:608-614; Tolstoshev et al., Cur Opin Biotechnol (1990); 1:55-61; Sharp, The Lancet (1991); 337:1277-78; Cornetta et al., Nucleic Acid Research and Molecular Biology 36:311-22, 1987; Anderson, Science (1984); 226:401-409; Moen, Blood Cells 17:407-16, 1991; Miller et al., Biotechnol (1989); 7:980-90; LeGal La Salle et al., Science (1993); 259:988-90; and Johnson, Chest (1995)107:77S-83S). Retroviral vectors are particularly well developed and have been used in clinical settings (Rosenberg et al., N Engl J Med (1990); 323:370, 1990; Anderson et al., U.S. Pat. . No. 5,399,346).

Non-viral approaches can also be employed for genetic modification of a cell. For example, a nucleic acid molecule can be introduced into a cell by administering the nucleic acid in the presence of lipofection (Feigner et al., Proc Natl Acad Sci U.S.A. (1987); 84:7413; Ono et al., Neurosci Lett (1990); 17:259; Brigham et al., Am J Med Sci (1989); 298:278; Staubinger et al., Methods in Enzymol (1983); 101:512, Wu et al., J Biol Chem (1988); 263:14621; Wu et al., J Biol Chem (1989); 264:16985), or by micro-injection under surgical conditions (Wolff et al., Science (1990); 247:1465). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases or targeted nucleases (e.g. Zinc finger nucleases, meganucleases, or TALE nucleases, CRISPR). Transient expression may be obtained by RNA electroporation.

Any targeted genome editing methods can also be used to deliver a presently disclosed antigen-recognizing receptor to a cell or a subject. In certain embodiments, a CRISPR system is used to deliver a presently disclosed antigen-recognizing receptor disclosed herein. In certain embodiments, zinc-finger nucleases are used to deliver the antigen-recognizing receptor. In certain embodiments, a TALEN system is used to deliver a presently disclosed antigen-recognizing receptor.

Clustered regularly-interspaced short palindromic repeats (CRISPR) system is a genome editing tool discovered in prokaryotic cells. When utilized for genome editing, the system includes Cas9 (a protein able to modify DNA utilizing crRNA as its guide), CRISPR RNA (crRNA, contains the RNA used by Cas9 to guide it to the correct section of host DNA along with a region that binds to tracrRNA (generally in a hairpin loop form) forming an active complex with Cas9), trans-activating crRNA (tracrRNA, binds to crRNA and forms an active complex with Cas9), and an optional section of DNA repair template (DNA that guides the cellular repair process allowing insertion of a specific DNA sequence). CRISPR/Cas9 often employs a plasmid to transfect the target cells. The crRNA needs to be designed for each application as this is the sequence that Cas9 uses to identify and directly bind to the target DNA in a cell. The repair template carrying CAR expression cassette need also be designed for each application, as it must overlap with the sequences on either side of the cut and code for the insertion sequence. Multiple crRNA's and the tracrRNA can be packaged together to form a single-guide RNA (sgRNA). This sgRNA can be joined together with the Cas9 gene and made into a plasmid in order to be transfected into cells.

A zinc-finger nuclease (ZFN) is an artificial restriction enzyme, which is generated by combining a zinc finger DNA-binding domain with a DNA-cleavage domain. A zinc finger domain can be engineered to target specific DNA sequences which allows a zinc-finger nuclease to target desired sequences within genomes. The DNA-binding domains of individual ZFNs typically contain a plurality of individual zinc finger repeats and can each recognize a plurality of basepairs. The most common method to generate new zinc-finger domain is to combine smaller zinc-finger “modules” of known specificity. The most common cleavage domain in ZFNs is the non-specific cleavage domain from the type IIs restriction endonuclease FokI. Using the endogenous homologous recombination (HR) machinery and a homologous DNA template carrying CAR expression cassette, ZFNs can be used to insert the CAR expression cassette into genome. When the targeted sequence is cleaved by ZFNs, the HR machinery searches for homology between the damaged chromosome and the homologous DNA template, and then copies the sequence of the template between the two broken ends of the chromosome, whereby the homologous DNA template is integrated into the genome.

Transcription activator-like effector nucleases (TALEN) are restriction enzymes that can be engineered to cut specific sequences of DNA. TALEN system operates on almost the same principle as ZFNs. They are generated by combining a transcription activator-like effectors DNA-binding domain with a DNA cleavage domain. Transcription activator-like effectors (TALEs) are composed of 33-34 amino acid repeating motifs with two variable positions that have a strong recognition for specific nucleotides. By assembling arrays of these TALEs, the TALE DNA-binding domain can be engineered to bind desired DNA sequence, and thereby guide the nuclease to cut at specific locations in genome. cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g. the elongation factor la enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

Methods for delivering the genome editing agents/systems can vary depending on the need. In certain embodiments, the components of a selected genome editing method are delivered as DNA constructs in one or more plasmids. In certain embodiments, the components are delivered via viral vectors. Common delivery methods include but is not limited to, electroporation, microinjection, gene gun, impalefection, hydrostatic pressure, continuous infusion, sonication, magnetofection, adeno-associated viruses, envelope protein pseudotyping of viral vectors, replication-competent vectors cis and trans-acting elements, herpes simplex virus, and chemical vehicles (e.g., oligonucleotides, lipoplexes, polymersomes, polyplexes, dendrimers, inorganic Nanoparticles, and cell-penetrating peptides).

### 5.6. Polypeptides

The presently disclosed subject matter provides methods for optimizing an amino acid sequence or a nucleic acid sequence by producing an alteration in the sequence. Such alterations may include certain mutations, deletions, insertions, or post-translational modifications. The presently disclosed subject matter further includes analogs of any naturally-occurring polypeptides disclosed herein (including, but not limited to, CD371, CD8, CD28, 4-1BB, and CD3ζ). Analogs can differ from a naturally-occurring polypeptide disclosed herein by amino acid sequence differences, by post-translational modifications, or by both. Analogs can exhibit at least about 85%, about 90%, about 91%, about 92%, about 93%, about 94%, about 95%, about 96%, about 97%, about 98%, about 99% or more homologous or identical to all or part of a naturally-occurring amino, acid sequence of the presently disclosed subject matter. The length of sequence comparison is at least 5, 10, 15 or 20 amino acid residues, e.g., at least 25, 50, or 75 amino acid residues, or more than 100 amino acid residues. Again, in an exemplary approach to determining the degree of identity, a BLAST program may be used, with a probability score between e⁻³ and e⁻¹⁰⁰ indicating a closely related sequence. Modifications include in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally-occurring polypeptides by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2d ed.), CSH Press, 1989, or Ausubel et al., supra). Also included are cyclized peptides, molecules, and analogs which contain residues other than L-amino acids, e.g., D-amino acids or non-naturally occurring or synthetic amino acids, e.g., β or γ amino acids.

In addition to full-length polypeptides, the presently disclosed subject matter also provides fragments of any of the polypeptides disclosed herein. As used herein, the term “a fragment” means at least 5, 10, 13, or 15 amino acids. In certain embodiments, a fragment comprises at least 20 contiguous amino acids, at least 30 contiguous amino acids, or at least 50 contiguous amino acids. In certain embodiments, a fragment comprises at least 60 to 80, 100, 200, 300 or more contiguous amino acids. Fragments can be generated by methods known to those skilled in the art or may result from normal protein processing (e.g., removal of amino acids from the nascent polypeptide that are not required for biological activity or removal of amino acids by alternative mRNA splicing or alternative protein processing events).

### 5.7 Formulations and Administration

The presently disclosed subject matter also provides compositions comprising the presently disclosed cells. Compositions comprising the presently disclosed cells can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the genetically modified cells in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON′S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the presently disclosed subject matter, however, any vehicle, diluent, or additive used would have to be compatible with the genetically modified cells.

The compositions can be isotonic, i.e., they can have the same osmotic pressure as blood and lacrimal fluid. The desired isotonicity of the compositions may be accomplished using sodium chloride, or other pharmaceutically acceptable agents such as dextrose, boric acid, sodium tartrate, propylene glycol or other inorganic or organic solutes. Sodium chloride can be particularly for buffers containing sodium ions.

Viscosity of the compositions, if desired, can be maintained at the selected level using a pharmaceutically acceptable thickening agent. For example, methylcellulose is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

Compositions comprising the presently disclosed cells can be provided systemically or directly to a subject for inducing and/or enhancing an immune response to an antigen and/or treating and/or preventing a neoplasia. In certain embodiments, the presently disclosed cells or compositions comprising thereof are directly injected into an organ of interest (e.g., an organ affected by a neoplasia). Alternatively, the presently disclosed cells or compositions comprising thereof are provided indirectly to the organ of interest, for example, by administration into the circulatory system (e.g., the tumor vasculature). Expansion and differentiation agents can be provided prior to, during or after administration of the cells or compositions to increase production of cells (e.g., T cells or NK cells) in vitro or in vivo.

The presently disclosed cells can be administered in any physiologically acceptable vehicle, normally intravascularly, although they may also be introduced into bone or other convenient site where the cells may find an appropriate site for regeneration and differentiation (e.g., thymus).

The quantity of cells to be administered can vary for the subject being treated. In certain embodiments, between about 10⁴ and about 10¹⁰, between about 10⁴ and about 10⁷, between about 10⁵ and about 10⁷, between about 10⁵ and about 10⁹, or between about 10⁶ and about 10⁸ of the presently disclosed cells are administered to a subject. More effective cells may be administered in even smaller numbers. Usually, at least about 1×10⁵ cells will be administered, eventually reaching about 1×10¹⁰ or more. In certain embodiments, at least about 1×10⁵, 5×10⁵, 1×10⁶, about 5×10⁶, about 1×10⁷, about 5×10⁷, about 1×10⁸, or about 5×10⁸ of the presently disclosed cells are administered to a subject. In certain embodiments, about 1×10⁶ of the presently disclosed cells are administered to a subject. The precise determination of what would be considered an effective dose can be based on factors individual to each subject, including their size, age, sex, weight, and condition of the particular subject. Dosages can be readily ascertained by those skilled in the art from this disclosure and the knowledge in the art.

The presently disclosed cells can comprise a purified population of cells. Those skilled in the art can readily determine the percentage of the presently disclosed cells in a population using various well-known methods, such as fluorescence activated cell sorting (FACS). Suitable ranges of purity in populations comprising the presently disclosed immunoresponsive cells are about 50% to about 55%, about 5% to about 60%, and about 65% to about 70%. In certain embodiments, the purity is about 70% to about 75%, about 75% to about 80%, or about 80% to about 85%. In certain embodiments, the purity is about 85% to about 90%, about 90% to about 95%, and about 95% to about 100%. Dosages can be readily adjusted by those skilled in the art (e.g., a decrease in purity may require an increase in dosage). The cells can be introduced by injection, catheter, or the like.

The skilled artisan can readily determine the amount of cells and optional additives, vehicles, and/or carrier in compositions and to be administered in methods. Typically, any additives (in addition to the active cell(s) and/or agent(s)) are present in an amount of 0.001 to 50% (weight) solution in phosphate buffered saline, and the active ingredient is present in the order of micrograms to milligrams, such as about 0.0001 to about 5 wt %, about 0.0001 to about 1 wt %, about 0.0001 to about 0.05 wt % or about 0.001 to about 20 wt %, about 0.01 to about 10 wt %, or about 0.05 to about 5 wt %. For any composition to be administered to an animal or human, the followings can be determined: toxicity such as by determining the lethal dose (LD) and LD50 in a suitable animal model e.g., rodent such as mouse; the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

In certain embodiments, the composition is a pharmaceutical composition comprising the presently disclosed cells and a pharmaceutically acceptable carrier.

Administration of the compositions can be autologous or heterologous. For example, cells can be obtained from one subject, and administered to the same subject or a different, compatible subject. Peripheral blood derived cells or their progeny (e.g., in vivo, ex vivo or in vitro derived) can be administered. When administering a presently disclosed composition (e.g., a pharmaceutical composition comprising presently disclosed cells), it can be formulated in a unit dosage injectable form (solution, suspension, emulsion).

The presently disclosed cells and compositions can be administered by any method known in the art including, but not limited to, oral administration, intravenous administration, subcutaneous administration, intranodal administration, intratumoral administration, intrathecal administration, intrapleural administration, intraosseous administration, intraperitoneal administration, pleural administration, and direct administration to the subject.

### 5.8. Methods of Treatment

The presently disclosed subject cells and compositions comprising thereof can be used for treating and/or preventing a tumor or neoplasm. Such cells can be administered to a subject (e.g., a human subject) in need thereof for treatment and/or prevention of a tumor or neoplasm (e.g., acute myeloid leukemia (AML), multiple myeloma, Non-Hodgkin's Lymphoma, Hodgkin's Lymphoma, Chronic Lymphocytic Leukemia (CLL), glioblastoma). In certain embodiments, the cell is a T cell. The T cell can be a CD4⁺ T cell or a CD8⁺ T cell. In certain embodiments, the T cell is a CD4⁺ T cell.

The presently disclosed subject matter provides methods for inducing and/or increasing an immune response in a subject in need thereof. The presently disclosed cells and compositions comprising thereof can be used in a therapy or medicament. The presently disclosed subject matter provides various methods of using the cells (e.g., T cells) or compositions comprising thereof. For example, the presently disclosed cells and compositions comprising thereof can be used for reducing tumor burden in a subject. The presently disclosed cell can reduce the number of tumor cells, reduce tumor size, and/or eradicate the tumor in the subject. The presently disclosed cells and compositions comprising thereof can be used for treating and/or preventing a tumor or neoplasm in a subject. The presently disclosed cells and compositions comprising thereof can be used for prolonging the survival of a subject suffering from a tumor or neoplasm. Such methods comprise administering the presently disclosed cells or a composition (e.g., a pharmaceutical composition) comprising thereof to achieve the desired effect, e.g., palliation of an existing condition or prevention of recurrence. For treatment, the amount administered is an amount effective in producing the desired effect. An effective amount can be provided in one or a series of administrations. An effective amount can be provided in a bolus or by continuous perfusion.

The presently disclosed subject matter provides various methods of using the cells (e.g., T cells) or compositions comprising thereof. For example, the presently disclosed subject matter provides methods of reducing tumor burden in a subject. In certain embodiments, the method of reducing tumor burden comprises administering the presently disclosed cells or a composition comprising thereof to the subject. The presently disclosed cell can reduce the number of tumor cells, reduce tumor size, and/or eradicate the tumor in the subject.

The tumor or neoplasm can be a solid tumor. Non-limiting examples of solid tumors include mesothelioma, lung cancer, pancreatic cancer, ovarian cancer, breast cancer, colon cancer, pleural tumor, glioblastoma, esophageal cancer, gastric cancer, synovial sarcoma, thymic carcinoma, endometrial carcinoma, stomach cancer, and cholangiocarcinoma.

The presently disclosed subject matter also provides methods of increasing or lengthening survival of a subject having a tumor or neoplasm. In certain embodiments, the method of increasing or lengthening survival of a subject having a tumor or neoplasm comprises administering the presently disclosed immunoresponsive cells or a composition comprising thereof to the subject. The method can reduce or eradicate tumor burden in the subject. Additionally, the presently disclosed subject matter provides methods for increasing an immune response in a subject, comprising administering the presently disclosed cell or a composition comprising thereof to the subject. The presently disclosed subject matter further provides methods for treating and/or preventing a tumor or neoplasm in a subject, comprising administering the presently disclosed cells or a composition comprising thereof to the subject.

Non-limiting examples of neoplasms or tumors include acute myeloid leukemia (AML), multiple myeloma, Chronic Lymphocytic Leukemia (CLL), lymphoma (Hodgkin's lymphoma, non-Hodgkin's lymphoma), glioblastoma, myelodysplastic syndrome (MDS), and chronic myelogenous leukemia (CML), bone cancer, intestinal cancer, liver cancer, skin cancer, cancer of the head or neck, melanoma (cutaneous or intraocular malignant melanoma), renal cancer (e.g. clear cell carcinoma), throat cancer, prostate cancer (e.g. hormone refractory prostate adenocarcinoma), blood cancers (e.g. leukemias, lymphomas, and myelomas), uterine cancer, rectal cancer, cancer of the anal region, bladder cancer, brain cancer, stomach cancer, testicular cancer, carcinoma of the fallopian tubes, carcinoma of the endometrium, carcinoma of the cervix, carcinoma of the vagina, carcinoma of the vulva, leukemias (e.g., acute leukemia, acute lymphocytic leukemia, acute myelocytic leukemia, acute myeloblastic leukemia, acute promyelocytic leukemia, acute monocytic leukemia, acute erythroleukemia, chronic leukemia, chronic myelocytic leukemia, polycythemia vera, cancer of the small intestine, cancer of the endocrine system, cancer of the thyroid gland, cancer of the parathyroid gland, cancer of the adrenal gland, sarcoma of soft tissue, cancer of the urethra, cancer of the penis, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, include Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, hepatoma, nile duct carcinoma, choriocarcinoma, seminoma, embryonal carcinoma, Wilm's tumor, cervical cancer, salivary gland cancer, uterine cancer, testicular cancer, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma).

In certain embodiments, the tumor or neoplasm is selected from the group consisting of acute myeloid leukemia (AML), multiple myeloma, Chronic Lymphocytic Leukemia (CLL), Hodgkin's lymphoma, non-Hodgkin's lymphoma, glioblastoma, myelodysplastic syndrome (MDS), and chronic myelogenous leukemia (CML). In certain embodiments, the tumor or neoplasm is AML.

The subjects can have an advanced form of disease, in which case the treatment objective can include mitigation or reversal of disease progression, and/or amelioration of side effects. The subjects can have a history of the condition, for which they have already been treated, in which case the therapeutic objective will typically include a decrease or delay in the risk of recurrence.

As a consequence of surface expression of a presently disclosed CD371-targeted antigen-recognizing receptor, adoptively transferred cells (e.g., immunoresponsive cells, e.g., T cells or NK cells) are endowed with augmented and selective cytolytic activity at the tumor site. Furthermore, subsequent to their localization to tumor or viral infection and their proliferation, the cells turn the tumor or viral infection site into a highly conductive environment for a wide range of immune cells involved in the physiological anti-tumor or antiviral response (tumor infiltrating lymphocytes, NK-, NKT-cells, dendritic cells, and macrophages).

Further modification can be introduced to the presently disclosed cells (e.g., T cells) to avert or minimize the risks of immunological complications (known as “malignant T-cell transformation”), e.g., graft versus-host disease (GvHD), or when healthy tissues express the same target antigens as the tumor cells, leading to outcomes similar to GvHD. A potential solution to this problem is engineering a suicide gene into the presently disclosed cells. Suitable suicide genes include, but are not limited to, Herpes simplex virus thymidine kinase (hsv-tk), inducible Caspase 9 Suicide gene (iCasp-9), and a truncated human epidermal growth factor receptor (EGFRt) polypeptide. In certain embodiments, the suicide gene is an EGFRt polypeptide. The EGFRt polypeptide can enable T cell elimination by administering anti-EGFR monoclonal antibody (e.g., cetuximab). EGFRt can be covalently joined to the upstream of the antigen-recognizing receptor (e.g., CAR). The suicide gene can be included within the vector comprising nucleic acids encoding a presently disclosed antigen-recognizing receptor (e.g., CAR). In this way, administration of a prodrug designed to activate the suicide gene (e.g., a prodrug (e.g., AP1903 that can activate iCasp-9) during malignant T-cell transformation (e.g., GVHD) triggers apoptosis in the suicide gene-activated cells expressing the antigen-recognizing receptor (e.g., CAR). The incorporation of a suicide gene into the a presently disclosed antigen-recognizing receptor (e.g., CAR) gives an added level of safety with the ability to eliminate the majority of receptor-expressing cells within a very short time period. A presently disclosed cell (e.g., a T cell) incorporated with a suicide gene can be pre-emptively eliminated at a given timepoint post the cell infusion, or eradicated at the earliest signs of toxicity.

### 5.9. Kits

The presently disclosed subject matter provides kits for inducing and/or enhancing an immune response in a subject, treating and/or preventing a tumor or neoplasm in a subject, reducing tumor burden in a subject, and/or increasing or lengthening survival of a subject having a tumor or neoplasm in a subject. In certain embodiments, the kit comprises the presently disclosed cells or a composition comprising thereof. In certain embodiments, the kit comprises a sterile container; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments. In certain non-limiting embodiments, the kit includes a nucleic acid molecule encoding a presently disclosed CD371-targeted antigen-recognizing receptor (e.g., a CAR or a TCR).

If desired, the cells and/or nucleic acid molecules are provided together with instructions for administering the cells or nucleic acid molecules to a subject having or at risk of developing a tumor or neoplasm. The instructions generally include information about the use of the composition for the treatment and/or prevention of a tumor or neoplasm. In certain embodiments, the instructions include at least one of the following: description of the therapeutic agent; dosage schedule and administration for treatment or prevention of a tumor or neoplasm,; precautions; warnings; indications; counter-indications; over-dosage information; adverse reactions; animal pharmacology; clinical studies; and/or references. The instructions may be printed directly on the container (when present), or as a label applied to the container, or as a separate sheet, pamphlet, card, or folder supplied in or with the container.

### EXAMPLES

The practice of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides disclosed herein, and, as such, may be considered in making and practicing the presently disclosed subject matter. Particularly useful techniques for particular embodiments will be discussed in the sections that follow.

The following examples are put forth so as to provide those of ordinary skill in the art with a complete disclosure and description of how to make and use the presently disclosed cells and compositions, and are not intended to limit the scope of what the inventors regard as their invention.

### Example 1

### Generation of CD371-Targeted CARs

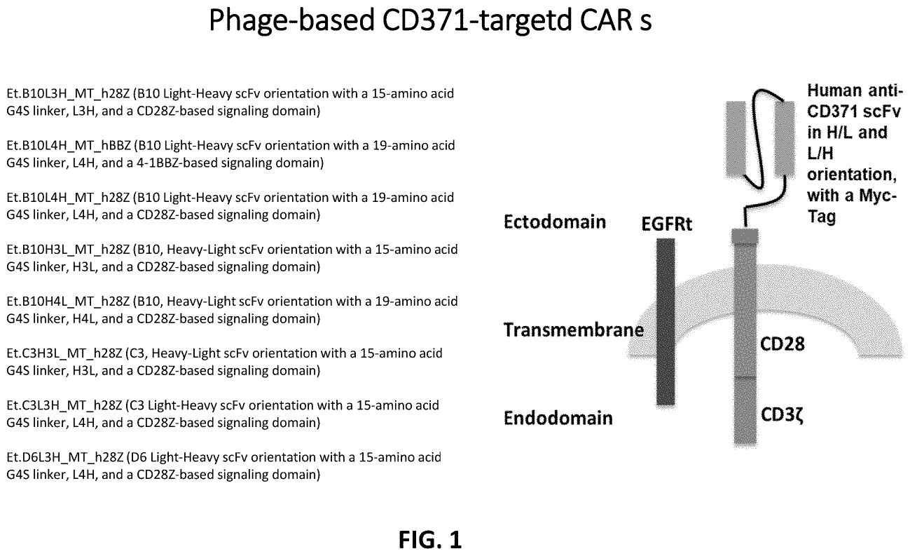

The following six CD371-targeted CARs were generated: “Et.B10L3H_MT_h28z”, “Et.B10L4H_MT_hBBz”, “Et.B10L4H_MT_h28z”, “Et.B10H3L_MT_h28z”, “Et.B10H4L_MT_h28z”, “Et.C3H3L_MT_h28z”, “Et.C3L3H_MT_h28z”, and “Et.D6L3H_MT_h28z”. The structures of the CARs are illustrated in FIG. 1.

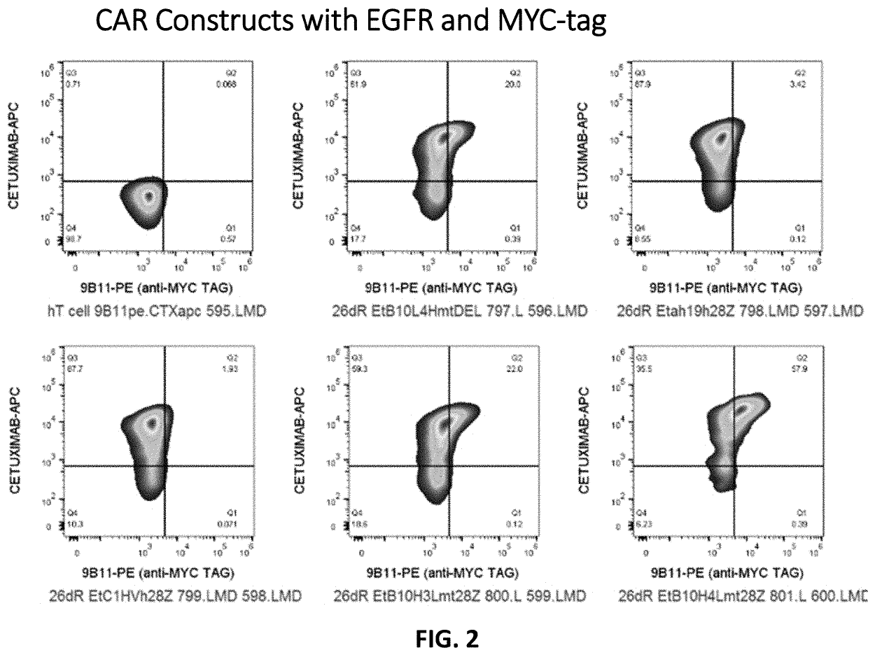

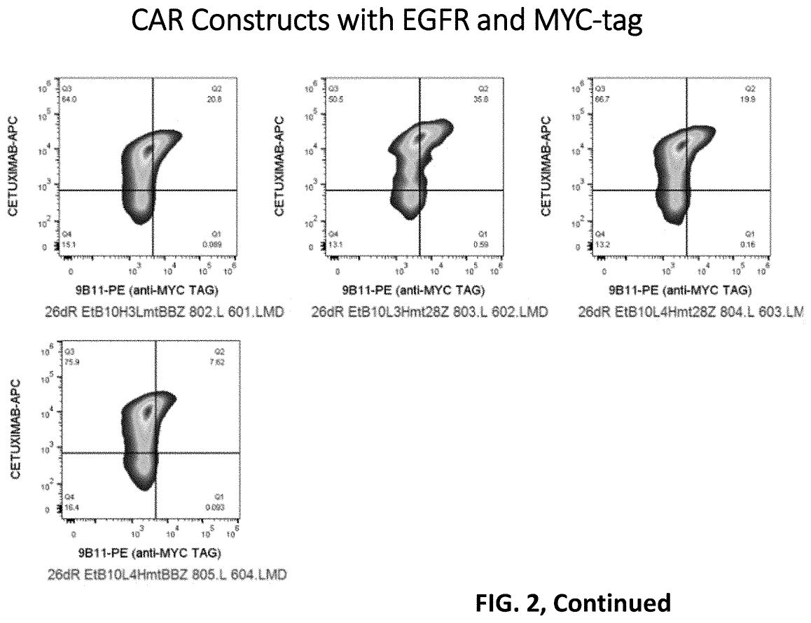

As shown in FIG. 2, the CARs were detected by EGFR and Myc-TAG on the surface of transduced human T cells. Antibodies against EGFRt (CETUXIMAB-APC) and Myc-tag (9B11-PE) were used to detect surface expression of EGFRt and human anti-human CAR in B10-based constructs. Controls included non-transduced human T cells not expressing EGFRt or Myc-tag, and non-Myc Tag containing CAR constructs Etah19h28Z (an anti-human CD19 CAR T cell) and EtC1HVh28Z (an anti-CD371 CAR T cell derived from a mouse anti-human CD371 antibody 1075.7)

### Example 2

### In Vitro Cytotoxicity Activities of CD371-Targeted CAT T Cells

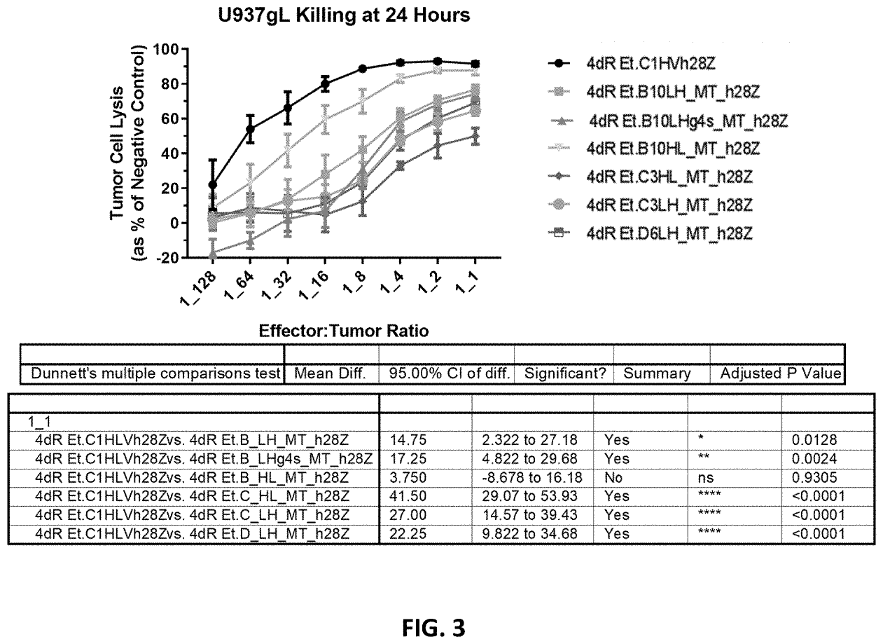

T cells were transfected with a number of CD371-targeted CARs (i.e., “Et.C1HVh28Z”, “Et.B10LH_MT_h28Z”, “Et.B10LHg4s_MT_h28Z”, “Et.B10HL_MT_h28Z”, “Et.C3HL_MT_h28Z”, “Et.C3LH_MT_h28Z”, and “Et.D6LH_MT_h28Z”). Et.C1HVh28Z was used as a positive control and is derived from a mouse anti-human CD371 antibody 1075.7. “Et.B10LHg4s_MT_hDEL” is a non-functional CD371-targeted CAR T cells (based on B10L4H, but without a CD28 or CD3ζ zeta signaling domain). These CD371-targeted CAR T cells were cocultured with CD371+ U937 cells expressing GFP and firefly luciferase (U937gL) at different effector:tumor ratios. 24 hours later, bioluminescence was measured and plotted as a percentage of signal detected in a coculture of a non-functional CD371-targeted CAR T cells (based on B10L4H, but without a CD28 or CD3ζ zeta signaling domain) and U937gL. The results are shown in FIG. 3. As shown in FIG. 3, CD371-targeted CAR T cells were able to lyse cells expressing CD371.

### Example 3

### In Vitro Cytotoxicity Activities of CD371-Targeted CAT T Cells

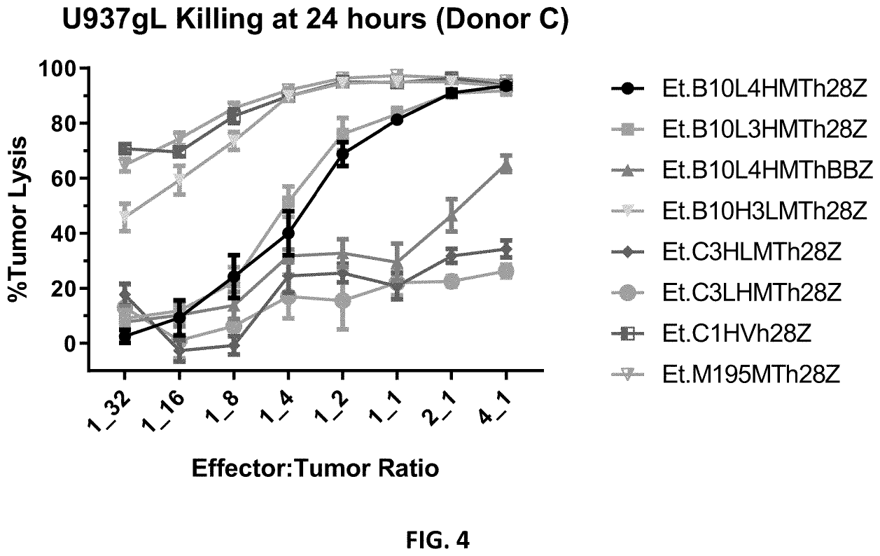

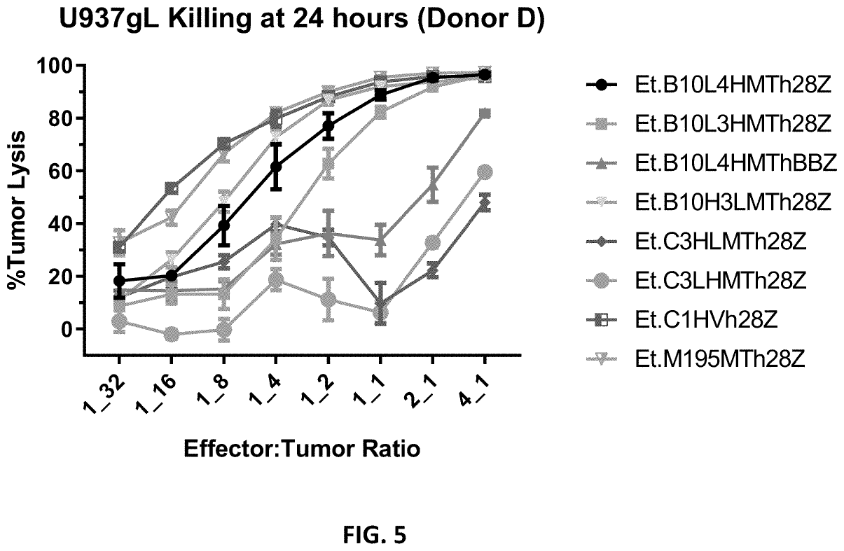

T cells were transfected with a number of CD371-targeted CARs (i.e., “Et.C1HVh28Z”, “Et.B10L4H_MT_h28Z”, “Et.B10L3H_MT_h28Z”, “Et.B10L4H_MT_hBBZ”, “Et.B10H3L_MT_h28Z”, “Et. C3HL_MT_h28Z”, “Et.C3LH_MT_h28Z”, and “Et.M195_MT_h28Z”). Et.C1HVh28Z was used as a positive control and is derived from a mouse anti-human antibody 1075.7). “Et.M195MTh28Z” represents a CD33-targeted CAR T cell derived from the murine anti-human CD33 antibody, M195. “Et.M195MTh28Z” used as a positive control as U937 cells also express CD33. Healthy human donor-derived (i.e., Donor C and Donor D) CD371-targeted CAR T cells were cocultured with CD33+/CD371+ U937 cells expressing GFP and firefly luciferase (U937gL) at different effector:tumor ratios. 24 hours later, bioluminescence was measured and plotted as a percentage of signal detected in a coculture of a non-functional CD371-targeted CAR T cells (based on B10L4H, but without a CD28 or CD3ζ zeta signaling domain) and U937gL. The results are shown in FIG. 4 (for Donor C) and FIG. 5 (for Donor D). As shown in FIGS. 4 and 5, B10-based CARs (both HL/LH) T cells outperformed C3-based CAR T cells.

### Example 4

### Antigen-Stimulation of CD371-Targeted CAR T Cells

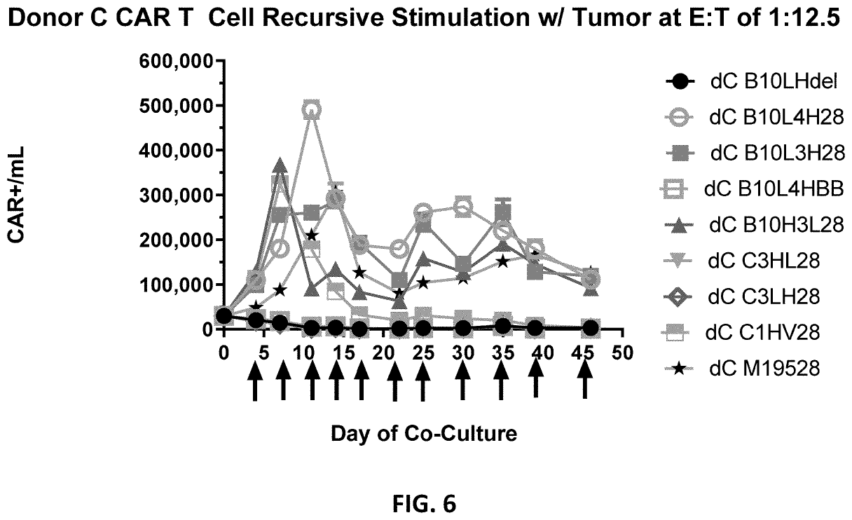

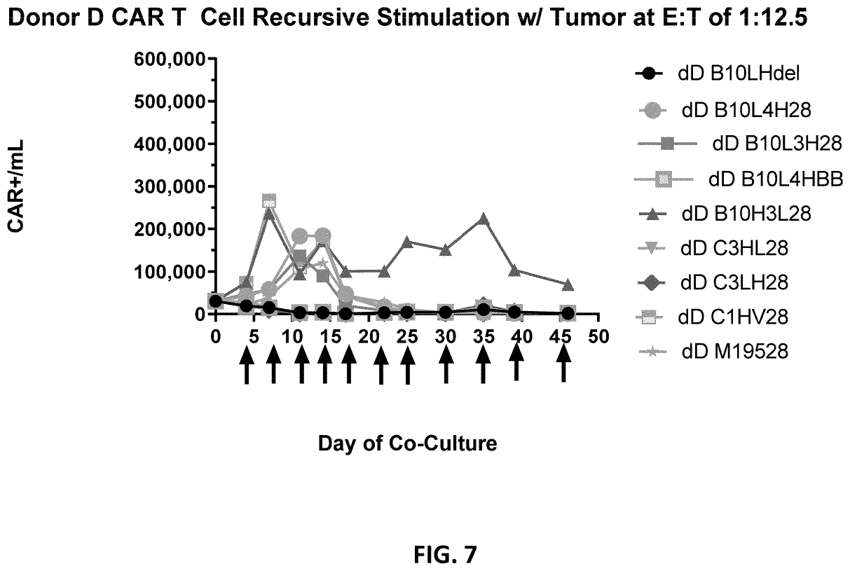

Healthy human donor-derived (i.e., Donor C and Donor D) CD371-targeted CAR T cells were cocultured with CD33+/CD371+ U937gL at an E:T ratio of 1:12.5 and at a concentration of 30,000 CAR+/mL. Roughly every 4-5 days, CAR T cells were counted and characterized by flow cytometry, and the starting number of tumor cells were added back into the culture (indicated by arrows). The results are shown in FIG. 6 (for Donor C) and FIG. 7 (for Donor D). “Et.B10LHdel” represents a non-functional CAR T cell (lacks signaling domains). “Et.C1HVh28Z” represents a CD371-targeted CAR derived from a mouse anti-human CD371antibody 1075.7), and “EtM195MTh28Z” or “M19528” represents a CD33-targeted CAR T cell derived from the murine anti-human CD33 antibody, M195. As shown in FIGS. 6 and 7, B10-based CAR T cells outperformed C3-based CAR T cells in a long-term repeated stimulation assay.

### Example 5

### In Vitro Cytotoxicity Activities of CD371-Targeted CAT T Cells

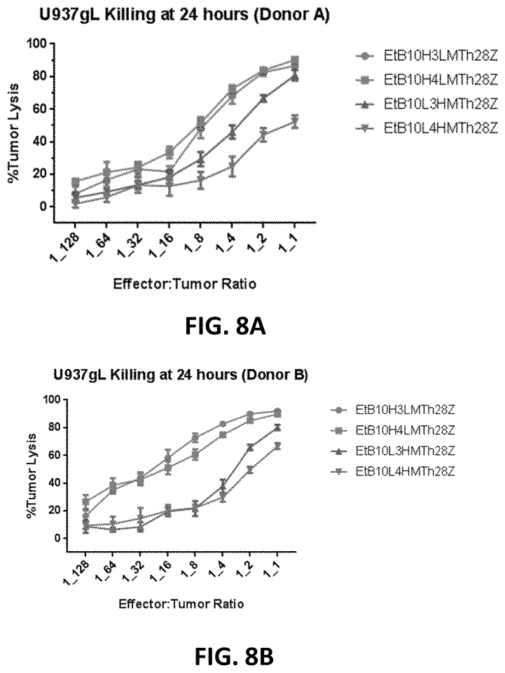

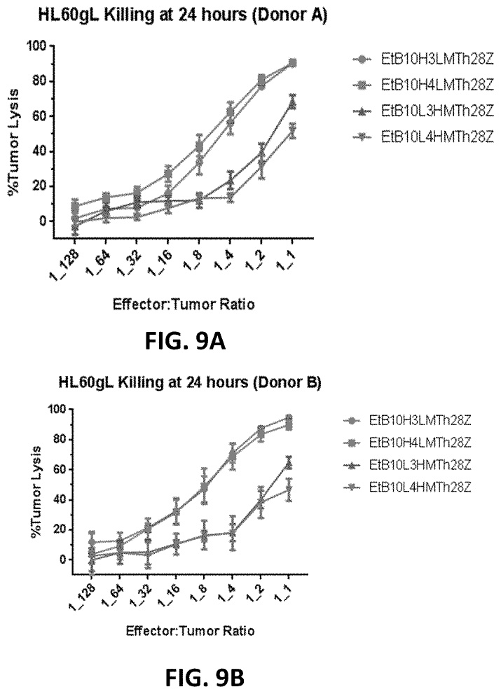

Healthy human donor-derived (i.e., Donor A and Donor B) CD371-targeted CAR T cells were cocultured with CD371-positive U937 cells or HL60 cells expressing GFP and firefly luciferase (U937gL or HL60gL) at different effector:tumor ratios. Bioluminescence was measured 24 hours after coculture, and plotted as a percentage of signal detected in a coculture of a non-functional CD371-targeted CAR T cells (based on B10L4H, but without a CD28 or CD3 zeta signaling domain) and U937gL. The results are shown in FIGS. 8A and 8B (for U937gL cells) and FIGS. 9A and 9B (for HL 60 cells). As shown in FIGS. 8A-8B and 9A-9B, B10HL-based CD371-targeted CAR T cells outperformed B10LH-based CD371-targeted CAR T cells.

### Example 6

### Antigen-Specific Cytotoxicity Activities of CD371-Targeted CAR T Cells

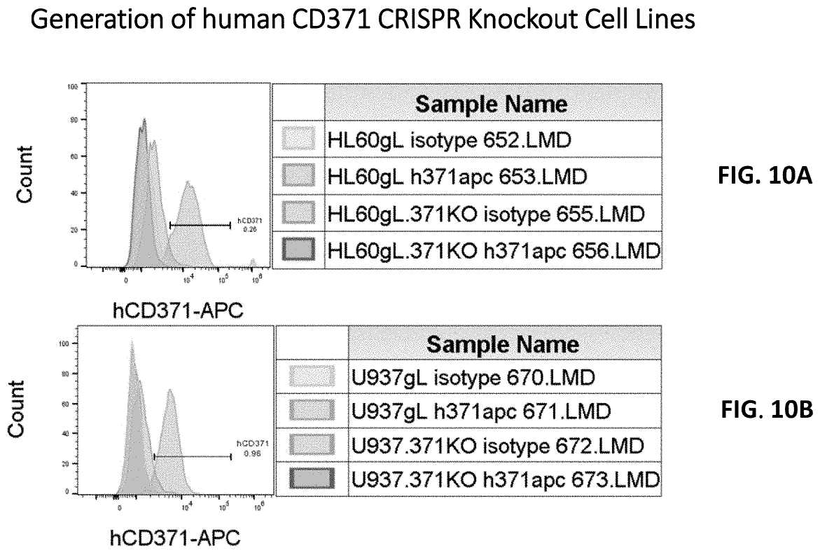

Whether the cytotoxicity activities of the CD371-targeted CAR T cells are specific to CD371 was assessed. Human CD371 (hCD371) CRISPR knockout cells lines were generated. Human CD371 was knocked out of HL60gL and U937gL with CRISPR, and knockout was confirmed by flow cytometry using anti-human CD371 APC-conjugated antibody. See FIG. 10.

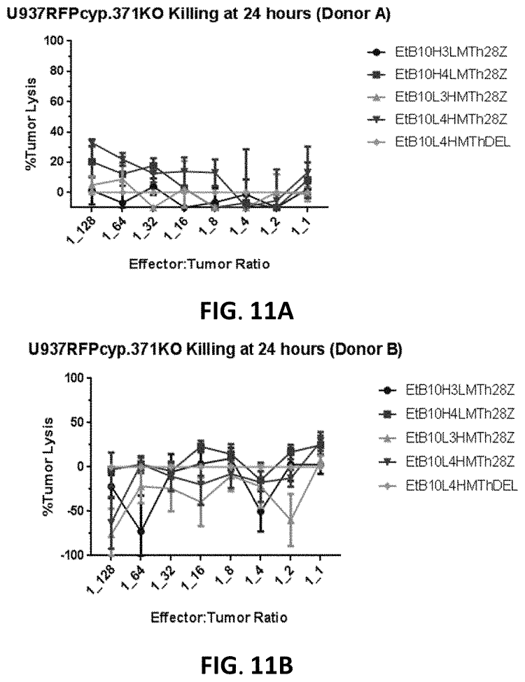

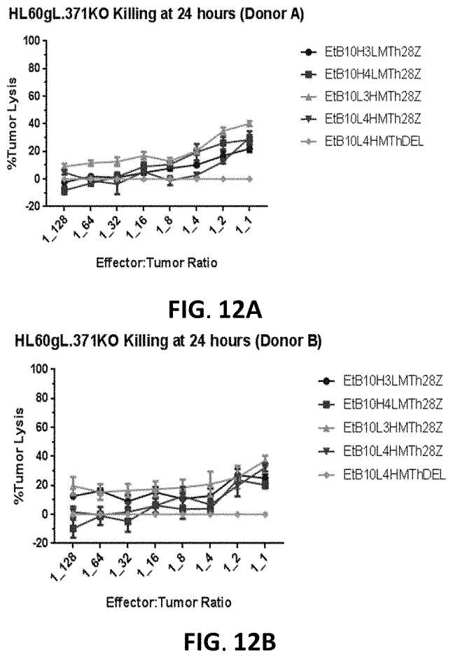

Healthy human donor-derived (i.e., Donor A and Donor B) CD371-targeted CAR T cells were cocultured with antigen-negative U937 cells or HL60 expressing RFP and cypridina luciferase (U937RFPcyp.371KO or HLgL.371KO) at different effector:tumor ratios. 24 hours later, bioluminescence was measured and plotted as a percentage of signal detected in a coculture of a non-functional CD371-targeted CAR T cells (based on B10L4H, but without a CD28 or CD3 zeta signaling domain) and U937RFPcyp.371KO. The results are shown in FIGS. 11A-11B (for U937 cells) and FIGS. 12A-12B (for HL60 cells). As shown in FIGS. 11A-11B and 12A-12B, both B10HL-based and B10LH-based CD371-targeted CAR T cells showed no significant cytotoxicity against CD371-negative U937 cells, and showed limited cytotoxicity against CD371-negative HL60 cells.

### Example 7

### Cytokines Secretion by the CD371-Targeted CAR T Cells

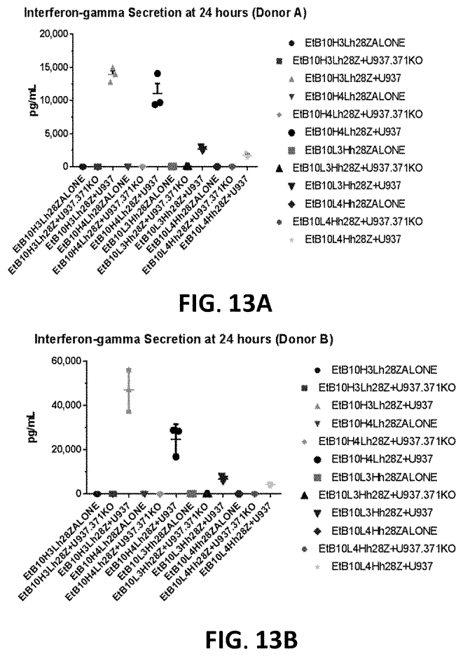

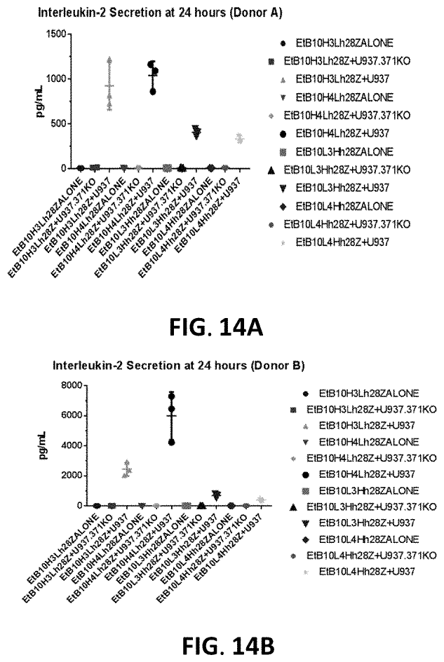

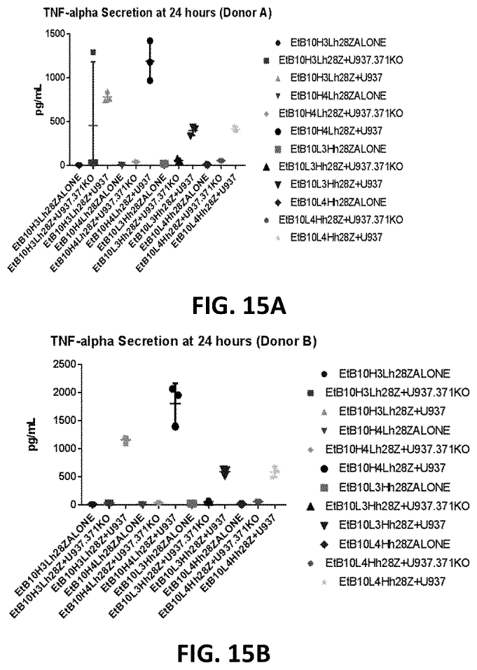

The ability for producing cytokines by the CD371-targeted CAR T cells was assessed. CD371-targeted CAR T cells were cocultured alone, with CD371-negative tumor cells, or CD371-positive U937 cells at an effector:tumor ratio of 1:1 (40,000:40,000 cells in 200 μL). 24 hours later, supernatant was collected from the culture and the secretion levels of IFN-γ, IL-2, and TNF-α were measured utilizing a bead-based multiplex assay. The results are shown in FIGS. 13A-13B (for IFN-γ), FIGS. 14A-14B (for IL-2), and FIGS. 15A-15B (for TNF-α). As shown in FIGS. 13A-13B, FIGS. 14A-14B, and FIGS. 15A-15B, B10HL-based CAQR T cells produced more cytokines in an antigen-dependent manner compared to B10LH-based CAR T cells.

### Example 8

### Proliferation Capacity of CD371-Targeted CAR T Cells

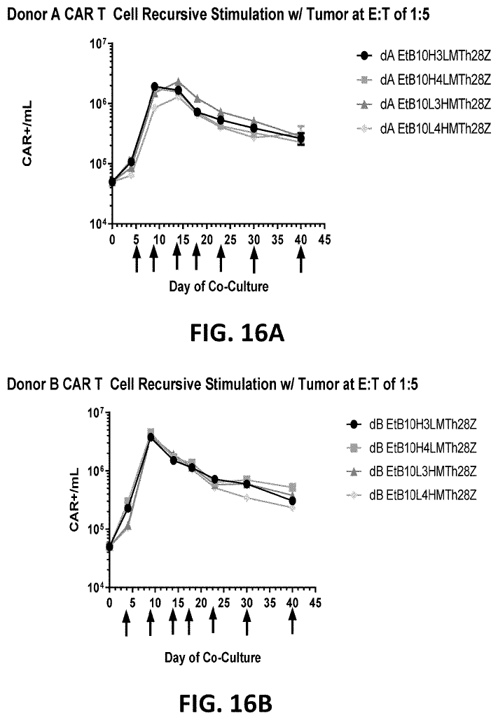

CAR T cells were generated from two healthy donors (i.e., Donor A and Donor B). CD371-targeted CAR T cells were cocultured with CD371+ U937gL at an E:T ratio of 1:5 and at a concentration of 50,000 CAR+/mL. CAR T cells were counted approximately every 5 days, and characterized by flow cytometry. The starting number of tumor cells were added back into the culture at the time point indicated by arrows. The results are shown in FIG. 16. As shown in FIG. 16, B10HL-based and B10-LH-based CD371-targeted CAR T cells showed near-comparative expansion capacities in vitro.

### Example 9

### In Vivo Anti-Tumor Activities of CD371-Targeted CAR T Cells

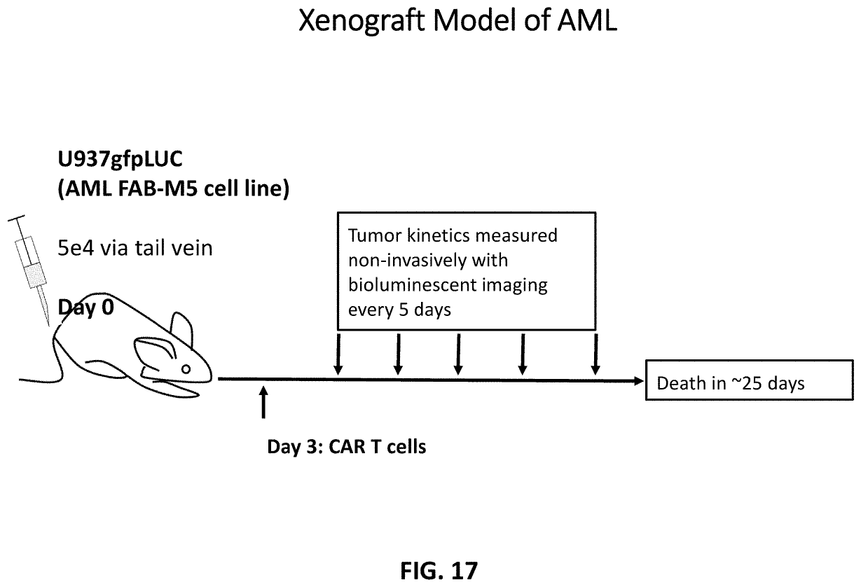

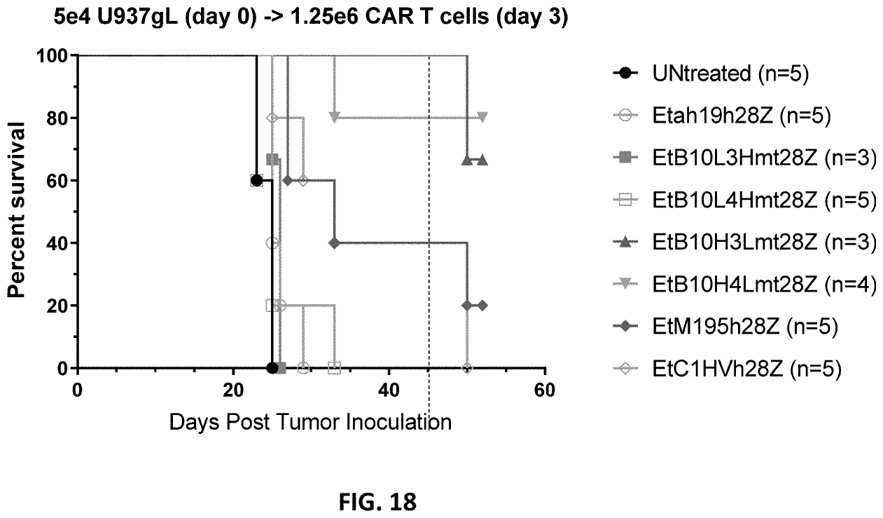

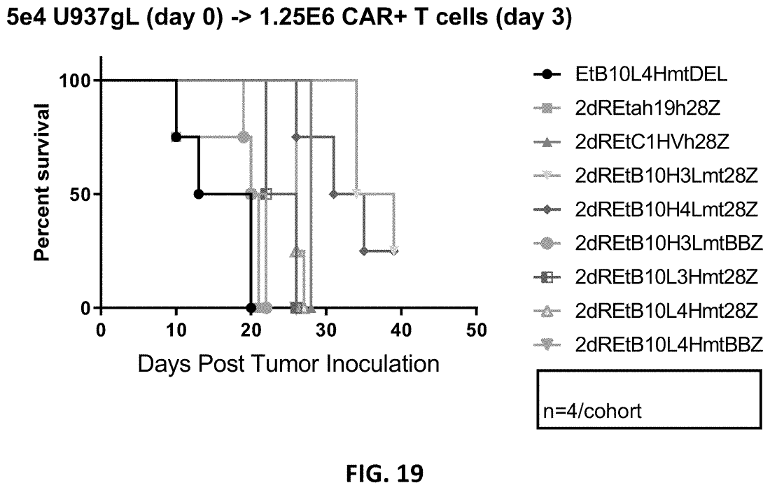

In vivo anti-tumor activities of the B10-based CD371-targeted CAR T cells were assessed. FIG. 17 shows the xenograft model of AML. NCG mice were inoculated with 5.0×10⁴ U937gL tumor cells, AML FAB-M5 cell line via tail vein, and treated with approximately 1.25×10⁶ CAR T cells three days later. Mice survival was monitored. The results are shown in FIG. 18 (for Donor 1) and FIG. 19 (for Donor 2). As shown in FIGS. 18 and 19, B10HL-based CAR T cells showed superior survival in an AML xenografted mice as compared to B10LH-based CAR T cells.

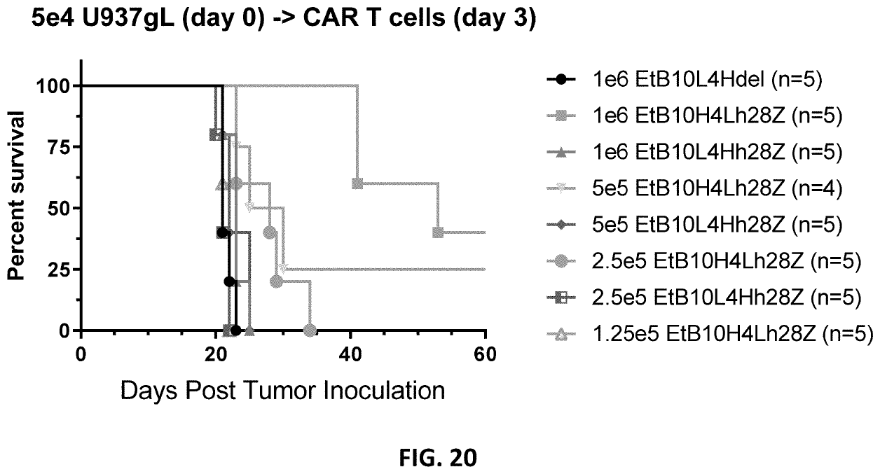

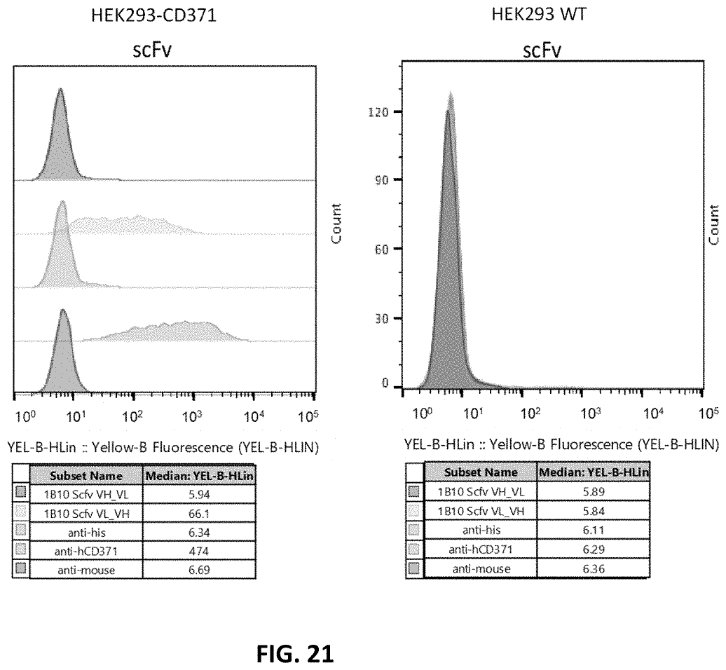

Next, whether the in vivo activities are dose-dependent was assessed. NCG mice were inoculated with 5.0×10⁴ U937gL tumor cells, and treated with various doses of CAR T cells (1.25×10⁵, 2.5×10⁵, 5.0×10⁵, and 1.0×10⁶) three days later. The results are shown in FIG. 20. As shown in FIG. 20, B10HL-based CAR T cells outperformed B10LH-based CAR T cells in a dose-response treatment model.

### Example 10

### B10 scFvs Binding to CD371-Expressing Cell Lines

The binding of B10 (also referred to as “1B10”) scFvs both orientations (V_{H}-V_{L} or V_{L}-V_{H}) to CD371-expressing cells was assessed. The results are shown in FIG. 21. As shown in FIG. 21, binding to cells was detected for the B10 scFv in the V_{L}-V_{H} orientation. However, binding of the B10 scFv in the V_{H}-V_{L} orientation was not observed by flow cytometry, suggesting a lower affinity and thus a requirement for bivalent binding.

### Example 11

### B10 scFvs Binding to Recombinant CD371 in Solution

For scFv affinity measurements, biotinylated CD371 was captured with streptavidin, and soluble scFv was used as analyte. Table 8 shows dissociation constants (K_{D}), on-rates (kₒₙ) and off-rates (k_{off}) for the different scFv formats. Consistent with the flow cytometry results, weak binding of the 1B10 scFv in the V_{H}-V_{L} orientation was observed but a dissociation constant could not be calculated by any curve fit method.

Thus, it is surprising that the CARs comprising the lower binding affinity scFv outperformed in vitro and in vivo than CARs comprising the higher binding affinity scFv, as shown in Examples 5, 7 and 9.

TABLE 8
Binding affinity of B10 scFvs in V_{H}-V_{L} or V_{L}-V_{H} orientation to soluble
CD371

| Format | KD (nM) | Kₒₙ (1/Ms) | K_{off} (Vs) |
|---|---|---|---|
| V_{L}-V_{H} scFv | 16 | 5.99 × 10⁵ | 9.52 × 10⁻³ |
| V_{H}-V_{L} scFv | Weak binding | NA | NA |

### Example 12

### In Vivo and In Vitro Activities of B10HL-Based Second-Generation CAR T Cells

B10HL-based second-generation CAR T cells having a truncated EGFR (Et), a Myc-tag (MT), and G4S linkers of varying lengths were successfully generated:
Et.B10H4L_MT_h28Z is described in Example 1.
Et.B10H1L_MT_h28Z comprised a B10 based scFv (designated as B10H1L) having V_{H}-V_{L} orientation and a 5 amino acids long G4S linker (GGGGS [SEQ ID NO: 93]), and a CD28Z-based signaling domain.
Et.B10H2L_MT_h28Z comprised a B10 based scFv (designated as B10H2L) having V_{H}-V_{L} orientation and a 10 amino acids long G4S linker (GGGGSGGGGS [SEQ ID NO: 94]), and a CD28Z-based signaling domain.
Et.B10H5L_MT_h28Z comprised a B10 based scFv (designated as B10H5L) having V_{H}-V_{L} orientation and a 24 amino acids long G4S linker (GGGGSGGGGSGGGGSGGGSGGGGS [SEQ ID NO: 91]), and a CD28Z-based signaling domain.
Et.B10H6L_MT_h28Z comprised a B10 based scFv (designated as B10H6L) having V_{H}-V_{L} orientation and a 29-amino acid G4S linker (GGGGSGGGGSGGGGSGGGGSGGGSGGGGS [SEQ ID NO: 92]), and a CD28Z-based signaling domain.

Additional B10H4L-based second-generation CAR T cells incorporating either constitutive IL18 (designated as “Et.B10H4Lmt28ZpIL18”) or IL33 (designated as “Et.B10H4Lmt28ZpIL33”) secretion were generated. T cells comprising Et.B10H4Lmt28ZpIL18 comprise the CAR designated as “Et.B10H4L_MT_h28Z” and an exogenous IL-18 polypeptide (e.g., an exogenous IL-18 polypeptide comprising the amino acid sequence set forth in SEQ ID NO 103). T cells comprising Et.B10H4Lmt28ZpIL33 comprise the CAR designated as “Et.B10H4L_MT_h28Z” and an exogenous IL-33 polypeptide (e.g., an exogenous IL-33 polypeptide comprising the amino acid sequence set forth in SEQ ID NO 105).

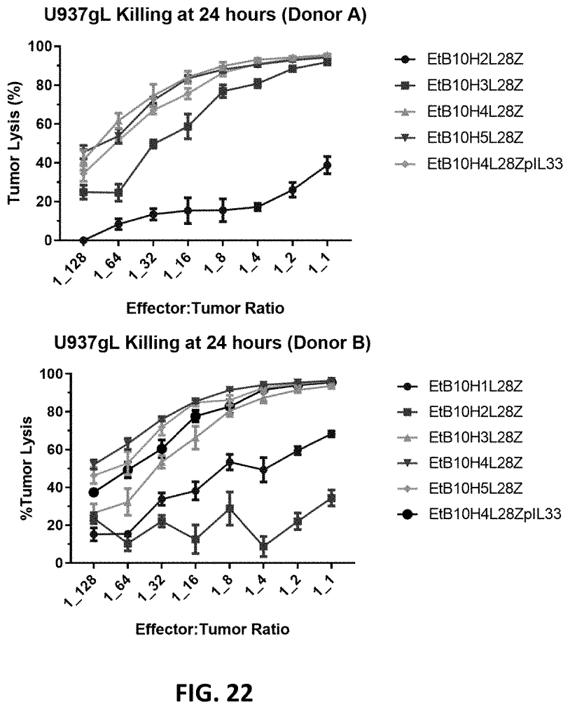

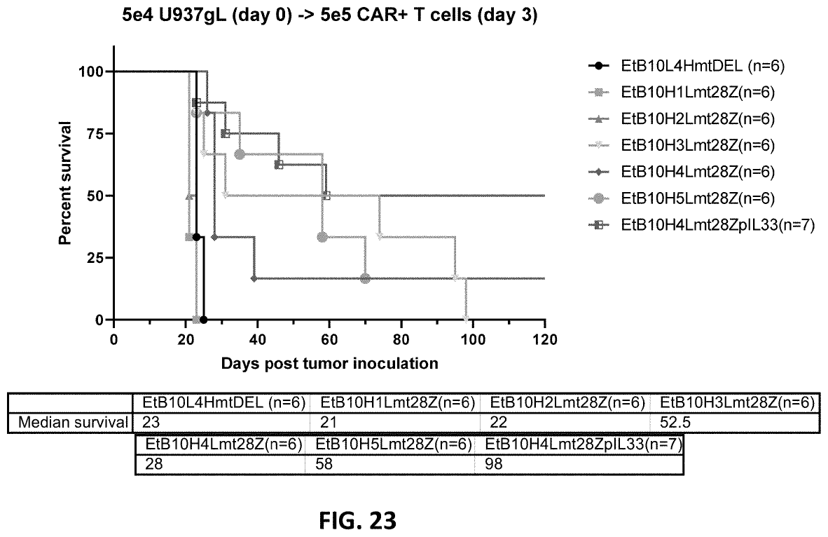

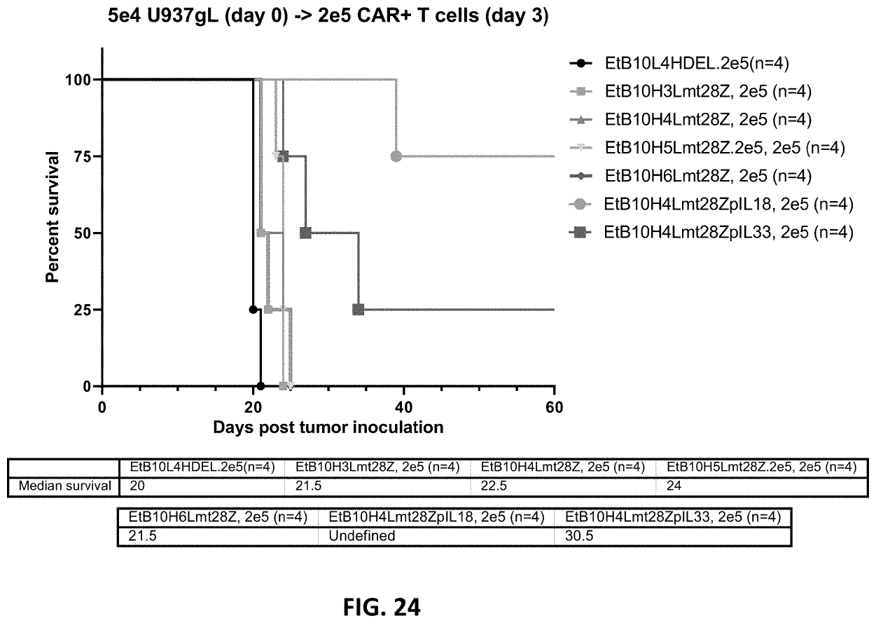

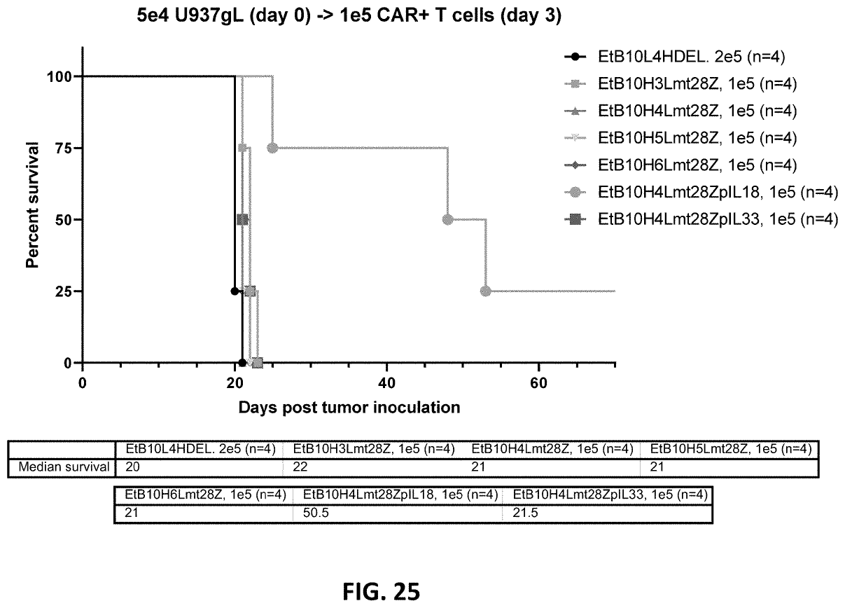

As shown in FIG. 22, CAR T cells having V_{H}-V_{L} oriented scFvs had superior cytotoxicity against U937 cells than CAR T cells having V_{L}-V_{H} oriented scFvs. To assess the in vivo activity of these B10 based CAR T cells, NCG mice were inoculated with 5×10⁴ U937gL tumor cells, and were treated with various doses of CAR T cells. Survival of the treated mice were monitored recorded. As shown in FIG. 23, at the dose of 5×10⁵ CAR T cells, B10H3L, B10H5L, and IL33-secreting B10H4L based human CD371-targeted CAR T cells outperformed B10H4L-based CAR T cells. As shown in FIG. 24, at the dose of 2.5×10⁵ CAR T cells, IL18- and IL33-secreting B10H4L based human CD371-targeted CAR T cells outperformed B10H4L-based CAR T cells. As shown in FIG. 25, at the low dose of 1.0×10⁵ CAR T cells, IL18-secreting B10H4L human CD371-targeted CAR T cells outperform other CAR T cells.

### Embodiments of the Presently Disclosed Subject Matter

From the foregoing description, it will be apparent that variations and modifications may be made to the presently disclosed subject matter to adopt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. An antigen-recognizing receptor comprising an extracellular antigen-binding domain, a transmembrane domain, and an intracellular signaling domain, wherein the extracellular antigen-binding domain specifically binds to CD371.

2. The antigen-recognizing receptor of claim 1, wherein the extracellular antigen-binding domain is a single-chain variable fragment (scFv), a human scFv, a Fab, or a F(ab)₂.

3. The antigen-recognizing receptor of claim 1, wherein one or more of the scFv, Fab and F(ab)₂ are comprised in a fusion protein with a heterologous sequence to form the extracellular antigen-binding domain.

4. The antigen-recognizing receptor of claim 1, wherein the extracellular antigen-binding domain comprises:
(a) a heavy chain variable region CDR1 comprising the amino acid sequence set forth in SEQ ID NO: 28 or a conservative modification thereof; a heavy chain variable region CDR2 comprising the amino acid sequence set forth in SEQ ID NO: 29 or a conservative modification thereof; and a heavy chain variable region CDR3 comprising the amino acid sequence set forth in SEQ ID NO: 30 or a conservative modification thereof;
(b) a heavy chain variable region CDR1 comprising the amino acid sequence set forth in SEQ ID NO: 34 or a conservative modification thereof; a heavy chain variable region CDR2 comprising the amino acid sequence set forth in SEQ ID NO: 35 or a conservative modification thereof; and a heavy chain variable region CDR3 comprising the amino acid sequence set forth in SEQ ID NO: 36 or a conservative modification thereof;
(c) a heavy chain variable region CDR1 comprising the amino acid sequence set forth in SEQ ID NO: 40 or a conservative modification thereof; a heavy chain variable region CDR2 comprising the amino acid sequence set forth in SEQ ID NO: 42 or a conservative modification thereof; and a heavy chain variable region CDR3 comprising the amino acid sequence set forth in SEQ ID NO: 43 or a conservative modification thereof;
(d) a heavy chain variable region CDR1 comprising the amino acid sequence set forth in SEQ ID NO: 46 or a conservative modification thereof, a heavy chain variable region CDR2 comprising the amino acid sequence set forth in SEQ ID NO: 47 or a conservative modification thereof; and a heavy chain variable region CDR3 comprising the amino acid sequence set forth in SEQ ID NO: 48 or a conservative modification thereof;
(e) a heavy chain variable region CDR1 comprising the amino acid sequence set forth in SEQ ID NO: 52 or a conservative modification thereof; a heavy chain variable region CDR2 comprising the amino acid sequence set forth in SEQ ID NO: 53 or a conservative modification thereof; and a heavy chain variable region CDR3 comprising the amino acid sequence set forth in SEQ ID NO: 54 or a conservative modification thereof; or
(f) a heavy chain variable region CDR1 comprising the amino acid sequence set forth in SEQ ID NO: 58 or a conservative modification thereof; a heavy chain variable region CDR2 comprising the amino acid sequence set forth in SEQ ID NO: 59 or a conservative modification thereof; and a heavy chain variable region CDR3 comprising the amino acid sequence set forth in SEQ ID NO: 60 or a conservative modification thereof.

5. The antigen-recognizing receptor of claim 1, wherein the extracellular antigen-binding domain comprises:
(a) a light chain variable region CDR1 comprising the amino acid sequence set forth in SEQ ID NO: 31 or a conservative modification thereof; a light chain variable region CDR2 comprising the amino acid sequence set forth in SEQ ID NO: 32 or a conservative modification thereof; and a light chain variable region CDR3 comprising the amino acid sequence set forth in SEQ ID NO: 33 or a conservative modification thereof;
(b) a light chain variable region CDR1 comprising the amino acid sequence set forth in SEQ ID NO: 37 or a conservative modification thereof; a light chain variable region CDR2 comprising the amino acid sequence set forth in SEQ ID NO: 38 or a conservative modification thereof; and a light chain variable region CDR3 comprising SEQ ID NO: 39 or a conservative modification thereof;
(c) a light chain variable region CDR1 comprising the amino acid sequence set forth in SEQ ID NO: 43 or a conservative modification thereof, a light chain variable region CDR2 comprising the amino acid sequence set forth in SEQ ID NO: 44 or a conservative modification thereof; and a light chain variable region CDR3 comprising the amino acid sequence set forth in SEQ ID NO: 45 or a conservative modification thereof;
(d) a light chain variable region CDR1 comprising the amino acid sequence set forth in SEQ ID NO: 49 or a conservative modification thereof; a light chain variable region CDR2 comprising SEQ ID NO: 50 or a conservative modification thereof; and a light chain variable region CDR3 comprising the amino acid sequence set forth in SEQ ID NO: 51 or a conservative modification thereof;
(e) a light chain variable region CDR1 comprising the amino acid sequence set forth in SEQ ID NO: 55 or a conservative modification thereof; a light chain variable region CDR2 comprising the amino acid sequence set forth in SEQ ID NO: 56 or a conservative modification thereof; and a light chain variable region CDR3 comprising the amino acid sequence set forth in SEQ ID NO: 57 or a conservative modification thereof; or
(f) a light chain variable region CDR1 comprising the amino acid sequence set forth in SEQ ID NO: 61 or a conservative modification thereof; a light chain variable region CDR2 comprising the amino acid sequence set forth in SEQ ID NO: 62 or a conservative modification thereof; and a light chain variable region CDR3 comprising the amino acid sequence set forth in SEQ ID NO: 63 or a conservative modification thereof.

6. The antigen-recognizing receptor of claim 1, wherein the extracellular antigen-binding domain comprises:
(a) a heavy chain variable region CDR1 comprising the amino acid sequence set forth in SEQ ID NO: 28; a heavy chain variable region CDR2 comprising the amino acid sequence set forth in SEQ ID NO: 29; a heavy chain variable region CDR3 comprising the amino acid sequence set forth in SEQ ID NO: 30; a light chain variable region CDR1 comprising the amino acid sequence set forth in SEQ ID NO: 31; a light chain variable region CDR2 comprising the amino acid sequence set forth in SEQ ID NO: 32; and a light chain variable region CDR3 comprising the amino acid sequence set forth in SEQ ID NO: 33;
(b) a heavy chain variable region CDR1 comprising the amino acid sequence set forth in SEQ ID NO: 34; a heavy chain variable region CDR2 comprising the amino acid sequence set forth in SEQ ID NO:35; a heavy chain variable region CDR3 comprising the amino acid sequence set forth in SEQ ID NO: 36; a light chain variable region CDR1 comprising the amino acid sequence set forth in SEQ ID NO: 37; a light chain variable region CDR2 comprising the amino acid sequence set forth in SEQ ID NO: 38; and a light chain variable region CDR3 comprising the amino acid sequence set forth in SEQ ID NO: 39;
(c) a heavy chain variable region CDR1 comprising the amino acid sequence set forth in SEQ ID NO: 40; a heavy chain variable region CDR2 comprising the amino acid sequence set forth in SEQ ID NO: 41; a heavy chain variable region CDR3 comprising the amino acid sequence set forth in SEQ ID NO: 42; a light chain variable region CDR1 comprising the amino acid sequence set forth in SEQ ID NO: 43; a light chain variable region CDR2 comprising the amino acid sequence set forth in SEQ ID NO: 44; and a light chain variable region CDR3 comprising the amino acid sequence set forth in SEQ ID NO: 45;
(d) a heavy chain variable region CDR1 comprising the amino acid sequence set forth in SEQ ID NO: 46; a heavy chain variable region CDR2 comprising the amino acid sequence set forth in SEQ ID NO: 47; a heavy chain variable region CDR3 comprising the amino acid sequence set forth in SEQ ID NO: 48; a light chain variable region CDR1 comprising the amino acid sequence set forth in SEQ ID NO: 49; a light chain variable region CDR2 comprising the amino acid sequence set forth in SEQ ID NO: 50; and a light chain variable region CDR3 comprising the amino acid sequence set forth in SEQ ID NO: 51;
(e) a heavy chain variable region CDR1 comprising the amino acid sequence set forth in SEQ ID NO: 52; a heavy chain variable region CDR2 comprising the amino acid sequence set forth in SEQ ID NO: 53; a heavy chain variable region CDR3 comprising the amino acid sequence set forth in SEQ ID NO: 54; a light chain variable region CDR1 comprising the amino acid sequence set forth in SEQ ID NO: 55; a light chain variable region CDR2 comprising the amino acid sequence set forth in SEQ ID NO: 56; and a light chain variable region CDR3 comprising the amino acid sequence set forth in SEQ ID NO: 57; or
(f) a heavy chain variable region CDR1 comprising the amino acid sequence set forth in SEQ ID NO: 58; a heavy chain variable region CDR2 comprising the amino acid sequence set forth in SEQ ID NO: 59; a heavy chain variable region CDR3 comprising the amino acid sequence set forth in SEQ ID NO: 60; a light chain variable region CDR1 comprising the amino acid sequence set forth in SEQ ID NO: 61; a light chain variable region CDR2 comprising the amino acid sequence set forth in SEQ ID NO: 62; and a light chain variable region CDR3 comprising the amino acid sequence set forth in SEQ ID NO: 63.

7. The antigen-recognizing receptor of claim 1, wherein the extracellular antigen-binding domain comprises: a heavy chain variable region CDR1 comprising the amino acid sequence set forth in SEQ ID NO: 28; a heavy chain variable region CDR2 comprising the amino acid sequence set forth in SEQ ID NO: 29; a heavy chain variable region CDR3 comprising the amino acid sequence set forth in SEQ ID NO: 30; a light chain variable region CDR1 comprising the amino acid sequence set forth in SEQ ID NO: 31; a light chain variable region CDR2 comprising the amino acid sequence set forth in SEQ ID NO: 32; and a light chain variable region CDR3 comprising the amino acid sequence set forth in SEQ ID NO: 33.

8. The antigen-recognizing receptor of claim 1, wherein the extracellular antigen-binding domain comprises: (a) a heavy chain variable region comprising an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence selected set forth in SEQ ID NO: 1, SEQ ID NO: 3, SEQ ID NO: 5, SEQ ID NO: 7, SEQ ID NO: 9, or SEQ ID NO: 11; and/or (b) a light chain variable region comprising an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 2, SEQ ID NO: 4, SEQ ID NO: 6, SEQ ID NO: 8, SEQ ID NO: 10, or SEQ ID NO: 12.

9. The antigen-recognizing receptor of claim 1, wherein the extracellular antigen-binding domain comprises:
(a) a heavy chain variable region comprising the amino acid sequence set forth in SEQ ID NO: 1, and a light chain variable region comprising the amino acid sequence set forth in SEQ ID NO: 2;
(b) a heavy chain variable region comprising the amino acid sequence set forth in SEQ ID NO: 3, and a light chain variable region comprising the amino acid sequence set forth in SEQ ID NO: 4;
(c) a heavy chain variable region comprising the amino acid sequence set forth in SEQ ID NO: 5, and a light chain variable region comprising the amino acid sequence set forth in SEQ ID NO: 6;
(d) a heavy chain variable region comprising the amino acid sequence set forth in SEQ ID NO: 7, and a light chain variable region comprising the amino acid sequence set forth in SEQ ID NO: 8;
(e) a heavy chain variable region comprising the amino acid sequence set forth in SEQ ID NO: 9, and a light chain variable region comprising the amino acid sequence set forth in SEQ ID NO: 10; or
(f) a heavy chain variable region comprising the amino acid sequence set forth in SEQ ID NO: 11, and a light chain variable region comprising the amino acid sequence set forth in SEQ ID NO: 12.

10. The antigen-recognizing receptor of claim 1, wherein the extracellular antigen-binding domain comprises a heavy chain variable region comprising the amino acid sequence set forth in SEQ ID NO: 1; and a light chain variable region comprising the amino acid sequence set forth in SEQ ID NO: 2.

11. The antigen-recognizing receptor of claim 1, wherein the extracellular antigen-binding domain comprises:
(i) a linker between a heavy chain variable region and a light chain variable region of the extracellular antigen-binding domain; and/or
(ii) a signal peptide that is covalently joined to the 5′ terminus of the extracellular antigen-binding domain.

12. The antigen-recognizing receptor of claim 11, wherein the linker consists of the amino acid sequence set forth in SEQ ID NO: 13, SEQ ID NO: 14, SEQ ID NO: 91, SEQ ID NO: 92, SEQ ID NO: 93, or SEQ ID NO: 94.

13. The antigen-recognizing receptor of claim 1, wherein the extracellular antigen-binding domain comprises a heavy chain variable region and a light chain variable region, which are positioned from the N- to the C-terminus: V_{H}-V_{L}.

14. The antigen-recognizing receptor of claim 1, wherein the extracellular antigen-binding domain binds to CD371 with (i) a low binding affinity, or (ii) a dissociation constant (K_{d}) of 1×10⁻⁸ M or more.

15. The antigen-recognizing receptor of claim 1, wherein
(i) the transmembrane domain comprises a CD8 polypeptide, a CD28 polypeptide, a CD3ζ polypeptide, a CD4 polypeptide, a 4-1BB polypeptide, an OX40 polypeptide, an ICOS polypeptide, a CTLA-4 polypeptide, a PD-1 polypeptide, a LAG-3 polypeptide, a 2B4 polypeptide, a BTLA polypeptide, or a combination thereof; and/or
(ii) the intracellular signaling domain comprises a CD3ζ polypeptide.

16. The antigen-recognizing receptor of claim 1, wherein the intracellular signaling domain further comprises at least one co-stimulatory signaling region.

17. The antigen-recognizing receptor of claim 16, wherein the at least one co-stimulatory signaling region comprises a CD28 polypeptide, a 4-1BB polypeptide, an OX40 polypeptide, an ICOS polypeptide, a DAP-10 polypeptide, or a combination thereof.

18. The antigen-recognizing receptor of claim 1, wherein the antigen-recognizing receptor is a chimeric antigen receptor (CAR), a T-cell Receptor (TCR), or a T-cell receptor-like fusion protein.

19. The antigen-recognizing receptor of claim 1, wherein the antigen-recognizing receptor is a CAR.

20. The antigen-recognizing receptor of claim 1, wherein the antigen-recognizing receptor is recombinantly expressed and/or expressed from a vector.

21. A cell comprising the antigen-recognizing receptor of claim 1.

22. The cell of claim 21, wherein the antigen-recognizing receptor is constitutively expressed on the surface of the cell.

23. The cell of claim 21, wherein the cell is engineered to express a cytokine or a fragment thereof.

24. The cell of claim 23, wherein the cell comprises an exogenous polypeptide of the cytokine or fragment thereof, and/or the cell comprises a nucleic acid molecule encoding the cytokine or fragment thereof.

25. The cell of claim 23, wherein the cytokine is selected from the group consisting of IL-18, IL-33, IL-36, and combinations thereof.

26. The cell of claim 21, wherein the cell is
(i) an immunoresponsive cell;
(ii) a cell of the lymphoid lineage or a cell of the myeloid lineage;
(iii) selected from the group consisting of a T cell, a Natural Killer (NK) cell, and a stem cell from which a lymphoid cell may be differentiated;
(iv) a T cell; and/or
(v) a cytotoxic T lymphocyte (CTL) or a regulatory T cell .

27. The cell of claim 26, wherein the stem cell is a pluripotent stem cell, an embryoid stem cell, or an induced pluripotent stem cell.

28. A nucleic acid molecule encoding the antigen-recognizing receptor of claim 1.

29. A vector comprising the nucleic acid molecule of claim 28.

30. A host cell expressing the nucleic acid molecule of claim 28.

31. A composition comprising the cell of claim 21.

32. The composition of claim 31, which is a pharmaceutical composition further comprising a pharmaceutically acceptable carrier.

33. A method of reducing tumor burden in a subject, comprising administering an effective amount of the cell of claim 21.

34. A method of increasing or lengthening survival of a subject having a tumor or neoplasm, comprising administering an effective amount of the cell of claim 21.

35. A method of treating and/or preventing a tumor or neoplasm in a subject, comprising administering an effective amount of the cell of claim 21.

36. A method for producing a cell comprising an antigen-recognizing receptor of claim 1, comprising introducing into the cell a nucleic acid molecule that encodes the antigen-recognizing receptor.

37. A kit for reducing tumor burden in a subject, treating and/or preventing a tumor or neoplasm in a subject, and/or increasing or lengthening survival of a subject having a tumor or neoplasm, comprising the cell of claim 21.


SEQUENCE LISTING (112 sequences)

SEQ ID NO: 1 - protein, 122 aa, Artificial Sequence
  Synthetic
EVQLLESGGGLVQPGGSLRLSCAASGFTFSDYQMSWVRQAPGKGLEWVSGIQGGGGSTYYADSVKGRFTISRDNSKNTLYLQMNSLRAEDTAVYYCAREMWRGDYYSGMDVWGQGTTVTVSS

SEQ ID NO: 2 - protein, 113 aa, Artificial Sequence
  Synthetic
DIVMTQSPDSLAVSLGERATINCKSSQSVLDSYNNENNLAWYQQKPGQPPKLLIYWASTRESGVPDRFSGSGSGTDFTLTISSLQAEDVAVYYCQQYTSEPITFGQGTKVEIK

SEQ ID NO: 3 - protein, 125 aa, Artificial Sequence
  Synthetic
EVQLLESGGGLVQPGGSLRLSCAASGFTFTSYAMSWVRQAPGKGLEWVSGIDGSGGGTNYADSVKGRFTISRDNSKNTLYLQMNSLRAEDTAVYYCARAYYDILTGYPVDGMDVWGQGTTVTVSS

SEQ ID NO: 4 - protein, 113 aa, Artificial Sequence
  Synthetic
DIVMTQSPDSLAVSLGERATINCKSSQSVLSSYNNENNLAWYQQKPGQPPKLLIYAASTRESGVPDRFSGSGSGTDFTLTISSLQAEDVAVYYCQQYYSEPYTFGQGTKVEIK

SEQ ID NO: 5 - protein, 117 aa, Artificial Sequence
  Synthetic
EVQLLESGGGLVQPGGSLRLSCAASGFTFTDYAMSWVRQAPGKGLEWVSDIDGSGGSTDYADSVKGRFTISRDNSKNTLYLQMNSLRAEDTAVYYCALELGATTVYWGQGTLVTVSS

SEQ ID NO: 6 - protein, 113 aa, Artificial Sequence
  Synthetic
DIVMTQSPDSLAVSLGERATINCKSSQSVLRSSNNKNNLAWYQQKPGQPPKLLIYAASTRESGVPDRFSGSGSGTDFTLTISSLQAEDVAVYYCQQYYREPLTFGQGTKVEIK

SEQ ID NO: 7 - protein, 120 aa, Artificial Sequence
  Synthetic
EVQLLESGGGLVQPGGSLRLSCAASGFTFTSTQMSWVRQAPGKGLEWVSEISGYGGSTYYADSVKGRFTISRDNSKNTLYLQMNSLRAEDTAVYYCAKDTEVSGDAFDIWGQGTMVTVSS

SEQ ID NO: 8 - protein, 108 aa, Artificial Sequence
  Synthetic
EIVLTQSPGTLSLSPGERATLSCRASQSVDSSNLAWYQQKPGQAPRLLIYGASSRATGIPDRFSGSGSGTDFTLTISRLEPEDFAVYYCQQYRSWPITFGQGTKVEIK

SEQ ID NO: 9 - protein, 122 aa, Artificial Sequence
  Synthetic
EVQLLESGGGLVQPGGSLRLSCAASGFTFTSYYMSWVRQAPGKGLEWVSGISGSGDSTSYADSVKGRFTISRDNSKNTLYLQMNSLRAEDTAVYYCAREAGGDYDSGAFDIWGQGTMVTVSS

SEQ ID NO: 10 - protein, 113 aa, Artificial Sequence
  Synthetic
DIVMTQSPDSLAVSLGERATINCKSSQSVLYSGNNKNYLAWYQQKPGQPPKLLIYGASTRESGVPDRFSGSGSGTDFTLTISSLQAEDVAVYYCQQYDYAPFTFGQGTKVEIK

SEQ ID NO: 11 - protein, 127 aa, Artificial Sequence
  Synthetic
EVQLLESGGGLVQPGGSLRLSCAASGFTFSSYAMSWVRQAPGKGLEWVSEIDGEGGYTNYADSVKGRFTISRDNSKNTLYLQMNSLRAEDTAVYYCAREGVDYDILTGYYPYGMDVWGQGTTVTVSS

SEQ ID NO: 12 - protein, 114 aa, Artificial Sequence
  Synthetic
DIVMTQSPDSLAVSLGERATINCKSSQSVLDSSNNKNYLAWYQQKPGQPPKLLIYDASTRESGVPDRFSGSGSGTDFTLTISSLQAEDVAVYYCQQGTSSPLTFGQGTKVEIKG

SEQ ID NO: 13 - protein, 19 aa, Artificial Sequence
  Synthetic
GGGGSGGGGSGGGSGGGGS

SEQ ID NO: 14 - protein, 15 aa, Artificial Sequence
  Synthetic
GGGGSGGGGSGGGGS

SEQ ID NO: 15 - protein, 265 aa, Homo sapiens
MSEEVTYADLQFQNSSEMEKIPEIGKFGEKAPPAPSHVWRPAALFLTLLCLLLLIGLGVLASMFHVTLKIEMKKMNKLQNISEELQRNISLQLMSNMNISNKIRNLSTTLQTIATKLCRELYSKEQEHKCKPCPRRWIWHKDSCYFLSDDVQTWQESKMACAAQNASLLKINNKNALEFIKSQSRSYDYWLGLSPEEDSTRGMRVDNIINSSAWVIRNAPDLNNMYCGYINRLYVQYYHCTYKKRMICEKMANPVQLGSTYFREA

SEQ ID NO: 16 - protein, 254 aa, Artificial Sequence
  Synthetic
DIVMTQSPDSLAVSLGERATINCKSSQSVLDSYNNENNLAWYQQKPGQPPKLLIYWASTRESGVPDRFSGSGSGTDFTLTISSLQAEDVAVYYCQQYTSEPITFGQGTKVEIKGGGGSGGGGSGGGSGGGGSEVQLLESGGGLVQPGGSLRLSCAASGFTFSDYQMSWVRQAPGKGLEWVSGIQGGGGSTYYADSVKGRFTISRDNSKNTLYLQMNSLRAEDTAVYYCAREMWRGDYYSGMDVWGQGTTVTVSS

SEQ ID NO: 17 - protein, 257 aa, Artificial Sequence
  Synthetic
DIVMTQSPDSLAVSLGERATINCKSSQSVLSSYNNENNLAWYQQKPGQPPKLLIYAASTRESGVPDRFSGSGSGTDFTLTISSLQAEDVAVYYCQQYYSEPYTFGQGTKVEIKGGGGSGGGGSGGGSGGGGSEVQLLESGGGLVQPGGSLRLSCAASGFTFTSYAMSWVRQAPGKGLEWVSGIDGSGGGTNYADSVKGRFTISRDNSKNTLYLQMNSLRAEDTAVYYCARAYYDILTGYPVDGMDVWGQGTTVTVSS

SEQ ID NO: 18 - protein, 249 aa, Artificial Sequence
  Synthetic
DIVMTQSPDSLAVSLGERATINCKSSQSVLRSSNNKNNLAWYQQKPGQPPKLLIYAASTRESGVPDRFSGSGSGTDFTLTISSLQAEDVAVYYCQQYYREPLTFGQGTKVEIKGGGGSGGGGSGGGSGGGGSEVQLLESGGGLVQPGGSLRLSCAASGFTFTDYAMSWVRQAPGKGLEWVSDIDGSGGSTDYADSVKGRFTISRDNSKNTLYLQMNSLRAEDTAVYYCALELGATTVYWGQGTLVTVSS

SEQ ID NO: 19 - protein, 247 aa, Artificial Sequence
  Synthetic
EIVLTQSPGTLSLSPGERATLSCRASQSVDSSNLAWYQQKPGQAPRLLIYGASSRATGIPDRFSGSGSGTDFTLTISRLEPEDFAVYYCQQYRSWPITFGQGTKVEIKGGGGSGGGGSGGGSGGGGSEVQLLESGGGLVQPGGSLRLSCAASGFTFTSTQMSWVRQAPGKGLEWVSEISGYGGSTYYADSVKGRFTISRDNSKNTLYLQMNSLRAEDTAVYYCAKDTEVSGDAFDIWGQGTMVTVSS

SEQ ID NO: 20 - protein, 254 aa, Artificial Sequence
  Synthetic
DIVMTQSPDSLAVSLGERATINCKSSQSVLYSGNNKNYLAWYQQKPGQPPKLLIYGASTRESGVPDRFSGSGSGTDFTLTISSLQAEDVAVYYCQQYDYAPFTFGQGTKVEIKGGGGSGGGGSGGGSGGGGSEVQLLESGGGLVQPGGSLRLSCAASGFTFTSYYMSWVRQAPGKGLEWVSGISGSGDSTSYADSVKGRFTISRDNSKNTLYLQMNSLRAEDTAVYYCAREAGGDYDSGAFDIWGQGTMVTVSS

SEQ ID NO: 21 - protein, 259 aa, Artificial Sequence
  Synthetic
DIVMTQSPDSLAVSLGERATINCKSSQSVLDSSNNKNYLAWYQQKPGQPPKLLIYDASTRESGVPDRFSGSGSGTDFTLTISSLQAEDVAVYYCQQGTSSPLTFGQGTKVEIKGGGGSGGGGSGGGSGGGGSEVQLLESGGGLVQPGGSLRLSCAASGFTFSSYAMSWVRQAPGKGLEWVSEIDGEGGYTNYADSVKGRFTISRDNSKNTLYLQMNSLRAEDTAVYYCAREGVDYDILTGYYPYGMDVWGQGTTVTVSS

SEQ ID NO: 22 - DNA, 762 bp, Artificial Sequence
  Synthetic
gacatcgtgatgacccagtctccagactccctggctgtgtctctgggcgagcgtgccaccatcaactgcaagtccagccagagtgttttagacagctataacaatgagaacaatttagcttggtatcagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatataccagcgaacctatcacgttcggccaaggtaccaaggtggaaatcaaaggtggtggtggttcaggtggtggtggttctggcggcggctccggtggtggtggatccgaggtgcagctgttggagtctgggggaggcttggtacagcctggggggtccctgcgactctcctgtgcagcctctggattcacctttagcgactatcagatgagctgggtccgccaggctccagggaaggggctggagtgggtgtcaggcattcagggtggcggtggtagcacatattacgcagactccgtgaagggccggttcaccatctcccgtgacaattccaagaacacgctgtatctgcaaatgaacagcctgcgtgccgaggacacggctgtgtattactgtgcgagagagatgtggcgtggggactactactccggtatggacgtctggggccaggggaccacggtcaccgtctcctca

SEQ ID NO: 23 - DNA, 771 bp, Artificial Sequence
  Synthetic
gacatcgtgatgacccagtctccagactccctggctgtgtctctgggcgagcgtgccaccatcaactgcaagtccagccagagtgttttaagcagctataacaatgagaacaatttagcttggtatcagcagaaaccaggacagcctcctaagctgctcatttacgccgcatctacccgggaatccggggtccctgaccgattcagtggcagcgggtctgggacagatttcactctcaccatcagcagcctgcaggctgaagatgtggcagtttattactgtcagcaatattatagcgaaccttatacgttcggccaaggtaccaaggtggaaatcaaaggtggtggtggttcaggtggtggtggttctggcggcggctccggtggtggtggatccgaggtgcagctgttggagtctgggggaggcttggtacagcctggggggtccctgcgactctcctgtgcagcctctggattcacctttaccagctatgccatgagctgggtccgccaggctccagggaaggggctggagtgggtgtcaggcattgacggtagcggtggtggcacaaattacgcagactccgtgaagggccggttcaccatctcccgtgacaattccaagaacacgctgtatctgcaaatgaacagcctgcgtgccgaggacacggctgtgtattactgtgcgagagcgtattacgatattttgactggttaccccgtggacggtatggacgtctggggccaagggaccacggtcaccgtctcctca

SEQ ID NO: 24 - DNA, 747 bp, Artificial Sequence
  Synthetic
gacatcgtgatgacccagtctccagactccctggctgtgtctctgggcgagcgtgccaccatcaactgcaagtccagccagagtgttttacgcagcagcaacaataaaaacaatttagcttggtatcagcagaaaccaggacagcctcctaagctgctcatttacgccgcatctacccgggaatccggggtccctgaccgattcagtggcagcgggtctgggacagatttcactctcaccatcagcagcctgcaggctgaagatgtggcagtttattactgtcagcaatattatcgcgaacctctgacgttcggccaaggtaccaaggtggaaatcaaaggtggtggtggttcaggtggtggtggttctggcggcggctccggtggtggtggatccgaggtgcagctgttggagtctgggggaggcttggtacagcctggggggtccctgcgactctcctgtgcagcctctggattcacctttaccgactatgccatgagctgggtccgccaggctccagggaaggggctggagtgggtgtcagacattgacggtagcggtggtagcacagactacgcagactccgtgaagggccggttcaccatctcccgtgacaattccaagaacacgctgtatctgcaaatgaacagcctgcgtgccgaggacacggctgtgtattactgtgcgctagagctgggagctactaccgtctactggggccagggaaccctggtcaccgtctcctca

SEQ ID NO: 25 - DNA, 741 bp, Artificial Sequence
  Synthetic
gaaattgtgttgacgcagtctccaggcaccctgtctttgtctccaggggaacgtgccaccctctcctgccgtgccagtcagagtgttgacagcagcaatttagcctggtatcagcagaaacctggccaggctccccgactcctcatctatggcgcatctagccgtgccactggtatcccagaccgtttcagtggcagtgggtctgggacagacttcactctcaccatcagcagactggagcctgaagattttgcagtgtattactgtcagcagtatcgcagctggcctatcacgttcggccaaggtaccaaggtggaaatcaaaggtggtggtggttcaggtggtggtggttctggcggcggctccggtggtggtggatccgaggtgcagctgttggagtctgggggaggcttggtacagcctggggggtccctgcgactctcctgtgcagcctctggattcacctttaccagcacccagatgagctgggtccgccaggctccagggaaggggctggagtgggtgtcagagattagcggttatggtggtagcacatactacgcagactccgtgaagggccggttcaccatctcccgtgacaattccaagaacacgctgtatctgcaaatgaacagcctgcgtgccgaggacacggctgtgtattactgtgcaaaagacacggaggtttcgggagatgcttttgatatctggggccaagggacaatggtcaccgtctcttca

SEQ ID NO: 26 - DNA, 762 bp, Artificial Sequence
  Synthetic
gacatcgtgatgacccagtctccagactccctggctgtgtctctgggcgagcgtgccaccatcaactgcaagtccagccagagtgttttatatagcggcaacaataaaaactatttagcttggtatcagcagaaaccaggacagcctcctaagctgctcatttacggcgcatctacccgggaatccggggtccctgaccgattcagtggcagcgggtctgggacagatttcactctcaccatcagcagcctgcaggctgaagatgtggcagtttattactgtcagcaatatgactatgccccttttacgttcggccaaggtaccaaggtggaaatcaaaggtggtggtggttcaggtggtggtggttctggcggcggctccggtggtggtggatccgaggtgcagctgttggagtctgggggaggcttggtacagcctggggggtccctgcgactctcctgtgcagcctctggattcacctttaccagctattatatgagctgggtccgccaggctccagggaaggggctggagtgggtgtcaggcattagcggtagcggtgacagcacaagctacgcagactccgtgaagggccggttcaccatctcccgtgacaattccaagaacacgctgtatctgcaaatgaacagcctgcgtgccgaggacacggctgtgtattactgtgcgagagaggcaggtggtgactacgatagtggtgcttttgatatctggggccaagggacaatggtcaccgtctcttca

SEQ ID NO: 27 - DNA, 777 bp, Artificial Sequence
  Synthetic
gacatcgtgatgacccagtctccagactccctggctgtgtctctgggcgagcgtgccaccatcaactgcaagtccagccagagtgttttagacagcagcaacaataaaaactatttagcttggtatcagcagaaaccaggacagcctcctaagctgctcatttacgacgcatctacccgggaatccggggtccctgaccgattcagtggcagcgggtctgggacagatttcactctcaccatcagcagcctgcaggctgaagatgtggcagtttattactgtcagcaaggcaccagcagccctctgacgttcggccaaggtaccaaggtggaaatcaaaggtggtggtggttcaggtggtggtggttctggcggcggctccggtggtggtggatccgaggtgcagctgttggagtctgggggaggcttggtacagcctggggggtccctgcgactctcctgtgcagcctctggattcacctttagcagctatgccatgagctgggtccgccaggctccagggaaggggctggagtgggtgtcagagattgacggtgagggtggttatacaaattacgcagactccgtgaagggccggttcaccatctcccgtgacaattccaagaacacgctgtatctgcaaatgaacagcctgcgtgccgaggacacggccgtgtattactgtgcgagagaaggggtagattacgatattttgactggttattatccttacggtatggacgtctggggccaagggaccacggtcaccgtctcctca

SEQ ID NO: 28 - protein, 8 aa, Artificial Sequence
  Synthetic
GFTFSDYQ

SEQ ID NO: 29 - skipped

SEQ ID NO: 30 - protein, 15 aa, Artificial Sequence
  Synthetic
AREMWRGDYYSGMDV

SEQ ID NO: 31 - protein, 12 aa, Artificial Sequence
  Synthetic
QSVLDSYNNENN

SEQ ID NO: 32 - protein, 3 aa, Artificial Sequence
  Synthetic
WAS

SEQ ID NO: 33 - protein, 9 aa, Artificial Sequence
  Synthetic
QQYTSEPIT

SEQ ID NO: 34 - protein, 8 aa, Artificial Sequence
  Synthetic
GFTFTSYA

SEQ ID NO: 35 - protein, 8 aa, Artificial Sequence
  Synthetic
IDGSGGGT

SEQ ID NO: 36 - protein, 8 aa, Artificial Sequence
  Synthetic
ARAYYDIL

SEQ ID NO: 37 - protein, 12 aa, Artificial Sequence
  Synthetic
QSVLSSYNNENN

SEQ ID NO: 38 - protein, 3 aa, Artificial Sequence
  Synthetic
AAS

SEQ ID NO: 39 - protein, 9 aa, Artificial Sequence
  Synthetic
QQYYSEPYT

SEQ ID NO: 40 - protein, 8 aa, Artificial Sequence
  Synthetic
GFTFTDYA

SEQ ID NO: 41 - protein, 8 aa, Artificial Sequence
  Synthetic
IDGSGGST

SEQ ID NO: 42 - protein, 10 aa, Artificial Sequence
  Synthetic
ALELGATTVY

SEQ ID NO: 43 - protein, 12 aa, Artificial Sequence
  Synthetic
QSVLRSSNNKNN

SEQ ID NO: 44 - protein, 3 aa, Artificial Sequence
  Synthetic
AAS

SEQ ID NO: 45 - protein, 9 aa, Artificial Sequence
  Synthetic
QQYYREPLT

SEQ ID NO: 46 - protein, 8 aa, Artificial Sequence
  Synthetic
GFTFTSTQ

SEQ ID NO: 47 - protein, 8 aa, Artificial Sequence
  Synthetic
ISGYGGST

SEQ ID NO: 48 - protein, 13 aa, Artificial Sequence
  Synthetic
AKDTEVSGDAFDI

SEQ ID NO: 49 - protein, 7 aa, Artificial Sequence
  Synthetic
QSVDSSN

SEQ ID NO: 50 - protein, 3 aa, Artificial Sequence
  Synthetic
GAS

SEQ ID NO: 51 - protein, 9 aa, Artificial Sequence
  Synthetic
QQYRSWPIT

SEQ ID NO: 52 - protein, 8 aa, Artificial Sequence
  Synthetic
GFTFTSYY

SEQ ID NO: 53 - protein, 8 aa, Artificial Sequence
  Synthetic
ISGSGDST

SEQ ID NO: 54 - protein, 15 aa, Artificial Sequence
  Synthetic
AREAGGDYDSGAFDI

SEQ ID NO: 55 - protein, 12 aa, Artificial Sequence
  Synthetic
QSVLYSGNNKNY

SEQ ID NO: 56 - protein, 3 aa, Artificial Sequence
  Synthetic
GAS

SEQ ID NO: 57 - protein, 9 aa, Artificial Sequence
  Synthetic
QQYDYAPFT

SEQ ID NO: 58 - protein, 8 aa, Artificial Sequence
  Synthetic
GFTFSSYA

SEQ ID NO: 59 - protein, 8 aa, Artificial Sequence
  Synthetic
IDGEGGYT

SEQ ID NO: 60 - protein, 20 aa, Artificial Sequence
  Synthetic
AREGVDYDILTGYYPYGMDV

SEQ ID NO: 61 - protein, 12 aa, Artificial Sequence
  Synthetic
QSVLDSSNNKNY

SEQ ID NO: 62 - protein, 3 aa, Artificial Sequence
  Synthetic
DAS

SEQ ID NO: 63 - protein, 9 aa, Artificial Sequence
  Synthetic
QQGTSSPLT

SEQ ID NO: 64 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 65 - protein, 247 aa, Mus musculus
MASPLTRFLSLNLLLMGESIILGSGEAKPQAPELRIFPKKMDAELGQKVDLVCEVLGSVSQGCSWLFQNSSSKLPQPTFVVYMASSHNKITWDEKLNSSKLFSAVRDTNNKYVLTLNKFSKENEGYYFCSVISNSVMYFSSVVPVLQKVNSTTTKPVLRTPSPVHPTGTSQPQRPEDCRPRGSVKGTGLDFACDIYIWAPLAGICVAPLLSLIITLICYHRSRKRVCKCPRPLVRQEGKPRPSEKIV

SEQ ID NO: 66 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 67 - DNA, 81 bp, Homo sapiens
ttttgggtgctggtggtggttggtggagtcctggcttgctatagcttgctagtaacagtggcctttattattttctgggtg

SEQ ID NO: 68 - protein, 218 aa, Mus musculus
MTLRLLFLALNFFSVQVTENKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRP

SEQ ID NO: 69 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 70 - DNA, 123 bp, Artificial Sequence
  Synthetic
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 71 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 72 - protein, 126 aa, Artificial Sequence
  Synthetic
AAACGGGGCAGAAAGAAACTCCTGTATATATTCAAACAACCATTTATGAGACCAGTACAAACTACTCAAGAGGAAGATGGCTGTAGCTGCCGATTTCCAGAAGAAGAAGAAGGAGGATGTGAACTG

SEQ ID NO: 73 - protein, 878 aa, Artificial Sequence
  Synthetic
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMGSGEGRGSLLTCGDVEENPGPMALPVTALLLPLALLLHADIVMTQSPDSLAVSLGERATINCKSSQSVLDSYNNENNLAWYQQKPGQPPKLLIYWASTRESGVPDRFSGSGSGTDFTLTISSLQAEDVAVYYCQQYTSEPITFGQGTKVEIKGGGGSGGGGSGGGGSEVQLLESGGGLVQPGGSLRLSCAASGFTFSDYQMSWVRQAPGKGLEWVSGIQGGGGSTYYADSVKGRFTISRDNSKNTLYLQMNSLRAEDTAVYYCAREMWRGDYYSGMDVWGQGTTVTVSSEQKLISEEDLAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 74 - DNA, 2637 bp, Artificial Sequence
  Synthetic
atgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggacccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgggttccggtgagggacgggggtcactgctcacctgcggagatgtagaagagaatcccggtcccatggctctcccagtgactgccctactgcttcccctagcgcttctcctgcatgcagacatcgtgatgacccagtctccagactccctggctgtgtctctgggcgagcgtgccaccatcaactgcaagtccagccagagtgttttagacagctataacaatgagaacaatttagcttggtatcagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatataccagcgaacctatcacgttcggccaaggtaccaaggtggaaatcaaaggcgggggtggtagtggcggtggaggtagcggaggtggcgggtctgaggtgcagctgttggagtctgggggaggcttggtacagcctggggggtccctgcgactctcctgtgcagcctctggattcacctttagcgactatcagatgagctgggtccgccaggctccagggaaggggctggagtgggtgtcaggcattcagggtggcggtggtagcacatattacgcagactccgtgaagggccggttcaccatctcccgtgacaattccaagaacacgctgtatctgcaaatgaacagcctgcgtgccgaggacacggctgtgtattactgtgcgagagagatgtggcgtggggactactactccggtatggacgtctggggccaggggaccacggtcaccgtctcctcagaacagaaactgatctctgaagaagacctggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctag

SEQ ID NO: 75 - protein, 883 aa, Artificial Sequence
  Synthetic
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMGSGEGRGSLLTCGDVEENPGPMALPVTALLLPLALLLHADIVMTQSPDSLAVSLGERATINCKSSQSVLDSYNNENNLAWYQQKPGQPPKLLIYWASTRESGVPDRFSGSGSGTDFTLTISSLQAEDVAVYYCQQYTSEPITFGQGTKVEIKGGGGSGGGGSGGGSGGGGSEVQLLESGGGLVQPGGSLRLSCAASGFTFSDYQMSWVRQAPGKGLEWVSGIQGGGGSTYYADSVKGRFTISRDNSKNTLYLQMNSLRAEDTAVYYCAREMWRGDYYSGMDVWGQGTTVTVSSEQKLISEEDLAAAIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 76 - DNA, 2652 bp, Artificial Sequence
  Synthetic
atgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggacccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgggttccggtgagggacgggggtcactgctcacctgcggagatgtagaagagaatcccggtcccatggctctcccagtgactgccctactgcttcccctagcgcttctcctgcatgcagacatcgtgatgacccagtctccagactccctggctgtgtctctgggcgagcgtgccaccatcaactgcaagtccagccagagtgttttagacagctataacaatgagaacaatttagcttggtatcagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatataccagcgaacctatcacgttcggccaaggtaccaaggtggaaatcaaaggtggtggtggttcaggtggtggtggttctggcggcggctccggtggtggtggatccgaggtgcagctgttggagtctgggggaggcttggtacagcctggggggtccctgcgactctcctgtgcagcctctggattcacctttagcgactatcagatgagctgggtccgccaggctccagggaaggggctggagtgggtgtcaggcattcagggtggcggtggtagcacatattacgcagactccgtgaagggccggttcaccatctcccgtgacaattccaagaacacgctgtatctgcaaatgaacagcctgcgtgccgaggacacggctgtgtattactgtgcgagagagatgtggcgtggggactactactccggtatggacgtctggggccaggggaccacggtcaccgtctcctcagaacagaaactgatctctgaagaagacctggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctag

SEQ ID NO: 77 - protein, 882 aa, Artificial Sequence
  Synthetic
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMGSGEGRGSLLTCGDVEENPGPMALPVTALLLPLALLLHADIVMTQSPDSLAVSLGERATINCKSSQSVLDSYNNENNLAWYQQKPGQPPKLLIYWASTRESGVPDRFSGSGSGTDFTLTISSLQAEDVAVYYCQQYTSEPITFGQGTKVEIKGGGGSGGGGSGGGSGGGGSEVQLLESGGGLVQPGGSLRLSCAASGFTFSDYQMSWVRQAPGKGLEWVSGIQGGGGSTYYADSVKGRFTISRDNSKNTLYLQMNSLRAEDTAVYYCAREMWRGDYYSGMDVWGQGTTVTVSSEQKLISEEDLAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 78 - DNA, 2649 bp, Artificial Sequence
  Synthetic
atgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggacccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgggttccggtgagggacgggggtcactgctcacctgcggagatgtagaagagaatcccggtcccatggctctcccagtgactgccctactgcttcccctagcgcttctcctgcatgcagacatcgtgatgacccagtctccagactccctggctgtgtctctgggcgagcgtgccaccatcaactgcaagtccagccagagtgttttagacagctataacaatgagaacaatttagcttggtatcagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatataccagcgaacctatcacgttcggccaaggtaccaaggtggaaatcaaaggtggtggtggttcaggtggtggtggttctggcggcggctccggtggtggtggatccgaggtgcagctgttggagtctgggggaggcttggtacagcctggggggtccctgcgactctcctgtgcagcctctggattcacctttagcgactatcagatgagctgggtccgccaggctccagggaaggggctggagtgggtgtcaggcattcagggtggcggtggtagcacatattacgcagactccgtgaagggccggttcaccatctcccgtgacaattccaagaacacgctgtatctgcaaatgaacagcctgcgtgccgaggacacggctgtgtattactgtgcgagagagatgtggcgtggggactactactccggtatggacgtctggggccaggggaccacggtcaccgtctcctcagaacagaaactgatctctgaagaagacctggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctag

SEQ ID NO: 79 - protein, 878 aa, Artificial Sequence
  Synthetic
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMGSGEGRGSLLTCGDVEENPGPMALPVTALLLPLALLLHAEVQLLESGGGLVQPGGSLRLSCAASGFTFSDYQMSWVRQAPGKGLEWVSGIQGGGGSTYYADSVKGRFTISRDNSKNTLYLQMNSLRAEDTAVYYCAREMWRGDYYSGMDVWGQGTTVTVSSGGGGSGGGGSGGGGSDIVMTQSPDSLAVSLGERATINCKSSQSVLDSYNNENNLAWYQQKPGQPPKLLIYWASTRESGVPDRFSGSGSGTDFTLTISSLQAEDVAVYYCQQYTSEPITFGQGTKVEIKEQKLISEEDLAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 80 - DNA, 2637 bp, Artificial Sequence
  Synthetic
atgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggacccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgggttccggtgagggacgggggtcactgctcacctgcggagatgtagaagagaatcccggtcccatggctctcccagtgactgccctactgcttcccctagcgcttctcctgcatgcagaggtgcagctgttggagtctgggggaggcttggtacagcctggggggtccctgcgactctcctgtgcagcctctggattcacctttagcgactatcagatgagctgggtccgccaggctccagggaaggggctggagtgggtgtcaggcattcagggtggcggtggtagcacatattacgcagactccgtgaagggccggttcaccatctcccgtgacaattccaagaacacgctgtatctgcaaatgaacagcctgcgtgccgaggacacggctgtgtattactgtgcgagagagatgtggcgtggggactactactccggtatggacgtctggggccaggggaccacggtcaccgtctcctcaggcgggggtggtagtggcggtggaggtagcggaggtggcgggtctgacatcgtgatgacccagtctccagactccctggctgtgtctctgggcgagcgtgccaccatcaactgcaagtccagccagagtgttttagacagctataacaatgagaacaatttagcttggtatcagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatataccagcgaacctatcacgttcggccaaggtaccaaggtggaaatcaaagaacagaaactgatctctgaagaagacctggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctag

SEQ ID NO: 81 - protein, 882 aa, Artificial Sequence
  Synthetic
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMGSGEGRGSLLTCGDVEENPGPMALPVTALLLPLALLLHAEVQLLESGGGLVQPGGSLRLSCAASGFTFSDYQMSWVRQAPGKGLEWVSGIQGGGGSTYYADSVKGRFTISRDNSKNTLYLQMNSLRAEDTAVYYCAREMWRGDYYSGMDVWGQGTTVTVSSGGGGSGGGGSGGGSGGGGSDIVMTQSPDSLAVSLGERATINCKSSQSVLDSYNNENNLAWYQQKPGQPPKLLIYWASTRESGVPDRFSGSGSGTDFTLTISSLQAEDVAVYYCQQYTSEPITFGQGTKVEIKEQKLISEEDLAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 82 - DNA, 2649 bp, Artificial Sequence
  Synthetic
atgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggacccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgggttccggtgagggacgggggtcactgctcacctgcggagatgtagaagagaatcccggtcccatggctctcccagtgactgccctactgcttcccctagcgcttctcctgcatgcagaggtgcagctgttggagtctgggggaggcttggtacagcctggggggtccctgcgactctcctgtgcagcctctggattcacctttagcgactatcagatgagctgggtccgccaggctccagggaaggggctggagtgggtgtcaggcattcagggtggcggtggtagcacatattacgcagactccgtgaagggccggttcaccatctcccgtgacaattccaagaacacgctgtatctgcaaatgaacagcctgcgtgccgaggacacggctgtgtattactgtgcgagagagatgtggcgtggggactactactccggtatggacgtctggggccaggggaccacggtcaccgtctcctcaggtggtggtggttcaggtggtggtggttctggcggcggctccggtggtggtggatccgacatcgtgatgacccagtctccagactccctggctgtgtctctgggcgagcgtgccaccatcaactgcaagtccagccagagtgttttagacagctataacaatgagaacaatttagcttggtatcagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatataccagcgaacctatcacgttcggccaaggtaccaaggtggaaatcaaagaacagaaactgatctctgaagaagacctggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctag

SEQ ID NO: 83 - protein, 881 aa, Artificial Sequence
  Synthetic
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMGSGEGRGSLLTCGDVEENPGPMALPVTALLLPLALLLHAEVQLLESGGGLVQPGGSLRLSCAASGFTFTSYAMSWVRQAPGKGLEWVSGIDGSGGGTNYADSVKGRFTISRDNSKNTLYLQMNSLRAEDTAVYYCARAYYDILTGYPVDGMDVWGQGTTVTVSSGGGGSGGGGSGGGGSDIVMTQSPDSLAVSLGERATINCKSSQSVLSSYNNENNLAWYQQKPGQPPKLLIYAASTRESGVPDRFSGSGSGTDFTLTISSLQAEDVAVYYCQQYYSEPYTFGQGTKVEIKEQKLISEEDLAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 84 - DNA, 2646 bp, Artificial Sequence
  Synthetic
atgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggacccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgggttccggtgagggacgggggtcactgctcacctgcggagatgtagaagagaatcccggtcccatggctctcccagtgactgccctactgcttcccctagcgcttctcctgcatgcagaggtgcagctgttggagtctgggggaggcttggtacagcctggggggtccctgcgactctcctgtgcagcctctggattcacctttaccagctatgccatgagctgggtccgccaggctccagggaaggggctggagtgggtgtcaggcattgacggtagcggtggtggcacaaattacgcagactccgtgaagggccggttcaccatctcccgtgacaattccaagaacacgctgtatctgcaaatgaacagcctgcgtgccgaggacacggctgtgtattactgtgcgagagcgtattacgatattttgactggttaccccgtggacggtatggacgtctggggccaagggaccacggtcaccgtctcctcaggcgggggtggtagtggcggtggaggtagcggaggtggcgggtctgacatcgtgatgacccagtctccagactccctggctgtgtctctgggcgagcgtgccaccatcaactgcaagtccagccagagtgttttaagcagctataacaatgagaacaatttagcttggtatcagcagaaaccaggacagcctcctaagctgctcatttacgccgcatctacccgggaatccggggtccctgaccgattcagtggcagcgggtctgggacagatttcactctcaccatcagcagcctgcaggctgaagatgtggcagtttattactgtcagcaatattatagcgaaccttatacgttcggccaaggtaccaaggtggaaatcaaagaacagaaactgatctctgaagaagacctggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctag

SEQ ID NO: 85 - protein, 881 aa, Artificial Sequence
  Synthetic
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMGSGEGRGSLLTCGDVEENPGPMALPVTALLLPLALLLHADIVMTQSPDSLAVSLGERATINCKSSQSVLSSYNNENNLAWYQQKPGQPPKLLIYAASTRESGVPDRFSGSGSGTDFTLTISSLQAEDVAVYYCQQYYSEPYTFGQGTKVEIKGGGGSGGGGSGGGGSEVQLLESGGGLVQPGGSLRLSCAASGFTFTSYAMSWVRQAPGKGLEWVSGIDGSGGGTNYADSVKGRFTISRDNSKNTLYLQMNSLRAEDTAVYYCARAYYDILTGYPVDGMDVWGQGTTVTVSSEQKLISEEDLAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 86 - DNA, 2646 bp, Artificial Sequence
  Synthetic
atgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggacccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgggttccggtgagggacgggggtcactgctcacctgcggagatgtagaagagaatcccggtcccatggctctcccagtgactgccctactgcttcccctagcgcttctcctgcatgcagacatcgtgatgacccagtctccagactccctggctgtgtctctgggcgagcgtgccaccatcaactgcaagtccagccagagtgttttaagcagctataacaatgagaacaatttagcttggtatcagcagaaaccaggacagcctcctaagctgctcatttacgccgcatctacccgggaatccggggtccctgaccgattcagtggcagcgggtctgggacagatttcactctcaccatcagcagcctgcaggctgaagatgtggcagtttattactgtcagcaatattatagcgaaccttatacgttcggccaaggtaccaaggtggaaatcaaaggcgggggtggtagtggcggtggaggtagcggaggtggcgggtctgaggtgcagctgttggagtctgggggaggcttggtacagcctggggggtccctgcgactctcctgtgcagcctctggattcacctttaccagctatgccatgagctgggtccgccaggctccagggaaggggctggagtgggtgtcaggcattgacggtagcggtggtggcacaaattacgcagactccgtgaagggccggttcaccatctcccgtgacaattccaagaacacgctgtatctgcaaatgaacagcctgcgtgccgaggacacggctgtgtattactgtgcgagagcgtattacgatattttgactggttaccccgtggacggtatggacgtctggggccaagggaccacggtcaccgtctcctcagaacagaaactgatctctgaagaagacctggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctag

SEQ ID NO: 87 - protein, 873 aa, Artificial Sequence
  Synthetic
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMGSGEGRGSLLTCGDVEENPGPMALPVTALLLPLALLLHADIVMTQSPDSLAVSLGERATINCKSSQSVLRSSNNKNNLAWYQQKPGQPPKLLIYAASTRESGVPDRFSGSGSGTDFTLTISSLQAEDVAVYYCQQYYREPLTFGQGTKVEIKGGGGSGGGGSGGGGSEVQLLESGGGLVQPGGSLRLSCAASGFTFTDYAMSWVRQAPGKGLEWVSDIDGSGGSTDYADSVKGRFTISRDNSKNTLYLQMNSLRAEDTAVYYCALELGATTVYWGQGTLVTVSSEQKLISEEDLAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 88 - DNA, 2622 bp, Artificial Sequence
  Synthetic
atgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggacccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgggttccggtgagggacgggggtcactgctcacctgcggagatgtagaagagaatcccggtcccatggctctcccagtgactgccctactgcttcccctagcgcttctcctgcatgcagacatcgtgatgacccagtctccagactccctggctgtgtctctgggcgagcgtgccaccatcaactgcaagtccagccagagtgttttacgcagcagcaacaataaaaacaatttagcttggtatcagcagaaaccaggacagcctcctaagctgctcatttacgccgcatctacccgggaatccggggtccctgaccgattcagtggcagcgggtctgggacagatttcactctcaccatcagcagcctgcaggctgaagatgtggcagtttattactgtcagcaatattatcgcgaacctctgacgttcggccaaggtaccaaggtggaaatcaaaggcgggggtggtagtggcggtggaggtagcggaggtggcgggtctgaggtgcagctgttggagtctgggggaggcttggtacagcctggggggtccctgcgactctcctgtgcagcctctggattcacctttaccgactatgccatgagctgggtccgccaggctccagggaaggggctggagtgggtgtcagacattgacggtagcggtggtagcacagactacgcagactccgtgaagggccggttcaccatctcccgtgacaattccaagaacacgctgtatctgcaaatgaacagcctgcgtgccgaggacacggctgtgtattactgtgcgctagagctgggagctactaccgtctactggggccagggaaccctggtcaccgtctcctcagaacagaaactgatctctgaagaagacctggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctag

SEQ ID NO: 89 - protein, 112 aa, Artificial Sequence
  Synthetic
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 90 - DNA, 336 bp, Artificial Sequence
  Synthetic
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 91 - protein, 24 aa, Artificial Sequence
  Synthetic
GGGGSGGGGSGGGGSGGGSGGGGS

SEQ ID NO: 92 - protein, 29 aa, Artificial Sequence
  Synthetic
GGGGSGGGGSGGGGSGGGGSGGGSGGGGS

SEQ ID NO: 93 - protein, 5 aa, Artificial Sequence
  Synthetic
GGGGS

SEQ ID NO: 94 - protein, 10 aa, Artificial Sequence
  Synthetic
GGGGSGGGGS

SEQ ID NO: 95 - protein, 868 aa, Artificial Sequence
  Synthetic
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMGSGEGRGSLLTCGDVEENPGPMALPVTALLLPLALLLHAEVQLLESGGGLVQPGGSLRLSCAASGFTFSDYQMSWVRQAPGKGLEWVSGIQGGGGSTYYADSVKGRFTISRDNSKNTLYLQMNSLRAEDTAVYYCAREMWRGDYYSGMDVWGQGTTVTVSSGGGGSDIVMTQSPDSLAVSLGERATINCKSSQSVLDSYNNENNLAWYQQKPGQPPKLLIYWASTRESGVPDRFSGSGSGTDFTLTISSLQAEDVAVYYCQQYTSEPITFGQGTKVEIKEQKLISEEDLAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 96 - DNA, 2607 bp, Artificial Sequence
  Synthetic
atgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggacccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgggttccggtgagggacgggggtcactgctcacctgcggagatgtagaagagaatcccggtcccatggctctcccagtgactgccctactgcttcccctagcgcttctcctgcatgcagaggtgcagctgttggagtctgggggaggcttggtacagcctggggggtccctgcgactctcctgtgcagcctctggattcacctttagcgactatcagatgagctgggtccgccaggctccagggaaggggctggagtgggtgtcaggcattcagggtggcggtggtagcacatattacgcagactccgtgaagggccggttcaccatctcccgtgacaattccaagaacacgctgtatctgcaaatgaacagcctgcgtgccgaggacacggctgtgtattactgtgcgagagagatgtggcgtggggactactactccggtatggacgtctggggccaggggaccacggtcaccgtctcctcaggcgggggtggtagtgacatcgtgatgacccagtctccagactccctggctgtgtctctgggcgagcgtgccaccatcaactgcaagtccagccagagtgttttagacagctataacaatgagaacaatttagcttggtatcagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatataccagcgaacctatcacgttcggccaaggtaccaaggtggaaatcaaagaacagaaactgatctctgaagaagacctggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctag

SEQ ID NO: 97 - protein, 873 aa, Artificial Sequence
  Synthetic
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMGSGEGRGSLLTCGDVEENPGPMALPVTALLLPLALLLHAEVQLLESGGGLVQPGGSLRLSCAASGFTFSDYQMSWVRQAPGKGLEWVSGIQGGGGSTYYADSVKGRFTISRDNSKNTLYLQMNSLRAEDTAVYYCAREMWRGDYYSGMDVWGQGTTVTVSSGGGGSGGGGSDIVMTQSPDSLAVSLGERATINCKSSQSVLDSYNNENNLAWYQQKPGQPPKLLIYWASTRESGVPDRFSGSGSGTDFTLTISSLQAEDVAVYYCQQYTSEPITFGQGTKVEIKEQKLISEEDLAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 98 - DNA, 2622 bp, Artificial Sequence
  Synthetic
atgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggacccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgggttccggtgagggacgggggtcactgctcacctgcggagatgtagaagagaatcccggtcccatggctctcccagtgactgccctactgcttcccctagcgcttctcctgcatgcagaggtgcagctgttggagtctgggggaggcttggtacagcctggggggtccctgcgactctcctgtgcagcctctggattcacctttagcgactatcagatgagctgggtccgccaggctccagggaaggggctggagtgggtgtcaggcattcagggtggcggtggtagcacatattacgcagactccgtgaagggccggttcaccatctcccgtgacaattccaagaacacgctgtatctgcaaatgaacagcctgcgtgccgaggacacggctgtgtattactgtgcgagagagatgtggcgtggggactactactccggtatggacgtctggggccaggggaccacggtcaccgtctcctcaggcgggggtggtagtggaggtggcgggtctgacatcgtgatgacccagtctccagactccctggctgtgtctctgggcgagcgtgccaccatcaactgcaagtccagccagagtgttttagacagctataacaatgagaacaatttagcttggtatcagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatataccagcgaacctatcacgttcggccaaggtaccaaggtggaaatcaaagaacagaaactgatctctgaagaagacctggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctag

SEQ ID NO: 99 - protein, 887 aa, Artificial Sequence
  Synthetic
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMGSGEGRGSLLTCGDVEENPGPMALPVTALLLPLALLLHAEVQLLESGGGLVQPGGSLRLSCAASGFTFSDYQMSWVRQAPGKGLEWVSGIQGGGGSTYYADSVKGRFTISRDNSKNTLYLQMNSLRAEDTAVYYCAREMWRGDYYSGMDVWGQGTTVTVSSGGGGSGGGGSGGGGSGGGSGGGGSDIVMTQSPDSLAVSLGERATINCKSSQSVLDSYNNENNLAWYQQKPGQPPKLLIYWASTRESGVPDRFSGSGSGTDFTLTISSLQAEDVAVYYCQQYTSEPITFGQGTKVEIKEQKLISEEDLAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 100 - DNA, 2664 bp, Artificial Sequence
  Synthetic
atgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggacccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgggttccggtgagggacgggggtcactgctcacctgcggagatgtagaagagaatcccggtcccatggctctcccagtgactgccctactgcttcccctagcgcttctcctgcatgcagaggtgcagctgttggagtctgggggaggcttggtacagcctggggggtccctgcgactctcctgtgcagcctctggattcacctttagcgactatcagatgagctgggtccgccaggctccagggaaggggctggagtgggtgtcaggcattcagggtggcggtggtagcacatattacgcagactccgtgaagggccggttcaccatctcccgtgacaattccaagaacacgctgtatctgcaaatgaacagcctgcgtgccgaggacacggctgtgtattactgtgcgagagagatgtggcgtggggactactactccggtatggacgtctggggccaggggaccacggtcaccgtctcctcaggtggtggtggttcaggtggtggtggttctggagggggcggttctggcggcggctccggtggtggtggatccgacatcgtgatgacccagtctccagactccctggctgtgtctctgggcgagcgtgccaccatcaactgcaagtccagccagagtgttttagacagctataacaatgagaacaatttagcttggtatcagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatataccagcgaacctatcacgttcggccaaggtaccaaggtggaaatcaaagaacagaaactgatctctgaagaagacctggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctag

SEQ ID NO: 101 - protein, 892 aa, Artificial Sequence
  Synthetic
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMGSGEGRGSLLTCGDVEENPGPMALPVTALLLPLALLLHAEVQLLESGGGLVQPGGSLRLSCAASGFTFSDYQMSWVRQAPGKGLEWVSGIQGGGGSTYYADSVKGRFTISRDNSKNTLYLQMNSLRAEDTAVYYCAREMWRGDYYSGMDVWGQGTTVTVSSGGGGSGGGGSGGGGSGGGGSGGGSGGGGSDIVMTQSPDSLAVSLGERATINCKSSQSVLDSYNNENNLAWYQQKPGQPPKLLIYWASTRESGVPDRFSGSGSGTDFTLTISSLQAEDVAVYYCQQYTSEPITFGQGTKVEIKEQKLISEEDLAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 102 - DNA, 2679 bp, Artificial Sequence
  Synthetic
atgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggacccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgggttccggtgagggacgggggtcactgctcacctgcggagatgtagaagagaatcccggtcccatggctctcccagtgactgccctactgcttcccctagcgcttctcctgcatgcagaggtgcagctgttggagtctgggggaggcttggtacagcctggggggtccctgcgactctcctgtgcagcctctggattcacctttagcgactatcagatgagctgggtccgccaggctccagggaaggggctggagtgggtgtcaggcattcagggtggcggtggtagcacatattacgcagactccgtgaagggccggttcaccatctcccgtgacaattccaagaacacgctgtatctgcaaatgaacagcctgcgtgccgaggacacggctgtgtattactgtgcgagagagatgtggcgtggggactactactccggtatggacgtctggggccaggggaccacggtcaccgtctcctcaggtggtggtggttcaggtggtggtggttctggtgggggcggtagtggagggggcggttctggcggcggctccggtggtggtggatccgacatcgtgatgacccagtctccagactccctggctgtgtctctgggcgagcgtgccaccatcaactgcaagtccagccagagtgttttagacagctataacaatgagaacaatttagcttggtatcagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatataccagcgaacctatcacgttcggccaaggtaccaaggtggaaatcaaagaacagaaactgatctctgaagaagacctggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctag

SEQ ID NO: 103 - protein, 179 aa, Artificial Sequence
  Synthetic
MGYRMQLLSCIALSLALVTNSGYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 104 - DNA, 540 bp, Artificial Sequence
  Synthetic
atgggttacaggatgcaactcctgtcttgcattgcactaagtcttgcacttgtcacaaacagtggctactttggcaagcttgaatctaaattatcagtcataagaaatttgaatgaccaagttctcttcattgaccaaggaaatcggcctctatttgaagatatgactgattctgactgtagagataatgcaccccggaccatatttattataagtatgtataaagatagccagcctagaggtatggctgtaactatctctgtgaagtgtgagaaaatttcaactctctcctgtgagaacaaaattatttcctttaaggaaatgaatcctcctgataacatcaaggatacaaaaagtgacatcatattctttcagagaagtgtcccaggacatgataataagatgcaatttgaatcttcatcatacgaaggatactttctagcttgtgaaaaagagagagacctttttaaactcattttgaaaaaagaggatgaattgggggatagatctataatgttcactgttcaaaacgaagactag

SEQ ID NO: 105 - protein, 179 aa, Artificial Sequence
  Synthetic
MYRMQLLSCIALSLALVTNSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 106 - DNA, 540 bp, Artificial Sequence
  Synthetic
atgtacaggatgcaactcctgtcttgcattgcactaagtcttgcacttgtcacaaacagtagtatcacaggaatttcacctattacagagtatcttgcttctctaagcacatacaatgatcaatccattacttttgctttggaggatgaaagttatgagatatatgttgaagacttgaaaaaagatgaaaagaaagataaggtgttactgagttactatgagtctcaacacccctcaaatgaatcaggtgacggtgttgatggtaagatgttaatggtaaccctgagtcctacaaaagacttctggttgcatgccaacaacaaggaacactctgtggagctccataagtgtgaaaaaccactgccagaccaggccttctttgtccttcataatatgcactccaactgtgtttcatttgaatgcaagactgatcctggagtgtttataggtgtaaaggataatcatcttgctctgattaaagtagactcttctgagaatttgtgtactgaaaatatcttgtttaagctctctgaaacttag

SEQ ID NO: 107 - protein, 254 aa, Artificial Sequence
  Synthetic
EVQLLESGGGLVQPGGSLRLSCAASGFTFSDYQMSWVRQAPGKGLEWVSGIQGGGGSTYYADSVKGRFTISRDNSKNTLYLQMNSLRAEDTAVYYCAREMWRGDYYSGMDVWGQGTTVTVSSGGGGSGGGGSGGGSGGGGSDIVMTQSPDSLAVSLGERATINCKSSQSVLDSYNNENNLAWYQQKPGQPPKLLIYWASTRESGVPDRFSGSGSGTDFTLTISSLQAEDVAVYYCQQYTSEPITFGQGTKVEIK

SEQ ID NO: 108 - protein, 257 aa, Artificial Sequence
  Synthetic
EVQLLESGGGLVQPGGSLRLSCAASGFTFTSYAMSWVRQAPGKGLEWVSGIDGSGGGTNYADSVKGRFTISRDNSKNTLYLQMNSLRAEDTAVYYCARAYYDILTGYPVDGMDVWGQGTTVTVSSGGGGSGGGGSGGGSGGGGSDIVMTQSPDSLAVSLGERATINCKSSQSVLSSYNNENNLAWYQQKPGQPPKLLIYAASTRESGVPDRFSGSGSGTDFTLTISSLQAEDVAVYYCQQYYSEPYTFGQGTKVEIK

SEQ ID NO: 109 - protein, 249 aa, Artificial Sequence
  Synthetic
EVQLLESGGGLVQPGGSLRLSCAASGFTFTDYAMSWVRQAPGKGLEWVSDIDGSGGSTDYADSVKGRFTISRDNSKNTLYLQMNSLRAEDTAVYYCALELGATTVYWGQGTLVTVSSGGGGSGGGGSGGGSGGGGSDIVMTQSPDSLAVSLGERATINCKSSQSVLRSSNNKNNLAWYQQKPGQPPKLLIYAASTRESGVPDRFSGSGSGTDFTLTISSLQAEDVAVYYCQQYYREPLTFGQGTKVEIK

SEQ ID NO: 110 - protein, 247 aa, Artificial Sequence
  Synthetic
EVQLLESGGGLVQPGGSLRLSCAASGFTFTSTQMSWVRQAPGKGLEWVSEISGYGGSTYYADSVKGRFTISRDNSKNTLYLQMNSLRAEDTAVYYCAKDTEVSGDAFDIWGQGTMVTVSSGGGGSGGGGSGGGSGGGGSEIVLTQSPGTLSLSPGERATLSCRASQSVDSSNLAWYQQKPGQAPRLLIYGASSRATGIPDRFSGSGSGTDFTLTISRLEPEDFAVYYCQQYRSWPITFGQGTKVEIK

SEQ ID NO: 111 - protein, 254 aa, Artificial Sequence
  Synthetic
EVQLLESGGGLVQPGGSLRLSCAASGFTFTSYYMSWVRQAPGKGLEWVSGISGSGDSTSYADSVKGRFTISRDNSKNTLYLQMNSLRAEDTAVYYCAREAGGDYDSGAFDIWGQGTMVTVSSGGGGSGGGGSGGGSGGGGSDIVMTQSPDSLAVSLGERATINCKSSQSVLYSGNNKNYLAWYQQKPGQPPKLLIYGASTRESGVPDRFSGSGSGTDFTLTISSLQAEDVAVYYCQQYDYAPFTFGQGTKVEIK

SEQ ID NO: 112 - protein, 259 aa, Artificial Sequence
  Synthetic
EVQLLESGGGLVQPGGSLRLSCAASGFTFSSYAMSWVRQAPGKGLEWVSEIDGEGGYTNYADSVKGRFTISRDNSKNTLYLQMNSLRAEDTAVYYCAREGVDYDILTGYYPYGMDVWGQGTTVTVSSGGGGSGGGGSGGGSGGGGSDIVMTQSPDSLAVSLGERATINCKSSQSVLDSSNNKNYLAWYQQKPGQPPKLLIYDASTRESGVPDRFSGSGSGTDFTLTISSLQAEDVAVYYCQQGTSSPLTFGQGTKVEIK